(19) United States
(12) Patent Application Publication
(10) Pub. No.: US 2014/0148622 A1
(43) Pub. Date: May 29, 2014

(54) Engineering Plants to Produce Farnesene and Other Terpenoids

(71) Applicants: Ramesh Nair, Naperville, IL (US); Oswald Crasta, Carmel, IN (US); Otto Folkerts, Urbana, IL (US); Joshua Blakeslee, Wooster, OH (US); Katrina Cornish, Wooster, OH (US)

(72) Inventors: Ramesh Nair, Naperville, IL (US); Oswald Crasta, Carmel, IN (US); Otto Folkerts, Urbana, IL (US); Joshua Blakeslee, Wooster, OH (US); Katrina Cornish, Wooster, OH (US)

(73) Assignees: THE OHIO STATE UNIVERSITY, Columbus, OH (US); CHROMATIN, INC., Chicago, IL (US)

(21) Appl. No.: 14/086,713

(22) Filed: Nov. 21, 2013

Related U.S. Application Data
(60) Provisional application No. 61/728,958, filed on Nov. 21, 2012.

(51) Int. Cl. C12P 5/00; C07C 11/21

(52) U.S. Cl. CPC C12P 5/007; C07C 11/21
USPC 585/16; 435/167; 435/419; 800/278; 800/298

(57) ABSTRACT

The present invention relates to engineering plants to express higher levels than endogenous amounts of terpenoids, such as farnesene. Plants that can be so engineered include those with large carbon stores, such as sweet sorghum and sugar cane.

### CROSS REFERENCE TO RELATED APPLICATION

This application claims priority to Nair, R., et al., U.S. Provisional Application No. 61/728,958, “ENGINEERING PLANTS TO PRODUCE FARNESENE AND OTHER TERPENOIODS,” filed Nov. 21, 2012, incorporated by reference herein in its entirety.

### FIELD OF THE INVENTION

The present invention relates to engineering plants to express higher levels than endogenous amounts of terpenoids, such as farnesene.

### GOVERNMENT SUPPORT

Not applicable.

### COMPACT DISC FOR SEQUENCE LISTINGS AND TABLES

Not applicable.

### BACKGROUND OF THE INVENTION

Agricultural and aquacultural crops have the potential to meet escalating global demands for affordable and sustainable production of food, fuels, fibers, therapeutics, and biofeedstocks.

Development of sustainable sources of domestic energy is crucial for the US to achieve energy independence. In 2010, the US produced 13.2 billion gallons of ethanol from corn grain and 315 million gallons of biodiesel from soybeans as the predominant forms of liquid biofuels (Board, 2011; RFA, 2011). It is expected that biofuels based on corn grain and soybeans will not exceed 15.8 billion gallons in the long term. Although efforts to convert biomass to biofuel by either enzymatic or thermochemical processes will continue to contribute towards energy independence (Lin and Tanaka, 2006; Nigam and Singh, 2011), this process alone is not enough to achieve the target goals of biofuel production. It is projected that only 12% of all liquid fuels produced in the US will be derived from renewable sources by 2035, far below the mandated 30% (Newell, 2011). To reach the target levels of 30% of all liquid fuels consumed in US by 2035, new and innovative biofuel production methodologies must be employed.

Because of their abundance and high energy content terpenoids provide an attractive alternative to current biofuels (Bohlmann and Keeling, 2008; Pourbafrani et al., 2010; Wu et al., 2006). The terpenoid biosynthetic pathway (see FIG. 1) is ubiquitous in plants and produces over 40,000 structures, forming the largest class of plant metabolites (Bohlmann and Keeling, 2008). Research on terpenoids has focused primarily on uses as flavor components or scent compounds (Cheng et al., 2007). Currently, terpene-based biofuel production has focused on using micro-organisms, including yeast and bacterial systems (Fischer et al., 2008; Nigam and Singh, 2011; Peralta-Yahya and Keasling, 2010). This approach is both energy-intensive and infrastructure-demanding, requiring a supply of sugars for large scale fermentation, constant temperature maintenance and other inputs, and immense infrastructure to support meaningful, large-scale microorganism culture. Attempts have been made to overcome these obstacles by engineering algal systems to produce biodiesel hydrocarbons, defraying some of the energy cost by harnessing algal photosynthetic capacity. Algal systems still require significant energy inputs to maintain temperature and salt equilibria. Such systems have yet to produce biodiesel in sufficient quantities to offset the costs of large-scale bioreactors necessary for algal biodiesel production.

### SUMMARY OF THE INVENTION

In a first aspect, the invention is directed to methods of increasing production of at least one terpenoid, the method comprising expressing in a plant cell a set of heterologous nucleic acids that encode polypeptides comprising enzymes necessary to carry out the mevalonic acid pathway or the methylerythritol 4-phosphate pathway, wherein production of the at least one terpenoid is increased when compared to a wild-type plant cell not encoding the set of heterologous nucleic acids. In additional aspects, both the mevalonic acid pathway and the methylerythritol 4-phosphate pathway are expressed from the heterologous nucleic acids in a plant cell. In additional aspects, the method further comprises expressing in a plant cell heterologous nucleic acids that encode at least one polypeptide comprising an enzyme selected from the group consisting of isopentenyl-diphosphate delta-isomerase, farnesyl diphosphate synthase, and farnesene synthase.

In some aspects, expressing heterologous nucleic acids encoding enzymes from the mevalonic acid pathway include those encoding methylerythritol 4-phosphate, as well as heterologous nucleic acids encoding at least one polypeptide comprising an enzyme selected from the group consisting of isopentenyl-diphosphate delta-isomerase, farnesyl diphosphate synthase, and farnesene synthase. In some aspects, isopentenyl-diphosphate delta-isomerase, a farnesyl diphosphate synthase; and a farnesene synthase are all expressed. The isopentenyl-diphosphate delta-isomerase can be an isopentenyl-diphosphate delta-isomerase I or isopentenyl-diphosphate delta-isomerase II, and the farnesene synthase is an α-farnesene synthase or a β-farnesene synthase.

In another aspect, the invention is directed to methods of increasing production of at least one terpenoid, wherein the at least one terpenoid is a sesquiterpenoid, such as farnesene.

In any aspect of the invention, sesquiterpenoid metabolism can be induced by an elicitor, such as methyl jasmonate, salicylic acid, ethephon and benzothiadiazole. In some embodiments, the elicitor is methyl jasmonate.

In any aspect of the invention wherein heterologous nucleic acids encoding enzymes of the mevalonic acid pathway are expressed, the pathway comprises nucleic acids encoding a(n): acetyl-CoA acetyltransferase, 3-hydroxy-3-methylglutaryl coenzyme A synthase, 3-hydroxy-3-methylglutaryl-coenzyme A reductase, mevalonate kinase, phosphomevalonate kinase, and mevalonate pyrophosphate decarboxylase. In additional aspects, the heterologous nucleic acids encoding enzymes of the mevalonic acid pathway encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) acetyl-CoA acetyltransferase: selected from the group consisting of SEQ ID NOs:1-4, 143;
(ii) 3-hydroxy-3-methylglutaryl coenzyme A synthase: selected from the group consisting of SEQ ID NOs:5-9, 144, 145;
(iii) 3-hydroxy-3-methylglutaryl-coenzyme A reductase: selected from the group consisting of SEQ ID NOs:10-16, 17-20, 146-150;
(iv) mevalonate kinase: selected from the group consisting of SEQ ID NOs:25-26;
(v) phosphomevalonate kinase: selected from the group consisting of SEQ ID NOs:27-33 and
(vi) mevalonate pyrophosphate decarboxylase: selected from the group consisting of SEQ ID NOs:34-40, 152; and
wherein the polypeptide retains functional activity in the MVA pathway.

In any aspect of the invention wherein heterologous nucleic acids encoding enzymes of the methylerythritol 4-phosphate pathway are expressed, the pathway comprises nucleic acids encoding a(n) 1-deoxy-D-xylulose-5-phosphate synthase, 1-deoxy-D-xylulose 5-phosphate reductoisomerase, 4-diphosphocytidyl-2-C-methyl-D-erythritol synthase, 4-diphosphocytidyl-2-C-methyl-D-erythritol kinase, 2-C-methyl-D-erythritol 2,4-cyclodiphosphate synthase, 4-hydroxy-3-methylbut-2-en-1-yl diphosphate synthase, and 4-hydroxy-3-methylbut-2-enyl diphosphate reductase. In additional aspects, the heterologous nucleic acids encoding enzymes of the methylerythritol 4-phosphate pathway encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) 1-deoxy-D-xylulose-5-phosphate synthase: selected from the group consisting of SEQ ID NOs:41-49, 153, 154, 169, 177-180;
(ii) 1-deoxy-D-xylulose 5-phosphate reductoisomerase: selected from the group consisting of SEQ ID NOs:50-58, 155, 156, 170, 181;
(iii) 4-diphosphocytidyl-2-C-methyl-D-erythritol synthase: selected from the group consisting of SEQ ID NOs:59-67, 157, 171, 182;
(iv) 4-diphosphocytidyl-2-C-methyl-D-erythritol kinase: selected from the group consisting of SEQ ID NOs:68-73, 158, 172, 183;
(v) 2-C-methyl-D-erythritol 2,4-cyclodiphosphate synthase: selected from the group consisting of SEQ ID NOs:74-82, 159, 173, 184;
(vi) 4-hydroxy-3-methylbut-2-en-1-yl diphosphate synthase, and: selected from the group consisting of SEQ ID NOs:83-89, 160, 174, 185; and
(vii) 4-hydroxy-3-methylbut-2-enyl diphosphate reductase: selected from the group consisting of SEQ ID NOs:90-97, 161-163, 175, 186 and
wherein the polypeptide retains functional activity in the MEP pathway.

In other aspects of the invention wherein heterologous nucleic acids encoding enzymes of the mevalonic acid pathway are expressed, the pathway comprises nucleic acids encoding a(n): acetyl-CoA acetyltransferase, 3-hydroxy-3-methylglutaryl coenzyme A synthase, 3-hydroxy-3-methylglutaryl-coenzyme A reductase, mevalonate kinase, phosphomevalonate kinase, and mevalonate pyrophosphate decarboxylase, these heterologous nucleic acids encode polypeptides from Archaea, bacteria, fungi, and plantae kingdoms. In additional aspects, the heterologous nucleic acids encoding enzymes from the plantae kingdom of the mevalonic acid pathway. In other aspects, the mevalonic acid pathway heterologous nucleic acids encoding polypeptides from the plantae kingdom have at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) acetyl-CoA acetyltransferase comprises SEQ ID NO: 4;
(ii) 3-hydroxy-3-methylglutaryl coenzyme A synthase selected from the group consisting of SEQ ID NOs: 8-9;
(iii) 3-hydroxy-3-methylglutaryl-coenzyme A reductase selected from the group consisting of SEQ ID NOs:15, 16, 20;
(iv) mevalonate kinase, comprising SEQ ID N0:26;
(v) phosphomevalonate kinase, selected from the group consisting of SEQ ID NOs:32-33 and
(vi) mevalonate pyrophosphate decarboxylase selected from the group consisting of SEQ ID NOs:39-40; and
wherein the polypeptide retains functional activity in the MVA pathway

In other aspects of the invention, wherein heterologous nucleic acids encoding enzymes of the methylerythritol 4-phosphate pathway are expressed, the pathway comprises nucleic acids encoding a(n) 1-deoxy-D-xylulose-5-phosphate synthase, 1-deoxy-D-xylulose 5-phosphate reductoisomerase, 4-diphosphocytidyl-2-C-methyl-D-erythritol synthase, 4-diphosphocytidyl-2-C-methyl-D-erythritol kinase, 2-C-methyl-D-erythritol 2,4-cyclodiphosphate synthase, 4-hydroxy-3-methylbut-2-en-1-yl diphosphate synthase, and 4-hydroxy-3-methylbut-2-enyl diphosphate reductase, these heterologous nucleic acids encode polypeptides from Archaea, bacteria, fungi, and plantae kingdoms. In additional aspects, the heterologous nucleic acids encoding enzymes from the plantae kingdom. In additional aspects, the heterologous nucleic acids encoding enzymes from the plantae kingdom of the methylerythritol 4-phosphate pathway. In other aspects, the methylerythritol 4-phosphate pathway heterologous nucleic acids encoding polypeptides from the plantae kingdom have of the methylerythritol 4-phosphate pathway encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) 1-deoxy-D-xylulose-5-phosphate synthase selected from the group consisting of SEQ ID NOs:41, 48-49;
(ii) 1-deoxy-D-xylulose 5-phosphate reductoisomerase selected from the group consisting of SEQ ID NOs:50, 56-58;
(iii) 4-diphosphocytidyl-2-C-methyl-D-erythritol synthase selected from the group consisting of SEQ ID NOs:59, 66-67;
(iv) 4-diphosphocytidyl-2-C-methyl-D-erythritol kinase selected from the group consisting of SEQ ID NOs:68, 73;
(v) 2-C-methyl-D-erythritol 2,4-cyclodiphosphate synthase selected from the group consisting of SEQ ID NOs:74, 80-82;
(vi) 4-hydroxy-3-methylbut-2-en-1-yl diphosphate synthase selected from the group consisting of SEQ ID NOs:83, 89; and
(vii) 4-hydroxy-3-methylbut-2-enyl diphosphate reductase selected from the group consisting of SEQ ID NOs:90, 96-97 and
wherein the polypeptide retains functional activity in the MEP pathway.
(viii) In additional aspects of the invention, in any method wherein the method comprises expressing heterologous nucleic acids encoding polypeptides for isopentenyl-diphosphate delta-isomerase, farnesyl diphosphate synthase, and farnesene synthase, the nucleic acids encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) isopentenyl-diphosphate delta-isomerase selected from the group consisting of SEQ ID NOs:98-101, 102-106, 188, 190-192;
(ii) farnesyl diphosphate synthase selected from the group consisting of SEQ ID NOs:107-111, 164, 165, 176, 187, 189; and
(iii) farnesene synthase selected from the group consisting of SEQ ID NOs:112-115, 116-117, 166-168 and
wherein the polypeptide retains functional activity.

In additional aspects of the invention, in any method wherein the method comprises expressing heterologous nucleic acids encoding polypeptides for isopentenyl-diphosphate delta-isomerase, farnesyl diphosphate synthase, and farnesene synthase, the nucleic acids encode polypeptides from the plantae kingdom. In other aspect, the isopentenyl-diphosphate delta-isomerase, farnesyl diphosphate synthase, and farnesene synthase polypeptides from the plantae kingdom have at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) isopentenyl-diphosphate delta-isomerase, having at least 70% sequence identity to at least one amino acid sequence selected from the group consisting of SEQ ID NOs:98-101, 102-106, 188, 190-192;
(ii) farnesyl diphosphate synthase, having at least 70% sequence identity to at least one amino acid sequence selected from the group consisting of SEQ ID NOs:107-111, 164, 165, 176, 187, 189; and
(iii) farnesene synthase, having at least 70% sequence identity to at least one amino acid sequence selected from the group consisting of SEQ ID NOs:112-115, 116-117, 166-168 and wherein the polypeptide retains a functional activity.

In any aspects of the invention expressing heterologous nucleic acids encoding polypeptides comprising enzymes necessary to carry out the mevalonic acid pathway or the methylerythritol 4-phosphate pathway, or isopentenyl-diphosphate delta-isomerase, farnesyl diphosphate synthase, and farnesene synthase activity, at least two of the heterologous nucleic acids are introduced into the plant cell on a single recombinant DNA construct. In some aspects, such a recombinant DNA construct may autonomously segregate to daughter cells during cell division, such as during mitosis or meiosis. In additional aspects, the autonomously segregating recombinant DNA construct comprises a plant centromere, such as a heterologous centromere or a centromere from the same plant as the cell in which the construct is introduced. In additional aspects, the recombinant DNA construct is a mini-chromosome. In yet other aspects, only plasmid constructs are used; in other aspects, a combination of mini-chromosomes and plasmid constructs are used.

In further aspects, the methods of the invention comprise expressing from a single mini-chromosome heterologous nucleic acids encoding enzymes of the mevalonic acid pathway or the methylerythritol 4-phosphate pathway; in other aspects, both the mevalonic acid pathway or the methylerythritol 4-phosphate pathway are expressed from a single mini-chromosome. In any of these aspects, the mini-chromosome may further comprise heterologous nucleic acids encoding polypeptides comprising at least one enzyme selected from the group consisting of isopentenyl-diphosphate delta-isomerase, farnesyl diphosphate synthase and farnesene synthase. In yet additional aspects, isopentenyl-diphosphate delta-isomerase, farnesyl diphosphate synthase and farnesene synthase are all expressed from the same mini-chromosome.

In further aspects, any of the methods and compositions as described above comprise plant cells wherein the production of at least one terpenoid is increased includes plant cells selected from the group consisting of a green algae, a vegetable crop plant, a fruit crop plant, a vine crop plant, a field crop plant, a biomass plant, a bedding plant, and a tree. In other aspects, the plant is selected from the group consisting of corn, soybean, Brassica, tomato, sorghum, sugar cane, miscanthus, guayle, switchgrass, wheat, barley, oat, rye, wheat, rice, (sugar) beet, green algae, Hevea and cotton. In some aspects, the plant is selected from the group consisting of sorghum, sugar cane, guayule, Hevea, and (sugar) beet.

In other aspects of the invention, any of the methods of the invention may further comprise isolating the farnesene. Such aspects may further comprise processing the farensene into farnesane.

In yet additional aspects, the invention comprises a plant made comprising a plant cell made by any of the methods of the invention.

In another aspect, the invention comprises a fuel comprising a terpenoid which production is increased by any of the methods of the invention, or made by a plant cell or plant made by any of the methods of the invention. Such terpenoids comprise sesquiterpenoids, such as farnesene and farnesane.

### BRIEF DESCRIPTION OF SEVERAL VIEWS OF THE DRAWINGS

FIG. 1 shows a schematic of the isoprenoid pathway in plants. Solid arrows, broken arrows with short dashes and broken arrows with long dashes represent single and multiple enzymatic steps and transport, respectively. Abbreviations: ABA, a bscissic acid; BRs, brassinosteroids; CYTP450, cytochrome P450 hydroxylases; DMADP; dimethylallyl diphosphate; DXP, deoxyxylulose-5-phosphate; DXR, DXP reductoisomerase; DXS, DXP synthase; FDP, farnesyl diphosphate; GDP, geranyl diphosphate; GGDP, geranylgeranyl diphosphate; GlyAld-3-P, glyceraldehydes 3-phopshate; HDR+, hydroxymethylbutenyl diphosphate reductase; IDP, isopentenyl diphosphate; MEP, methylerythritol 4-phosphate; MVA, mevalonic acid. Terpenes includes terpenes from all classes and originating from the various organelles (Adapted from (2005) Trends in Plant Science 10 (12):591-599. See also Table of Abbreviations at the end of the Detailed Description for additional abbreviations used through the specification.

FIGS. 2-7 show just a few constructs that are useful in various aspects of the invention. FIGS. 2A, 3A, 4A, 5A, 6A, and 7A (upper portion of each figure) show examples of constructs with specific transgenes operably linked to various control elements, such as promoters and terminators. FIGS. 2B, 3B, 4B, 5B, 6B, and 7B (lower portion of each figure) show generic examples of the constructs exemplified in part A of each figure.

FIG. 8 shows GC analysis of sugar cane leaf samples. (A) Sugar cane leaf samples that are induced with 4 mM methyl jasmonate shows production of caryophyllene, farnesene and other sesquiterpenes after 30 hrs of MeJ induction. (B). Sugar cane leaf samples that are treated with water for 30 hrs do not show any indication of farnesene and caryophyllene production.

### DETAILED DESCRIPTION OF THE INVENTION

### I. Introduction

The present invention represents a novel approach to produce liquid biofuels from plants. The invention provides crop systems that can generate liquid sesquiterpenoid, such as β-farnesene, resins which can then be converted to biodiesel molecules, such as β-farnesane. This approach offers several advantages over current biofuel technologies. Unlike starch- or cellulose-based ethanol production, which includes saccharification and fermentation, producing such resins for fuel has fewer steps, thus reducing necessary production infrastructure. Sesquiterpenoids have useful properties, such as immiscibility with water, which enables concentrating the fuel without distillation—which is otherwise needed to concentrate fuel produced by starch and cellulosic biofuel production technologies. Compared to current biodiesel production, extraction of β-farnesene from biomass and conversion to farnesane is a one-step hydrogenation process, reducing the overall production cost. Unlike biodiesel currently produced from soy or canola seed oil, the whole plant, not just the seeds, can be used in the present invention.

The invention takes a unique approach to overcome hurdles encountered in current efforts to generate biofuels from terpenoid and biodiesel production in microorganisms, such as yeasts and algae. Energy inputs are drastically reduced by utilizing the photosynthetic capacity of an entire plant and funneling all non-essential carbon into the production of β-farnesene-enriched resins, such as is possible in plants like sweet sorghum, sugar cane, Hevea sp. and guayule. These resins can be used as a readily-extractable liquid biofuel. Furthermore production of biofuel in crops does not require the cost associated with developing microbial fermentation processes and facilities and can capitalize on a vast existing agricultural infrastructure.

The present invention describes methods of expressing the enzymes of the mevalonic acid (MVA) pathway needed for the conversion of Acetyl CoA into β-farnesene in the cytosol of modified plants and plant cells. The present invention also describes methods of expressing enzymes of the methylerythritol 4-phosphate (MEP) pathway for the conversion of pyruvate CoA into β-farnesene in chloroplast of plants. Furthermore, the invention describes methods wherein isopentenyl-diphosphate delta-isomerase (IDDI), farnesyl diphosphate synthase (FDS) and farnesene synthase (FS; (collectively “IFF”)) activities are expressed to accumulate farnesene. The present invention describes how the genes that code for MVA and MEP pathway enzymes are regulated in plants to produce β-farnesene without severely affecting plant growth and development. The present invention also describes how plants that accumulate sucrose and other sugar molecules, such as sorghum, sugar cane, sugar beet, etc., can be engineered to produce sesquiterpenes and other high energy terpenoid compounds that can be readily used as biofuels or converted to biodiesel.

The invention provides methods, plant cells and plants that produce β-farnesene and related alkene sesquiterpenes in high yields that can be readily extracted and converted to low-cost liquid biofuels. In some embodiments, mini-chromosome (MC) gene-stacking technology is used to advantageously engineer β-farnesene production into plant cells and plants; in further embodiments, such plants are sugar cane (Saccharum sp.), guayule (Parthenium argentatum), Hevea and sweet sorghum (Sorghum bicolor). In other embodiments, the heterologous genes are carried on one or more plasmids, or, a combination of MCs and plasmids is used. The invention also provides for methods to extract and process farnesene produced by such engineered plant cells and plants into the biofuel molecule farnesane. While there is a report that the MVA pathway has been expressed in tobacco plant cells (Kumar, S. et al. Remodeling the isoprenoid pathway in tobacco by expressing the cytoplasmic mevalonate pathway in chloroplasts. Metabolic Engineering 14:19-28 (2012), the present invention is the first to describe the MVA, MEP and “IFF” pathways in sorghum and sugar cane plant cells.

The present invention describes engineering plants, such as sweet sorghum and sugar cane, to produce β-farnesene and other energy rich terpenoid molecules that can be readily used as biofuels or converted to biofuels, and primarily relies on rerouting sucrose stored in the plant into energy rich sesquiterpenes during normal growth and development. Sorghum generally produces sesquiterpenes in small amounts during stress conditions such as insect damage and/or during disease outbreak. This suggests that the genes required for sesquiterpene production are developmentally regulated and are induced during stress situations such as insect attack.

Sorghum, a C4 monocotyledonous grass grown in the southwestern, central and Midwestern US, has high photosynthetic efficiency, water and nutrient efficiency, stress tolerance, and is unmatched in its diversity of germplasm including starch (grain) types, high sugar (sweet) types, and high-biomass photoperiod sensitive (forage) types. Sorghum outperforms corn in regions with low annual rainfall, making it an ideal crop for semi-arid regions (Zhan et al., 2003).

Sorghum can be grown on more than 70 million Ha where bioenergy crops are currently farmed. Production of liquid β-farnesene biofuel in sorghum can produce low-cost transportation fuel and allow diversification of feedstock supply and land use with minimal impact on food crops. In contrast, 1 Ha of soybeans can produce about 150-250 gallons of biodiesel, while engineered sorghum, sugar cane or guayle that contain, for example, 20% by dry weight farnesene at 39-56 t/Ha of harvested yield have the production potential of 1800-2800 gallons of biofuel/Ha. Further, engineered plants containing 20% farnesene by dry weight when processed, can produce 250-388 GJ/Ha/year of biofuel with an energy density of 47.5 MJ/L, with an estimated process cost at scale of $8.46-9.14/GJ. Production of high farnesene biofuel from guayule and sorghum on 110 million Ha has the theoretical potential to produce over 30 EJ/yr (approximately 30% of the current US annual energy requirement).

In embodiments of the invention, the entire cytosolic MVA pathway or the entire chloroplastic MEP pathway, or both pathways, are introduced into plant cells, such as sweet sorghum cells. In cytosolic terpenoid synthesis, pyruvate formed from the glycolysis of sucrose molecules is converted into Acetyl-CoA which is incorporated into hydroxymethylglutaryl-coenzyme A (HMG-CoA) by the enzyme 3-hydroxy-3-methylglutaryl-coenzyme A reductase (Bach et al., 1991; Enjuto et al., 1994). HMG-CoA is then processed through the MVA pathway and used to generate dimethylallyl pyrophosphate (DMAPP) and isopentenyl pyrophosphate (IPP), both 5-carbon isoprene monomers for terpenoid biosynthesis (Bach et al., 1991; Cheng et al., 2007; Enjuto et al., 1994). In chloroplastic terpenoid synthesis, pyruvate and glyceraldehydes 3-phosphate are converted to 1-Deoxy-D-xylulose-5-P by 1-Deoxy-D-xylulose-5-P synthase which is then processed by MEP pathway enzymes to Dimethylallyl pyrophosphate (DMAPP) and isopentenyl pyrophosphate (IPP). These monomers are assembled together in a series of head-to-tail condensation reactions to generate farnesyl pyrophosphate (FPP, C15), a reaction catalyzed by the enzyme farnesyl diphosphate synthase (FPP synthase/FDPS). The final reaction is catalyzed by the enzyme β-farnesene synthase which converts FPP into β-farnesene.

### II. Making and Using the Invention

### Note: Definitions are Found at the End of the Detailed Description, Before the Examples

### A. Selected Embodiments

To maximize production of terpenoids, the enzymes (or their activities) of the MVA or the MEP or both pathways are transgenically expressed in plant cells to increase terpenoid production over non-transgenic plant cells. Furthermore, the IFF pathway can also be expressed to drive the production of farnesene. Plants with high, free carbon stores, high-energy density, such as sorghum genotypes with high-sugar content and sugar cane, as well as Hevea sp. and guayule, can be used to maximize flux distribution into the sesquiterpenoid metabolic pathway.

The invention also provides for extraction of farnesene from biomass (from plant cells and plants) and efficient processing technology to convert farnesene into the biofuel molecule farnesane. Such engineered plants, such as sorghum and sugar cane, can be intergressed into elite germplasm or into publicly available (and alternatively, improved) lines, to facilitate commercial production.

Thus, In a first embodiment, the invention is directed to methods of increasing production of at least one terpenoid, the method comprising expressing in a plant cell a set of heterologous nucleic acids that encode polypeptides comprising enzymes necessary to carry out the mevalonic acid pathway or the methylerythritol 4-phosphate pathway, wherein production of the at least one terpenoid is increased when compared to a wild-type plant cell not encoding the set of heterologous nucleic acids. In additional embodiments, both the mevalonic acid pathway and the methylerythritol 4-phosphate pathway are expressed from the heterologous nucleic acids in a plant cell. In additional embodiments, the method further comprises expressing in a plant cell heterologous nucleic acids that encode at least one polypeptide comprising an enzyme selected from the group consisting of isopentenyl-diphosphate delta-isomerase, farnesyl diphosphate synthase, and farnesene synthase.

In some embodiments, expressing heterologous nucleic acids encoding enzymes from the mevalonic acid pathway include those encoding methylerythritol 4-phosphate, as well as heterologous nucleic acids encoding at least one polypeptide comprising an enzyme selected from the group consisting of isopentenyl-diphosphate delta-isomerase, farnesyl diphosphate synthase, and farnesene synthase. In some embodiments, isopentenyl-diphosphate delta-isomerase, a farnesyl diphosphate synthase; and a farnesene synthase are all expressed. The isopentenyl-diphosphate delta-isomerase can be an isopentenyl-diphosphate delta-isomerase I or isopentenyl-diphosphate delta-isomerase II, and the farnesene synthase is an α-farnesene synthase or a β-farnesene synthase.

In another embodiment, the invention is directed to methods of increasing production of at least one terpenoid, wherein the at least one terpenoid is a sesquiterpenoid, such as farnesene.

In any embodiment of the invention wherein heterologous nucleic acids encoding enzymes of the mevalonic acid pathway are expressed, the pathway comprises nucleic acids encoding a(n): acetyl-CoA acetyltransferase, 3-hydroxy-3-methylglutaryl coenzyme A synthase, 3-hydroxy-3-methylglutaryl-coenzyme A reductase, mevalonate kinase, phosphomevalonate kinase, and mevalonate pyrophosphate decarboxylase. In additional embodiments, the heterologous nucleic acids encoding enzymes of the mevalonic acid pathway encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) acetyl-CoA acetyltransferase: selected from the group consisting of SEQ ID NOs:1-4, 143;
(ii) 3-hydroxy-3-methylglutaryl coenzyme A synthase: selected from the group consisting of SEQ ID NOs:5-9, 144, 145;
(iii) 3-hydroxy-3-methylglutaryl-coenzyme A reductase: selected from the group consisting of SEQ ID NOs:10-16, 17-20, 146-150;
(iv) mevalonate kinase: selected from the group consisting of SEQ ID NOs:25-26;
(v) phosphomevalonate kinase: selected from the group consisting of SEQ ID NOs:27-33 and
(vi) mevalonate pyrophosphate decarboxylase: selected from the group consisting of SEQ ID NOs:34-40, 152; and
wherein the polypeptide retains functional activity in the MVA pathway.

In any embodiment of the invention wherein heterologous nucleic acids encoding enzymes of the methylerythritol 4-phosphate pathway are expressed, the pathway comprises nucleic acids encoding a(n) 1-deoxy-D-xylulose-5-phosphate synthase, 1-deoxy-D-xylulose 5-phosphate reductoisomerase, 4-diphosphocytidyl-2-C-methyl-D-erythritol synthase, 4-diphosphocytidyl-2-C-methyl-D-erythritol kinase, 2-C-methyl-D-erythritol 2,4-cyclodiphosphate synthase, 4-hydroxy-3-methylbut-2-en-1-yl diphosphate synthase, and 4-hydroxy-3-methylbut-2-enyl diphosphate reductase. In additional embodiments, the heterologous nucleic acids encoding enzymes of the methylerythritol 4-phosphate pathway encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) 1-deoxy-D-xylulose-5-phosphate synthase: selected from the group consisting of SEQ ID NOs:41-49, 153, 154, 169, 177-180;
(ii) 1-deoxy-D-xylulose 5-phosphate reductoisomerase: selected from the group consisting of SEQ ID NOs:50-58, 155, 156, 170, 181;
(iii) 4-diphosphocytidyl-2-C-methyl-D-erythritol synthase: selected from the group consisting of SEQ ID NOs:59-67, 157, 171, 182;
(iv) 4-diphosphocytidyl-2-C-methyl-D-erythritol kinase: selected from the group consisting of SEQ ID NOs:68-73, 158, 172, 183;
(v) 2-C-methyl-D-erythritol 2,4-cyclodiphosphate synthase: selected from the group consisting of SEQ ID NOs:74-82, 159, 173, 184;
(vi) 4-hydroxy-3-methylbut-2-en-1-yl diphosphate synthase, and: selected from the group consisting of SEQ ID NOs:83-89, 160, 174, 185; and
(vii) 4-hydroxy-3-methylbut-2-enyl diphosphate reductase: selected from the group consisting of SEQ ID NOs:90-97, 161-163, 175, 186 and
wherein the polypeptide retains functional activity in the MEP pathway.

In other embodiments of the invention wherein heterologous nucleic acids encoding enzymes of the mevalonic acid pathway are expressed, the pathway comprises nucleic acids encoding a(n): acetyl-CoA acetyltransferase, 3-hydroxy-3-methylglutaryl coenzyme A synthase, 3-hydroxy-3-methylglutaryl-coenzyme A reductase, mevalonate kinase, phosphomevalonate kinase, and mevalonate pyrophosphate decarboxylase, these heterologous nucleic acids encode polypeptides from Archaea, bacteria, fungi, and plantae kingdoms. In additional embodiments, the heterologous nucleic acids encoding enzymes from the plantae kingdom of the mevalonic acid pathway. In other embodiments, the mevalonic acid pathway heterologous nucleic acids encoding polypeptides from the plantae kingdom have at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) acetyl-CoA acetyltransferase comprises SEQ ID NO: 4;
(ii) 3-hydroxy-3-methylglutaryl coenzyme A synthase selected from the group consisting of SEQ ID NOs: 8-9;
(iii) 3-hydroxy-3-methylglutaryl-coenzyme A reductase selected from the group consisting of SEQ ID NOs:15, 16, 20;
(iv) mevalonate kinase, comprising SEQ ID NO:26;
(v) phosphomevalonate kinase, selected from the group consisting of SEQ ID NOs:32-33 and
(vi) mevalonate pyrophosphate decarboxylase selected from the group consisting of SEQ ID NOs:39-40; and
wherein the polypeptide retains functional activity in the MVA pathway

In other embodiments of the invention, wherein heterologous nucleic acids encoding enzymes of the methylerythritol 4-phosphate pathway are expressed, the pathway comprises nucleic acids encoding a(n) 1-deoxy-D-xylulose-5-phosphate synthase, 1-deoxy-D-xylulose 5-phosphate reductoisomerase, 4-diphosphocytidyl-2-C-methyl-D-erythritol synthase, 4-diphosphocytidyl-2-C-methyl-D-erythritol kinase, 2-C-methyl-D-erythritol 2,4-cyclodiphosphate synthase, 4-hydroxy-3-methylbut-2-en-1-yl diphosphate synthase, and 4-hydroxy-3-methylbut-2-enyl diphosphate reductase, these heterologous nucleic acids encode polypeptides from Archaea, bacteria, fungi, and plantae kingdoms. In additional embodiments, the heterologous nucleic acids encoding enzymes from the plantae kingdom. In additional embodiments, the heterologous nucleic acids encoding enzymes from the plantae kingdom of the methylerythritol 4-phosphate pathway. In other embodiments, the methylerythritol 4-phosphate pathway heterologous nucleic acids encoding polypeptides from the plantae kingdom have of the methylerythritol 4-phosphate pathway encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) 1-deoxy-D-xylulose-5-phosphate synthase selected from the group consisting of SEQ ID NOs:41, 48-49;
(ii) 1-deoxy-D-xylulose 5-phosphate reductoisomerase selected from the group consisting of SEQ ID NOs:50, 56-58;
(iii) 4-diphosphocytidyl-2-C-methyl-D-erythritol synthase selected from the group consisting of SEQ ID NOs:59, 66-67;
(iv) 4-diphosphocytidyl-2-C-methyl-D-erythritol kinase selected from the group consisting of SEQ ID NOs:68, 73;
(v) 2-C-methyl-D-erythritol 2,4-cyclodiphosphate synthase selected from the group consisting of SEQ ID NOs:74, 80-82;
(vi) 4-hydroxy-3-methylbut-2-en-1-yl diphosphate synthase selected from the group consisting of SEQ ID NOs:83, 89; and
(vii) 4-hydroxy-3-methylbut-2-enyl diphosphate reductase selected from the group consisting of SEQ ID NOs:90, 96-97 and
wherein the polypeptide retains functional activity in the MEP pathway.
(viii) In additional embodiments of the invention, in any method wherein the method comprises expressing heterologous nucleic acids encoding polypeptides for isopentenyl-diphosphate delta-isomerase, farnesyl diphosphate synthase, and farnesene synthase, the nucleic acids encode polypeptides having at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) isopentenyl-diphosphate delta-isomerase selected from the group consisting of SEQ ID NOs:98-101, 102-106, 188, 190-192;
(ii) farnesyl diphosphate synthase selected from the group consisting of SEQ ID NOs:107-111, 164, 165, 176, 187, 189; and
(iii) farnesene synthase selected from the group consisting of SEQ ID NOs:112-115, 116-117, 166-168 and
wherein the polypeptide retains functional activity.

In additional embodiments of the invention, in any method wherein the method comprises expressing heterologous nucleic acids encoding polypeptides for isopentenyl-diphosphate delta-isomerase, farnesyl diphosphate synthase, and farnesene synthase, the nucleic acids encode polypeptides from the plantae kingdom. In other embodiment, the isopentenyl-diphosphate delta-isomerase, farnesyl diphosphate synthase, and farnesene synthase polypeptides from the plantae kingdom have at least 70%-99% sequence identity, including 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity as follows:
(i) isopentenyl-diphosphate delta-isomerase, having at least 70% sequence identity to at least one amino acid sequence selected from the group consisting of SEQ ID NOs:98-101, 102-106, 188, 190-192;
(ii) farnesyl diphosphate synthase, having at least 70% sequence identity to at least one amino acid sequence selected from the group consisting of SEQ ID NOs:107-111, 164, 165, 176, 187, 189; and
(iii) farnesene synthase, having at least 70% sequence identity to at least one amino acid sequence selected from the group consisting of SEQ ID NOs:112-115, 116-117, 166-168 and
wherein the polypeptide retains a functional activity.

In any embodiments of the invention expressing heterologous nucleic acids encoding polypeptides comprising enzymes necessary to carry out the mevalonic acid pathway or the methylerythritol 4-phosphate pathway, or isopentenyl-diphosphate delta-isomerase, farnesyl diphosphate synthase, and farnesene synthase activity, at least two of the heterologous nucleic acids are introduced into the plant cell on a single recombinant DNA construct. In some embodiments, such a recombinant DNA construct may autonomously segregate to daughter cells during cell division, such as during mitosis or meiosis. In additional embodiments, the autonomously segregating recombinant DNA construct comprises a plant centromere, such as a heterologous centromere or a centromere from the same plant as the cell in which the construct is introduced. In additional embodiments, the recombinant DNA construct is a mini-chromosome. In yet other embodiments, only plasmid constructs are used; in other embodiments, a combination of mini-chromosomes and plasmid constructs are used.

In further embodiments, the methods of the invention comprise expressing from a single mini-chromosome heterologous nucleic acids encoding enzymes of the mevalonic acid pathway or the methylerythritol 4-phosphate pathway; in other embodiments, both the mevalonic acid pathway or the methylerythritol 4-phosphate pathway are expressed from a single mini-chromosome. In any of these embodiments, the mini-chromosome may further comprise heterologous nucleic acids encoding polypeptides comprising at least one enzyme selected from the group consisting of isopentenyl-diphosphate delta-isomerase, farnesyl diphosphate synthase and farnesene synthase. In yet additional embodiments, isopentenyl-diphosphate delta-isomerase, farnesyl diphosphate synthase and farnesene synthase are all expressed from the same mini-chromosome.

In further embodiments, any of the methods and compositions as described above comprise plant cells wherein the production of at least one terpenoid is increased includes plant cells selected from the group consisting of a green algae, a vegetable crop plant, a fruit crop plant, a vine crop plant, a field crop plant, a biomass plant, a bedding plant, and a tree. In other embodiments, the plant is selected from the group consisting of corn, soybean, Brassica, tomato, sorghum, sugar cane, miscanthus, guayle, switchgrass, wheat, barley, oat, rye, wheat, rice, (sugar) beet, green algae, Hevea and cotton. In some embodiments, the plant is selected from the group consisting of sorghum, sugar cane, guayule, Hevea, and (sugar) beet.

In other embodiments of the invention, any of the methods of the invention may further comprise isolating the farnesene. Such embodiments may further comprise processing the farensene into farnesane.

In yet additional embodiments, the invention comprises a plant made comprising a plant cell made by any of the methods of the invention.

In another embodiment, the invention comprises a fuel comprising a terpenoid which production is increased by any of the methods of the invention, or made by a plant cell or plant made by any of the methods of the invention. Such terpenoids comprise sesquiterpenoids, such as farnesene and farnesane.

Genes for Terpenoid Metabolic Engineering.

To maximize the production of terpenoids in plants, such as sorghum and sugar cane, the MVA pathway, or the MEP pathway, or both pathways enzymes, are simultaneously expressed in a plant cell. In addition, to propel production of sesquiterpenoids to farnesene, IFF enzymes can also be expressed in the plant cell. Exemplary polypeptides of these pathways are shown in Tables 1 (MVA), 2 (MEP) and 3 (IFF). In addition to the polypeptides contemplated in Tables 1-3 and further described in Tables 4-7, one of skill in the art will understand that other polypeptides and polynucleotides can be used that encode polypeptides having similar enzymatic activity. Furthermore, polypeptides having active domains having the enzymatic activities of the polypeptides shown in Tables 1-3 and further described in Tables 4-7 can be used, including those polypeptides having at least approximately 70%-99% amino acid sequence identity with the polypeptides listed in Table 1-3, including those having at least approximately 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% amino acid sequence identity wherein the polypeptide retains an activity. Likewise, nucleic acid sequences encoding such functional polypeptides or active domains, including those polynucleotides derived from the amino acid sequences shown in Tables 1-3 and further described in Tables 4-7, including those polynucleotides that are codon optimized for expression in plants, such as monocots, using the OptimumGene™ Gene Design system (GenScript, New Jersy, USA; Burgess-Brown NA, Sharma S, Sobott F, Loenarz C, Oppermann U, Gileadi O. Codon optimization can improve expression of human genes in Escherichia coli: A multi-gene study. Protein Expr Purif. May 2008; 59(1): 94-102) (such polynucleotides are shown in Table 7 below) and those polynucleotides having at least approximately 70%-99% nucleic acid sequence identity to such polynucleotides derived from the amino acid sequences in Tables 1-3 and further described in Tables 4-7, (such as those shown in Table 7) including those having at least approximately 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% nucleic acid sequence identity wherein the encoded polypeptide retains an activity. Furthermore, the genomic and non-genomic forms of such nucleic acid sequences can be used, and in some embodiments, one or the other may be advantageous.

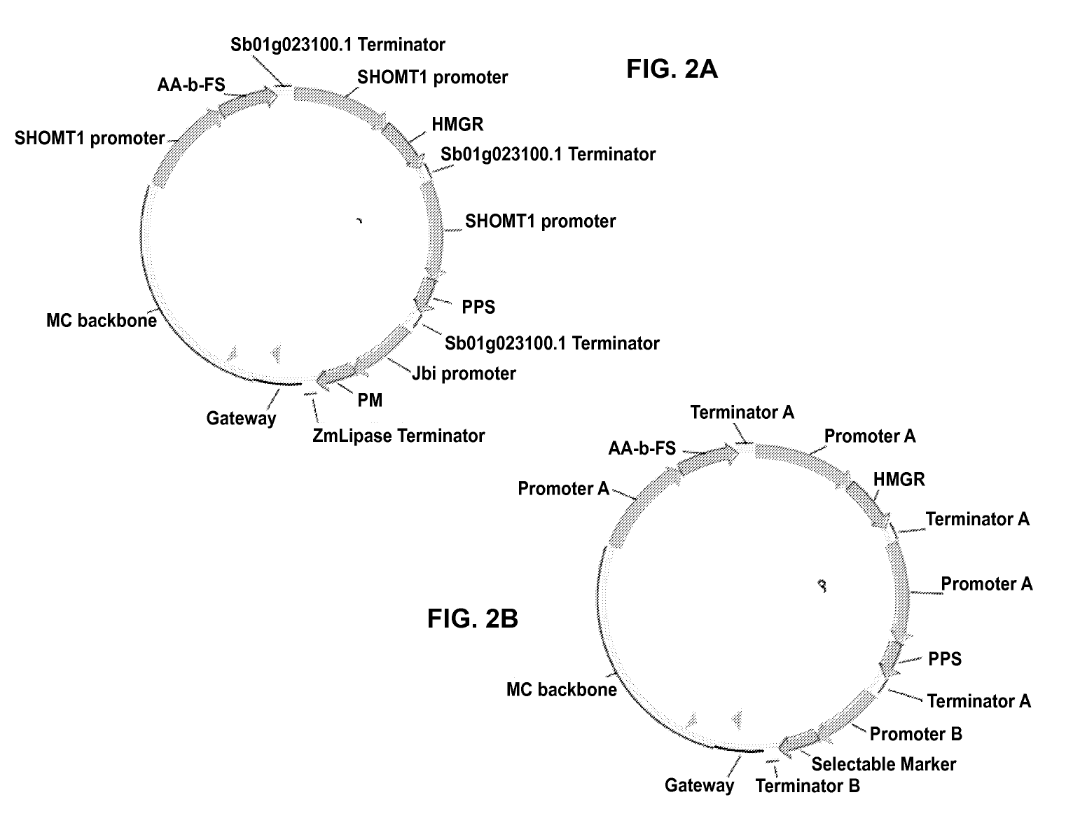

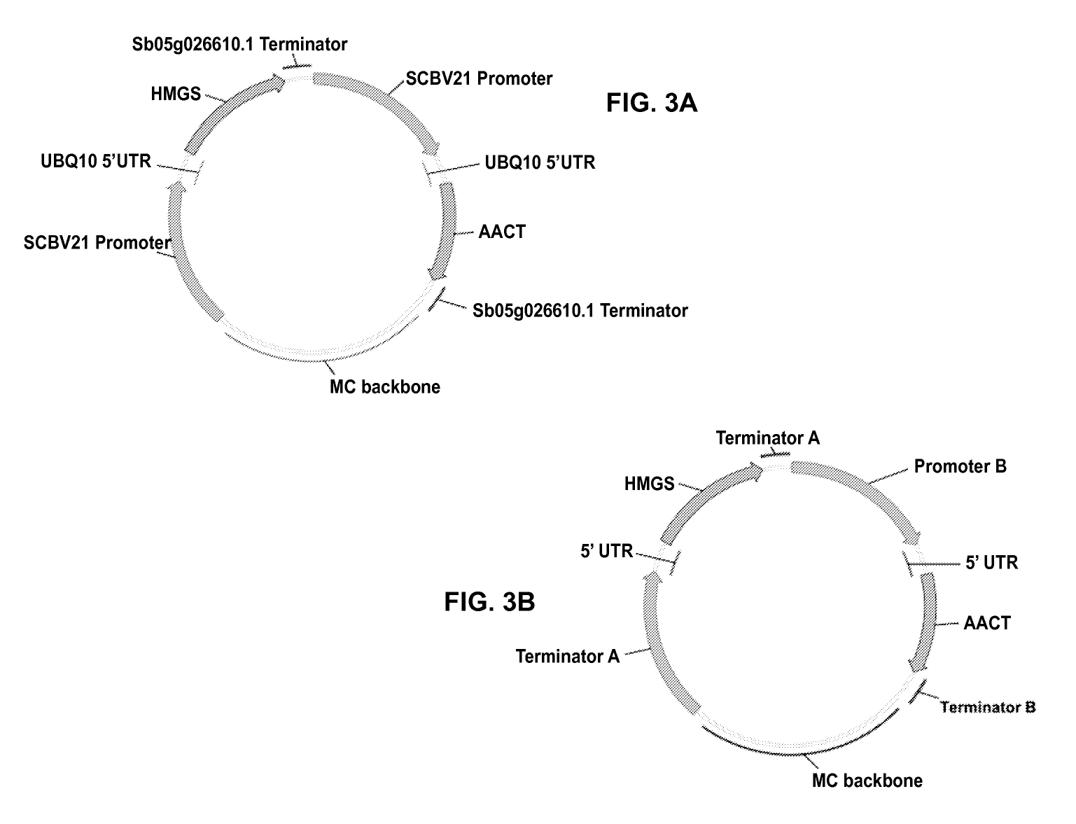

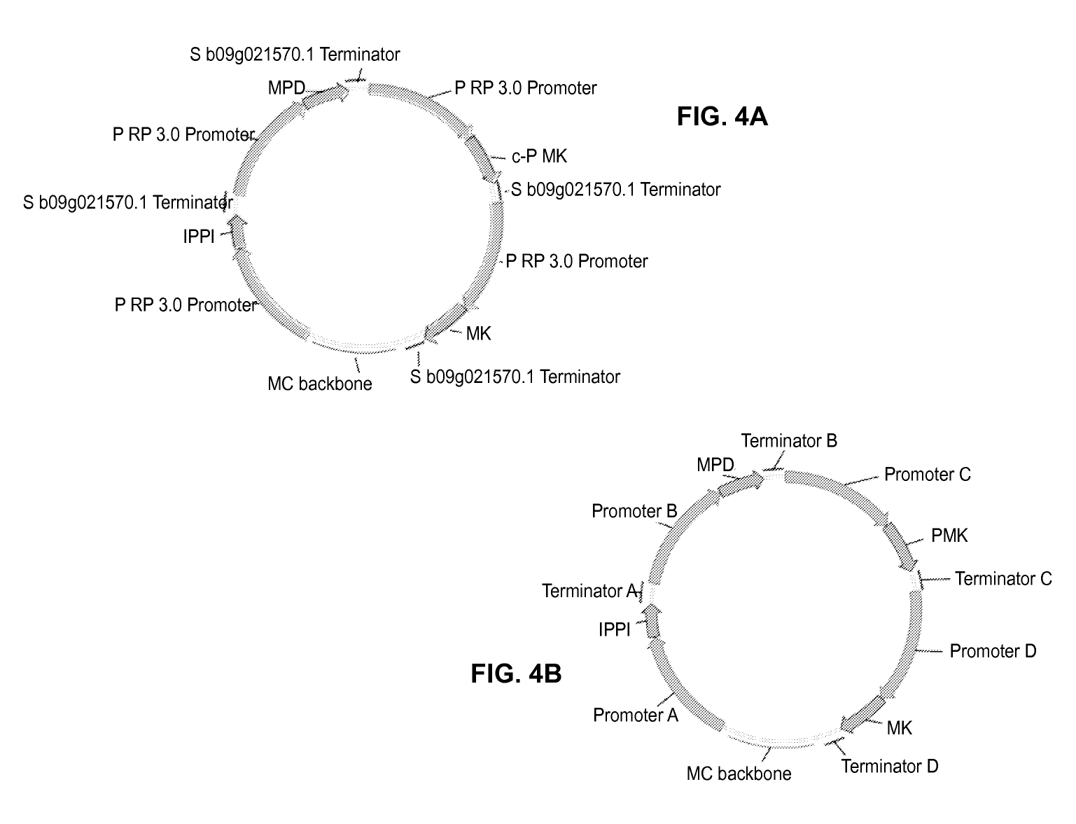

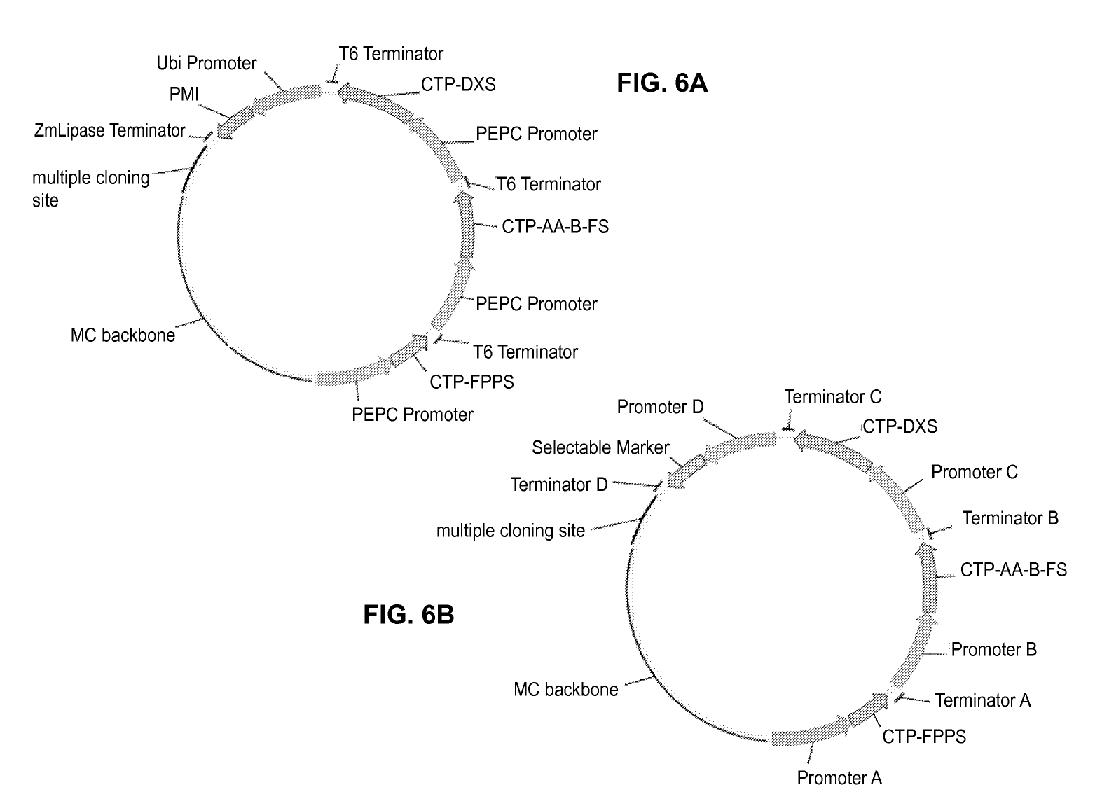

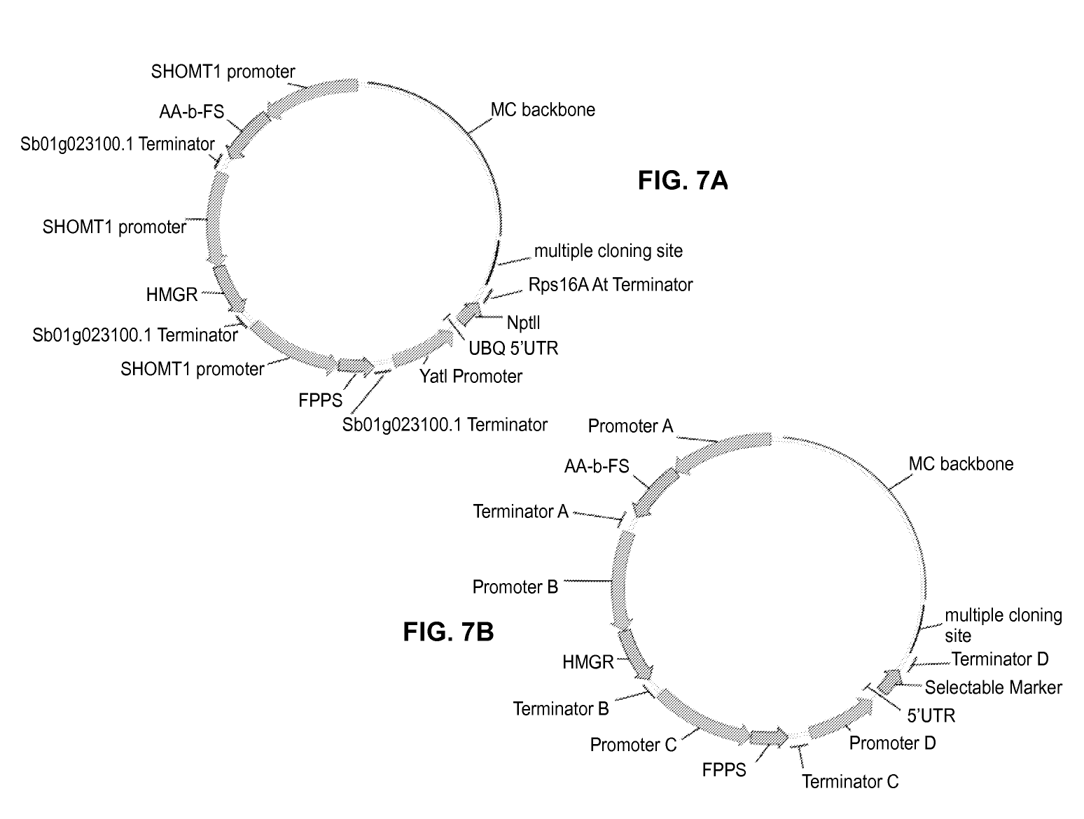

The details for the SEQ ID NOs listed in Tables 1-3 and further described in Tables 4-7 are shown in Table 4-6, showing the sequence of an exemplary polypeptide for each class of polypeptides. The polypeptide amino acid sequences are represented by accession numbers and are from the UNIPROT database (The UniProt Consortium (2011) Ongoing and future developments at the Universal Protein Resource. Nucleic Acids Research 39 (suppl 1): D214-D219), or in some cases, and as indicated, are GenBank mRNA polynucleotide sequences which have had the longest open reading frame translated. Polynucleotides encoding the polypeptides, or active domain of such polypeptides, shown in Tables 1-3 are transformed into a plant cells; in some embodiments, the plant cells are from sugar cane or sorghum, to up-regulate terpenoid synthesis and in some embodiments, to route carbon into the production of β-farnesene-enriched resins. FIGS. 2-7 give just a few of the constructs that can be useful in the invention, using the sequences shown and described in Tables 1-7. See also the Examples for additional constructs.

TABLE 1
Mevalonic acid pathway exemplary polypeptides

| Name | SEQ ID NO |
|---|---|
| acetyl-CoA acetyltransferase | 1-4, 143 |
| 3-hydroxy-3-methylglutaryl coenzyme A synthase | 5-9, 144, 145 |
| 3-hydroxy-3-methylglutaryl-coenzyme A | 10-16, 17-20, 146-150 |
| reductase | |
| mevalonate kinase | 21-26, 151 |
| phosphomevalonate kinase | 27-33 |
| mevalonate pyrophosphate decarboxylase | 34-40, 152 |

TABLE 2
Methylerthritol 4-phosphate pathway exemplary polypeptides

| Name | SEQ ID NO |
|---|---|
| 1-deoxy-D-xyulose-5-phosphate synthase | 41-49, 153, 154, |
| | 169, 177-180 |
| 1-deoxy-D-xyulose-5-phosphate reductoisomerase | 50-58, 155, 156, |
| | 170, 181 |
| 4-diphosphocytidyl-2-C-methyl-D-erythritol synthase | 59-67, 157, 171, |
| | 182 |
| 4-diphosphocytidyl-2-C-methyl-D-erythritol kinase | 68-73, 158, 172, |
| | 183 |
| 2-C-methyl-D-erythritol 2,4-cyclodiphosphate synthase | 74-82, 159, 173, |
| | 184 |
| (E)-4-Hydroxy-3-methyl-but-2-enyl pyrophosphate | 83-89, 160, 174, |
| synthase | 185 |
| (E)-4-Hydroxy-3-methyl-but-2-enyl pyrophosphate | 90-97, 161-163, |
| reductase | 175, 186 |

TABLE 3
IFF exemplary polypeptides

| Name | SEQ ID NO |
|---|---|
| isopentenyl-diphosphate-δ-isomerase I | 98-101, 190-192 |
| isopentenyl-diphosphate-δ-isomerase II | 102-106, 188 |
| farnesyl diphosphate synthase | 107-111, 164, 165, |
| | 176, 187, 189 |
| β-farnesene synthase | 112-115, 166, 167 |
| α-farnesene synthase | 116-117, 168 |

TABLE 4
Exemplary MVA pathway sequences
Acetyl-CoA acetyltransferase
Sequence example (SEQ ID NO: 1, microbial):

| MKNCVIVSAV RTAIGSFNGS LASTSAIDLG ATVIKAAIER AKIDSQHVDE VIMGNVLQAG | | | | | | 60 | |
|---|---|---|---|---|---|---|---|
| LGQNPARQAL LKSGLAETVC GFTVNKVCGS GLKSVALAAQ AIQAGQAQSI VAGGMENMSL | | | | | | 120 | |
| APYLLDAKAR SGYRLGDGQV YDVILRDGLM CATHGYHMGI TAENVAKEYG ITREMQDELA | | | | | | 180 | |
| LHSQRKAAAA IESGAFTAEI VPVNVVTRKK TFVFSQDEFP KANSTAEALG ALRPAFDKAG | | | | | | 240 | |
| TVTAGNASGI NDGAAALVIM EESAALAAGL TPLARIKSYA SGGVPPALMG MGPVPATQKA | | | | | | 300 | |
| LQLAGLQLAD IDLIEANEAF AAQFLAVGKN LGFDSEKVNV NGGAIALGHP IGASGARILV | | | | | | 360 | |
| TLLHAMQARD KTLGLATLCI GGGQGIAMVI ERLN | | | | | | 394 | |
| SEQ ID | | | | | | | |
| NO. | Taxon | Entry | Entry name | Protein names | Organism | Length | Gene |
| 2 | Bacteria | P76461 | ATOB_ECOLI | Acetyl-CoA | Escherichia coli | 394 | atoB |
| | | | | acetyltransferase (EC | (strain K12) | | b2224 |
| | | | | 2.3.1.9) (Acetoacetyl- | | | JW2218 |
| | | | | CoA thiolase) | | | |
| 3 | Fungi | P41338 | THIL_YEAST | Acetyl-CoA | Saccharomyces | 398 | ERG10 |
| | | | | acetyltransferase (EC | cerevisiae (strain | | YPL028W |
| | | | | 2.3.1.9) (Acetoacetyl- | ATCC 204508 / | | LPB3 |
| | | | | CoA thiolase) | S288c)(Baker's | | |
| | | | | (Ergosterol | yeast) | | |
| | | | | biosynthesis protein | | | |
| | | | | 10) | | | |
| 4 | Plantae | A9ZMZ4 | A9ZMZ4_HEVBR | Acetyl-CoA C- | Hevea brasiliensis | 404 | HbAACT |
| | | | | acetyltransferase (EC | (Para rubber | | |
| | | | | 2.3.1.9) | tree)(Siphonia | | |
| | | | | | brasiliensis) | | |
| 143 | Plantae | | EZ239563 | Acetyl-coA- | Artemisia annua | 453 | |
| | | | (GenBank | acetyltransferase | | | |
| | | | mRNA | | | | |
| | | | polynuc- | | | | |
| | | | leotide | | | | |
| | | | sequence) | | | | |

3-hydroxy-3-methylglutaryl-ACP synthase pksG
Sequence example (SEQ ID NO: 5, microbial):

| MTIGIDKINF YVPKYYVDMA KLAEARQVDP NKFLIGIGQT EMAVSPVNQD IVSMGANAAK | | | | | | 60 | |
|---|---|---|---|---|---|---|---|
| DIITDEDKKK IGMVIVATES AVDAAKAAAV QIHNLLGIQP FARCFEMKEA CYAATPAIQL | | | | | | 120 | |
| AKDYLATRPN EKVLVIATDT ARYGLNSGGE PTQGAGAVAM VIAHNPSILA LNEDAVAYTE | | | | | | 180 | |
| DVYDFWRPTG HKYPLVDGAL SKDAYIRSFQ QSWNEYAKRQ GKSLADFASL CFHVPFTKMG | | | | | | 240 | |
| KKALESIIDN ADETTQERLR SGYEDAVDYN RYVGNIYTGS LYLSLISLLE NRDLQAGETI | | | | | | 300 | |
| GLFSYGSGSV GEFYSATLVE GYKDHLDQAA HKALLNNRTE VSVDAYETFF KRFDDVEFDE | | | | | | 360 | |
| EQDAVHEDRH IFYLSNIENN VREYHRPE | | | | | | 388 | |
| SEQ ID | | | | | | | |
| NO: | Taxon | Entry | Entry name | Protein names | Organism | Length | Gene |
| 6 | Bacteria | Q99R90 | Q99R90_STAAM | 3-hydroxy-3- | Staphylococcus | 388 | mvaSSAV2546 |
| | | | | methylglutaryl CoA | aureus (strain | | |
| | | | | synthase | Mu50 / ATCC | | |
| | | | | | 700699) | | |
| 7 | Fungi | P54839 | HMCS_YEAST | Hydroxymethylglutaryl- | Saccharomyces | 491 | ERG13 |
| | | | | CoA synthase | cerevisiae (strain | | HMGS |
| | | | | (HMG-CoA synthase) | ATCC 204508/ | | YML126C |
| | | | | (EC 2.3.3.10) (3- | S288c)(Baker's | | YM4987.09C |
| | | | | hydroxy-3- | yeast) | | |
| | | | | methylglutaryl | | | |
| | | | | coenzyme A | | | |
| | | | | synthase) | | | |
| 8 | Plantae | Q944F8 | Q944F8_HEVBR | Hydroxymethylglutaryl | Hevea brasiliensis | 464 | |
| | | | | coenzyme A | (Para rubber | | |
| | | | | synthase | tree)(Siphonia | | |
| | | | | | brasiliensis) | | |
| 9 | Plantae | Q6QLW8 | Q6QLW8_HEVBR | HMG-CoA synthase 2 | Hevea brasiliensis | 464 | HMGS2 |
| | | | | | (Para rubber | | |
| | | | | | tree)(Siphonia | | |
| | | | | | brasiliensis) | | |
| 144 | Plantae | D2WS91 | D2WS91_ARTAN | HMG-CoA-synthase- | Artemisia annua | 458 | |
| | | | | 1 | | | |
| 145 | Plantae | | ACY74340.1 | HMG-CoA synthase-2 | Artemisia annua | 458 | |
| | | | (GenBank) | | | | |

3-hydroxy-3-methylglutaryl-coenzyme A reductase
Sequence example (SEQ ID NO: 10, microbial):

| MVLTNKTVIS GSKVKSLSSA QSSSSGPSSS SEEDDSRDIE SLDKKIRPLE ELEALLSSGN | | | | | | 60 | |
|---|---|---|---|---|---|---|---|
| TKQLKNKEVA ALVIHGKLPL YALEKKLGDT TRAVAVRRKA LSILAEAPVL ASDRLPYKNY | | | | | | 120 | |
| DYDRVFGACC ENVIGYMPLP VGVIGPLVID GTSYHIPMAT IEGCLVASAM RGCKAINAGG | | | | | | 180 | |
| GATTVLTKDG MIRGPVVRFP TLKRSGACKI WLDSEEGQNA IKKAFNSTSR FARLQHIQTC | | | | | | 240 | |
| LAGDLLFMRF RTTTGDAMGM NMISKGVEYS LKQMVEEYGW EDMEVVSVSG NYCIDKKPAA | | | | | | 300 | |
| INWIEGRGKS VVAEATIPGD VVRKVLKSDV SALVELNIAK NLVGSAMAGS VGGFNAHAAN | | | | | | 360 | |
| LVTAVFLALG QDPAQNVESS NCITLMKEVD GDLRISVSMP SIEVGTIGGG IVLEPQGAML | | | | | | 420 | |
| DLLGVRGPHA TAPGTNARQL ARIVACAVLA GELSLCAALA AGHLVQSHMT HNRKPAEPTK | | | | | | 480 | |
| PNNLDATDIN RLKDGSVTCI KS | | | | | | 502 | |
| SEQ ID | | | | | | | |
| NO: | Taxon | Entry | Entry name | Protein names | Organism | Length | Gene |
| 11 | Bacteria | Q5KSM8 | Q5KSM8_9ACTO | 3-hydroxy-3- | Streptomyces sp. | 353 | hmgr |
| | | | | methylglutaryl-CoA | KO-3988 | | |
| | | | | reductase | | | |
| 12 | Bacteria | B2HGT7 | B2HGT7_MYCMM | Hydroxymethylglutaryl- | Mycobacterium | 351 | MMAR_3214 |
| | | | | coenzyme A | marinum (strain | | |
| | | | | (HMG-CoA) | ATCC BAA-535 / | | |
| | | | | reductase | M) | | |
| 13 | Bacteria | A1ZZS8 | A1ZZS8_9BACT | Hydroxymethylglutaryl- | Microscilla | 424 | M23134_ |
| | | | | coenzyme A | marina ATCC | | 02465 |
| | | | | reductase (EC | 23134 | | |
| | | | | 1.1.1.34) | | | |
| 14 | Fungi | P12683 | HMDH1_YEAST | 3-hydroxy-3- | Saccharomyces | 1054 | HMG1YML075C |
| | | | | methylglutaryl- | cerevisiae (strain | | |
| | | | | coenzyme A | ATCC 204508 / | | |
| | | | | reductase 1 (HMG- | S288c)(Baker's | | |
| | | | | CoA reductase 1)(EC | yeast) | | |
| | | | | 1.1.1.34) | | | |
| 15 | Plantae | A9ZMZ9 | A9ZMZ9_HEVBR | Hydroxymethylglutaryl- | Hevea brasiliensis | 606 | HbHMGR |
| | | | | CoA reductase (EC | (Para rubber | | |
| | | | | 1.1.1.34) | tree)(Siphonia | | |
| | | | | | brasiliensis) | | |
| 16 | Plantae | Q00583 | HMDH3_HEVBR | 3-hydroxy-3- | Hevea brasiliensis | 586 | HMGR3 |
| | | | | methylglutaryl- | (Para rubber | | |
| | | | | coenzyme A | tree)(Siphonia | | |
| | | | | reductase 3 (HMG- | brasiliensis) | | |
| | | | | CoA reductase 3)(EC | | | |
| | | | | 1.1.1.34) | | | |
| 146 | Plantae | Q9SWQ3 | Q9SWQ3_ARTAN | 3-hydroxy-3- | Artemisia annua | 567 | |
| | | | | methylglutaryl- | | | |
| | | | | coenzyme A | | | |
| | | | | reductase | | | |

3-hydroxy-3-methylglutaryl-coenzyme A reductase
Sequence example (SEQ ID NO: 15, microbial):

| MQSLDKNFRH LSRQQKLQQL VDKQWLSEDQ FDILLNHPLI DEEVANSLIE NVIAQGALPV | | | | | | 60 | |
|---|---|---|---|---|---|---|---|
| GLLPNIIVDD KAYVVPMMVE EPSVVAAASY GAKLVNQTGG FKTVSSERIM IGQIVFDGVD | | | | | | 120 | |
| DTEKLSADIK ALEKQIHKIA DEAYPSIKAR GGGYQRIAID TFPEQQLLSL KVFVDTKDAM | | | | | | 180 | |
| GANMLNTILE AITAFLKNES PQSDILMSIL SNHATASVVK VQGEIDVKDL ARGERTGEEV | | | | | | 240 | |
| AKRMERASVL AQVDIHRAAT HNKGVMNGIH AVVLATGNDT RGAEASAHAY ASRDGQYRGI | | | | | | 300 | |
| ATWRYDQKRQ RLIGTIEVPM TLAIVGGGTK VLPIAKASLE LLNVDSAQEL GHVVAAVGLA | | | | | | 360 | |
| QNFAACRALV SEGIQQGHMS LQYKSLAIVV GAKGDEIAQV AEALKQEPRA NTQVAERILQ | | | | | | 420 | |
| EIRQQ | | | | | | 425 | |
| SEQ ID | | | | | | | |
| NO: | Taxon | Entry | Entry name | Protein names | Organism | Length | Gene |
| 18 | Bacteria | Q9FD86 | Q9FD86_STAAU | HMG-CoA reductase | Staphylococcus | 425 | mvaA |
| | | | | | aureus | | |
| 19 | Fungi | P12683 | HMDH1_YEAST | 3-hydroxy-3- | Saccharomyces | 1054 | HMG1YML075C |
| | | | | methylglutaryl- | cerevisiae (strain | | |
| | | | | coenzyme A | ATCC 204508 / | | |
| | | | | reductase 1 (HMG- | S288c)(Baker's | | |
| | | | | CoA reductase 1)(EC | yeast) | | |
| | | | | 1.1.1.34) | | | |
| 20 | Plantae | Q00583 | HMDH3_HEVBR | 3-hydroxy-3- | Hevea brasiliensis | 586 | HMGR3 |
| | | | | methylglutaryl- | (Para rubber | | |
| | | | | coenzyme A | tree)(Siphonia | | |
| | | | | reductase 3 (HMG- | brasiliensis) | | |
| | | | | CoA reductase 3)(EC | | | |
| | | | | 1.1.1.34) | | | |
| 147 | Plantae | Q43318 | Q43318_ARTAN | 3-hydroxy-3- | Artemisia annua | 566 | |
| | | | | methylglutaryl- | | | |
| | | | | coenzyme A | | | |
| | | | | reductase | | | |
| 148 | Plantae | Q43319 | Q43319_ARTAN | 3-hydroxy-3- | Artemisia annua | 560 | |
| | | | | methylglutaryl- | | | |
| | | | | coenzyme A | | | |
| | | | | reductase | | | |
| 149 | Plantae | | EZ228778.1 | 3-hydroxy-3- | Artemisia annua | 565 | |
| | | | (GenBank | methylglutaryl- | | | |
| | | | mRNA | coenzyme A | | | |
| | | | polynuc- | reductase-1 | | | |
| | | | leotide | | | | |
| | | | sequence) | | | | |
| 150 | Plantae | | EZ235445 | 3-hydroxy-3- | Artemisia annua | 585 | |
| | | | (GenBank | methylglutaryl- | | | |
| | | | mRNA | coenzyme A | | | |
| | | | polynuc- | reductase-3 | | | |
| | | | leotide | | | | |
| | | | sequence) | | | | |

Mevalonate kinase
Sequence example (SEQ ID NO: 21, microbial):

| MSLPFLTSAP GKVIIFGEHS AVYNKPAVAA SVSALRTYLL ISESSAPDTI ELDFPDISFN | | | | | | 60 | |
|---|---|---|---|---|---|---|---|
| HKWSINDFNA ITEDQVNSQK LAKAQQATDG LSQELVSLLD PLLAQLSESF HYHAAFCFLY | | | | | | 120 | |
| MFVCLCPHAK NIKFSLKSTL PIGAGLGSSA SISVSLALAM AYLGGLIGSN DLEKLSENDK | | | | | | 180 | |
| HIVNQWAFIG EKCIHGTPSG IDNAVATYGN ALLFEKDSHN GTINTNNFKF LDDFPAIPMI | | | | | | 240 | |
| LTYTRIPRST KDLVARVRVL VTEKFPEVMK PILDAMGECA LQGLEIMTKL SKCKGTDDEA | | | | | | 300 | |
| VETNNELYEQ LLELIRINHG LLVSIGVSHP GLELIKNLSD DLRIGSTKLT GAGGGGCSLT | | | | | | 360 | |
| LLRRDITQEQ IDSFKKKLQD DFSYETFETD LGGTGCCLLS AKNLNKDLKI KSLVFQLFEN | | | | | | 420 | |
| KTTTKQQIDD LLLPGNTNLP WTS | | | | | | 443 | |
| SEQ ID | | | | | | | |
| NO: | Taxon | Entry | Entry name | Protein names | Organism | Length | Gene |
| 22 | Bacteria | E8N5A6 | E8N5A6_ANATU | Mevalonate kinase | Anaerolinea | 313 | mvk |
| | | | | (EC 2.7.1.36) | thermophila | | ANT_ 159 |
| | | | | | (strain DSM | 40 | |
| | | | | | 14523 /JCM | | |
| | | | | | 11388 / NBRC | | |
| | | | | | 100420 / UNI-1) | | |
| 23 | Bacteria | A6G138 | A6G138_9DELT | Mevalonate kinase | Plesiocystis | 320 | PPSIR1_1175 |
| | | | | | pacifica SIR-1 | | |
| 24 | Bacteria | A9AY65 | A9AY65_HERA2 | Mevalonate kinase | Herpetosiphon | 313 | Haur_4315 |
| | | | | | aurantiacus | | |
| | | | | | (strain ATCC | | |
| | | | | | 23779 / DSM | | |
| | | | | | 785) | | |
| 25 | Fungi | P07277 | KIME_YEAST | Mevalonate kinase | Saccharomyces | 443 | ERG12 |
| | | | | (MK)(MvK)(EC | cerevisiae (strain | | RAR1 |
| | | | | 2.7.1.36) (Ergosterol | ATCC 204508 / | | YMR208W |
| | | | | biosynthesis protein | S288c)(Baker's | | YM8261.02 |
| | | | | 12) (Regulation of | yeast) | | |
| | | | | autonomous | | | |
| | | | | replication protein 1) | | | |
| 26 | Plantae | Q944G2 | Q944G2_HEVBR | Mevalonate kinase | Hevea brasiliensis | 386 | HbMVK |
| | | | | | (Para rubber | | |
| | | | | | tree)(Siphonia | | |
| | | | | | brasiliensis) | | |
| 151 | Plantae | | EZ251421 | Mevalonate kinase | Artemisia annua | 389 | |
| | | | (GenBank | | | | |
| | | | mRNA | | | | |
| | | | polynuc- | | | | |
| | | | leotide | | | | |
| | | | sequence) | | | | |

Phosphomevalonate kinase
Sequence example (SEQ ID NO: 27, microbial):

| MSELRAFSAP GKALLAGGYL VLDPKYEAFV VGLSARMHAV AHPYGSLQES DKFEVRVKSK | | | | | | 60 | |
|---|---|---|---|---|---|---|---|
| QFKDGEWLYH ISPKTGFIPV SIGGSKNPFI EKVIANVFSY FKPNMDDYCN RNLFVIDIFS | | | | | | 120 | |
| DDAYHSQEDS VTEHRGNRRL SFHSHRIEEV PKTGLGSSAG LVTVLTTALA SFFVSDLENN | | | | | | 180 | |
| VDKYREVIHN LSQVAHCQAQ GKIGSGFDVA AAAYGSIRYR RFPPALISNL PDIGSATYGS | | | | | | 240 | |
| KLAHLVNEED WNITIKSNHL PSGLTLWMGD IKNGSETVKL VQKVKNWYDS HMPESLKIYT | | | | | | 300 | |
| ELDHANSRFM DGLSKLDRLH ETHDDYSDQI FESLERNDCT CQKYPEITEV RDAVATIRRS | | | | | | 360 | |
| FRKITKESGA DIEPPVQTSL LDDCQTLKGV LTCLIPGAGG YDAIAVIAKQ DVDLRAQTAD | | | | | | 420 | |
| DKRFSKVQWL DVTQADWGVR KEKDPETYLD K | | | | | | 451 | |
| SEQ ID | | | | | | | |
| NO | Taxon | Entry | Entry name | Protein names | Organism | Length | Gene |
| 28 | Bacteria | C2ES75 | C2ES75_9LACO | Phosphomevalonate | Lactobacillus | 376 | HMPREF0549_ |
| | | | | kinase (EC 2.7.4.2) | vaginalis ATCC | | 0311 |
| | | | | | 49540 | | |
| 29 | Bacteria | C8P8V5 | C8P8V5_9LACO | Phosphomevalonate | Lactobacillus | 377 | mvaK |
| | | | | kinase (EC 2.7.4.2) | antri DSM 16041 | | HMPREF0494_ |
| | | | | | | | 1749 |
| 30 | Bacteria | COWXW9 | COWXW9_LACFE | Phosphomevalonate | Lactobacillus | 369 | HMPREF0511_ |
| | | | | kinase | fermentum ATCC | | 0970 |
| | | | | | 14931 | | |
| 31 | Fungi | A6ZMT2 | A6ZMT2_YEAS7 | Phosphomevalonate | Saccharomyces | 451 | ERG8SCY_ |
| | | | | kinase | cerevisiae (strain | | 4398 |
| | | | | | YJM789)(Baker's | | |
| | | | | | yeast) | | |
| 32 | Plantae | Q944G1 | Q944G1_HEVBR | Phosphomevalonate | Hevea brasiliensis | 503 | |
| | | | | kinase | (Para rubber | | |
| | | | | | tree)(Siphonia | | |
| | | | | | brasiliensis) | | |
| 33 | Plantae | A9ZN02 | A9ZN02_HEVBR | 5- | Hevea brasiliensis | 503 | HbMVD |
| | | | | phosphomevelonate | (Para rubber | | |
| | | | | kinase (EC 2.7.4.2) | tree)(Siphonia | | |
| | | | | | brasiliensis) | | |

Mevalonate pyrophosphate decarboxylase
Sequence examples (SEQ ID NO: 34, microbial):

| MTVYTASVTA PVNIATLKYW GKRDTKLNLP TNSSISVTLS QDDLRTLTSA ATAPEFERDT | | | | | | 60 | |
|---|---|---|---|---|---|---|---|
| LWLNGEPHSI DNERTQNCLR DLRQLRKEME SKDASLPTLS QWKLHIVSEN NFPIAAGLAS | | | | | | 120 | |
| SAAGFAALVS AIAKLYQLPQ STSEISRIAR KGSGSACRSL FGGYVAWEMG KAEDGHDSMA | | | | | | 180 | |
| VQIADSSDWP QMKACVLVVS DIKKDVSSTQ GMQLTVATSE LFKERIEHVV PKRFEVMRKA | | | | | | 240 | |
| IVEKDFATFA KETMMDSNSF HATCLDSFPP IFYMNDTSKR IISWCHTINQ FYGETIVAYT | | | | | | 300 | |
| FDAGPNAVLY YLAENESKLF AFIYKLFGSV PGWDKKFTTE QLEAFNHQFE SSNFTARELD | | | | | | 360 | |
| LELQKDVARV ILTQVGSGPQ ETNESLIDAK TGLPKE | | | | | | 396 | |
| SEQ ID | | | | | | | |
| NO | Taxon | Entry | Entry name | Protein names | Organism | Length | Gene |
| 35 | Bacteria | Q8ETN2 | Q8ETN2_OCEIH | Mevalonate | Oceanobacillus | 324 | OB0226 |
| | | | | diphosphate | iheyensis (strain | | |
| | | | | decarboxylase | DSM 14371 /JCM | | |
| | | | | | 11309 / KCTC | | |
| | | | | | 3954 / HTE831) | | |
| 36 | Bacteria | E8N6F3 | E8N6F3_ANATU | Diphosphomevalonate | Anaerolinea | 326 | mvaD |
| | | | | decarboxylase (EC | thermophila | | ANT_19910 |
| | | | | 4.1.1.33) | (strain DSM | | |
| | | | | | 14523 /JCM | | |
| | | | | | 11388 / NBRC | | |
| | | | | | 100420 / UNI-1) | | |
| 37 | Bacteria | C1PCJ6 | C1PCJ6_BACCO | Diphosphomevalonate | Bacillus | 326 | BcoaDRAFT_ |
| | | | | decarboxylase (EC | coagulans 36D1 | | 4576 |
| | | | | 4.1.1.33) | | | |
| 38 | Fungi | P32377 | MVD1_YEAST | Diphosphomevalonate | Saccharomyces | 396 | MVD1 |
| | | | | decarboxylase (EC | cerevisiae (strain | | ERG19 |
| | | | | 4.1.1.33) (Ergosterol | ATCC 204508 / | | MPD |
| | | | | biosynthesis protein | S288c)(Baker's | | YNR043W |
| | | | | 19)(Mevalonate | yeast) | | N3427 |
| | | | | pyrophosphate | | | |
| | | | | decarboxylase) | | | |
| | | | | (Mevalonate-5- | | | |
| | | | | diphosphate | | | |
| | | | | decarboxylase) | | | |
| | | | | (MDD)(MDDase) | | | |
| 39 | Plantae | Q944G0 | Q944G0_HEVBR | Mevalonate | Hevea brasiliensis | 415 | |
| | | | | disphosphate | (Para rubber | | |
| | | | | decarboxylase | tree)(Siphonia | | |
| | | | | | brasiliensis) | | |
| 40 | Plantae | A9ZN03 | A9ZN03_HEVBR | Diphosphomevelona | Hevea brasiliensis | 415 | HbPMD |
| | | | | to decarboxylase (EC | (Para rubber | | |
| | | | | 4.1.1.33) | tree)(Siphonia | | |
| | | | | | brasiliensis) | | |
| 152 | Plantae | | EZ207331 | Mevalonate | Artemisia annua | 414 | |
| | | | (GenBank | diphosphate | | | |
| | | | mRNA | decarboxylase | | | |
| | | | polynucleo- | | | | |
| | | | tide sequence) | | | | |

TABLE 5
Exemplary MEP pathway sequences
Deoxyxylulose-5-phosphate synthase
Sequence example (SEQ ID NO: 41, Arabidopsis thaliana):

| MASSAFAFPS YIITKGGLST DSCKSTSLSS SRSLVTDLPS PCLKPNNNSH SNRRAKVCAS | | | | | | 60 | |
|---|---|---|---|---|---|---|---|
| LAEKGEYYSN RPPTPLLDTI NYPIHMKNLS VKELKQLSDE LRSDVIFNVS KTGGHLGSSL | | | | | | 120 | |
| GVVELTVALH YIFNTPQDKI LWDVGHQSYP HKILTGRRGK MPTMRQTNGL SGFTKRGESE | | | | | | 180 | |
| HDCFGTGHSS TTISAGLGMA VGRDLKGKNN NVVAVIGDGA MTAGQAYEAM NNAGYLDSDM | | | | | | 240 | |
| IVILNDNKQV SLPTATLDGP SPPVGALSSA LSRLQSNPAL RELREVAKGM TKQIGGPMHQ | | | | | | 300 | |
| LAAKVDEYAR GMISGTGSSL FEELGLYYIG PVDGHNIDDL VAILKEVKST RTTGPVLIHV | | | | | | 360 | |
| VTEKGRGYPY AERADDKYHG VVKFDPATGR QFKTTNKTQS YTTYFAEALV AEAEVDKDVV | | | | | | 420 | |
| AIHAAMGGGT GLNLFQRRFP TRCFDVGIAE QHAVTFAAGL ACEGLKPFCA IYSSFMQRAY | | | | | | 480 | |
| DQVVHDVDLQ KLPVRFAMDR AGLVGADGPT HCGAFDVTFM ACLPNMIVMA PSDEADLFNM | | | | | | 540 | |
| VATAVAIDDR PSCFRYPRGN GIGVALPPGN KGVPIEIGKG RILKEGERVA LLGYGSAVQS | | | | | | 600 | |
| CLGAAVMLEE RGLNVTVADA RFCKPLDRAL IRSLAKSHEV LITVEEGSIG GFGSHVVQFL | | | | | | 660 | |
| ALDGLLDGKL KWRPMVLPDR YIDHGAPADQ LAEAGLMPSH IAATALNLIG APREALF | | | | | | 717 | |
| SEQ ID | | | | | | | |
| NO: | Taxon | Entry | Entry name | Protein names | Organism | Length | Gene |
| 42 | Bacteria | A8U2Y0 | A8U2Y0_ | 1-deoxy-D-xylulose- | Alpha | 638 | dxs |
| | | | 9PROT | 5-phosphate | proteobacterium | | BAL199_2_ |
| | | | | synthase (EC 2.2.1.7) | BAL199 | | 2207 |
| | | | | (1-deoxyxylulose-5- | | | |
| | | | | phosphate synthase) | | | |
| 43 | Bacteria | A7HR71 | A7HR71_ | 1-deoxy-D-xylulose- | Parvibaculum | 650 | dxs |
| | | | PARL1 | 5-phosphate | lavamentivorans | | Plav_0781 |
| | | | | synthase (EC 2.2.1.7) | (strain DS-1 / | | |
| | | | | (1-deoxyxylulose-5- | DSM 13023 / | | |
| | | | | phosphate synthase) | NCIMB 13966) | | |
| 44 | Bacteria | Q2W367 | DXS_MAGSA | 1-deoxy-D-xylulose- | Magnetospirillum | 644 | dxs |
| | | | | 5-phosphate | magneticum | | amb2904 |
| | | | | synthase (EC 2.2.1.7) | (strain AMB-1 / | | |
| | | | | (1-deoxyxylulose-5- | ATCC 700264) | | |
| | | | | phosphate synthase) | | | |
| | | | | (DXP synthase) | | | |
| | | | | (DXPS) | | | |
| 45 | Fungi | C4Y4H6 | C4Y4H6_ | Putative | Clavispora | 362 | CLUG_ |
| | | | CLAL4 | uncharacterized | lusitaniae (strain | | 02548 |
| | | | | protein | ATCC 42720) | | |
| | | | | | (Yeast)(Candida | | |
| | | | | | lusitaniae) | | |
| 46 | Fungi | F9FXE5 | F9FXE5_ | Putative | Fusarium | 404 | FOXB_ |
| | | | FUSOX | uncharacterized | oxysporum | | 11077 |
| | | | | protein | Fo5176 | | |
| 47 | Fungi | Q5A5V6 | Q5A5V6_ | Putative | Candida albicans | 379 | PDB1 |
| | | | CANAL | uncharacterized | (strain SC5314 / | | CaO19.12753 |
| | | | | protein PDB1 | ATCC MYA-2876) | | CaO19.5294 |
| | | | | | (Yeast) | | |
| 48 | Plantae | A9ZN06 | A9ZN06_HEVBR | 1-deoxy-D-xylulose | Hevea brasiliensis | 720 | HbDXS1 |
| | | | | 5-phosphate | (Para rubber | | |
| | | | | synthase (EC 2.2.1.7) | tree)(Siphonia | | |
| | | | | | brasiliensis) | | |
| 49 | Plantae | A1KXW4 | A1KXW4_HEVBR | Putative 1-deoxy-D- | Hevea brasiliensis | 720 | DXS |
| | | | | xylulose 5-phosphate | (Para rubber | | |
| | | | | synthase | tree)(Siphonia | | |
| | | | | | brasiliensis) | | |
| 153 | Plantae | Q9SP65 | Q9SP65_ARTAN | 1-deoxy-D-xylulose | Artemisia annua | 713 | |
| | | | | 5-phosphate | | | |
| | | | | synthase | | | |
| 154 | Plantae | | EZ167196 | 1-deoxy-D-xylulose | Artemisia annua | 728 | |
| | | | (Genbank | 5-phosphate | | | |
| | | | polynucleo- | synthase | | | |
| | | | tide mRNA | | | | |
| | | | sequence) | | | | |
| 169 | Bacteria | | AAC73523 | 1-deoxy-D-xylulose | E. coli | 620 | |
| | | | (GenBank | 5-phosphate | | | |
| | | | polynucleo- | synthase | | | |
| | | | tide sequence) | | | | |
| 177 | Algae | O81954 | 081954_CHRLE | 1-deoxy-D-xylulose | Chlamydomonas | 735 | |
| | | | | 5-phosphate | reinhardtii | | |
| | | | | synthase | | | |
| 178 | Algae | | AEZ35185 | 1-deoxy-D-xylulose | Botryococcus | 770 | |
| | | | (GenBank | 5-phosphate | braunii | | |
| | | | polynucleo- | synthase | | | |
| | | | tide sequence) | | | | |
| 179 | Algae | | AEZ35186 | 1-deoxy-D-xylulose | Botryococcus | 771 | |
| | | | (GenBank | 5-phosphate | braunii | | |
| | | | polynucleo- | synthase | | | |
| | | | tide sequence) | | | | |
| 180 | Algae | | AEZ35187 | 1-deoxy-D-xylulose | Botryococcus | 730 | |
| | | | (GenBank | 5-phosphate | braunii | | |
| | | | polynucleo- | synthase | | | |
| | | | tide sequence) | | | | |

1-deoxy-D-xylulose 5-phosphate reductoisomerase
Sequence example (SEQ ID NO: 50, Arabidopsis thaliana):

| MMTLNSLSPA ESKAISFLDT SRFNPIPKLS GGFSLRRRNQ GRGFGKGVKC SVKVQQQQQP | | | | | | 60 | |
|---|---|---|---|---|---|---|---|
| PPAWPGRAVP EAPRQSWDGP KPISIVGSTG SIGTQTLDIV AENPDKFRVV ALAAGSNVTL | | | | | | 120 | |
| LADQVRRFKP ALVAVRNESL INELKEALAD LDYKLEIIPG EQGVIEVARH PEAVTVVTGI | | | | | | 180 | |
| VGCAGLKPTV AAIEAGKDIA LANKETLIAG GPFVLPLANK HNVKILPADS EHSAIFQCIQ | | | | | | 240 | |
| GLPEGALRKI ILTASGGAFR DWPVEKLKEV KVADALKHPN WNMGKKITVD SATLFNKGLE | | | | | | 300 | |
| VIEAHYLFGA EYDDIEIVIH PQSIIHSMIE TQDSSVLAQL GWPDMRLPIL YTMSWPDRVP | | | | | | 360 | |
| CSEVTWPRLD LCKLGSLTFK KPDNVKYPSM DLAYAAGRAG GTMTGVLSAA NEKAVEMFID | | | | | | 420 | |
| EKISYLDIFK VVELTCDKHR NELVTSPSLE EIVHYDLWAR EYAANVQLSS GARPVHA | | | | | | 477 | |
| SEQ ID | | | | | | | |
| NO: | Taxon | Entry | Entry name | Protein names | Organism | Length | Gene |
| 51 | Bacteria | D8FYL0 | D8FYL0_9CYAN | 1-deoxy-D-xylulose | Oscillatoria sp. | 396 | dxr |
| | | | | 5-phosphate | PCC 6506 | | OSCI_1910010 |
| | | | | reductoisomerase | | | |
| | | | | (DXP | | | |
| | | | | reductoisomerase) | | | |
| | | | | (EC 1.1.1.267) (1- | | | |
| | | | | deoxyxylulose-5- | | | |
| | | | | phosphate | | | |
| | | | | reductoisomerase) | | | |
| | | | | (2-C-methyl-D- | | | |
| | | | | erythritol 4- | | | |
| | | | | phosphate synthase) | | | |
| 52 | Bacteria | D7E0Y7 | D7E0Y7_NOSA0 | 1-deoxy-D-xylulose | Nostoc azollae | 398 | dxr |
| | | | | 5-phosphate | (strain 0708) | | Aazo_0646 |
| | | | | reductoisomerase | (Anabaena | | |
| | | | | (DXP | azollae (strain | | |
| | | | | reductoisomerase) | 0708)) | | |
| | | | | (EC 1.1.1.267) (1- | | | |
| | | | | deoxyxylulose-5- | | | |
| | | | | phosphate | | | |
| | | | | reductoisomerase) | | | |
| | | | | (2-C-methyl-D- | | | |
| | | | | erythritol 4- | | | |
| | | | | phosphate synthase) | | | |
| 53 | Bacteria | B4WQ44 | B4WQ44_9SYNE | 1-deoxy-D-xylulose | Synechococcus | 389 | dxr |
| | | | | 5-phosphate | sp. PCC 7335 | | S7335_4035 |
| | | | | reductoisomerase | | | |
| | | | | (DXP | | | |
| | | | | reductoisomerase) | | | |
| | | | | (EC 1.1.1.267) (1- | | | |
| | | | | deoxyxylulose-5- | | | |
| | | | | phosphate | | | |
| | | | | reductoisomerase) | | | |
| | | | | (2-C-methyl-D- | | | |
| | | | | erythritol 4- | | | |
| | | | | phosphate synthase) | | | |
| 54 | Fungi | Q4PFD0 | Q4PFD0_USTMA | Putative | Ustilago maydis | 1692 | UM01183.1 |
| | | | | uncharacterized | (strain 521 / FGSC | | |
| | | | | protein | 9021)(Smut | | |
| | | | | | fungus) | | |
| 55 | Fungi | Q96UP6 | RAD52_EMENI | DNA repair and | Emericella | 582 | radC |
| | | | | recombination | nidulans | | AN4407 |
| | | | | protein radC (RAD52 | (Aspergillus | | |
| | | | | homolog) | nidulans) | | |
| 56 | Plantae | Q0GYS3 | Q0GYS3_HEVBR | 1-deoxy-D-xylulose | Hevea brasiliensis | 471 | DXR |
| | | | | 5-phosphate | (Para rubber | | DXR2 |
| | | | | reductoisomerase | tree)(Siphonia | | |
| | | | | (Putative 1-deoxy-D- | brasiliensis) | | |
| | | | | xylulose 5-phosphate | | | |
| | | | | reductoisomerase) | | | |
| 57 | Plantae | A9ZN08 | A9ZN08_HEVBR | 1-deoxy-D-xylulose- | Hevea brasiliensis | 471 | HbDXR |
| | | | | 5-phosphate | (Para rubber | | |
| | | | | reductoisomerase | tree)(Siphonia | | |
| | | | | (EC 1.1.1.267) | brasiliensis) | | |
| 58 | Plantae | A1KXW2 | A1KXW2_HEVBR | 1-deoxy-D-xylulose | Hevea brasiliensis | 471 | DXR |
| | | | | 5-phosphate | (Para rubber | | |
| | | | | reductoisomerase | tree)(Siphonia | | |
| | | | | | brasiliensis) | | |
| 155 | Plantae | Q9SP64 | Q9SP64_ARTAN | 1-deoxy-D-xylulose | Artemisia annua | 472 | |
| | | | | 5-phosphate | | | |
| | | | | reductoisomerase | | | |
| 156 | Plantae | | EZ240020 | 1-deoxy-D-xylulose | Artemisia annua | 453 | |
| | | | (GenBank | 5-phosphate | | | |
| | | | mRNA | reductoisomerase | | | |
| | | | polynucleo- | | | | |
| | | | tide sequence) | | | | |
| 170 | Bacteria | | AAC73284 | 1-deoxy-D-xylulose | E. coli | 398 | |
| | | | (GenBank | 5-phosphate | | | |
| | | | polynucleo- | reductoisomerase | | | |
| | | | tide sequence) | | | | |
| 181 | Algae | | KA123067 | 1-deoxy-D-xylulose | Botrycoccus | 479 | |
| | | | (GenBank | 5-phosphate | braunii | | |
| | | | polynucleo- | reductoisomerase | | | |
| | | | tide sequence) | | | | |

2-C-methyl-D-erythritol 4-phosphate cytidylyltransferase
Sequence example (SEQ ID NO: 59, Arabidopsis thaliana):

| MAMLQTNLGF ITSPTFLCPK LKVKLNSYLW FSYRSQVQKL DFSKRVNRSY KRDALLLSIK | | | | | | 60 | |
|---|---|---|---|---|---|---|---|
| CSSSTGFDNS NVVVKEKSVS VILLAGGQGK RMKMSMPKQY IPLLGQPIAL YSFFIFSRMP | | | | | | 120 | |
| EVKEIVVVCD PFFRDIFEEY EESIDVDLRF AIPGKERQDS VYSGLQEIDV NSELVCIHDS | | | | | | 180 | |
| ARPLVNTEDV EKVLKDGSAV GAAVLGVPAK ATIKEVNSDS LVVKTLDRKT LWEMQTPQVI | | | | | | 240 | |
| KPELLKKGFE LVKSEGLEVT DDVSIVEYLK HPVYVSQGSY TNIKVTTPDD LLLAERILSE | | | | | | 300 | |
| DS | | | | | | 302 | |
| SEQ ID | | | | | | | |
| NO: | Taxon | Entry | Entry name | Protein names | Organism | Length | Gene |
| 60 | Bacteria | F8KVL1 | F8KVL1_PARAV | 2-C-methyl-D- | Parachlamydia | 229 | isPD ispD |
| | | | | erythritol 4- | acanthamoebae | | PUV_01970 |
| | | | | phosphate | (strain UV7) | | |
| | | | | cytidylyltransferase | | | |
| | | | | (EC 2.7.7.60)(4- | | | |
| | | | | diphosphocytidy1-2C- | | | |
| | | | | methyl-D-erythritol | | | |
| | | | | synthase)(MEP | | | |
| | | | | cytidylyltransferase) | | | |
| 61 | Bacteria | F8L5L7 | F8L5L7_SIMNZ | 2-C-methyl-D- | Simkania | 226 | isPD |
| | | | | erythritol 4- | negevensis | | ispD1 |
| | | | | phosphate | (strain ATCC VR- | | SNE_A18880 |
| | | | | cytidylyltransferase 1 | 1471 / Z) | | |
| | | | | (EC 2.7.7.60)(4- | | | |
| | | | | diphosphocytidy1-2C- | | | |
| | | | | methyl-D-erythritol | | | |
| | | | | synthase 1)(MEP | | | |
| | | | | cytidylyltransferase | | | |
| | | | | 1) | | | |
| 62 | Bacteria | Q6MEE8 | ISPD_PARUW | 2-C-methyl-D- | Protochlamydia | 230 | ispD |
| | | | | erythritol 4- | amoebophila | | pc0327 |
| | | | | phosphate | (strain UWE25) | | |
| | | | | cytidylyltransferase | | | |
| | | | | (EC 2.7.7.60)(4- | | | |
| | | | | diphosphocytidy1-2C- | | | |
| | | | | methyl-D-erythritol | | | |
| | | | | synthase)(MEP | | | |
| | | | | cytidylyltransferase) | | | |
| | | | | (MCT) | | | |
| 63 | Fungi | Q2U5Q5 | Q2U5Q5_ASPOR | Putative | Aspergillus | 420 | A009011 |
| | | | | uncharacterized | oryzae (strain | | 3000049 |
| | | | | protein | ATCC 42149 / RIB | | |
| | | | | AO090113000049 | 40) | | |
| 64 | Fungi | Q6FTD7 | Q6FTD7_CANGA | Strain CBS138 | Candida glabrata | 1072 | CAGLOGO |
| | | | | chromosome G | (strain ATCC 2001 / | | 3311g |
| | | | | complete sequence | CBS 138 / JCM | | |
| | | | | | 3761 / NBRC | | |
| | | | | | 0622 / NRRL Y- | | |
| | | | | | 65)(Yeast) | | |
| | | | | | (Torulopsis | | |
| | | | | | glabrata) | | |
| 65 | Fungi | P09436 | SYIC_YEAST | Isoleucyl-tRNA | Saccharomyces | 1072 | ILS1 |
| | | | | synthetase, | cerevisiae (strain | | YBL076C |
| | | | | cytoplasmic (EC | ATCC 204508 / | | YBL0734 |
| | | | | 6.1.1.5)(Isoleucine-- | S288c)(Baker's | | |
| | | | | tRNA ligase)(IleRS) | yeast) | | |
| 66 | Plantae | A9ZN10 | A9ZN10_HEVBR | 2-C-methyl-D- | Hevea brasiliensis | 311 | HbCMS |
| | | | | erythritol 4- | (Para rubber | | |
| | | | | phosphate | tree)(Siphonia | | |
| | | | | cytidylyltransferase | brasiliensis) | | |
| | | | | (EC 2.7.7.60) | | | |
| 67 | Plantae | A9ZN09 | A9ZN09_HEVBR | 2-C-methyl-D- | Hevea brasiliensis | 311 | HbCMS |
| | | | | erythritol 4- | (Para rubber | | |
| | | | | phosphate | tree)(Siphonia | | |
| | | | | cytidylyltransferase | brasiliensis) | | |
| | | | | (EC 2.7.7.60) | | | |
| 157 | Plantae | | EZ222881 | 2-C-methyl-D- | Artemisia annua | 302 | |
| | | | (GenBank | erythritol 4- | | | |
| | | | mRNA | phosphate | | | |
| | | | polynucleo- | cytidylyltransferase | | | |
| | | | tide sequence) | | | | |
| 171 | Bacteria | | AAC75789 | 2-C-methyl-D- | E. coli | 236 | |
| | | | (GenBank | erythritol 4- | | | |
| | | | polynucleo- | phosphate | | | |
| | | | tide sequence) | cytidylyltransferase | | | |
| 182 | Algae | | KA659949 | 2-C-methyl-D- | Botrycoccus | 298 | |
| | | | (GenBank | erythritol 4- | braunii | | |
| | | | polynucleo- | phosphate | | | |
| | | | tide sequence) | cytidylyltransferase | | | |

4-diphosphocytidyl-2C-methyl-D-erythritol kinase
Sequence example (SEQ ID NO: 68, Arabidopsis thaliana):

| MHHHHHHASM DREAGLSRLT LFSPCKINVF LRITSKRDDG YHDLASLFHV ISLGDKIKFS | | | | | | 60 | |
|---|---|---|---|---|---|---|---|
| LSPSKSKDRL STNVAGVPLD ERNLIIKALN LYRKKTGTDN YFWIHLDKKV PTGAGLGGGS | | | | | | 120 | |
| SNAAIILWAA NQFSGCVATE KELQEWSGEI GSDIPFFFSH GAAYCTGRGE VVQDIPSPIP | | | | | | 180 | |
| FDIPMVLIKP QQACSTAEVY KRFQLDLSSK VDPLSLLEKI STSGISQDVC VNDLEPPAFE | | | | | | 240 | |
| VLPSLKRLKQ RVIAAGRGQY DAVFMSGSGS TIVGVGSPDP PQFVYDDEEY KDVFLSEASF | | | | | | 300 | |
| ITRPANEWYV EPVSGSTIGD QPEFSTSFDM S | | | | | | 331 | |
| SEQ ID | | | | | | | |
| NO: | Taxon | Entry | Entry name | Protein names | Organism | Length | Gene |
| 69 | Bacteria | Q6MAT6 | ISPE_PARUW | 4-diphosphocytidyl- | Protochlamydia | 288 | ispE |
| | | | | 2-C-methyl-D- | amoebophila | | pc1589 |
| | | | | erythritol kinase | (strain UWE25) | | |
| | | | | (CMK)(EC 2.7.1.148) | | | |
| | | | | (4-(cytidine-5′- | | | |
| | | | | diphospho)-2-C- | | | |
| | | | | methyl-D-erythritol | | | |
| | | | | kinase) | | | |
| 70 | Bacteria | F8L344 | F8L344_SIMNZ | 4-diphosphocytidyl- | Simkania | 294 | ispE |
| | | | | 2-C-methyl-D- | negevensis | | SNE_A18050 |
| | | | | erythritol kinase | (strain ATCC VR- | | |
| | | | | (CMK)(EC 2.7.1.148) | 1471 / Z) | | |
| | | | | (4-(cytidine-5′- | | | |
| | | | | diphospho)-2-C- | | | |
| | | | | methyl-D-erythritol | | | |
| | | | | kinase) | | | |
| 71 | Fungi | D8PTC7 | D8PTC7_SCHCM | Putative | Schizophyllum | 556 | SCHCODRAFT_ |
| | | | | uncharacterized | commune (strain | | 256250 |
| | | | | protein | H4-8 / FGSC | | |
| | | | | | 9210) (Split gill | | |
| | | | | | fungus) | | |
| 72 | Fungi | Q8SRR7 | Q8SRR7_ENCCU | MEVALONATE | Encephalitozoon | 303 | ECU060_490 |
| | | | | PYROPHOSPHATE | cuniculi (strain | | |
| | | | | DECARBOXYLASE | GB-M1) | | |
| | | | | | (Microsporidian | | |
| | | | | | parasite) | | |
| 73 | Plantae | A9ZN11 | A9ZN11_HEVBR | 4-(Cytidine 5′- | Hevea brasiliensis | 388 | HbCMK |
| | | | | diphospho)-2-C- | (Para rubber | | |
| | | | | methyl-D-erythritol | tree) (Siphonia | | |
| | | | | kinase (EC 2.7.1.148) | brasiliensis) | | |
| | | | | (4-diphosphocytidy1- | | | |
| | | | | 2C-methyl-D- | | | |
| | | | | erythritol kinase) | | | |
| 158 | Plantae | | EZ157809 | 4-diphosphocytidyl- | Artemisia annua | 396 | |
| | | | (GenBank | 2C-methyl-D- | | | |
| | | | mRNA | erythritol kinase | | | |
| | | | polynucleo- | | | | |
| | | | tide sequence) | | | | |
| 172 | Bacteria | | AAC74292 | 4-diphosphocytidyl- | E. coli | 283 | |
| | | | (GenBank | 2C-methyl-D- | | | |
| | | | polynucleo- | erythritol kinase | | | |
| | | | tide sequence) | | | | |
| 183 | Algae | | KA659950 | 4-diphosphocytidyl- | Botrycoccus | 357 | |
| | | | (GenBank | 2C-methyl-D- | braunii | | |
| | | | polynucleo- | erythritol kinase | | | |
| | | | tide | | | | |
| | | | sequence) | | | | |

2-C-methyl-D-erythritol 2,4-cyclodiphosphate synthase
Sequence example (SEQ ID NO: 74) (Arabidopsis thaliana):

| MATSSTQLLL SSSSLFHSQI TKKPFLLPAT KIGVWRPKKS LSLSCRPSAS VSAASSAVDV | | | | | | 60 | |
|---|---|---|---|---|---|---|---|
| NESVTSEKPT KTLPFRIGHG FDLHRLEPGY PLIIGGIVIP HDRGCEAHSD GDVLLHCVVD | | | | | | 120 | |
| AILGALGLPD IGQIFPDSDP KWKGAASSVF IKEAVRLMDE AGYEIGNLDA TLILQRPKIS | | | | | | 180 | |
| PHKETIRSNL SKLLGADPSV VNLKAKTHEK VDSLGENRSI AAHIVILLMK K | | | | | | 231 | |
| SEQ ID | | | | | | | |
| NO: | Taxon | Entry | Entry name | Protein names | Organism | Length | Gene |
| 75 | Bacteria | Q2NAE1 | ISPDF_ERYLH | Bifunctional enzyme | Erythrobacter | 386 | ispDF |
| | | | | IspD/IspF [Includes: | litoralis (strain | | ELI_06290 |
| | | | | 2-C-methyl-D- | HTCC2594) | | |
| | | | | erythritol 4- | | | |
| | | | | phosphate | | | |
| | | | | cytidylyltransferase | | | |
| | | | | (EC 2.7.7.60)(4- | | | |
| | | | | diphosphocytidy1-2C- | | | |
| | | | | methyl-D-erythritol | | | |
| | | | | synthase)(MEP | | | |
| | | | | cytidylyltransferase) | | | |
| | | | | (MCT); 2-C-methyl-D- | | | |
| | | | | erythritol 2,4- | | | |
| | | | | cyclodiphosphate | | | |
| | | | | synthase (MECDP- | | | |
| | | | | synthase)(MECPS) | | | |
| | | | | (EC 4.6.1.12)] | | | |
| 76 | Bacteria | B9E8S0 | B9E8S0_MACCJ | 2-C-methyl-D- | Macrococcus | 159 | ispF |
| | | | | erythritol 2,4- | caseolyticus | | MCCL_1881 |
| | | | | cyclodiphosphate | (strain JCSC5402) | | |
| | | | | synthase (MECDP- | | | |
| | | | | synthase)(MECPS) | | | |
| | | | | (EC 4.6.1.12) | | | |
| 77 | Fungi | Q2U5Q5 | Q2U5Q5_ASPOR | Putative | Aspergillus | 420 | AO090113000049 |
| | | | | uncharacterized | oryzae (strain | | |
| | | | | protein | ATCC 42149 / RIB | | |
| | | | | AO090113000049 | 40) | | |
| 78 | Fungi | Q0CZ74 | Q0CZ74_ASPTN | 2-C-methyl-D- | Aspergillus | 933 | ATEG_01010 |
| | | | | erythritol 2,4- | terreus (strain | | |
| | | | | cyclodiphosphate | NIH 2624 / FGSC | | |
| | | | | synthase | A1156) | | |
| 79 | Plantae | A9ZN13 | A9ZN13_HEVBR | 2-C-methyl-D- | Hevea brasiliensis | 241 | HbMCS |
| | | | | erythritol 2,4- | (Para rubber | | |
| | | | | cyclodiphosphate | tree)(Siphonia | | |
| | | | | synthase (EC | brasiliensis) | | |
| | | | | 4.6.1.12) | | | |
| 80 | Plantae | B6E1X5 | B6E1X5_HEVBR | 2-C-methyl-D- | Hevea brasiliensis | 238 | |
| | | | | erythritol 2,4- | (Para rubber | | |
| | | | | cyclodiphosphate | tree)(Siphonia | | |
| | | | | synthase (EC | brasiliensis) | | |
| | | | | 4.6.1.12) | | | |
| 81 | Plantae | A1KXW3 | A1KXW3_HEVBR | 2-C-methyl-D- | Hevea brasiliensis | 238 | ISPF |
| | | | | erythritol 2,4- | (Para rubber | | |
| | | | | cyclodiphosphate | tree)(Siphonia | | |
| | | | | synthase (EC | brasiliensis) | | |
| | | | | 4.6.1.12) | | | |
| 82 | Plantae | A9ZN12 | A9ZN12_HEVBR | 2-12-methyl-D- | Hevea brasiliensis | 237 | HbMCS |
| | | | | erythritol 2,4- | (Para rubber | | |
| | | | | cyclodiphosphate | tree)(Siphonia | | |
| | | | | synthase (EC | brasiliensis) | | |
| | | | | 4.6.1.12) | | | |
| 159 | Plantae | | EZ228118 | 2-12-methyl-D- | Artemisia annua | 226 | |
| | | | (GenBank | erythritol 2,4- | | | |
| | | | mRNA | cyclodiphosphate | | | |
| | | | polynucleo- | synthase | | | |
| | | | tide sequence) | | | | |
| 173 | Bacteria | | AAC75788 | 2-12-methyl-D- | E. coli | 159 | |
| | | | (GenBank | erythritol 2,4- | | | |
| | | | polynucleo- | cyclodiphosphate | | | |
| | | | tide sequence) | synthase | | | |
| 184 | Algae | | KA659951 | 2-12-methyl-D- | Botrycoccus | 239 | |
| | | | (GenBank | erythritol 2,4- | braunii | | |
| | | | polynucleo- | cyclodiphosphate | | | |
| | | | tide | synthase | | | |
| | | | sequence) | | | | |

4-hydroxy-3-methylbut-2-en-1-yl diphosphate synthase
Sequence example (SEQ ID NO: 83, Arabidopsis thaliana):

| MATGVLPAPV SGIKIPDSKV GFGKSMNLVR ICDVRSLRSA RRRVSVIRNS NQGSDLAELQ | | | | | | 60 | |
|---|---|---|---|---|---|---|---|
| PASEGSPLLV PRQKYCESLH KTVRRKTRTV MVGNVALGSE HPIRIQTMTT SDTKDITGTV | | | | | | 120 | |
| DEVMRIADKG ADIVRITVQG KKEADACFEI KDKLVQLNYN IPLVADIHFA PTVALRVAEC | | | | | | 180 | |
| FDKIRVNPGN FADRRAQFET IDYTEDEYQK ELQHIEQVFT PLVEKCKKYG RAMRIGINHG | | | | | | 240 | |
| SLSDRIMSYY GDSPRGMVES AFEFARICRK LDYHNFVFSM KASNPVIMVQ AYRLLVAEMY | | | | | | 300 | |
| VHGWDYPLHL GVTEAGEGED GRMKSAIGIG TLLQDGLGDT IRVSLTEPPE EEIDPCRRLA | | | | | | 360 | |
| NLGTKAAKLQ QGAPFEEKHR HYFDFQRRTG DLPVQKEGEE VDYRNVLHRD GSVLMSISLD | | | | | | 420 | |
| QLKAPELLYR SLATKLVVGM PFKDLATVDS ILLRELPPVD DQVARLALKR LIDVSMGVIA | | | | | | 480 | |
| PLSEQLTKPL PNAMVLVNLK ELSGGAYKLL PEGTRLVVSL RGDEPYEELE ILKNIDATMI | | | | | | 540 | |
| LHDVPFTEDK VSRVHAARRL FEFLSENSVN FPVIHHINFP TGIHRDELVI HAGTYAGGLL | | | | | | 600 | |
| VDGLGDGVML EAPDQDFDFL RNTSFNLLQG CRMRNIKIEY VSCPSCGRTL FDLQEISAEI | | | | | | 660 | |
| REKTSHLPGV SIAIMGCIVN GPGEMADADF GYVGGSPGKI DLYVGKTVVK RGIAMIEAID | | | | | | 720 | |
| ALIGLIKEHG RWVDPPVADE | | | | | | 740 | |
| SEQ ID | | | | | | | |
| NO: | Taxon | Entry | Entry name | Protein names | Organism | Length | Gene |
| 84 | Bacteria | F8L1N8 | F8L1N8_PARAV | 4-hydroxy-3- | Parachlamydia | 656 | ispG |
| | | | | methylbut-2-en-1-yl | acanthamoebae | | PUV_22380 |
| | | | | diphosphate | (strain UV7) | | |
| | | | | synthase (EC | | | |
| | | | | 1.17.7.1) | | | |
| 85 | Bacteria | Q6MD85 | ISPG_PARUW | 4-hydroxy-3- | Protochlamydia | 654 | ispG gcpE |
| | | | | methylbut-2-en-1-yl | amoebophila | | pc0740 |
| | | | | diphosphate | (strain UWE25) | | |
| | | | | synthase (EC | | | |
| | | | | 1.17.7.1)(1-hydroxy- | | | |
| | | | | 2-methy1-2-(E)- | | | |
| | | | | butenyl 4- | | | |
| | | | | diphosphate | | | |
| | | | | synthase) | | | |
| 86 | Bacteria | F8L7U6 | F8L7U6_SIMNZ | 4-hydroxy-3- | Simkania | 604 | ispG |
| | | | | methylbut-2-en-1-yl | negevensis | | SNE_A09 |
| | | | | diphosphate | (strain ATCC VR- | | 710 |
| | | | | synthase (EC | 1471 / Z) | | |
| | | | | 1.17.7.1) | | | |
| 87 | Fungi | F4SDS6 | F4SDS6_MELLP | Putative | Melampsora | 570 | MELLADRAFT_ |
| | | | | uncharacterized | larici-populina | | 70141 |
| | | | | protein | (strain 98AG31 / | | |
| | | | | | pathotype 3-4-7) | | |
| | | | | | (Poplar leaf rust | | |
| | | | | | fungus) | | |
| 88 | Fungi | Q6CV00 | Q6CV00_KLULA | KLLA0C01001p | Kluyveromyces | 429 | KLLA0C01001g |
| | | | | | lactis (strain ATCC | | |
| | | | | | 8585 / CBS 2359 / | | |
| | | | | | DSM 70799 / | | |
| | | | | | NBRC 1267 / | | |
| | | | | | NRRL Y-1140 / | | |
| | | | | | WM37)(Yeast) | | |
| | | | | | (Candida | | |
| | | | | | sphaerica) | | |
| 89 | Plantae | A9ZN14 | A9ZN14_HEVBR | 4-hydroxy-3- | Hevea brasiliensis | 740 | HbHDS |
| | | | | methylbut-2-en-1-yl | (Para rubber | | |
| | | | | diphosphate | tree)(Siphonia | | |
| | | | | synthase (EC | brasiliensis) | | |
| | | | | 1.17.4.3) | | | |
| 160 | Plantae | | EZ235247 | 4-hydroxy-3- | Artemisia annua | 742 | |
| | | | (GenBank | methylbut-2-en-1-yl | | | |
| | | | mRNA | diphosphate | | | |
| | | | polynucleo- | synthase | | | |
| | | | tide sequence) | | | | |
| 174 | Bacteria | | AAC75568 | 4-hydroxy-3- | E. coli | 372 | |
| | | | (GenBank | methylbut-2-en-1-yl | | | |
| | | | polynucleo- | diphosphate | | | |
| | | | tide sequence) | synthase | | | |
| 185 | Algae | | KA659952 | 4-hydroxy-3- | Botrycoccus | 737 | |
| | | | (Gen Bank | methylbut-2-en-1-yl | braunii | | |
| | | | polynucleo- | diphosphate | | | |
| | | | tide | synthase | | | |
| | | | sequence) | | | | |

4-hydroxy-3-methylbut-2-enyl diphosphate reductase
Sequence example (SEQ ID NO: 90, Arabidopsis thaliana):

| MAVALQFSRL CVRPDTFVRE NHLSGSGSLR RRKALSVRCS SGDENAPSPS VVMDSDFDAK | | | | | | 60 | |
|---|---|---|---|---|---|---|---|
| VFRKNLTRSD NYNRKGFGHK EETLKLMNRE YTSDILETLK TNGYTYSWGD VTVKLAKAYG | | | | | | 120 | |
| FCWGVERAVQ IAYEARKQFP EERLWITNEI IHNPTVNKRL EDMDVKIIPV EDSKKQFDVV | | | | | | 180 | |
| EKDDVVILPA FGAGVDEMYV LNDKKVQIVD TTCPWVTKVW NTVEKHKKGE YTSVIHGKYN | | | | | | 240 | |
| HEETIATASF AGKYIIVKNM KEANYVCDYI LGGQYDGSSS TKEEFMEKFK YAISKGFDPD | | | | | | 300 | |
| NDLVKVGIAN QTTMLKGETE EIGRLLETTM MRKYGVENVS GHFISFNTIC DATQERQDAI | | | | | | 360 | |
| YELVEEKIDL MLVVGGWNSS NTSHLQEISE ARGIPSYWID SEKRIGPGNK IAYKLHYGEL | | | | | | 420 | |
| VEKENFLPKG PITIGVTSGA STPDKVVEDA LVKVFDIKRE ELLQLA | | | | | | 466 | |
| SEQ ID | | | | | | | |
| NO: | Taxon | Entry | Entry name | Protein names | Organism | Length | Gene |
| 91 | Bacteria | B1WTZ2 | ISPH_CYAA5 | 4-hydroxy-3- | Cyanothece sp. | 402 | ispH |
| | | | | methylbut-2-enyl | (strain ATCC | | cce_1108 |
| | | | | diphosphate | 51142) | | |
| | | | | reductase (EC | | | |
| | | | | 1.17.1.2) | | | |
| 92 | Bacteria | D8FV73 | D8FV73_9CYAN | 4-hydroxy-3- | Oscillatoria sp. | 397 | ispH |
| | | | | methylbut-2-enyl | PCC 6506 | | OSCI_750007 |
| | | | | diphosphate | | | |
| | | | | reductase (EC | | | |
| | | | | 1.17.1.2) | | | |
| 93 | Bacteria | B0JVA7 | ISPH_MICAN | 4-hydroxy-3- | Microcystis | 402 | ispH |
| | | | | methylbut-2-enyl | aeruginosa (strain | | MAE_16190 |
| | | | | diphosphate | NIES-843) | | |
| | | | | reductase (EC | | | |
| | | | | 1.17.1.2) | | | |
| 94 | Fungi | Q5A2S3 | Q5A253_CANAL | Putative | Candida albicans | 1056 | GDH2 |
| | | | | uncharacterized | (strain SC5314 / | | Cao19.2192 |
| | | | | protein GDH2 | ATCC MYA-2876) | | |
| | | | | | (Yeast) | | |
| 95 | Fungi | Q10172 | PAN1_SCHPO | Actin cytoskeleton- | Schizosaccharomyces | 1794 | pan1 |
| | | | | regulatory complex | pombe | | SPAC25G10.09c |
| | | | | protein panl | (strain 972 / | | SPAC27F1.01c |
| | | | | | ATCC 24843) | | |
| | | | | | (Fission yeast) | | |
| 96 | Plantae | A9ZN15 | A9ZN15_HEVBR | 4-hydroxy-3- | Hevea brasiliensis | 462 | HbHDR |
| | | | | methylbut-2-enyl | (Para rubber | | |
| | | | | diphosphate | tree)(Siphonia | | |
| | | | | reductase (EC | brasiliensis) | | |
| | | | | 1.17.1.2) | | | |
| 97 | Plantae | BSAZS1 | B5AZS1_HEVBR | 4-hydroxy-3- | Hevea brasiliensis | 462 | |
| | | | | methylbut-2-enyl | (Para rubber | | |
| | | | | diphosphate | tree)(Siphonia | | |
| | | | | reductase | brasiliensis) | | |
| 161 | Plantae | | EZ205940 | 4-hydroxy-3- | Artemisia annua | 455 | |
| | | | (GenBank | methylbut-2-enyl | | | |
| | | | mRNA | diphosphate | | | |
| | | | polynucleo- | reductase | | | |
| | | | tide sequence) | | | | |
| 162 | Plantae | | EZ232255 | 4-hydroxy-3- | Artemisia annua | 454 | |
| | | | (GenBank | methylbut-2-enyl | | | |
| | | | mRNA | diphosphate | | | |
| | | | polynucleo- | reductase | | | |
| | | | tide sequence) | | | | |
| 163 | Plantae | | EZ245831 | 4-hydroxy-3- | Artemisia annua | 459 | |
| | | | (GenBank | methylbut-2-enyl | | | |
| | | | mRNA | diphosphate | | | |
| | | | polynucleo- | reductase | | | |
| | | | tide sequence) | | | | |
| 175 | Bacteria | | AAC73140 | 4-hydroxy-3- | E. coli | 316 | |
| | | | (GenBank | methylbut-2-enyl | | | |
| | | | polynucleo- | diphosphate | | | |
| | | | tide sequence) | reductase | | | |
| 186 | Algae | | KA659953 | 4-hydroxy-3- | Botrycoccus | 502 | |
| | | | (GenBank | methyl but-2-enyl | braunii | | |
| | | | polynucleo- | diphosphate | | | |
| | | | tide sequence) | reductase | | | |

TABLE 6
Exemplary IFF pathway sequences
Isopentenyl-diphosphate Delta-isomerase I
Sequence example (SEQ ID NO: 98, Artemisia annua):

| MSTASLFSFP SFHLRSLLPS LSSSSSSSSS RFAPPRLSPI RSPAPRTQLS VRAFSAVTMT | | | | | | 60 | |
|---|---|---|---|---|---|---|---|
| DSNDAGMDAV QRRLMFEDEC ILVDENDRVV GHDTKYNCHL MEKIEAENLL HRAFSVFLFN | | | | | | 120 | |
| SKYELLLQQR SKTKVTFPLV WTNTCCSHPL YRESELIEEN VLGVRNAAQR KLFDELGIVA | | | | | | 180 | |
| EDVPVDEFTP LGRMLYKAPS DGKWGEHEVD YLLFIVRDVK LQPNPDEVAE IKYVSREELK | | | | | | 240 | |
| ELVKKADAGD EAVKLSPWFR LVVDNFLMKW WDHVEKGTIT EAADMKTIHK L | | | | | | 291 | |
| SEQ ID | | | | | | | |
| NO | Taxon | Entry | Entry name | Protein names | Organism | Length | Gene |
| 99 | Plantae | A8DPG2 | A8DPG2_ARTAN | Isopenteyl | Artemisia annua | 284 | |
| | | | | diphosphate | (Sweet | | |
| | | | | isomerase | wormwood) | | |
| 100 | Plantae | A9ZN05 | A9ZN05_HEVBR | Isopentenyl- | Hevea brasiliensis | 234 | HblPI I |
| | | | | diphosphate Delta- | (Para rubber | | |
| | | | | isomerase (EC | tree) (Siphonia | | |
| | | | | 5.3.3.2) | brasiliensis) | | |
| 101 | Plantae | A9ZN04 | A9ZN04_HEVBR | Isopentenyl- | Hevea brasiliensis | 306 | HblPI II |
| | | | | diphosphate Delta- | (Para rubber | | |
| | | | | isomerase (EC | tree) (Siphonia | | |
| | | | | 5.3.3.2) | brasiliensis) | | |
| 190 | Plantae | | EZ203680 | Isopentenyl- | Artemisia annua | 281 | |
| | | | (GenBank | diphosphate Delta- | | | |
| | | | polynucleo- | isomerase | | | |
| | | | tide | | | | |
| | | | sequence) | | | | |
| 191 | Plantae | A8DPG2 | A8DPG2_ARTAN | Isopentenyl- | Artemisia annua | 284 | |
| | | | | diphosphate Delta- | | | |
| | | | | isomerase | | | |
| 192 | Bacteria | | AAC75927 | Isopentenyl- | E. coli | 182 | |
| | | | (GenBank | diphosphate Delta- | | | |
| | | | polynucleo- | isomerase | | | |
| | | | tide sequence) | | | | |

Isopentenyl-diphosphate Delta-isomerase II
Sequence example (SEQ ID NO: 102, Artemisia annua):

| MSASSLFNLP LIRLRSLALS SSFSSFRFAH RPLSSISPRK LPNFRAFSGT AMTDTKDAGM | | | | | | 60 | |
|---|---|---|---|---|---|---|---|
| DAVQRRLMFE DECILVDETD RVVGHDSKYN CHLMENIEAK NLLHRAFSVF LFNSKYELLL | | | | | | 120 | |
| QQRSNTKVTF PLVWTNTCCS HPLYRESELI QDNALGVRNA AQRKLLDELG IVAEDVPVDE | | | | | | 180 | |
| FTPLGRMLYK APSDGKWGEH ELDYLLFIVR DVKVQPNPDE VAEIKYVSRE ELKELVKKAD | | | | | | 240 | |
| AGEEGLKLSP WFRLVVDNFL MKWWDHVEKG TLVEAIDMKT IHKL | | | | | | 284 | |
| SEQ ID | | | | | | | |
| NO: | Taxon | Entry | Entry name | Protein names | Organism | Length | Gene |
| 103 | Plantae | A9ZN05 | A9ZN05_HEVBR | Isopentenyl- | Hevea brasiliensis | 234 | HblPI I |
| | | | | diphosphate Delta- | (Para rubber | | |
| | | | | isomerase (EC | tree)(Siphonia | | |
| | | | | 5.3.3.2) | brasiliensis) | | |
| 104 | Plantae | A8DPG2 | A8DPG2_ARTAN | Isopenteyl | Artemisia annua | 284 | |
| | | | | diphosphate | (Sweet | | |
| | | | | isomerase | wormwood) | | |
| 105 | Plantae | A9ZN04 | A9ZN04_HEVBR | Isopentenyl- | Hevea brasiliensis | 306 | HblPI II |
| | | | | diphosphate Delta- | (Para rubber | | |
| | | | | isomerase (EC | tree)(Siphonia | | |
| | | | | 5.3.3.2) | brasiliensis) | | |
| 106 | Plantae | Q9S7C4 | Q9S7C4_HEVBR | Isopentenyl | Hevea brasiliensis | 234 | IPI2 IPI1 |
| | | | | pyrophosphate | (Para rubber | | |
| | | | | isomerase (EC | tree)(Siphonia | | |
| | | | | 5.3.3.2) | brasiliensis) | | |
| 188 | Fungi | P15496 | IDI1_YEAST | Isopentenyl | S. cerevisiae | 288 | |
| | | | | pyrophosphate | | | |
| | | | | isomerase | | | |

Farnesyl diphosphate synthase
Sequence example (SEQ ID NO: 107, Artemisia annua):

| MASEKEIRRE RFLNVFPKLV EELNASLLAY GMPKEACDWY AHSLNYNTPG GKLNRGLSVV | | | | | | 60 | |
|---|---|---|---|---|---|---|---|
| DTYAILSNKT VEQLGQEEYE KVAILGWCIE LLQAYFLVAD DMMDKSITRR GQPCWYKVPE | | | | | | 120 | |
| VGEIAINDAF MLEAAIYKLL KSHFRNEKYY IDITELFHEV TFQTELGQLM DLITAPEDKV | | | | | | 180 | |
| DLSKFSLKKH SFIVTFKTAY YSFYLPVALA MYVAGITDEK DLKQARDVLI PLGEYFQIQD | | | | | | 240 | |
| DYLDCFGTPE QIGKIGTDIQ DNKCSWVINK ALELASAEQR KTLDENYGKK DSVAEAKCKK | | | | | | 300 | |
| IFNDLKIEQL YHEYEESIAK DLKAKISQVD ESRGFKADVL TAFLNKVYKR SK | | | | | | 352 | |
| SEQ ID | | | | | | | |
| NO | Taxon | Entry | Entry name | Protein names | Organism | Length | Gene |
| 108 | Plantae | Q8L7F4 | Q8L7F4_HEVBR | Farnesyl diphosphate | Hevea brasiliensis | 342 | FDP |
| | | | | synthase | (Para rubber | | |
| | | | | | tree)(Siphonia | | |
| | | | | | brasiliensis) | | |
| 109 | Plantae | A6N2H2 | A6N2H2_HEVBR | Farnesyl diphosphate | Hevea brasiliensis | 342 | |
| | | | | synthase isoform | (Para rubber | | |
| | | | | | tree)(Siphonia | | |
| | | | | | brasiliensis) | | |
| 110 | Plantae | P49350 | FPPS_ARTAN | Farnesyl | Artemisia annua | 343 | FPS1 |
| | | | | pyrophosphate | (Sweet | | |
| | | | | synthase(FPP | wormwood) | | |
| | | | | synthase)(FPS)(EC | | | |
| | | | | 2.5.1.10)((2E,6E)- | | | |
| | | | | farnesyl diphosphate | | | |
| | | | | synthase) | | | |
| | | | | (Dimethylallyltrans- | | | |
| | | | | transferase)(EC | | | |
| | | | | 2.5.1.1)(Farnesyl | | | |
| | | | | diphosphate | | | |
| | | | | synthase) | | | |
| | | | | (Geranyltranstrans- | | | |
| | | | | ferase) | | | |
| 111 | Plantae | Q9ZPJ3 | Q9ZPJ3_ARTAN | Farnesyl diphosphate | Artemisia annua | 343 | |
| | | | | synthase | (Sweet | | |
| | | | | | wormwood) | | |
| 164 | Plantae | | EZ240258 | Farnesyl diphosphate | Artemisia annua | 343 | |
| | | | (GenBank | | synthase | | |
| | | | mRNA | | | | |
| | | | polynucleo- | | | | |
| | | | tide sequence) | | | | |
| 165 | Plantae | | EZ204727 | Farnesyl diphosphate | Artemisia annua | 342 | |
| | | | (GenBank | | synthase | | |
| | | | mRNA | | | | |
| | | | polynucleo- | | | | |
| | | | tide sequence) | | | | |
| 176 | Bacteria | P22939 | P22939_ECOLI | Farnesyl diphosphate | E. coli | 299 | |
| | | | | synthase | | | |
| 187 | Algae | | KA659963 | Farnesyl diphosphate | Botrycoccus | 362 | |
| | | | (GenBank | synthase | braunii | | |
| | | | polynucleo- | | | | |
| | | | tide sequence) | | | | |
| 189 | Fungi | P08524 | FPPS_YEAST | Farnesyl diphosphate | S. cerevisiae | 352 | |
| | | | synthase | | | | |

β-farnesene synthase
Sequence example (SEQ ID NO: 112, Artemisia annua)

| MDTLPISSVS FSSSTSPLVV DDKVSTKPDV IRHTMNFNAS IWGDQFLTYD EPEDLVMKKQ | | | | | | 60 | |
|---|---|---|---|---|---|---|---|
| LVEELKEEVK KELITIKGSN EPMQHVKLIE LIDAVQRLGI AYHFEEEIEE ALQHIHVTYG | | | | | | 120 | |
| EQWVDKENLQ SISLWFRLLR QQGFNVSSGV FKDFMDEKGK FKESLCNDAQ GILALYEAAF | | | | | | 180 | |
| MRVEDETILD NALEFTKVHL DIIAKDPSCD SSLRTQIHQA LKQPLRRRLA RIEALHYMPI | | | | | | 240 | |
| YQQETSHDEV LLKLAKLDFS VLQSMHKKEL SHICKWWKDL DLQNKLPYVR DRVVEGYFWI | | | | | | 300 | |
| LSIYYEPQHA RTRMFLMKTC MWLVVLDDTF DNYGTYEELE IFTQAVERWS ISCLDMLPEY | | | | | | 360 | |
| MKLIYQELVN LHVEMEESLE KEGKTYQIHY VKEMAKELVR NYLVEARWLK EGYMPTLEEY | | | | | | 420 | |
| MSVSMVTGTY GLMIARSYVG RGDIVTEDTF KWVSSYPPII KASCVIVRLM DDIVSHKEEQ | | | | | | 480 | |
| ERGHVASSIE CYSKESGASE EEACEYISRK VEDAWKVINR ESLRPTAVPF PLLMPAINLA | | | | | | 540 | |
| RMCEVLYSVN DGFTHAEGDM KSYMKSFFVH PMVV | | | | | | 574 | |
| SEQ ID | | | | | | | |
| NO: | Taxon | Entry | Entry name | Protein names | Organism | Length | Gene |
| 113 | Plantae | E7BTW6 | E7BTW6_ARTAN | E-beta-farnesene | Artemisia annua | 574 | betaFS1 |
| | | | | synthase 1 | (Sweet | | |
| | | | | | wormwood) | | |
| 114 | Plantae | Q9AXP5 | Q9AXP5_ARTAN | Sesquiterpene | Artemisia annua | 573 | |
| | | | | cyclase | (Sweet | | |
| | | | | | wormwood) | | |
| 115 | Plantae | Q8SA63 | CARS_ARTAN | Beta-caryophyllene | Artemisia annua | 548 | QHS1 |
| | | | | synthase(EC | (Sweet | | |
| | | | | 4.2.3.57) | wormwood) | | |
| 166 | Plantae | Q9FXY7 | Q9FXY7_ARTAN | Beta-farnesene | Artemisia annua | 574 | |
| | | | | synthase | | | |
| 167 | Plantae | O48935 | 048935_MENPI | Beta farnesene | Mentha piperita | 550 | |
| | | | | synthase | | | |

α-farnesene synthase
Sequence example (SEQ ID NO: 116, Picea abies):

| MDLAVEIAMD LAVDDVERRV GDYHSNLWDD DFIQSLSTPY GASSYRERAE RLVGEVKEMF | | | | | | 60 | |
|---|---|---|---|---|---|---|---|
| TSISIEDGEL TSDLLQRLWM VDNVERLGIS RHFENEIKAA IDYVYSYWSD KGIVRGRDSA | | | | | | 120 | |
| VPDLNSIALG FRTLRLHGYT VSSDVFKVFQ DRKGEFACSA IPTEGDIKGV LNLLRASYIA | | | | | | 180 | |
| FPGEKVMEKA QIFAAIYLKE ALQKIQVSSL SREIEYVLEY GWLTNFPRLE ARNYIDVFGE | | | | | | 240 | |
| EICPYFKKPC IMVDKLLELA KLEFNLFHSL QQTELKHVSR WWKDSGFSQL TFTRHRHVEF | | | | | | 300 | |
| YTLASCIAIE PKHSAFRLGF AKVCYLGIVL DDIYDTFGKM KELELFIAAI KRWDPSTTEC | | | | | | 360 | |
| LPEYMKGVYM AFYNCVNELA LQAEKTQGRD MLNYARKAWE ALFDAFLEEA KWISSGYLPT | | | | | | 420 | |
| FEEYLENGKV SFGYRAAILQ PILTLDIPLP LHILQQIDFP SRFNDLASSI LRLRGDICGY | | | | | | 480 | |
| QAERSRGEEA SSISCYMKDN PGSTEEDALS HINAMISDNI NELNWELLKP NSNVPISSKK | | | | | | 540 | |
| HAFDILRAFY HLYKYRDGFS IAKIETKNLV MRTVLEPVPM | | | | | | 580 | |
| SEQ ID | | | | | | | |
| NO: | Taxon | Entry | Entry name | Protein names | Organism | Length | Gene |
| 117 | Plantae | Q94G53 | Q94G53_ARTAN | (-)-beta-pinene | Artemisia annua | 582 | QH6 |
| | | | | synthase | (Sweet | | |
| | | | | | wormwood) | | |
| 168 | Plantae | Q675K8 | Q675K8_PICAB | Alpha-farnesene | Picea abies | 580 | |
| | | | | synthase | | | |

TABLE 7
Examples of plant-optimized polynucleotide sequences

| | |
|---|---|
| SEQ | |
| ID | |
| NO | Sequence |

MVA Pathway

| | | | |
|---|---|---|---|
| 118 | Acetyl-CoA | GGATCCGAGC TCATGTCGCA AAATGTTTAT ATCGTTTCAA CTGCCCGCAC TCCAATCGGT | 60 |
| | acetyltransferase | TCCTTTCAGG GTTCTCTGTC GTCCAAGACT GCTGTCGAAC TTGGTGCAGT TGCCCTTAAG | 120 |
| | | GGAGCTTTGG CGAAGGTGCC CGAGCTGGAC GCCTCCAAGG ACTTCGATGA AATCATTTTT | 180 |
| | | GGTAACGTGC TCAGCGCTAA TCTGGGACAA GCACCAGCAA GACAGGTCGC ACTTGCAGCT | 240 |
| | | GGATTGTCTA ACCACATCGT TGCATCAACG GTTAATAAGG TGTGCGCTAG CGCGATGAAG | 300 |
| | | GCTATCATTC TCGGCGCGCA ATCTATTAAG TGCGGGAACG CAGATGTGGT CGTTGCCGGC | 360 |
| | | GGGTGTGAGT CCATGACCAA TGCGCCATAC TATATGCCAG CAGCAAGAGC AGGAGCAAAG | 420 |
| | | TTCGGGCAGA CAGTTCTCGT GGACGGCGTC GAGAGAGATG GGCTCAACGA CGCTTACGAT | 480 |
| | | GGTCTGGCGA TGGGAGTGCA CGCAGAAAAG TGTGCCCGGG ACTGGGATAT CACCAGAGAG | 540 |
| | | CAGCAAGACA ACTTCGCTAT TGAAAGCTAT CAGAAGTCCC AAAAGAGCCA GAAGGAGGGC | 600 |
| | | AAGTTCGATA ACGAGATCGT CCCAGTTACG ATTAAGGGCT TTAGGGGGAA GCCGGACACG | 660 |
| | | CAAGTGACTA AGGATGAGGA ACCTGCACGC CTTCATGTCG AGAAGTTGAG GTCTGCCCGC | 720 |
| | | ACTGTGTTCC AGAAGGAAAA CGGCACCGTC ACAGCCGCTA ACGCCTCTCC GATCAATGAC | 780 |
| | | GGGGCGGCAG CCGTCATTCT CGTTTCAGAG AAGGTCCTGA AGGAAAAGAA TCTCAAGCCC | 840 |
| | | CTGGCCATCA TTAAGGGTTG GGGAGAGGCT GCACACCAGC CAGCTGATTT CACCTGGGCT | 900 |
| | | CCTTCGCTTG CGGTTCCCAA GGCATTGAAG CATGCCGGTA TCGAGGACAT TAACTCAGTC | 960 |
| | | GATTACTTCG AGTTCAACGA GGCCTTCTCC GTGGTCGGCC TCGTGAACAC CAAGATCCTT | 1020 |
| | | AAGTTGGACC CGTCAAAAGT GAATGTCTAT GGTGGAGCTG TGGCACTCGG ACATCCTCTG | 1080 |
| | | GGTTGCTCGG GAGCACGCGT TGTGGTCACA CTCCTGTCCA TCCTGCAGCA AGAGGGCGGG | 1140 |
| | | AAGATTGGCG TTGCGGCTAT TTGTAACGGT GGGGGGGGGG CGTCCTCCAT CGTGATTGAA | 1200 |
| | | AAGATTTGAG GTACCTCTAG AAAGCTT | 1227 |
| 119 | Acetyl-CoA | CTGGATCCGA GCTCATGGCT CCCGTCGCCG CCGCTGAAAT CAAGCCGAGA GATGTGTGTA | 60 |
| | acetyltransferase | TTGTTGGTGT GGCACGCACT CCTATGGGTG GGTTCCTGGG TCTCCTGTCC ACGCTGCCTG | 120 |
| | | CGACTAAGCT CGGCAGCATC GCAATTGAGG CAGCTCTGAA GAGGGCATCG GTGGACCCAT | 180 |
| | | CCCTCGTTCA GGAAGTGTTC TTTGGTAACG TCTTGTCCGC AAATCTCGGA CAGGCTCCTG | 240 |
| | | CAAGACAAGC AGCACTGGGT GCAGGAATCC CCAACAGCGT GGTCTGCACC ACAGTCAATA | 300 |
| | | AGGTTTGTGC GTCAGGCATG AAGGCAACCA TGCTGGCCGC TCAGTCGATC CAACTTGGGA | 360 |
| | | TTAACGATGT TGTGGTCGCC GGCGGGATGG AGTCTATGTC AAATGCTCCA AAGTACCTCG | 420 |
| | | CAGAAGCCCG GAAGGGTAGC AGATTGGGAC ACGACTCTCT CGTGGATGGC ATGCTGAAGG | 480 |
| | | ACGGGCTTTG GGATGTTTAT AACGACGTGG GCATGGGGTC TTGCGCCGAG ATTTGCGCTG | 540 |
| | | ACAATCACTC AATTACGCGG GAAGACCAGG ATAAGTTCGC CATCCATTCG TTTGAGAGAG | 600 |
| | | GTATTGCGGC ACAAGAATCC GGAGCTTTCG CGTGGGAGAT CGTGCCAGTC GAAGTTTCTG | 660 |
| | | GTGGACGGGG CAAGCCGCTG ACTATTGTGG ACAAGGATGA GGGTCTCGGA AAGTTCGATC | 720 |
| | | CTGTCAAGCT GAGGAAGCTC CGCCCCTCCT TTAAGGAAAA CGGCGGGACC GTGACAGCGG | 780 |
| | | GCAATGCATC CAGCATCAGC GACGGAGCAG CTGCACTCAT TCTGGTTTCT GGCGAGACCG | 840 |
| | | CGCTTAAGTT GGGGCTCCAG GTCATCGCAA AGATTAGGGG ATACGCAGAC GCAGCACAAG | 900 |
| | | CTCCAGAGTT GTTCACGACT GCACCAGCCC TCGCTATCCC GAAGACAATT GCGAACGCAG | 960 |
| | | GCCTGGATGC CTCCCAGGTG GACTACTATG AGATCAACGA AGCCTTTGCT GTTGTGGCGT | 1020 |
| | | TGGCAAATCA AAAGCTCTTG GGCCTTAACC CAGAGAAAGT GAATGTCCAC GGTGGAGCCG | 1080 |
| | | TCTCATTGGG ACATCCACTC GGATGCTCGG GGGCTAGGAT TCTGGTCACA CTCCTGGGTG | 1140 |
| | | TTCTTCGCAA GAAGAACGCT AAGTATGGAG TGGGAGGAGT CTGTAATGGT GGAGGAGGAG | 1200 |
| | | CAAGCGCTCT CGTCGTTGAG CTTTTGTGAG GTACCTCTAG AAAGCTT | 1247 |
| 120 | Acetyl-CoA | GGATCCGAGC TCATGAAGAA CTGTGTTATT GTGTCAGCGG TTAGGACTGC CATTGGGTCT | 60 |
| | acetyltransferase | TTCAACGGGT CACTCGCCAG CACCTCTGCC ATCGACTTGG GCGCGACAGT CATCAAGGCC | 120 |
| | | GCTATTGAGA GGGCAAAGAT CGACTCTCAG CACGTGGATG AAGTCATTAT GGGTAACGTT | 180 |
| | | CTTCAGGCGG GGTTGGGTCA AAATCCTGCA CGCCAGGCCC TCCTGAAGTC CGGTCTCGCA | 240 |
| | | GAGACCGTTT GCGGATTCAC AGTTAACAAG GTCTGTGGAT CTGGCCTTAA GTCAGTGGCC | 300 |
| | | TTGGCAGCAC AGGCTATCCA AGCAGGACAG GCACAAAGCA TTGTCGCCGG CGGGATGGAG | 360 |
| | | AATATGTCTC TCGCTCCCTA CCTTTTGGAT GCTAAGGCAA GGAGCGGCTA CCGCCTGGGG | 420 |
| | | GACGGTCAGG TCTATGATGT TATCCTCAGG GACGGACTGA TGTGCGCAAC CCACGGATAC | 480 |
| | | CATATGGGCA TCACAGCGGA GAACGTCGCA AAGGAATATG GCATTACGCG GGAGATGCAA | 540 |
| | | GATGAACTTG CTTTGCATTC ACAGAGAAAG GCAGCTGCAG CAATCGAGTC GGGAGCCTTT | 600 |
| | | ACTGCTGAAA TTGTTCCAGT GAACGTGGTC ACGCGGAAGA AGACTTTCGT GTTTTCGCAG | 660 |
| | | GACGAGTTCC CAAAGGCCAA TTCCACGGCA GAAGCCCTTG GCGCCTTGAG ACCGGCTTTT | 720 |
| | | GATAAGGCGG GGACCGTTAC AGCGGGGAAC GCATCCGGTA TCAATGACGG AGCCGCTGCG | 780 |
| | | CTTGTGATTA TGGAGGAAAG CGCAGCATTG GCTGCAGGAC TCACCCCACT GGCGCGGATC | 840 |
| | | AAGTCCTATG CAAGCGGTGG AGTGCCACCA GCACTCATGG GAATGGGACC TGTCCCCGCA | 900 |
| | | ACACAGAAGG CCCTCCAACT GGCTGGCCTT CAATTGGCGG ACATCGATCT GATTGAGGCC | 960 |
| | | AACGAGGCCT TCGCAGCCCA GTTTCTCGCT GTCGGCAAGA ATCTGGGGTT CGATTCTGAG | 1020 |
| | | AAGGTCAACG TTAATGGCGG GGCTATCGCG CTGGGACACC CAATTGGAGC ATCAGGCGCC | 1080 |
| | | CGCATCCTCG TCACCCTCCT GCATGCCATG CAAGCTCGCG ACAAGACGCT CGGTCTGGCC | 1140 |
| | | ACTCTCTGTA TTGGTGGAGG CCAGGGAATC GCTATGGTCA TCGAGAGGCT GAATTAAGGT | 1200 |
| | | ACCAAGCTT | 1209 |
| 121 | 3-hydroxy-3- | GGATCCGAGC TCATGGCAAA GAATGTTGGT ATCCTGGCTA TGGACATCTA TTTCCCGCCC | 60 |
| | methylglutaryl | ACCTACGTTC AGCAAGAAGC ACTGGAGGCA CACGACGGCG CTTCCAAGGG CAAGTACACA | 120 |
| | coenzyme A synthase | ATCGGCCTTG GGCAGGACTG CATGGCGTTC TGTACGGAGG TCGAAGATGT TATTTCTATG | 180 |
| | | TCACTCACCG CAGTGACATC GCTCCTGGAG AAGTACAACA TCGACCCTAA TCAGATTGGT | 240 |
| | | CGGCTGGAGG TTGGATCTGA AACAGTGATC GATAAGTCGA AGTCCATTAA GACGTTCCTT | 300 |
| | | ATGCAAATCT TCGAGAAGTT TGGTAACACA GACATTGAAG GAGTGGATAG CGCTAATGCA | 360 |
| | | TGCTACGGAG GGACGGCAGC TTTGTTCAAC TGTGTGAATT GGGTCGAGAG CAACTCTTGG | 420 |
| | | GACGGCCGCT ACGGGCTGGT GGTCTGCACT GATAGCGCAG TCTATGCAGA AGGACCTGCT | 480 |
| | | AGACCAACCG GTGGAGCAGC AGCCATCGCG ATGCTGATTG GCCCAGAGGC TCCGATCGCG | 540 |
| | | TTCGAATCCA AGTTTAGGGG GTCTCACATG TCACATGCAT ACGACTTCTA TAAGCCAAAC | 600 |
| | | CTGGCCTCGG AGTACCCGGT TGTGGACGGC AAGCTCTCCC AGACCTGTTA TCTCATGGCA | 660 |
| | | CTGGATAGCT GCTACAAGCA CTTTTGTGCC AAGTATGAGA AGCTCGAAGG GAAGCAGTTC | 720 |
| | | TCAATCTCGG ACGCCGAGTA CTTCGTGTTT CATTCTCCAT ATAACAAGCT GGTCCAAAAG | 780 |
| | | TCATTTGCTC GGCTTGTCTT CAACGATTTT GTTAGAAATG CGTCCAGCAT TGACGATGCT | 840 |
| | | GCGAAGGAGA AGCTCGCCCC TTTCTCGACC TTGTCCGGCG ACGAGTCTTA CCAGAATAGG | 900 |
| | | GATCTGGAAA AGGTCTCACA GCAAGTTGCT AAGCCCTTGT ATGACGCGAA GGTTCAGCCT | 960 |
| | | ACCACACTCA TCCCCAAGCA AGTGGGTAAC ATGTACACTG CTTCCCTCTA TGCAGCCTTC | 1020 |
| | | GCGAGCCTTT TGCACAATAA GCATACCGAG CTGGCCGGCA AGCGCGTGAT CCTGTTCAGC | 1080 |
| | | TACGGTTCTG GACTTACGGC TACTATGTTT TCCCTTAGAT TGCACGAGGG CCAGCATCCA | 1140 |
| | | TTCTCCTTGA GCAACATTGC AACTGTTATG AATGTGGCCG GGAAGCTCAA GACCAGGCAC | 1200 |
| | | GAGTTCCCAC CGGAAAAGTT TGCAGTCATC ATGAAGCTGA TGGAGCATCG CTACGGTGCC | 1260 |
| | | AAGGACTTTG TTACATCAAA GGATTGCTCG ATTTTGGCGC CGGGAACGTA CTATCTCACT | 1320 |
| | | GAGGTCGACA CCATGTACAG GCGCTTCTAT GCACAAAAGG CCGTGGGCGA TACGGTCGAA | 1380 |
| | | AACGGCCTCC TGGCTAATGG GCACTGAGGT ACCTCTAGAA AGCTT | 1425 |
| 122 | 3-hydroxy-3- | GGATCCGAGC TCATGAAGCT GTCCACGAAG CTGTGCTGGT GCGGTATCAA GGGTAGACTG | 60 |
| | methylglutaryl | CGCCCCCAAA AGCAACAACA ACTCCATAAC ACGAATCTCC AAATGACGGA GCTGAAGAAG | 120 |
| | coenzyme A synthase | CAGAAGACGG CCGAACAAAA GACTCGGCCT CAGAACGTGG GCATCAAGGG CATCCAAATC | 180 |
| | | TACATCCCCA CTCAGTGCGT GAATCAATCG GAGCTTGAAA AGTTCGACGG TGTCTCCCAG | 240 |
| | | GGAAAGTATA CCATCGGCCT CGGGCAGACA AACATGTCTT TTGTCAATGA CCGGGAGGAT | 300 |
| | | ATCTACTCCA TGAGCCTCAC GGTTCTGTCC AAGCTCATCA AGTCATACAA CATCGACACT | 360 |
| | | AATAAGATCG GTAGATTGGA AGTGGGAACC GAAACACTCA TCGATAAGTC TAAGTCAGTC | 420 |
| | | AAGAGCGTTT TGATGCAGCT CTTCGGCGAG AACACGGACG TCGAAGGGAT TGATACTCTC | 480 |
| | | AACGCGTGCT ACGGCGGGAC AAATGCATTG TTTAACTCTC TCAATTGGAT CGAGTCAAAT | 540 |
| | | GCGTGGGACG GTCGGGATGC AATTGTGGTC TGTGGAGACA TTGCTATCTA CGATAAGGGA | 600 |
| | | GCAGCTAGAC CTACCGGTGG AGCAGGTACA GTGGCAATGT GGATCGGACC AGACGCCCCG | 660 |
| | | ATTGTCTTCG ATTCCGTTAG GGCCAGCTAC ATGGAGCACG CTTACGACTT CTATAAGCCA | 720 |
| | | GATTTTACCA GCGAATACCC GTATGTCGAC GGCCATTTCT CTCTGACATG CTATGTGAAG | 780 |
| | | GCCCTTGATC AGGTCTACAA GTCGTATTCC AAGAAGGCTA TCTCGAAGGG ACTGGTTTCC | 840 |
| | | GACCCTGCAG GGAGCGATGC TCTGAACGTG CTTAAGTACT TCGACTATAA TGTGTTTCAC | 900 |
| | | GTCCCCACGT GTAAGCTCGT TACTAAGTCC TACGGCCGGC TCCTGTATAA CGACTTCAGA | 960 |
| | | GCCAATCCTC AATTGTTTCC CGAGGTCGAT GCCGAACTGG CTACCAGGGA CTACGATGAG | 1020 |
| | | TCACTGACCG ACAAGAACAT CGAAAAGACA TTCGTTAATG TGGCGAAGCC ATTTCATAAG | 1080 |
| | | GAGCGCGTTG CACAGAGCCT CATTGTGCCG ACGAACACTG GCAATATGTA CACAGCCAGC | 1140 |
| | | GTGTATGCGG CATTCGCTTC TCTTTTGAAC TACGTCGGCT CAGACGATTT GCAAGGCAAG | 1200 |
| | | CGCGTTGGGC TCTTTAGCTA CGGTTCTGGA CTGGCCGCTT CACTTTATTC GTGTAAGATC | 1260 |
| | | GTTGGCGACG TGCAGCACAT CATTAAGGAG TTGGATATCA CGAACAAGCT CGCGAAGAGG | 1320 |
| | | ATTACCGAGA CACCAAAGGA CTACGAAGCG GCAATCGAGC TGCGCGAAAA CGCACACCTT | 1380 |
| | | AAGAAGAATT TCAAGCCGCA AGGGTCGATC GAGCATCTGC AGTCCGGTGT GTACTATCTT | 1440 |
| | | ACCAACATTG ACGATAAGTT CAGGCGCTCC TACGATGTCA AGAAGTAAGG TACCAAGCTT | 1500 |
| 123 | 3-hydroxy-3- | GGATCCGAGC TCATGGATGT TAGGAGAAGA CCAACCAGCG GCAAGACGAT TCATTCCGTT | 60 |
| | methylglutaryl | AAGCCCAAGT CAGTGGAGGA CGAGTCGGCA CAGAAGCCCT CCGACGCCTT GCCACTCCCG | 120 |
| | coenzyme A reductase | CTGTACCTTA TCAACGCTCT CTGCTTCACA GTGTTCTTTT ACGTGGTCTA TTTTCTCCTG | 180 |
| | | TCGCGGTGGA GAGAAAAGAT TCGCACGTCC ACTCCCCTTC ACGTTGTGGC TTTGAGCGAG | 240 |
| | | ATCGCCGCTA TTGTCGCGTT CGTTGCATCT TTTATCTATC TTTTGGGGTT CTTTGGTATC | 300 |
| | | GATTTCGTCC AGTCATTGAT TCTCCGGCCA CCGACGGACA TGTGGGCCGT TGACGATGAC | 360 |
| | | GAGGAAGAGA CAGAAGAGGG CATTGTGCTC CGGGAGGATA CGAGAAAGCT GCCGTGCGGG | 420 |
| | | CAAGCCCTTG ACTGTTCATT GTCGGCGCCT CCCCTCTCTA GGGCAGTCGT TTCCAGCCCC | 480 |
| | | AAGGCCATGG ACCCAATCGT CCTGCCTAGC CCCAAGCCAA AGGTTTTCGA CGAAATTCCG | 540 |
| | | TTTCCTACCA CAACGACTAT CCCCATTCTC GGCGATGAGG ACGAAGAGAT CATTAAGTCG | 600 |
| | | GTGGTCGCGG GCACTATCCC ATCCTACAGC CTCGAATCCA AGCTGGGGGA TTGCAAGAGA | 660 |
| | | GCAGCAGCAA TCAGGAGAGA GGCACTCCAG AGGATTACCG GAAAGTCTCT GTCAGGCCTG | 720 |
| | | CCCCTTGAAG GGTTCGACTA CGAGAGCATC CTGGGCCAGT GCTGTGAGAT GCCAGTGGGG | 780 |
| | | TATGTCCAAA TCCCGGTGGG AATTGCCGGC CCTCTCCTGC TTGATGGCAA GGAATATAGC | 840 |
| | | GTGCCAATGG CCACCACAGA GGGTTGCCTG GTCGCTTCTA CCAACCGCGG CTGTAAGGCC | 900 |
| | | ATCCATCTTT CCGGAGGAGC TACGAGCGTC TTGCTCAGGG ATGGCATGAC TAGGGCCCCA | 960 |
| | | GTTGTGCGGT TCGGGACCGC AAAGAGAGCT GCACAGTTGA AGCTCTACCT GGAAGACCCT | 1020 |
| | | GCCAACTTTG AGACCCTCTC GACATCCTTC AATAAGTCTT CAAGGTTTGG TCGCCTTCAA | 1080 |
| | | TCCATCAAGT GCGCAATTGC CGGAAAGAAT CTCTATATGC GCTTCTGCTG TTCTACAGGG | 1140 |
| | | GACGCCATGG GTATGAACAT GGTGTCAAAG GGCGTTCAGA ACGTGCTCAA TTTCCTGCAA | 1200 |
| | | AATGATTTTC CGGATATGGA CGTGATCGGG CTGTCTGGTA ACTTCTGCTC AGACAAGAAG | 1260 |
| | | CCTGCAGCCG TCAATTGGAT TGAAGGAAGG GGCAAGAGCG TCGTTTGTGA GGCGATCATT | 1320 |
| | | AAGGGCGACG TGGTCAAGAA GGTGCTCAAG ACTAACGTGG AAGCACTTGT CGAGTTGAAC | 1380 |
| | | ATGCTCAAGA ATCTGACCGG TTCAGCTATG GCGGGAGCAC TGGGTGGATT CAACGCCCAC | 1440 |
| | | GCTTCGAATA TCGTCACCGC CATCTACATT GCTACAGGCC AGGACCCAGC GCAAAACGTC | 1500 |
| | | GAATCGTCCA ATTGCATCAC AATGATGGAG GCAGTTAATG ATGGTCAGGA CCTCCATGTT | 1560 |
| | | TCGGTGACGA TGCCATCCAT TGAGGTCGGC ACGGTTGGCG GGGGTACTCA GCTTGCGAGC | 1620 |
| | | CAATCTGCAT GTTTGAACCT GCTTGGAGTG AAGGGAGCAT CCAAGGAGAC CCCAGGTGCA | 1680 |
| | | AATAGCAGAG TCCTTGCCTC TATCGTTGCT GGATCAGTGT TGGCTGCGGA GCTTTCATTG | 1740 |
| | | ATGTCGGCCA TTGCAGCCGG CCAGCTGGTT AACTCCCACA TGAAGTACAA CAGGGCTAAT | 1800 |
| | | AAGGAGGCTG CGGTCAGCAA GCCTAGCTCT TGAGGTACCT CTAGAAAGCT T | 1851 |
| 124 | 3-hydroxy-3- | GGATCCGAGC TCATGGCTGC CGATCAACTG GTGAAGACCG AGGTTACTAA GAAGTCGTTT | 60 |
| | methylglutaryl | ACTGCCCCTG TCCAAAAGGC GTCCACTCCC GTGCTGACCA ACAAGACCGT TATCTCGGGT | 120 |
| | coenzyme A reductase | TCCAAGGTGA AGTCCCTCTC CAGCGCCCAG TCTTCATCGT CCGGACCATC CTCCTCCTCC | 180 |
| | | GAGGAAGACG ATTCGCGGGA CATCGAGTCC CTGGATAAGA AGATTAGACC TCTCGAGGAA | 240 |
| | | CTGGAAGCCC TCCTGTCCAG CGGCAACACA AAGCAACTCA AGAATAAGGA GGTTGCCGCT | 300 |
| | | CTCGTGATCC ACGGCAAGCT CCCCTTGTAC GCTCTTGAAA AGAAGTTGGG AGACACCACA | 360 |
| | | AGGGCGGTTG CAGTGAGGCG CAAGGCGCTT TCGATTTTGG CCGAGGCTCC GGTGCTCGCA | 420 |
| | | TCAGATAGGC TGCCTTATAA GAACTACGAC TATGATCGCG TGTTCGGCGC CTGCTGTGAG | 480 |
| | | AATGTCATCG GGTACATGCC ACTTCCGGTC GGTGTTATCG GACCCCTCGT GATCGACGGC | 540 |
| | | ACATCTTATC ATATCCCAAT GGCGACGACT GAGGGTTGCC TCGTCGCAAG CGCAATGAGA | 600 |
| | | GGCTGTAAGG CCATTAACGC TGGCGGGGGT GCAACCACAG TGCTGACTAA GGACGGTATG | 660 |
| | | ACCAGGGGAC CAGTGGTCCG CTTCCCTACG CTTAAGCGCT CTGGCGCCTG CAAGATTTGG | 720 |
| | | CTCGATTCAG AGGAAGGGCA GAACGCGATT AAGAAGGCAT TCAATAGCAC ATCTAGGTTT | 780 |
| | | GCGCGCCTCC AGCACATCCA AACGTGTCTG GCAGGTGACC TTTTGTTCAT GCGGTTTAGA | 840 |
| | | ACAACTACCG GCGATGCTAT GGGGATGAAT ATGATTTCAA AGGGCGTTGA GTACTCGCTC | 900 |
| | | AAGCAAATGG TGGAGGAATA TGGTTGGGAG GACATGGAAG TTGTGTCAGT GTCGGGAAAC | 960 |
| | | TACTGCACTG ATAAGCCCGC GGCAATCAAT TGGATTGAGG GAAGGGGGAA GTCCGTCGTT | 1020 |
| | | GCAGAAGCTA CCATCCCAGG CGACGTGGTC AGAAAGGTCC TGAAGTCTGA TGTCTCAGCC | 1080 |
| | | CTCGTTGAGC TGAACATTGC TAAGAATCTT GTCGGTAGCG CGATGGCAGG ATCTGTTGGA | 1140 |
| | | GGCTTCAACG CCCATGCCGC TAATCTGGTG ACAGCCGTCT TTCTCGCTCT GGGCCAGGAC | 1200 |
| | | CCTGCTCAAA ACGTGGAGTC TTCAAATTGC ATCACGCTCA TGAAGGAAGT CGACGGGGAT | 1260 |
| | | CTGCGGATTT CCGTCAGCAT GCCGAGCATC GAGGTTGGCA CAATTGGGGG TGGAACGGTT | 1320 |
| | | CTTGAACCTC AGGGGGCGAT GTTGGATCTC CTGGGCGTCA GAGGACCACA CGCAACAGCT | 1380 |
| | | CCAGGCACGA ACGCGCGGCA ACTCGCAAGA ATCGTGGCAT GCGCAGTCCT GGCAGGAGAG | 1440 |
| | | CTTTCCTTGT GTGCGGCACT TGCCGCTGGG CATTTGGTGC AGAGCCACAT GACTCATAAC | 1500 |
| | | AGGAAGCCTG CCGAGCCCAC TAAGCCAAAC AATCTTGACG CTACCGATAT CAATCGCTTG | 1560 |
| | | AAGGACGGCT CCGTCACCTG CATTAAGAGC TAAGGTACCA AGCTT | 1605 |
| 125 | Mevalonate kinase | GGATCCGAGC TCATGGAAGT CAAGGCAAGG GCTCCGGGCA AGATTATTCT CAGCGGGGAA | 60 |
| | | CACGCAGTCG TTCACGGGTC TACAGCGGTG GCGGCATCGA TCAACCTGTA CACGTATGTC | 120 |
| | | ACTCTTTCGT TCGCCACCGC TGAGAATGAC GATTCTCTTA AGTTGCAGCT CAAGGACCTG | 180 |
| | | GCGCTTGAAT TTTCATGGCC AATCGGAAGG ATTCGCGAGG CCTTGTCCAA CCTCGGCGCT | 240 |
| | | CCGTCCAGCT CTACGAGGAC TTCTTGCTCC ATGGAGTCTA TCAAGACAAT TTCAGCCCTG | 300 |
| | | GTGGAGGAAG AGAATATCCC GGAGGCCAAG ATTGCTCTCA CCTCAGGGGT CTCGGCGTTC | 360 |
| | | TTGTGGCTCT ACACAAGCAT CCAAGGTTTT AAGCCTGCAA CCGTGGTCGT TACAAGCGAT | 420 |
| | | CTGCCCCTTG GCTCTGGGCT GGGTTCATCG GCCGCTTTCT GTGTCGCCCT TTCCGCGGCA | 480 |
| | | CTCCTGGCTT TTTCGGACTC CGTTAACGTG GATACCAAGC ACCTGGGGTG GTCGATCTTC | 540 |
| | | GGTGAATCCG ACTTGGAGCT TTTGAATAAG TGGGCCCTCG AAGGCGAGAA GATCATTCAT | 600 |
| | | GGAAAGCCTT CAGGCATTGA TAACACGGTG TCGGCTTATG GAAATATGAT CAAGTTCAAG | 660 |
| | | TCTGGCAACC TCACTCGGAT TAAGTCAAAT ATGCCCCTGA AGATGCTTGT TACCAACACA | 720 |
| | | CGGGTGGGGA GAAATACGAA GGCGTTGGTC GCAGGTGTTA GCGAGAGGAC TCTCCGCCAC | 780 |
| | | CCAAACGCGA TGTCTTTCGT GTTTAATGCA GTCGACAGCA TCTCTAACGA GCTGGCCAAT | 840 |
| | | ATCATTCAGT CCCCAGCTCC GGACGATGTG AGCATTACGG AAAAGGAAGA GAAGTTGGAA | 900 |
| | | GAGCTGATGG AGATGAACCA GGGGCTCCTG CAATGCATGG GTGTCTCCCA TGCTAGCATC | 960 |
| | | GAGACCGTTC TGCGCACCAC ACTTAAGTAC AAGTTGGCAT CCAAGCTCAC AGGAGCAGGA | 1020 |
| | | GGAGGTGGAT GTGTTCTCAC GCTTTTGCCA ACTCTCCTGT CCGGCACCGT GGTCGATAAG | 1080 |
| | | GCGATTGCAG AACTGGAGTC CTGCGGCTTC CAATGTCTTA TCGCCGGAAT TGGCGGGAAC | 1140 |
| | | GGCGTGGAGT TCTGCTTTGG TGGCTCCTCC TGAGGTACCT CTAGAAAGCT T | 1191 |
| 126 | Mevalonate kinase | GGATCCGAGC TCATGTCTCT CCCATTTCTT ACTTCCGCCC CAGGCAAGGT CATTATTTTT | 60 |
| | | GGTGAACACT CAGCAGTCTA CAACAAGCCA GCAGTCGCAG CTTCGGTCTC CGCGCTGAGG | 120 |
| | | ACTTACCTCC TGATCTCGGA GTCCAGCGCC CCTGACACCA TCGAACTCGA CTTCCCCGAT | 180 |
| | | ATTTCTTTTA ACCACAAGTG GTCAATCAAC GACTTCAATG CAATTACTGA GGATCAGGTC | 240 |
| | | AATTCTCAAA AGCTGGCGAA GGCACAGCAA GCCACCGACG GCCTGTCCCA GGAGCTTGTT | 300 |
| | | AGCCTTCTCG ACCCACTCCT GGCTCAACTC AGCGAATCTT TCCACTACCA TGCCGCTTTC | 360 |
| | | TGCTTTTTGT ATATGTTTGT TTGCCTCTGT CCACATGCTA AGAACATCAA GTTCAGCTTG | 420 |
| | | AAGTCTACCC TCCCGATTGG CGCTGGGCTG GGTTCTTCAG CGTCAATCTC GGTGTCCTTG | 480 |
| | | GCCCTCGCTA TGGCGTATTT GGGCGGGCTC ATTGGGTCGA ACGACCTGGA GAAGCTCTCC | 540 |
| | | GAAAACGATA AGCACATCGT GAATCAGTGG GCCTTCATCG GCGAGAAGTG TATTCATGGA | 600 |
| | | ACACCTTCTG GCATTGACAA CGCAGTCGCC ACGTACGGAA ATGCTCTTTT GTTTGAGAAG | 660 |
| | | GATTCACACA ACGGCACAAT CAATACGAAC AATTTCAAGT TTCTCGACGA TTTCCCAGCG | 720 |
| | | ATCCCGATGA TTCTGACTTA TACCCGCATC CCACGCAGCA CAAAGGACCT GGTTGCACGG | 780 |
| | | GTGAGAGTCC TTGTTACGGA GAAGTTCCCT GAAGTGATGA AGCCCATTCT GGATGCAATG | 840 |
| | | GGAGAGTGCG CCTTGCAGGG CCTCGAAATC ATGACAAAGC TCTCCAAGTG TAAGGGTACA | 900 |
| | | GACGATGAGG CCGTCGAAAC GAACAATGAG TTGTACGAAC AACTCCTGGA GCTTATCCGG | 960 |
| | | ATTAACCACG GCCTTTTGGT GTCAATCGGG GTCTCGCATC CGGGTCTGGA ACTTATCAAG | 1020 |
| | | AATCTGAGCG ACGATCTTCG CATTGGGTCT ACTAAGCTCA CCGGTGCAGG TGGAGGAGGA | 1080 |
| | | TGCTCCCTCA CTCTCCTGAG GAGAGACATC ACCCAGGAGC AAATTGATTC CTTCAAGAAG | 1140 |
| | | AAGCTCCAGG ACGATTTCTC GTATGAGACA TTTGAAACGG ACCTCGGTGG AACGGGCTGC | 1200 |
| | | TGTCTTTTGT CCGCAAAGAA CTTGAATAAG GATCTCAAGA TTAAGAGCCT GGTTTTCCAG | 1260 |
| | | CTTTTTGAGA ACAAGACCAC AACGAAGCAG CAAATCGACG ATCTCCTGCT TCCAGGCAAC | 1320 |
| | | ACTAATCTCC CGTGGACCAG CTAAGGTACC AAGCTT | 1356 |
| 127 | Phosphomevalonate | GGATCCGAGC TCATGGCAGT CGTTGCGTCC GCTCCAGGGA AGGGTGTTAT GACAGGGGGC | 60 |
| | kinase | TATCTTATTC TTGAGAGACC AAATGCAGGT ATCGTGCTTT CCACGAACGC TAGGTTCTAC | 120 |
| | | GCGATCGTTA AGCCTATGTA TGACGAAATT AAGCCCGATT CTTGGGCATG GGCCTGGACC | 180 |
| | | GACGTGAAGC TCACATCACC ACAGCTGGCC AGGGAGTCGC TTTACAAGCT CTCCCTCAAG | 240 |
| | | AACCTCGCAC TGCAATGCGT CTCCAGCTCT GCCTCCCGCA ATCCGTTCGT TGAGCAGGCA | 300 |
| | | GTGCAATTTG CAGTCGCAGC TGCACACGCA ACCCTGGACA AGGATAAGAA CAATGTGCTT | 360 |
| | | AACAAGCTCC TGCTTCAGGG CTTGGACATC ACGATTCTGG GGACTTCCGA TTGCTATAGC | 420 |
| | | TGTCGCAATG AGATCGAAGC GTGCGGCCTT CCTTTGACGC CCGAATCACT CGCAGCCCTG | 480 |
| | | CCTTCGTTCT CATCGATTAC TTTTAACGTC GAGGAAGCTA ACGGGCAGAA TTGTAAGCCA | 540 |
| | | GAGGTTGCAA AGACCGGACT GGGGTCCAGC GCTGCAATGA CCACAGCTGT GGTCGCAGCC | 600 |
| | | TTGCTCCACC ATCTCGGCCT GGTGGACCTC TCTTCATCGT GCAAGGAGAA GAAGTTCAGC | 660 |
| | | GACCTTGATT TGGTGCACAT CATTGCACAG ACAGCCCATT GTATCGCACA AGGCAAGGTC | 720 |
| | | GGTTCTGGAT TCGATGTTTC CAGCGCCGTG TACGGATCTC ACAGGTATGT TCGCTTTTCA | 780 |
| | | CCAGAGGTGC TGTCTTCAGC TCAGGACGCG GGCAAGGGGA TTCCGCTGCA AGAAGTCATC | 840 |
| | | AGCAACATTC TCAAGGGCAA GTGGGATCAT GAGCGGACGA TGTTCTCCCT TCCACCGTTG | 900 |
| | | ATGAGCCTGC TTTTGGGCGA GCCAGGAACG GGAGGGTCGT CCACTCCATC CATGGTGGGC | 960 |
| | | GCCCTCAAGA AGTGGCAGAA GAGCGACACC CAGAAGTCTC AAGAGACATG GAGGAAGCTC | 1020 |
| | | TCTGAGGCAA ACTCAGCCCT CGAAACTCAG TTCAACATCC TCAGCAAGCT GGCTGAGGAA | 1080 |
| | | CACTGGGACG CGTACAAGTG CGTCATCGAT TCATGTTCGA CCAAGAACTC CGAGAAGTGG | 1140 |
| | | ATTGAACAGG CTACAGAGCC TTCCAGGGAA GCTGTTGTGA AGGCGCTCCT GGGCAGCCGC | 1200 |
| | | AACGCAATGC TGCAGATCCG GAATTATATG AGACAAATGG GAGAGGCTGC AGGGGTGCCA | 1260 |
| | | ATTGAGCCGG AATCCCAGAC CCGGCTTTTG GACACGACTA TGAACATGGA TGGAGTCCTC | 1320 |
| | | CTGGCAGGCG TTCCGGGAGC AGGTGGATTC GACGCTGTCT TTGCGGTTAC GCTCGGCGAC | 1380 |
| | | AGCGGAACTA ACGTCGCTAA GGCCTGGTCC TCCCTCAACG TGTTGGCCCT TTTGGTCCGG | 1440 |
| | | GAGGACCCTA ATGGTGTTCT CCTGGAATCG GGAGATCCCA GAACAAAGGA GATCACCACA | 1500 |
| | | GCAGTGTCCG CCGTCCATAT TTGAGGTACC TCTAGAAAGC TT | 1542 |
| 128 | Phosphomevalonate | GGATCCGAGC TCATGTCGGA ACTCAGAGCA TTTTCGGCAC CGGGGAAGGC ACTGTTGGCA | 60 |
| | kinase | GGTGGTTATC TTGTTTTGGA CCCTAAGTAT GAAGCATTTG TGGTCGGACT TAGCGCAAGA | 120 |
| | | ATGCACGCAG TCGCTCATCC TTACGGGTCG TTGCAGGAGT CCGACAAGTT CGAAGTTAGA | 180 |
| | | GTGAAGAGCA AGCAGTTCAA GGATGGCGAG TGGCTGTATC ACATCTCTCC AAAGACAGGA | 240 |
| | | TTCATCCCGG TGAGCATTGG CGGGTCTAAG AACCCTTTTA TCGAGAAGGT CATCGCCAAC | 300 |
| | | GTCTTCTCAT ACTTTAAGCC CAATATGGAC GATTATTGCA ACAGGAATCT CTTCGTTATC | 360 |
| | | GACATCTTCT CCGACGATGC TTACCACTCA CAGGAGGATT CGGTGACCGA ACATCGGGGC | 420 |
| | | AATAGGCGCC TTTCTTTCCA CTCACATAGA ATCGAGGAAG TCCCAAAGAC TGGCTTGGGG | 480 |
| | | TCCAGCGCTG GGTTGGTCAC CGTTCTCACC ACAGCGCTGG CATCCTTCTT TGTGAGCGAC | 540 |
| | | CTCGAGAACA ATGTGGATAA GTACAGGGAG GTCATCCACA ACCTGTCTCA GGTGGCGCAT | 600 |
| | | TGTCAGGCAC AAGGCAAGAT CGGTTCGGGA TTCGACGTCG CAGCTGCAGC ATACGGCTCC | 660 |
| | | ATTCGCTATC GGAGATTTCC ACCGGCCCTT ATCAGCAACT TGCCAGACAT TGGCTCTGCC | 720 |
| | | ACATACGGGT CAAAGCTCGC TCACCTGGTC AACGAGGAAG ATTGGAATAT CACAATTAAG | 780 |
| | | TCGAATCATC TTCCGTCCGG CCTTACGTTG TGGATGGGTG ACATCAAGAA CGGCTCCGAG | 840 |
| | | ACGGTGAAGC TCGTCCAGAA GGTTAAGAAT TGGTACGACA GCCACATGCC AGAGTCTCTC | 900 |
| | | AAGATATACA CTGAACTGGA TCATGCGAAC TCCAGGTTCA TGGACGGTCT TAGCAAGTTG | 960 |
| | | GATCGCCTCC ACGAGACCCA TGACGATTAC TCAGACCAGA TTTTCGAGTC GCTCGAACGG | 1020 |
| | | AATGATTGCA CCTGTCAAAA GTATCCGGAG ATTACAGAAG TTAGGGACGC CGTGGCTACG | 1080 |
| | | ATCAGGCGCT CTTTCCGCAA GATTACTAAG GAGTCAGGCG CAGATATCGA ACCTCCCGTC | 1140 |
| | | CAGACCTCCC TCCTGGACGA TTGCCAAACG CTGAAGGGCG TTCTGACTTG TCTTATTCCT | 1200 |
| | | GGGGCGGGTG GATACGACGC GATCGCAGTT ATTGCAAAGC AGGACGTGGA TCTCCGGGCC | 1260 |
| | | CAAACCGCTG ACGATAAGAG ATTCTCCAAG GTCCAGTGGC TGGACGTTAC ACAAGCCGAT | 1320 |
| | | TGGGGCGTGC GCAAGGAGAA GGACCCCGAA ACGTATCTCG ATAAGTAAGG TACCAAGCTT | 1380 |
| 129 | Mevalonate | GGATCCGAGC TCATGGCAGA ATCATGGGTC ATTATGGTCA CCGCACAAAC TCCTACAAAC | 60 |
| | pyrophosphate | ATTGCTGTCA TCAAGTATTG GGGAAAGAGG GACGAGAAGT TGATTCTCCC TGTGAACGAC | 120 |
| | decarboxylase | AGCATCTCTG TGACCCTCGA CCCAGTCCAC CTCTGCACCA CAACGACTGT CGCGGTTTCA | 180 |
| | | CCATCGTTCG CACAGGATCG GATGTGGCTG AACGGCAAGG AGATTTCCCT TAGCGGCGGG | 240 |
| | | CGCTACCAGA ATTGCCTTCG CGAAATCAGG GCACGCGCCT GTGACGTTGA GGATAAGGAA | 300 |
| | | AGAGGGATTA AGATCAGCAA GAAGGACTGG GAGAAGCTCC ACGTGCATAT TGCTTCTTAT | 360 |
| | | AACAATTTCC CAACAGCAGC TGGTTTGGCC TCCAGCGCAG CAGGATTCGC TTGCCTCGTG | 420 |
| | | TTTGCTCTGG CGAAGCTCAT GAACGCTAAG GAGGATCATA GCGAATTGTC TGCAATCGCA | 480 |
| | | AGACAGGGCT CTGGGTCAGC ATGTAGATCC CTGTTCGGTG GATTTGTGAA GTGGAAGATG | 540 |
| | | GGCAAGGTCG AGGACGGGTC GGATTCCCTG GCAGTTCAGG TGGTCGACGA AAAGCACTGG | 600 |
| | | GACGATCTTG TGATCATTAT CGCCGTTGTG TCTTCAAGGC AAAAGGAGAC GTCGTCCACC | 660 |
| | | ACCGGTATGC GCGAGACGGT CGAAACTTCC CTCCTGCTTC AGCATAGGGC AAAGGAGATT | 720 |
| | | GTTCCTAAGC GCATCGTGCA GATGGAGGAA TCGATTAAGA ACAGGAATTT CGCTTCCTTT | 780 |
| | | GCGCACCTGA CTTGCGCGGA CTCTAACCAG TTCCATGCAG TCTGCATGGA TACGTGTCCA | 840 |
| | | CCGATCTTTT ACATGAACGA CACTTCCCAC CGGATTATCA GCTGTGTTGA GAAGTGGAAT | 900 |
| | | AGAAGCGTCG GCACCCCACA AGTTGCGTAT ACATTCGATG CAGGACCGAA CGCCGTCCTG | 960 |
| | | ATCGCTCATA ATCGCAAGGC CGCTGCGCAG TTGCTCCAAA AGCTGCTTTT CTACTTTCCT | 1020 |
| | | CCCAACTCTG ACACCGAGCT GAACTCCTAC GTGCTTGGCG ACAAGAGCAT TCTCAAGGAT | 1080 |
| | | GCCGGGATCG AGGACTTGAA GGATGTCGAA GCTCTCCCAC CACCTCCAGA GATTAAGGAC | 1140 |
| | | GCACCAAGAT ACAAGGGCGA TGTCTCATAT TTCATCTGCA CCCGGCCAGG TAGAGGACCG | 1200 |
| | | GTTTTGCTCT CAGACGAGTC GCAGGCCCTG CTTTCGCCTG AAACAGGCCT CCCCAAGTGA | 1260 |
| | | GGTACCTCTA GAAAGCTT | 1278 |
| 130 | Mevalonate | GGATCCGAGC TCATGACTGT CTACACCGCC AGCGTTACCG CACCTGTGAA CATTGCCACG | 60 |
| | pyrophosphate | TTGAAGTATT GGGGGAAGAG AGATACGAAG TTGAACCTGC CAACGAACTC CAGCATCAGC | 120 |
| | decarboxylase | GTCACTCTCT CTCAGGACGA TCTGCGCACG CTTACTTCCG CAGCTACCGC ACCTGAGTTC | 180 |
| | | GAAAGAGATA CACTCTGGCT GAATGGTGAA CCCCACTCCA TTGACAACGA ACGCACCCAG | 240 |
| | | AATTGCTTGA GGGATCTCCG CCAACTGCGG AAGGAGATGG AATCAAAGGA CGCTTCGCTT | 300 |
| | | CCTACTTTGT CTCAGTGGAA GCTGCATATC GTGTCAGAGA ACAATTTCCC CACCGCGGCA | 360 |
| | | GGTCTTGCGT CTTCAGCCGC TGGATTTGCG GCATTGGTCA GCGCCATTGC TAAGCTCTAC | 420 |
| | | CAGCTGCCGC AATCCACCAG CGAGATCAGC AGAATTGCGA GGAAGGGTTC TGGATCAGCA | 480 |
| | | TGCCGGTCGC TTTTCGGCGG GTATGTCGCC TGGGAGATGG GCAAGGCTGA AGACGGGCAC | 540 |
| | | GATTCCATGG CCGTTCAGAT CGCTGACTCG TCCGATTGGC CTCAGATGAA GGCCTGCGTT | 600 |
| | | CTGGTGGTCT CTGACATTAA GAAGGATGTG TCCTCCACAC AGGGCATGCA ACTCACCGTC | 660 |
| | | GCCACAAGCG AGCTGTTCAA GGAGAGAATC GAACATGTTG TGCCCAAGCG CTTTGAGGTC | 720 |
| | | ATGCGGAAGG CTATTGTCGA AAAGGATTTC GCGACGTTTG CAAAGGAGAC TATGATGGAC | 780 |
| | | TCGAACTCCT TCCACGCGAC GTGCCTCGAT TCCTTCCCAC CGATCTTTTA CATGAACGAC | 840 |
| | | ACATCCAAGA GGATCATTAG CTGGTGTCAT ACGATCAATC AGTTCTACGG CGAGACCATT | 900 |
| | | GTTGCTTATA CATTTGATGC GGGGCCAAAC GCAGTGCTTT ACTATTTGGC CGAGAACGAG | 960 |
| | | TCCAAGCTCT TCGCTTTTAT CTATAAGTTG TTCGGTTCTG TTCCGGGATG GGACAAGAAG | 1020 |
| | | TTTACCACAG AGCAGCTCGA AGCGTTCAAC CACCAATTTG AGTCATCGAA TTTCACAGCA | 1080 |
| | | AGAGAGCTTG ACTTGGAACT CCAGAAGGAT GTCGCCAGGG TTATCCTGAC GCAAGTGGGC | 1140 |
| | | TCGGGGCCAC AAGAGACTAA CGAGTCCCTC ATTGACGCCA AGACCGGCCT GCCGAAGGAG | 1200 |
| | | TAAGGTACCA AGCTT | 1215 |

MEPPathway

| | | | |
|---|---|---|---|
| 131 | 1-deoxy-D-xylulose-5- | GGATCCGAGC TCATGGCGTT GACTACATTT TCGATTTCAC GGGGGGGTTT CGTTGGAGCC | 60 |
| | phosphate synthase | CTGCCGCAAG AAGGACACTT TGCACCTGCC GCTGCTGAGC TTTCGTTGCA CAAGCTGCAG | 120 |
| | with chloroplast | TCCCGGCCTC ATAAGGCAAG GAGACGGTCC AGCTCTTCAA TCAGCGCATC TCTCTCAACG | 180 |
| | targeting sequence | GAGCGGGAAG CCGCTGAGTA CCACTCTCAA AGACCACCGA CGCCTCTCCT GGACACTGTG | 240 |
| | | AACTATCCCA TCCATATGAA GAATCTCAGC CTGAAGGAGC TTCAGCAATT GGCGGACGAA | 300 |
| | | CTGCGCTCCG ATGTCATTTT CCACGTTAGC AAGACGGGCG GGCATCTTGG ATCGTCCTTG | 360 |
| | | GGAGTGGTCG AGCTGACGGT GGCACTGCAC TACGTCTTTA ACACTCCGCA GGACAAGATC | 420 |
| | | CTCTGGGATG TCGGACACCA ATCCTATCCT CATAAGATTC TGACTGGCAG AAGGGACAAG | 480 |
| | | ATGCCCACGA TGAGGCAGAC TAATGGTCTC TCCGGATTCA CCAAGCGCTC GGAGTCCGAA | 540 |
| | | TACGATTCGT TTGGAACAGG CCATAGCTCT ACCACAATCT CCGCAGCATT GGGAATGGCA | 600 |
| | | GTGGGTAGGG ACCTCAAGGG TGGAAAGAAC AATGTTGTGG CAGTCATTGG GGATGGTGCG | 660 |
| | | ATGACCGCAG GACAGGCCTA CGAGGCTATG AACAATGCCG GCTATCTGGA CAGCGATATG | 720 |
| | | ATCGTTATTC TTAACGACAA TAAGCAAGTG TCTCTGCCTA CCGCAACACT TGATGGACCA | 780 |
| | | GCACCTCCAG TGGGTGCGCT GTCATCGGCA CTCAGCAAGC TGCAGTCCAG CCGCCCTCTT | 840 |
| | | CGGGAGTTGA GAGAAGTGGC CAAGGGCGTC ACCAAGCAAA TCGGCGGGTC CGTTCACGAG | 900 |
| | | CTGGCCGCTA AGGTGGACGA ATACGCTCGG GGGATGATTA GCGGATCTGG CTCAACACTC | 960 |
| | | TTCGAGGAAC TTGGCTTGTA CTATATCGGA CCCGTGGATG GCCATAACAT TGACGATCTT | 1020 |
| | | ATCACGATTT TGAGAGAGGT GAAGTCCACT AAGACGACTG GCCCAGTCCT CATCCACGTC | 1080 |
| | | GTTACGGAGA AGGGGAGGGG TTACCCGTAT GCGGAACGCG CGGCAGACAA GTACCATGGG | 1140 |
| | | GTCGCGAAGT TCGATCCAGC AACTGGCAAG CAGTTTAAGA GCCCGGCAAA GACCTTGTCT | 1200 |
| | | TACACAAACT ATTTCGCCGA GGCTCTTATC GCGGAGGCAG AACAAGACAA TAGGGTGGTC | 1260 |
| | | GCTATTCACG CAGCTATGGG TGGAGGCACC GGCCTCAACT ATTTCCTGCG CCGGTTTCCA | 1320 |
| | | AATCGCTGCT TCGATGTCGG CATCGCCGAG CAGCATGCTG TTACATTTGC GGCAGGATTG | 1380 |
| | | GCCTGCGAAG GCCTCAAGCC GTTCTGTGCT ATCTACTCTT CATTTCTGCA GAGGGGCTAT | 1440 |
| | | GACCAAGTTG TGCACGACGT CGATCTCCAG AAGCTGCCTG TTCGGTTCGC GATGGACAGA | 1500 |
| | | GCAGGACTCG TCGGAGCTGA TGGTCCAACC CATTGCGGAG CCTTTGACGT TACATACATG | 1560 |
| | | GCTTGTCTTC CAAACATGGT CGTTATGGCC CCGTCCGATG AGGCTGAACT CTGCCACATG | 1620 |
| | | GTGGCAACCG CAGCTGCAAT CGACGATAGA CCAAGCTGTT TCCGCTACCC ACGCGGAAAC | 1680 |
| | | GGCATTGGGG TCCCTCTGCC ACCGAATTAT AAGGGCGTTC CCCTTGAGGT CGGCAAGGGA | 1740 |
| | | CGGGTGCTTT TGGAGGGTGA AAGAGTCGCG CTCCTGGGCT ACGGGTCTGC AGTTCAGTAT | 1800 |
| | | TGCCTGGCAG CCGCTTCACT TGTGGAGAGA CACGGACTGA AGGTGACGGT CGCCGACGCT | 1860 |
| | | AGATTCTGTA AGCCACTTGA TCAAACTTTG ATCAGAAGGC TCGCCTCGTC CCACGAGGTC | 1920 |
| | | CTTTTGACCG TTGAGGAAGG ATCAATTGGG GGTTTCGGCT CGCATGTGGC CCAGTTTATG | 1980 |
| | | GCTTTGGACG GGCTCCTGGA TGGCAAGCTC AAGTGGAGGC CTCTCGTCCT GCCCGACCGC | 2040 |
| | | TACATCGATC ACGGGTCACC AGCAGACCAG TTGGCAGAGG CAGGTCTCAC CCCGTCGCAT | 2100 |
| | | ATCGCGGCAA CAGTTTTCAA CGTGCTGGGA CAAGCAAGAG AAGCCCTTGC TATTATGACA | 2160 |
| | | GTGCCGAATG CTTGAGGTAC CTCTAGAAAG CTT | 2193 |
| 132 | 1-deoxy-D-xylulose-5- | GGATCCGAGC TCATGGCCCT CTCTGCGTGT TCGTTCCCTG CTCATGTTGA CAAGGCGACT | 60 |
| | phosphate synthase | ATCAGCGACC TCCAAAAGTA TGGTTATGTG CCCAGCCGCA GCCTCTGGAG AACGGACCTC | 120 |
| | | CTGGCCCAGA GCTTGGGAAG GCTCAACCAG GCTAAGTCTA AGAAGGGACC TGGAGGAATC | 180 |
| | | TGCGCTTCCC TGAGCGAGAG AGGCGAATAC CACTCACAGA GGCCACCGAC TCCTCTTTTG | 240 |
| | | GACACCACAA ACTATCCCAT CCATATGAAG AATCTTAGCA TTAAGGAGCT GAAGCAACTT | 300 |
| | | GCCGACGAAT TGCGCTCGGA TGTGATCTTC AACGTCTCCC GGACGGGTGG ACACTTGGGC | 360 |
| | | TCCTCCCTCG GAGTGGTCGA GCTGACTGTT GCGCTTCATT ACGTGTTCTC AGCACCTCGG | 420 |
| | | GACAAGATCC TTTGGGATGT GGGGCACCAG TCCTACCCCC ATAAGATCCT CACCGGTAGG | 480 |
| | | CGCGAGAAGA TGTATACGAT TCGCCAAACT AATGGCCTCT CTGGGTTCAC CAAGCGGTCT | 540 |
| | | GAGTCAGAAT ACGACTGCTT TGGAACAGGC CACTCTTCAA CGACTATCTC CGCAGGACTC | 600 |
| | | GGTATGGCAG TGGGAAGGGA CCTGAAGGGC AAGAAGAACA ACGTTGTGGC AGTCATTGGA | 660 |
| | | GATGGCGCGA TGACAGCAGG GCAGGCCTAC GAGGCTATGA ACAATGCCGG TTATCTTGAC | 720 |
| | | TCAGATATGA TCGTTATCTT GAACGACAAT AAGCAAGTGT CGCTCCCTAC CGCCACACTG | 780 |
| | | GATGGACCAA TCCCTCCAGT GGGCGCGCTG TCGTCCGCAT TGTCGAGACT CCAGTCCAAC | 840 |
| | | AGGCCTCTGC GCGAGCTTCG GGAAGTTGCA AAGGGCGTGA CCAAGCAAAT CGGAGGACCA | 900 |
| | | ATGCACGAGT GGGCAGCTAA GGTGGACGAA TACGCCCGCG GCATGATTTC GGGGTCCGGT | 960 |
| | | AGCACACTCT TCGAGGAACT TGGCTTGTAC TATATCGGGC CTGTCGATGG TCATAATATT | 1020 |
| | | GACGATTTGA TCGCTATTCT CAAGGAGGTG AAGTCCACGA AGACCACAGG CCCAGTCCTG | 1080 |
| | | ATCCACGTCG TTACTGAGAA GGGACGCGGC TACCCGTATG CGGAAAAGGC GGCAGACAAG | 1140 |
| | | TACCATGGCG TCACCAAGTT CGATCCCGCG ACAGGAAAGC AGTTTAAGGG CTCAGCAATC | 1200 |
| | | ACGCAATCGT ACACGACTTA TTTCGCCGAG GCTCTCATTG CGGAGGCAGA AGTCGACAAG | 1260 |
| | | GATATCGTTG CCATTCACGC AGCTATGGGT GGAGGCACGG GGCTCAACCT GTTCCTTCGG | 1320 |
| | | AGATTTCCAA CTCGCTGCTT CGACGTCGGC ATCGCCGAGC AGCATGCTGT TACCTTTGCG | 1380 |
| | | GCAGGGCTTG CCTGCGAAGG TTTGAAGCCG TTCTGTGCTA TCTACAGCTC TTTTATGCAG | 1440 |
| | | CGGGCGTATG ATCAAGTGGT CCACGACGTG GATTTGCAGA AGCTCCCAGT CCGCTTCGCG | 1500 |
| | | ATGGACAGAG CAGGTCTCGT GGGAGCAGAT GGACCAACCC ATTGCGGAGC ATTCGACGTC | 1560 |
| | | ACCTTCATGG CTTGTCTGCC AAATATGGTT GTGATGGCCC CGAGCGATGA GGCTGAACTT | 1620 |
| | | TTCCACATGG TGGCAACCGC AGCTGCAATC GACGATAGAC CATCTTGTTT TAGATACCCG | 1680 |
| | | AGGGGGAACG GTGTCGGAGT TCAGCTGCCA CCGGGGAATA AGGGTATTCC GCTCGAGGTC | 1740 |
| | | GGCAAGGGAC GCATCCTGAT TGAGGGCGAA CGGGTTGCGC TCCTGGGTTA TGGAACCGCA | 1800 |
| | | GTGCAGTCCT GCCTCGCAGC AGCTAGCCTG GTCGAGCCTC ACGGCCTTTT GATCACCGTT | 1860 |
| | | GCCGACGCTA GATTCTGTAA GCCCCTGGAT CACACACTTA TTAGGAGCTT GGCCAAGTCT | 1920 |
| | | CATGAGGTCC TCATCACAGT TGAGGAAGGG TCTATTGGGG GTTTCGGTTC ACACGTGGCC | 1980 |
| | | CACTTCCTCG CTCTCGACGG ACTCCTGGAT GGCAAGCTGA AGTGGAGACC TCTGGTTCTT | 2040 |
| | | CCCGACAGGT ACATCGATCA CGGATCTCCA TCAGTCCAGC TTATTGAGGC TGGATTGACG | 2100 |
| | | CCAAGCCATG TGGCAGCAAC TGTCCTGAAC ATCCTTGGCA ATAAGAGGGA AGCGCTGCAA | 2160 |
| | | ATTATGTCAT CGTGAGGTAC CTCTAGAAAG CTT | 2193 |
| 133 | 1-deoxy-D-xyulose-5- | GGATCCGAGC TCATGGCGTT GACTACATTT TCGATTTCAC GGGGGGGTTT CGTTGGAGCC | 60 |
| | phosphate synthase | CTGCCGCAAG AAGGACACTT TGCACCTGCC GCTGCTGAGC TTTCGTTGCA CAAGCTGCAG | 120 |
| | with chloroplast | TCCCGGCCTC ATAAGGCAAG GAGACGGTCC AGCTCTTCAA TCAGCGCGTC TCTGTCAGAG | 180 |
| | targeting sequence | AGAGGCGAAT ACCACAGCCA GAGGCCACCG ACACCTCTTT TGGACACGAC TAACTATCCC | 240 |
| | | ATCCATATGA AGAATCTTTC TATTAAGGAG CTGAAGCAAC TTGCCGACGA ACTCCGCTCC | 300 |
| | | GATGTGATCT TCAACGTCAG CCGGACCGGA GGACACTTGG GGTCCAGCCT CGGTGTGGTC | 360 |
| | | GAGCTGACAG TTGCGCTTCA TTACGTGTTC AGCGCACCTC GCGACAAGAT CCTGTGGGAT | 420 |
| | | GTCGGACACC AGTCTTACCC CCATAAGATC CTTACGGGCA GGCGCGAGAA GATGTATACC | 480 |
| | | ATTAGACAAA CAAATGGTCT CTCCGGATTC ACGAAGAGGT CGGAGTCCGA ATACGACTGC | 540 |
| | | TTTGGGACTG GTCACTCTTC AACCACAATC TCCGCAGGAC TCGGAATGGC AGTGGGAAGG | 600 |
| | | GACCTGAAGG GCAAGAAGAA CAATGTTGTG GCAGTCATTG GGGATGGTGC CATGACCGCT | 660 |
| | | GGACAGGCGT ACGAGGCCAT GAACAACGCC GGCTATCTTG ACTCGGATAT GATCGTTATT | 720 |
| | | TTGAACGACA ATAAGCAAGT GTCCCTCCCT ACGGCTACTC TGGATGGACC AATCCCTCCA | 780 |
| | | GTGGGTGCCC TGTCGTCCGC TTTGTCCCGC CTCCAGAGCA ACCGGCCACT GAGAGAGCTT | 840 |
| | | CGCGAAGTTG CAAAGGGCGT GACCAAGCAA ATCGGTGGAC CGATGCACGA GTGGGCCGCT | 900 |
| | | AAGGTGGACG AATACGCCCG GGGGATGATT AGCGGATCTG GCTCAACACT CTTCGAGGAA | 960 |
| | | CTTGGTTTGT ACTATATCGG ACCTGTCGAT GGCCATAATA TTGACGATTT GATCGCTATT | 1020 |
| | | CTCAAGGAGG TGAAGTCCAC CAAGACGACT GGCCCAGTCC TGATCCACGT CGTTACAGAG | 1080 |
| | | AAGGGGCGCG GTTACCCGTA TGCGGAAAAG GCGGCAGACA AGTACCATGG CGTCACGAAG | 1140 |
| | | TTCGATCCGG CGACTGGGAA GCAGTTTAAG GGTTCGGCAA TCACCCAATC CTACACCACA | 1200 |
| | | TATTTCGCCG AGGCTCTCAT TGCGGAGGCA GAAGTCGACA AGGATATCGT TGCCATTCAC | 1260 |
| | | GCAGCTATGG GAGGAGGCAC CGGCCTCAAC CTGTTCCTTC GGAGATTTCC TACAAGATGC | 1320 |
| | | TTCGACGTCG GCATCGCGGA GCAGCATGCA GTTACATTTG CGGCAGGACT TGCCTGCGAA | 1380 |
| | | GGCTTGAAGC CCTTCTGTGC TATCTACAGC TCTTTTATGC AGAGGGCGTA TGATCAAGTG | 1440 |
| | | GTCCACGACG TGGATTTGCA GAAGCTCCCA GTCCGCTTCG CCATGGACAG AGCTGGACTC | 1500 |
| | | GTGGGAGCAG ATGGTCCAAC GCATTGCGGA GCCTTCGACG TCACTTTTAT GGCTTGTCTC | 1560 |
| | | CCAAACATGG TTGTGATGGC CCCGTCAGAT GAGGCTGAAC TGTTCCACAT GGTGGCTACC | 1620 |
| | | GCAGCTGCAA TCGACGATAG ACCATCCTGT TTTCGCTACC CGAGAGGAAA CGGCGTCGGA | 1680 |
| | | GTTCAGCTGC CACCGGGAAA TAAGGGCATT CCGCTCGAGG TCGGCAAGGG ACGCATCCTG | 1740 |
| | | ATTGAGGGCG AACGGGTTGC GCTCCTGGGC TATGGGACGG CAGTGCAGAG CTGCCTCGCA | 1800 |
| | | GCAGCTTCTC TGGTCGAGCC TCATGGCCTT TTGATCACGG TTGCCGACGC TCGCTTCTGT | 1860 |
| | | AAGCCCCTGG ATCACACTCT TATTCGGTCT TTGGCCAAGT CACATGAGGT CCTCATCACT | 1920 |
| | | GTTGAGGAAG GATCAATTGG AGGCTTCGGC TCGCACGTGG CGCACTTCCT CGCACTCGAC | 1980 |
| | | GGGCTCCTGG ATGGCAAGCT CAAGTGGAGA CCTCTGGTTC TTCCCGACAG GTACATCGAT | 2040 |
| | | CACGGGTCGC CATCCGTGCA GCTTATTGAG GCTGGTTTGA CCCCGAGCCA TGTGGCGGCA | 2100 |
| | | ACAGTCCTGA ACATCCTTGG CAATAAGAGG GAAGCGCTGC AAATTATGTC ATCGTGAGGT | 2160 |
| | | ACCTCTAGAA AGCTT | 2175 |

IFF Pathway

| | | | |
|---|---|---|---|
| 134 | Isopentenyl- | GGATCCGAGC TCATGGGTGA CGCCCCCGAT ACTGGCATGG ACGCCGTGCA AAGGAGACTG | 60 |
| | diphosphate | ATGTTTGAAG ACGAGTGTAT TCTGGTTGAC GAAAATGATC GGGCGGTCGG TCACGCATCC | 120 |
| | Delta-isomerase | AAGTACAGCT GCCATCTGTG GGAGAATATC CTTAAGGGAA ACTCTTTGCA CAGGGCGTTC | 180 |
| | | TCAGTTTTCC TCTTTAATTC GAAGTATGAA CTCCTGCTTC AGCAACGCTC CGCAACGAAA | 240 |
| | | GTGACTTTTC CTCTTGTCTG GACCAACACA TGCTGTTCCC ATCCCTTGTA CAGGGAGAGC | 300 |
| | | GAACGCATCG ACGAGGATGC CCTTGGCGTG CGGAATGCCG CTCAGAGAAA GTTGCTCGAC | 360 |
| | | GAGCTGGGGA TTCCTGCCGA AGACGTTCCC GTGGATCAAT TCACGCCATT GGGCAGGATG | 420 |
| | | CTCTACAAGG CTCCGTCTGA TGGCAAGTGG GGGGAGCACG AACTCGACTA TCTGCTTTTT | 480 |
| | | ATCGTCCGGG ATGTCAACGT TAATCCAAAC CCGGACGAGG TTGCTGATAT TAAGTATGTG | 540 |
| | | AACAGAGACG AGCTGAAGGA ATTGCTCAAG AAGGCCGATG CTGGCGAGGA AGGACTGAAG | 600 |
| | | CTCTCCCCTT GGTTCCGCCT CGTGGTCGAC AATTTCCTGT TTAAGTGGTG GGAGCACGTG | 660 |
| | | GAAAAGGGGA CACTCAAGGA GGCGGCAGAT ATGAAGACCA TTCATAAGCT GACATGAGGT | 720 |
| | | ACCTCTAGAA AGCTT | 735 |
| 135 | Isopentenyl- | GGATCCGAGC TCATGACTGC CGACAACAAC TCTATGCCTC ACGGTGCGGT TTCGTCCTAT | 60 |
| | diphosphate | GCCAAGCTGG TTCAAAATCA AACGCCCGAA GACATCCTCG AGGAGTTCCC AGAGATCATT | 120 |
| | Delta-isomerase | CCGCTCCAGC AAAGGCCTAA TACGCGCTCC AGCGAGACTT CTAACGACGA GTCAGGCGAA | 180 |
| | | ACGTGCTTCA GCGGGCACGA TGAGGAACAG ATCAAGTTGA TGAACGAGAA TTGTATTGTC | 240 |
| | | CTCGACTGGG ACGATAATGC GATCGGCGCA GGGACTAAGA AGGTTTGCCA CCTGATGGAG | 300 |
| | | AACATCGAAA AGGGCCTCCT GCATCGGGCC TTCAGCGTGT TCATTTTTAA TGAGCAGGGG | 360 |
| | | GAACTTTTGC TCCAGCAAAG AGCTACCGAG AAGATCACAT TTCCTGATCT GTGGACCAAC | 420 |
| | | ACATGCTGTT CTCACCCCCT TTGTATTGAC GATGAGCTGG GTCTTAAGGG CAAGCTCGAC | 480 |
| | | GATAAGATCA AGGGCGCCAT TACCGCCGCT GTCCGGAAGC TGGACCATGA GCTTGGTATC | 540 |
| | | CCAGAGGATG AAACGAAGAC TAGGGGAAAG TTCCACTTTC TGAATCGCAT TCATTACATG | 600 |
| | | GCGCCTTCCA ACGAGCCCTG GGGCGAGCAC GAAATCGACT ACATCTTGTT CTATAAGATC | 660 |
| | | AATGCAAAGG AGAACCTCAC AGTTAACCCA AATGTGAACG AAGTCCGCGA TTTCAAGTGG | 720 |
| | | GTGTCGCCGA ATGACCTGAA GACCATGTTT GCTGATCCAT CCTACAAGTT CACACCGTGG | 780 |
| | | TTCAAGATCA TTTGCGAGAA CTATCTTTTC AACTGGTGGG AACAGTTGGA CGATCTCTCC | 840 |
| | | GAGGTTGAAA ACGACCGGCA AATTCATAGA ATGTTGTAAG GTACCAAGCT T | 891 |
| 136 | Farnesyl diphosphate | GGATCCGAGC TCATGGCACC GACAGTTATG GCATCATCCG CTACAGCCGT TGCTCCTTTC | 60 |
| | synthase with | CAGGGGTTGA AGTCCACCGC TACTCTTCCC GTTGCGAGGA GGTCCACCAC CTCCTTCGCG | 120 |
| | chloroplast | AAGGTGTCAA ACGGCGGGAG GATCAGGTGC ATGGCATCGG AGAAGGAAAT TAGGCGCGAG | 180 |
| | targeting sequence | CGCTTCCTGA ACGTCTTTCC TAAGCTGGTT GAGGAACTTA ATGCCTCGCT CCTGGCTTAC | 240 |
| | | GGCATGCCCA AGGAGGCCTG TGACTGGTAC GCTCACTCCC TCAACTATAA TACGCCAGGT | 300 |
| | | GGAAAGTTGA ACAGGGGGCT CAGCGTGGTC GATACGTACG CCATCCTGTC TAATAAGACT | 360 |
| | | GTCGAGCAGC TTGGTCAAGA GGAATATGAA AAGGTTGCTA TCTTGGGATG GTGCATTGAG | 420 |
| | | CTTTTGCAGG CGTACTTCCT GGTCGCAGAC GATATGATGG ACAAGTCCAT CACCCGGAGA | 480 |
| | | GGCCAACCAT GTTGGTATAA GGTTCCGGAA GTGGGGGAAA TCGCGATTAA CGACGCATTC | 540 |
| | | ATGCTGGAGG CCGCTATCTA CAAGCTCCTG AAGTCACACT TTCGCAACGA GAAGTACTAT | 600 |
| | | ATCGACATTA CGGAGCTGTT CCATGAAGTT ACGTTTCAGA CTGAGCTGGG CCAACTGATG | 660 |
| | | GATCTTATCA CTGCGCCCGA AGACAAGGTG GATCTGTCTA AGTTCTCACT TAAGAAGCAC | 720 |
| | | TCCTTCATTG TCACCTTTAA GACAGCCTAC TATAGCTTTT ACCTGCCTGT GGCGCTTGCA | 780 |
| | | ATGTATGTCG CCGGCATCAC AGACGAGAAG GATCTTAAGC AGGCTCGGGA CGTGTTGATC | 840 |
| | | CCGCTCGGCG AGTACTTCCA GATTCAAGAC GATTATCTCG ATTGCTTTGG AACCCCTGAG | 900 |
| | | CAGATCGGCA AGATTGGGAC AGACATCCAA GATAACAAGT GTTCTTGGGT TATTAATAAG | 960 |
| | | GCCCTTGAGT TGGCCTCAGC TGAACAGAGA AAGACCCTGG ACGAGAACTA CGGCAAGAAG | 1020 |
| | | GATAGCGTGG CGGAAGCAAA GTGCAAGAAG ATTTTCAACG ACTTGAAGAT TGAGCAGCTC | 1080 |
| | | TACCATGAAT ATGAGGAATC TATCGCCAAG GATCTCAAGG CTAAGATTTC GCAAGTCGAC | 1140 |
| | | GAGTCCCGGG GCTTCAAGGC GGATGTTTTG ACAGCATTTC TCAATAAGGT GTACAAGAGA | 1200 |
| | | TCCAAGTGAG GTACCTCTAG AAAGCTT | 1227 |
| 137 | Farnesyl diphosphate | GGATCCGAGC TCATGGCTGA TCTGAAGTCG ACGTTTTTGA AGGTGTATTC CGTTCTGAAG | 60 |
| | synthase | CAGGAGTTGC TGGAGGACCC CGCATTTGAG TGGACCCCTG ACTCCAGGCA GTGGGTCGAG | 120 |
| | | CGCATGCTCG ATTACAACGT TCCTGGCGGG AAGCTCAATC GGGGCCTGTC TGTGATTGAC | 180 |
| | | TCATATAAGC TCCTGAAGGA GGGGCAAGAA CTTACCGAGG AAGAGATTTT CCTCGCGTCC | 240 |
| | | GCATTGGGTT GGTGCATTGA GTGGTTGCAG GCCTACTTTC TCGTCCTGGA CGATATCATG | 300 |
| | | GACTCCAGCC ACACAAGGCG CGGCCAACCT TGTTGGTTCA GGGTGCCCAA GGTCGGACTG | 360 |
| | | ATCGCAGCTA ACGATGGGAT TCTTTTGCGG AATCACATCC CCCGCATCCT CAAGAAGCAT | 420 |
| | | TTTCGCGGCA AGGCTTACTA TGTTGACCTC CTGGATTTGT TCAACGAAGT GGAGTTTCAG | 480 |
| | | ACCGCGTCTG GTCAAATGAT CGACCTCATT ACCACACTGG AAGGAGAGAA GGATCTCTCG | 540 |
| | | AAGTACACCC TTTCCTTGCA CCGGAGAATC GTCCAGTACA AGACAGCATA CTATAGCTTC | 600 |
| | | TATCTGCCAG TTGCCTGCGC TCTTTTGATT GCCGGCGAGA ACCTCGACAA TCATATCGTG | 660 |
| | | GTCAAGGATA TTCTGGTGCA GATGGGTATC TACTTCCAGG TCCAAGACGA TTATCTCGAC | 720 |
| | | TGTTTTGGAG ATCCGGAGAC GATCGGCAAG ATCGGAACTG ACATCGAAGA TTTCAAGTGC | 780 |
| | | TCCTGGCTCG TTGTGAAGGC ACTCGAGCTG TGTAACGAGG AGCAGAAGAA GGTGCTGTAC | 840 |
| | | GAACACTATG GCAAGGCCGA CCCAGCAAGC GTCGCCAAGG TCAAGGTTCT TTACAACGAG | 900 |
| | | CTTAAGTTGC AAGGGGTTTT CACGGAATAC GAGAACGAGT CATATAAGAA GCTGGTCACT | 960 |
| | | AGCATCGAGG CTCATCCATC TAAGCCGGTT CAGGCTGTGC TTAAGTCGTT TTTGGCGAAG | 1020 |
| | | ATATACAAGA GGCAAAAGTG AGGTACCTCT AGAAAGCTT | 1059 |
| 138 | Farnesyl diphosphate | GGATCCGAGC TCATGGCACC AACCGTCATG GCATCGTCCG CAACCGCCGT CGCACCTTTC | 60 |
| | synthase with | CAGGGTCTGA AGTCAACAGC AACACTCCCA GTCGCAAGAA GGTCTACCAC ATCATTCGCA | 120 |
| | chloroplast | AAGGTGTCCA ACGGCGGGAG GATCAGGTGC ATGGCCGACC TTAAGTCCAC GTTCTTGAAG | 180 |
| | targeting sequence | GTGTACAGCG TCCTCAAGCA GGAGCTGCTC GAGGACCCAG CTTTTGAGTG GACTCCCGAT | 240 |
| | | TCACGGCAAT GGGTGGAAAG AATGCTGGAC TACAACGTCC CAGGTGGCAA GCTCAATCGC | 300 |
| | | GGTTTGTCCG TGATCGATTC CTACAAGCTC TTGAAGGAGG GACAGGAACT TACCGAGGAA | 360 |
| | | GAGATTTTCC TCGCGTCCGC ACTGGGCTGG TGCATTGAGT GGTTGCAGGC CTACTTTCTT | 420 |
| | | GTCTTGGACG ATATCATGGA CTCCAGCCAC ACAAGGCGCG GGCAACCATG TTGGTTCCGG | 480 |
| | | GTTCCGAAAG TGGGTCTCAT CGCCGCTAAC GATGGCATCC TCCTGAGGAA TCACATCCCG | 540 |
| | | CGCATTCTTA AGAAGCATTT TAGAGGCAAG GCATACTATG TCGACCTTTT GGATTTGTTC | 600 |
| | | AACGAAGTTG AGTTTCAGAC GGCCAGCGGC CAAATGATCG ACCTTATTAC GACTTTGGAA | 660 |
| | | GGGGAGAAGG ATCTTAGCAA GTACACGCTC TCTCTGCACC GGAGAATCGT GCAGTACAAG | 720 |
| | | ACTGCTTACT ATTCTTTCTA TCTGCCTGTC GCCTGCGCTC TCCTGATTGC GGGCGAGAAC | 780 |
| | | CTCGACAATC ATATCGTGGT CAAGGATATT CTGGTTCAGA TGGGCATCTA CTTCCAGGTG | 840 |
| | | CAAGACGATT ATCTGGACTG TTTTGGCGAC CCAGAGACCA TCGGCAAGAT TGGGACAGAC | 900 |
| | | ATCGAAGATT TCAAGTGCTC GTGGCTCGTT GTGAAGGCTC TTGAGTTGTG TAACGAGGAG | 960 |
| | | CAGAAGAAGG TTCTGTACGA GCACTATGGC AAGGCGGACC CAGCATCCGT CGCCAAGGTC | 1020 |
| | | AAGGTTCTCT ACAACGAGCT GAAGCTGCAA GGAGTGTTCA CCGAATACGA GAACGAGTCT | 1080 |
| | | TATAAGAAGC TGGTCACATC AATCGAGGCG CATCCATCGA AGCCGGTCCA GGCTGTTCTC | 1140 |
| | | AAGTCATTTC TGGCGAAGAT ATACAAGCGG CAAAAGTGAG GTACCTCTAG AAAGCTT | 1197 |
| 139 | Farnesyl diphosphate | GGATCCGAGC TCATGGCGTC AGAGAAGGAG ATTAGAAGGG AGAGGTTTTT GAATGTTTTC | 60 |
| | synthase | CCCAAGCTGG TTGAAGAGTT GAATGCGTCA CTGCTGGCAT ACGGTATGCC TAAGGAGGCG | 120 |
| | | TGCGACTGGT ACGCACACTC CCTGAACTAT AATACCCCCG GCGGGAAGTT GAACCGGGGA | 180 |
| | | CTCTCGGTGG TCGATACCTA CGCCATCCTG TCCAATAAGA CAGTTGAGCA GCTTGGCCAA | 240 |
| | | GAGGAATATG AAAAGGTGGC TATCTTGGGG TGGTGCATTG AGCTGCTGCA GGCCTACTTC | 300 |
| | | CTCGTTGCTG ACGATATGAT GGACAAGTCT ATCACAAGGC GCGGTCAACC ATGTTGGTAT | 360 |
| | | AAGGTTCCGG AAGTGGGAGA AATCGCCATT AACGACGCTT TCATGCTGGA GGCCGCTATC | 420 |
| | | TACAAGCTCT TGAAGAGCCA CTTTCGCAAC GAGAAGTACT ATATCGACAT TACCGAGCTG | 480 |
| | | TTCCATGAAG TCACCTTTCA GACAGAGCTT GGTCAATTGA TGGATCTCAT CACAGCCCCT | 540 |
| | | GAAGACAAGG TCGATCTGTC CAAGTTCAGC CTTAAGAAGC ACAGCTTCAT TGTTACGTTT | 600 |
| | | AAGACTGCGT ACTATTCTTT CTACCTGCCG GTCGCGCTTG CAATGTATGT TGCGGGCATC | 660 |
| | | ACGGACGAGA AGGATCTGAA GCAGGCAAGG GACGTGCTGA TCCCACTTGG CGAGTACTTC | 720 |
| | | CAGATTCAAG ACGATTATCT TGATTGCTTT GGGACGCCGG AGCAGATCGG CAAGATCGGA | 780 |
| | | ACTGACATCC AAGATAACAA GTGTTCATGG GTCATCAACA AGGCCCTCGA GCTGGCATCG | 840 |
| | | GCTGAACAGC GCAAGACGCT GGACGAGAAC TACGGCAAGA AGGATTCCGT CGCGGAAGCA | 900 |
| | | AAGTGCAAGA AGATTTTCAA CGACTTGAAG ATTGAGCAGC TCTACCATGA ATATGAGGAA | 960 |
| | | AGCATCGCGA AGGATCTCAA GGCAAAGATT TCTCAAGTCG ACGAGTCACG GGGGTTCAAG | 1020 |
| | | GCCGATGTGT TGACTGCTTT TCTCAACAAG GTCTACAAGA GATCCAAGTA AGGTACCAAG | 1080 |
| | | CTT | 1083 |
| 140 | β-farnesene synthase | GGATCCGAGC TCATGGCCCC TACGGTCATG GCGTCCTCAG CGACTGCGGT TGCACCCTTT | 60 |
| | with chloroplast | CAAGGTCTCA AGAGCACGGC GACACTCCCT GTGGCACGGA GATCGACCAC ATCCTTCGCC | 120 |
| | targeting sequence | AAGGTTTCCA ACGGCGGGAG AATCAGGTGC ATGGACACGC TGCCAATTTC CAGCGTCTCA | 180 |
| | | TTTTCTTCAT CGACTTCGCC TCTTGTGGTC GACGATAAGG TTTCGACGAA GCCCGACGTG | 240 |
| | | ATCAGGCACA CTATGAACTT CAATGCTTCA ATTTGGGGCG ATCAGTTTCT GACCTACGAC | 300 |
| | | GAGCCAGAGG ACCTCGTGAT GAAGAAGCAA CTCGTTGAGG AACTGAAGGA GGAAGTGAAG | 360 |
| | | AAGGAGCTGA TCACAATTAA GGGTAGCAAT GAGCCGATGC AGCACGTGAA GCTCATCGAG | 420 |
| | | TTGATTGACG CGGTCCAACG CTTGGGAATC GCATACCATT TCGAGGAAGA GATCGAAGAG | 480 |
| | | GCCCTTCAGC ACATTCATGT CACCTACGGC GAGCAGTGGG TTGATAAGGA AAACTTGCAA | 540 |
| | | TCAATTTCGC TCTGGTTCCG CCTCCTGCGG CAGCAAGGTT TTAATGTGTC CAGCGGAGTC | 600 |
| | | TTCAAGGACT TTATGGATGA GAAGGGCAAG TTCAAGGAAT CTCTCTGCAA CGACGCGCAG | 660 |
| | | GGAATCCTTG CATTGTACGA GGCCGCTTTC ATGCGGGTGG AGGACGAAAC CATTCTTGAT | 720 |
| | | AATGCGTTGG AGTTTACAAA GGTCCACTTG GATATCATTG CAAAGGACCC GTCATGTGAT | 780 |
| | | TCTTCACTCA GAACCCAGAT CCATCAAGCC CTCAAGCAGC CACTGAGGAG AAGACTTGCA | 840 |
| | | AGGATCGAGG CACTGCACTA CATGCCGATC TACCAGCAAG AGACATCCCA TGACGAAGTT | 900 |
| | | CTTTTGAAGC TCGCTAAGCT GGATTTCTCG GTGTTGCAGT CCATGCACAA GAAGGAGCTG | 960 |
| | | AGCCATATCT GCAAGTGGTG GAAGGACCTC GATCTGCAAA ACAAGCTGCC TTACGTGCGC | 1020 |
| | | GACCGGGTTG TGGAGGGCTA TTTCTGGATT CTCTCCATCT ACTATGAGCC CCAGCACGCG | 1080 |
| | | AGAACCAGGA TGTTTCTGAT GAAGACATGC ATGTGGCTTG TCGTTTTGGA CGATACGTTC | 1140 |
| | | GACAATTACG GTACTTATGA AGAGCTGGAG ATTTTCACCC AAGCAGTGGA ACGCTGGTCC | 1200 |
| | | ATTAGCTGTC TCGATATGCT GCCTGAGTAC ATGAAGCTCA TCTATCAGGA GCTTGTTAAC | 1260 |
| | | TTGCACGTGG AGATGGAGGA GAGCCTGGAG AAGGAAGGGA AGACGTACCA AATTCATTAT | 1320 |
| | | GTCAAGGAGA TGGCCAAGGA ACTGGTGAGA AATTACCTTG TCGAGGCTAG GTGGCTGAAG | 1380 |
| | | GAAGGCTACA TGCCCACCCT TGAAGAGTAT ATGTCTGTCT CAATGGTTAC GGGCACTTAC | 1440 |
| | | GGGCTCATGA TCGCGCGCTC TTATGTGGGT CGGGGAGACA TTGTCACCGA GGATACATTC | 1500 |
| | | AAGTGGGTCT CGTCCTACCC ACCGATCATT AAGGCGTCCT GCGTTATCGT GCGCCTGATG | 1560 |
| | | GACGATATTG TCAGCCACAA GGAAGAGCAG GAGCGGGGCC ATGTTGCAAG CTCTATCGAG | 1620 |
| | | TGCTACAGCA AGGAATCTGG GGCCTCCGAA GAGGAGGCCT GCGAGTATAT CTCTCGCAAG | 1680 |
| | | GTTGAAGACG CCTGGAAGGT CATCAACAGA GAGTCACTGA GGCCAACGGC TGTGCCTTTC | 1740 |
| | | CCCCTCCTGA TGCCGGCCAT CAACTTGGCT CGGATGTGTG AGGTCCTCTA CAGCGTTAAT | 1800 |
| | | GACGGCTTCA CTCACGCCGA GGGGGATATG AAGAGCTATA TGAAGTCTTT CTTTGTCCAT | 1860 |
| | | CCTATGGTGG TCTGAGGTAC CTCTAGAAAG CTT | 1893 |
| 141 | β-farnesene synthase | GGATCCGAGC TCATGGATAC CCTGCCTATT TCGTCCGTCT CGTTCTCCTC TTCTACGTCG | 60 |
| | | CCACTGGTCG TCGATGATAA GGTGTCTACA AAGCCTGATG TGATCCGCCA CACGATGAAC | 120 |
| | | TTCAATGCCT CTATCTGGGG CGACCAGTTT CTGACTTACG ACGAGCCTGA GGACCTCGTG | 180 |
| | | ATGAAGAAGC AACTCGTCGA GGAACTGAAG GAAGAAGTCA AGAAGGAGCT GATCACGATT | 240 |
| | | AAGGGCTCAA ACGAGCCCAT GCAGCACGTG AAGCTCATCG AGTTGATTGA CGCGGTGCAA | 300 |
| | | AGGCTGGGGA TCGCATACCA TTTCGAGGAA GAGATCGAAG AGGCTCTTCA GCACATTCAT | 360 |
| | | GTGACATACG GCGAGCAGTG GGTCGATAAG GAAAACTTGC AATCAATTTC GCTCTGGTTC | 420 |
| | | AGACTCCTGA GGCAGCAAGG CTTTAATGTC TCCAGCGGGG TTTTCAAGGA CTTTATGGAT | 480 |
| | | GAGAAGGGCA AGTTCAAGGA ATCGCTCTGC AACGACGCGC AGGGCATCCT CGCATTGTAC | 540 |
| | | GAGGCCGCTT TCATGCGCGT TGAGGACGAA ACCATTCTTG ATAATGCGTT GGAGTTTACA | 600 |
| | | AAGGTCCACT TGGATATCAT TGCAAAGGAC CCTTCTTGTG ATTCTTCACT CCGCACGCAG | 660 |
| | | ATCCATCAAG CCCTCAAGCA GCCTCTGAGG AGAAGACTTG CAAGAATCGA GGCACTGCAC | 720 |
| | | TACATGCCCA TCTACCAGCA AGAGACTTCC CATGACGAAG TCCTTTTGAA GCTCGCTAAG | 780 |
| | | CTGGATTTCT CTGTTTTGCA GTCAATGCAC AAGAAGGAGC TGAGCCATAT CTGCAAGTGG | 840 |
| | | TGGAAGGACC TCGATCTGCA AAACAAGTTG CCATACGTGA GAGACAGGGT GGTCGAGGGG | 900 |
| | | TATTTCTGGA TTCTCTCCAT CTACTATGAG CCGCAGCACG CGCGCACGCG GATGTTTCTG | 960 |
| | | ATGAAGACTT GCATGTGGCT TGTTGTGTTG GACGATACCT TCGACAATTA CGGCACATAT | 1020 |
| | | GAAGAGCTGG AGATTTTCAC CCAAGCAGTG GAAAGGTGGT CCATTAGCTG TCTCGATATG | 1080 |
| | | CTGCCAGAGT ACATGAAGCT CATCTATCAG GAGCTTGTGA ACTTGCACGT CGAGATGGAG | 1140 |
| | | GAGAGCCTGG AGAAGGAAGG AAAGACCTAC CAAATTCATT ATGTCAAGGA GATGGCCAAG | 1200 |
| | | GAACTGGTCC GCAATTACCT TGTTGAGGCT CGGTGGCTGA AGGAAGGCTA CATGCCGACA | 1260 |
| | | CTTGAAGAGT ATATGTCTGT TTCAATGGTG ACCGGTACAT ACGGACTCAT GATCGCCAGA | 1320 |
| | | TCCTATGTTG GCAGGGGGGA CATTGTGACG GAGGATACTT TCAAGTGGGT GTCGTCCTAC | 1380 |
| | | CCACCGATCA TTAAGGCGAG CTGCGTGATC GTCAGACTGA TGGACGATAT TGTGTCTCAC | 1440 |
| | | AAGGAAGAGC AGGAGAGGGG TCATGTCGCA AGCTCTATCG AGTGCTACTC GAAGGAATCC | 1500 |
| | | GGAGCCAGCG AAGAGGAGGC CTGCGAGTAT ATCTCAAGAA AGGTCGAAGA TGCCTGGAAG | 1560 |
| | | GTTATTAATA GAGAGTCGCT GAGACCAACC GCTGTGCCTT TCCCACTCCT GATGCCGGCC | 1620 |
| | | ATCAACTTGG CTCGGATGTG TGAGGTTCTC TACAGCGTGA ATGACGGTTT TACACACGCC | 1680 |
| | | GAGGGAGATA TGAAGTCGTA TATGAAGTCC TTCTTTGTCC ATCCAATGGT CGTTTAAGGT | 1740 |
| | | ACCAAGCTT | 1749 |
| 142 | α-farnesene synthase | GGATCCGAGC TCATGGACTT GGCGGTGGAG ATTGCTATGG ACCTGGCTGT TGACGATGTT | 60 |
| | | GAACGGCGGG TGGGGGACTA TCACTCGAAC CTGTGGGACG ACGATTTCAT TCAGTCGCTC | 120 |
| | | TCCACGCCAT ATGGCGCATC CAGCTACAGG GAGAGAGCAG AAAGACTGGT GGGAGAGGTC | 180 |
| | | AAGGAAATGT TCACCAGCAT CTCTATTGAG GACGGTGAAC TCACATCCGA CCTCCTGCAG | 240 |
| | | AGACTGTGGA TGGTTGACAA CGTGGAGCGG CTCGGAATCT CGAGACACTT CGAGAACGAG | 300 |
| | | ATCAAGGCCG CTATTGACTA CGTCTATTCA TACTGGTCGG ATAAGGGCAT TGTTCGGGGG | 360 |
| | | AGAGACTCTG CTGTGCCGGA TCTCAACTCA ATCGCGCTGG GCTTCCGGAC CCTCAGACTG | 420 |
| | | CATGGGTACA CAGTGTCTTC AGACGTCTTC AAGGTTTTTC AGGATAGGAA GGGCGAGTTC | 480 |
| | | GCCTGCTCAG CTATTCCAAC CGAAGGCGAC ATCAAGGGAG TTCTGAATCT TTTGCGCGCA | 540 |
| | | TCCTATATCG CCTTCCCGGG CGAGAAGGTC ATGGAGAAGG CTCAAACCTT TGCGGCAACA | 600 |
| | | TACCTTAAGG AGGCGTTGCA GAAGATTCAA GTGTCGTCCC TCAGCCGCGA GATCGAATAT | 660 |
| | | GTCCTTGAGT ACGGCTGGTT GACAAACTTC CCTAGGCTGG AGGCACGCAA TTATATTGAC | 720 |
| | | GTCTTCGGGG AGGAAATCTG CCCATACTTT AAGAAGCCGT GTATCATGGT TGATAAGCTC | 780 |
| | | CTGGAGCTGG CCAAGCTGGA GTTCAACCTC TTTCACAGCC TGCAGCAAAC CGAGCTGAAG | 840 |
| | | CATGTCTCTA GGTGGTGGAA GGACTCCGGC TTCAGCCAGC TTACGTTTAC TAGGCACCGC | 900 |
| | | CATGTGGAGT TCTACACACT CGCTTCTTGC ATCGCGATTG AGCCGAAGCA CTCAGCTTTC | 960 |
| | | CGGCTGGGTT TTGCGAAAGT GTGTTATCTT GGAATTGTCT TGGACGATAT CTACGACACG | 1020 |
| | | TTCGGCAAGA TGAAGGAGCT TGAATTGTTT ACTGCCGCTA TTAAGCGCTG GGACCCATCC | 1080 |
| | | ACCACAGAGT GCCTCCCGGA ATATATGAAG GGCGTCTATA TGGCCTTCTA CAACTGTGTT | 1140 |
| | | AACGAGCTGG CGCTGCAGGC AGAAAAGACG CAAGGGAGGG ACATGCTGAA CTACGCCCGC | 1200 |
| | | AAGGCTTGGG AGGCGCTCTT CGATGCATTT CTGGAGGAAG CCAAGTGGAT CAGCTCTGGC | 1260 |
| | | TATCTTCCTA CTTTCGAGGA ATACTTGGAG AACGGCAAGG TGTCCTTCGG ATACAGGGCG | 1320 |
| | | GCAACGCTCC AGCCTATTCT TACTTTGGAC ATCCCACTCC CGCTGCACAT CCTTCAGCAA | 1380 |
| | | ATTGACTTCC CCTCCCGCTT TAACGATTTG GCTTCATCGA TTCTTCGGTT GAGAGGCGAT | 1440 |
| | | ATCTGCGGGT ATCAAGCAGA GAGGTCGCGC GGCGAGGAAG CCTCCAGCAT CTCCTGTTAC | 1500 |
| | | ATGAAGGACA ATCCCGGATC GACCGAGGAA GATGCACTGT CCCATATCAA CGCCATGATT | 1560 |
| | | AGCGACAACA TCAATGAGCT TAATTGGGAA CTTTTGAAGC CTAACAGCAA TGTGCCCATT | 1620 |
| | | TCTTCAAAGA AGCACGCTTT CGACATCCTT CGGGCGTTTT ACCATTTGTA TAAGTACAGA | 1680 |
| | | GATGGCTTCT CTATCGCCAA GATTGAGACG AAGAACCTCG TGATGAGGAC TGTCCTGGAG | 1740 |
| | | CCTGTTCCCA TGTAAGGTAC CAAGCTT | 1767 |

Preferably, a plant selected to be transformed with such polynucleotides has endogenously a large reserve of carbon-rich energy-storage molecules, in the form of sucrose (such as sweet sorghum and sugar cane) or resin (such as Hevea species and guayule), which are readily available for diversion into the production of terpenoids, and in some embodiments, the production of β-farnesene.

In sorghum, for example and as in many other plants, terpenoid synthesis occurs through the cytosolic MVA pathway and the MEP pathway, the latter of which is localized to the plastidic compartment (Cheng et al., 2007). In some embodiments, increasing the expression of the MVA pathway polypeptides, and/or the MEP pathway polypeptides directs the already large carbon reserves destined in some resin-rich, stored carbon-rich, and stored sugar-rich plants, such as in sorghum, to stored sucrose into increased production of terpenoids, and in some embodiments, where IFF polypeptides are expressed, β-farnesene. In these embodiments, the sum total of carbon flux through photosynthesis into the formation of sucrose and downstream secondary metabolites remain unchanged, with alterations in carbon flux occurring only in pathways involved in secondary metabolites (e.g., terpenoids). As these fluxes can be difficult to quantify using standard metabolic labeling/flux analysis techniques, such diversion of carbon can be quantified through the terpenoid synthesis pathways by: (1) assaying the expression levels and activities of up-regulated enzymes in modified plants or plant cells, (2) determining the amounts of terpenoids and precursors (IPP, FPP), and (3) quantifying amounts, and species as desired, of the produced secondary compounds, including HMG-CoA, methylerythritol phosphate, GPP, FPP, β-farnesene, and any other sesquiterpenoid moieties through liquid chromatography/mass spectrometry (LC/MS). By fully defining and quantifying all of the intermediates involved in the pathways being engineered, this approach allows for determining the relative carbon flux in transgenic plant cells and plants, as well as identify any potential bottlenecks that could result in accumulation of “upstream” precursors. Near Infra-Red spectroscopy (NIR) models can be developed to allow high throughput screening of high terpenoid transgenics (Cornish, 2004).

In some embodiments, β-farnesene synthesis in the cytosol is engineered to be up-regulated. These embodiments take advantage of the fact that the enzymes encoding terpenoid synthesis up to farnesene pyrophosphate are already present and functional in this cellular compartment. In cytosolic terpenoid synthesis, pyruvate formed from the glycolysis of sucrose molecules is converted into Acetyl-CoA which is itself incorporated into 3-hydroxy-3-methylglutaryl-coenzyme A (HMG-CoA) by the enzyme 3-hydroxy-3-methylglutaryl-coenzyme A reductase (Bach et al., 1991; Enjuto et al., 1994). As 3-hydroxy-3-methylglutaryl-coenzyme A reductase catalyzes the rate-limiting step in terpenoid production in the cytosol, this gene is over-expressed to funnel carbon from photosynthate into terpenoid production. HMG-CoA involved in terpenoid synthesis is then processed through the MVA pathway and used to generate dimethylallyl pyrophosphate (DMAPP) and isopentenyl pyrophosphate (IPP), both 5-carbon isoprene monomers for terpenoid biosynthesis (Bach et al., 1991; Cheng et al., 2007; Enjuto et al., 1994). These monomers are assembled together in a series of head-to-tail condensation reactions to generate farnesyl pyrophosphate (FPP, C15), a reaction catalyzed by the enzyme farnesyl diphosphate synthase (FDPS). To specifically direct the increased partitioning of carbon resulting from elevation of HMG-CoA synthesis into production of C15 sesquiterpenoids, expression of FDPS is increased in some embodiments (Cunillera et al., 1996).

Simultaneously up-regulating the expression of the enzymes catalyzing FPP and β-farnesene synthesis results in a dramatically increased pool of cytosolic FPP available for conversion into 3-farnesene. This final reaction is catalyzed by the enzyme β-farnesene synthase, which in some embodiments, is also exogenously expressed. Many characterized sesquiterpene synthases exhibit some degree of promiscuity, i.e., they are able to accept multiple isoprenoid substrates and/or produce multiple products from FPP (Schnee et al., 2006) (Tholl, 2006). To ensure that β-farnesene is the predominant product produced by the modified plant cells and plants of the invention, a β-farnesene synthase gene can be introduced, or the endogenous β-farnesene synthase gene up-regulated. This gene has been demonstrated to function in both monocot (maize) and dicot (Arabidopsis) systems, and to produce primarily β-farnesene (as well as α-bergamotene, β-sesquiphellandrene, β-bisabolene, α-zingiberene, and sesquisabinene in lesser amounts) (Schnee et al., 2006). These sesquiterpenoid molecules exhibit hydrocarbon structures (and therefore energetic yields) almost identical to those of 3-farnesene.

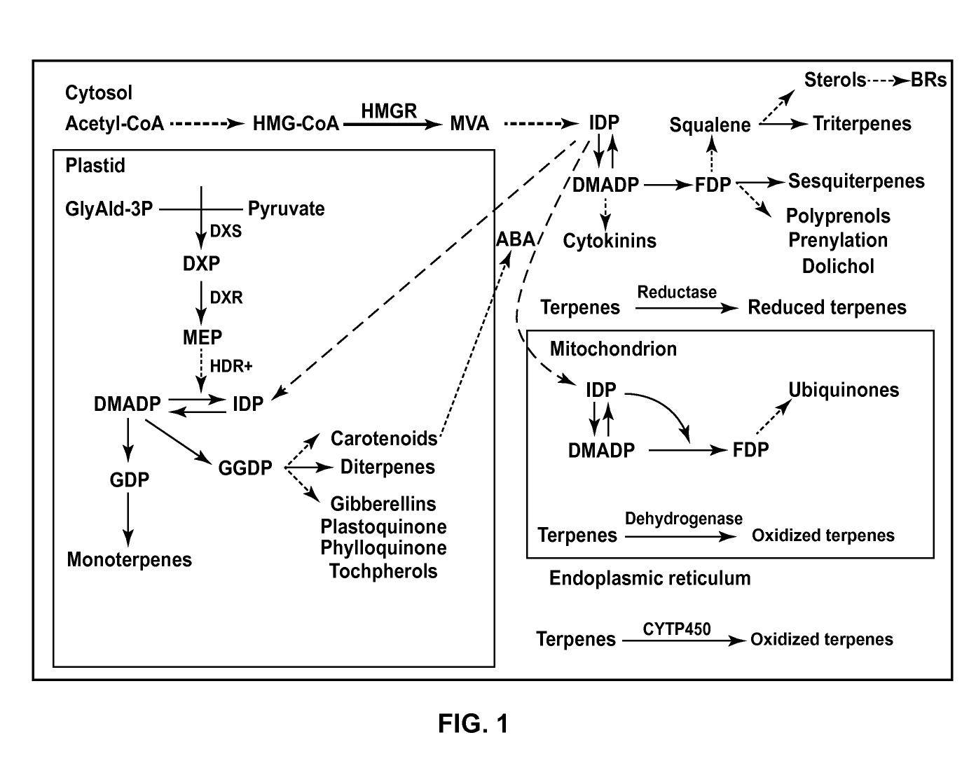

In some embodiments, β-farnesene synthesis is up-regulated in the non-photosynthetic pro-plastids of stem cortical tissues. In previous studies, sugar cane pro-plastids have successfully produced and stored the secondary compound polyhydroxybutyric acid (a bioplastic) (Petrasovits, 2007), thus in some embodiments of the invention, β-farnesene can be stored in this cellular compartment. Plastidic IPP synthesis occurs via the MEP pathway (FIG. 1) (Cheng et al., 2007; Estevez et al., 2000). In this pathway, pyruvate from the glycolysis of sucrose in the cytosol is imported into the plastid and funneled through the MEP pathway to generate the IPP/DMAPP 5-carbon isoprene building blocks of polyterpenoid molecules. GPP synthase enzymes then use these precursors to make C-10 geranyl pyrophosphate. Unlike the cytosol, however, no FPP synthase enzyme is present in the plastid and, instead, two GPP molecules are linked together to form diterpene geranylgeranyl pyrophosphate (GGPP, C20). In some embodiments, to ensure that terpenoid accumulation remains confined to the plastid and limit putative toxic effects, all cytosol-expressed proteins (except 3-hydroxy-3-methylglutaryl-coenzyme A reductase) can be routed to this subcellular compartment by adding an N-terminal signal sequence targeting them to the chloroplast (Bohlmann, 1998; Van den Broeck, 1985; von Heijne, 1989; Wienk, 2000). Thus in some embodiments where the engineered plant cell or plant produces β-farnesene in the plastid, a similar strategy to engineering β-farnesene cytosolic synthesis, is used. In further embodiments, the 1-deoxy-D-xylulose-5-phosphate synthase (DXS), which is the rate limited step in the MEP pathway limiting the production of IPP, is expressed (in lieu of the 3-hydroxy-3-methylglutaryl-coenzyme A reductase involved in cytosolic terpenoid production) and targeted to the plastids (Estevez et al., 2000).

In species like sorghum that do not possess specialized resin storage cells, tissue localization of β-farnesene synthesis can be preferable in some embodiments to generate a high farnesene sorghum plant cell or plant. In some embodiments, the transgenes encoding the enzymes of β-farnesene synthesis are operably linked to a global promoter, such as the PEPC promoter. Under these conditions, β-farnesene accumulates in part in all tissues. In alternative embodiments, β-farnesene production is targeted to mature stem cells involved in actively recruiting carbon-rich photosynthate to maximize production and minimize possible toxic effects. To ensure that the targeted internode regions have enough sucrose or other carbon source available for substantial β-farnesene production, those plant cells and plants producing large stores of carbon, such as high-sucrose sorghum lines, are preferably used. In such embodiments, the β-farnesene synthesis genes can be operably linked to promoters involved in secondary cell wall synthesis (Bell-Lelong et al., 1997; Liang et al., 1989; Maury et al., 1999; Nair et al., 2002) (for example, promoters for sorghum cinnamate 4-hydroxylase, coumarate 3-hydroxylase, and caffeic acid O-methyl transferase). At 30-40% of the stem internode mass, these cells represent a considerable storage volume. In lemon grass, an analogous system, limonene is stored in similar cells with secondary cell walls (LEWINSOHN et al., 1998). In some embodiments, especially in those instances where such an approach results in funneling of carbon away from cell wall production and reducing plant structural integrity, β-farnesene production can be localized to another plant compartment, such as the ground tissue cortical cells of sorghum internodes; this is accomplished by operably-linking the transgenese to promoters specific to that plant compartment. Such promoters are readily identified by those of skill in the art. For example, in sweet sorghum, the internode ground tissue cortical cells make up the majority of the internode mass (50-60%) and are involved in sucrose storage, so that a ready supply of carbon flux is available. In some embodiments, global and tissue-specific transgenes are used in the same plant cell or plant; these embodiments can be produced either by introducing all such transgenes into one host plant, or combined through crossing transgenic plants using conventional techniques.

Alternative Embodiments for Modulating β-Farnesene Synthase

β-farnesene synthase isoforms with increased substrate specificity can be engineered for increased substrate using rational engineering of the active site, which has been demonstrated for other terpene synthases (Greenhagen et al., 2006; Yoshikuni and University of California, 2007). Such engineering focuses on β-farnesene synthases previously isolated and characterized from maize and wild teosinte relatives (Köller et al., 2009). β-farnesene synthases from other plant species, including Artemisia annua (Picaud S, 2005), Japanese citrus (Maruyama T, 2001), mint (Crock J, 1997), and Douglas fir (Huber D P, 2005), have been expressed in multiple expression systems (including E. coli and yeast) and have been characterized. Such expressed proteins are modeled against known sesquiterpene synthase three-dimensional structures, and residues in and around the active site are identified and altered, generating specificity variants which are screened for improved performance.

Chloroplast Targeting

In some embodiments, instead of using signal peptides to target nuclear-encoded enzymes to pro-plastids, genes involved in β-farnesene synthesis are introduced directly into the chloroplast genome of the target plant cell or plant. In such embodiments, IPP levels are increased by transforming with MEV genes cassette, and include FDPS and β-farnesene synthase. These embodiments are especially attractive when the chloroplast genome is known or otherwise suitable insertion sites have been identified to engineer the chloroplast genome.

Generally, in the embodiments of the invention, the engineered plants producing sesquiterpenoids, including farnesene, produce such sesquiterpenoids, by dry weight, at 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, and 20% and more.

### B. Vector Compositions and Structure

In some embodiments, mini-chromosomes, or other large DNA constructs that can be used to introduce large numbers of genes simultaneously into the genome of a plant cell, are exploited to express the multiple genes involved in terpenoid production, such as those encoding the polypeptides shown in Tables 1-3 and further described in Tables 4-7, or the polynucleotides of Table 7. A main advantage of using mini-chromosomes, which when autonomously maintained by plant cells, is that the expression of genes carried on mini-chromosomes is not affected by position effects commonly observed in traditional engineered crops. Large gene payloads and stable expression are ideal for pathway engineering projects, and require fewer transgenic lines to be screened for commercial applications.

One aspect of the invention is related to plants containing functional, stable, autonomous MCs, preferably carrying one or more exogenous nucleic acids, such as MVA pathway and/or MEP pathway and, alternatively, IFF gene stacks. Such plants carrying MCs are contrasted to transgenic plants with genomes that have been altered by chromosomal integration of an exogenous nucleic acid. Expression of the exogenous nucleic acid results in an altered phenotype of the plant. MCs can comprise at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 250, 500, 1000 or more exogenous nucleic acids.

MCs can be transmitted to subsequent generations of viable daughter cells during mitotic cell division with a transmission efficiency of at least 1%, 5%, 10%, 20%, 30%, 40%, 50%, 60%, 70%, 80%, 85%, 90%, 95%, 96%, 97%, 98%, or 99%. The MC is transmitted to viable gametes during meiotic cell division with a transmission efficiency of at least 1%, 5%, 10%, 20%, 30%, 40%, 50%, 60%, 70%, 80%, 85%, 90%, 95%, 96%, 97%, 98%, or 99% when more than one copy of the MC is present in the gamete mother cells of the plant. The MC is transmitted to viable gametes during meiotic cell division with a transmission frequency of at least 1%, 5%, 10%, 20%, 30%, 40%, 45%, 46%, 47%, 48%, or 49% when one copy of the MC is present in the gamete mother cells of the plant and meiosis produces four viable products (e.g. typical male meiosis). When meiosis produces fewer than four viable products (e.g. typical female meiosis) a phenomenon called meiotic drive can cause the preferential segregation of particular chromosomes into the viable product resulting in higher than expected transmission frequencies of monosomes through meiosis including at least 51%, 60%, 70%, 80%, 90% 95%, 96%, 97%, 98%, or 99%. For production of seeds via sexual reproduction or by apomyxis, the MC can be transferred into at least 1%, 5%, 10%, 20%, 25%, 30%, 40%, 50%, 60%, 70%, 80%, 85%, 90%, 95%, 96%, 97%, 98%, or 99% of viable embryos when cells of the plant contain more than one copy of the MC. For sexual seed production or apomyxitic seed production from plants with one MC per cell, the MC can be transferred into at least 1%, 5%, 10%, 20%, 30%, 40%, 50%, 60%, 70%, 71%, 72%, 73%, 74%, 75% of viable embryos.

Transmission efficiency can be measured as the percentage of progeny cells or plants that carry the MC by one of several assays, including detecting expression of a reporter gene (e.g., a gene encoding a fluorescent protein), PCR detection of a sequence that is carried by the MC, RT-PCR detection of a gene transcript for a gene carried on the MC, Western analysis of a protein produced by a gene carried on the MC, Southern analysis of the DNA (either in total or a portion thereof) carried by the MC, fluorescence in situ hybridization (FISH) or in situ localization by repressor binding. Efficient transmission as measured by some benchmark percentage indicates the degree to which the MC is stable through the mitotic and meiotic cycles. Plants of the invention can also contain chromosomally integrated exogenous nucleic acid in addition to the autonomous MCs. The mini-chromosome-containing plants or plant parts, including plant tissues, can include plants that have chromosomal integration of some portion of the MC (e.g., exogenous nucleic acid or centromere sequence) in some or all cells of the plant. The plant, including plant tissue or plant cell, is still characterized as mini-chromosome-containing, despite the occurrence of some chromosomal integration. A mini-chromosome-containing plant can also have a MC plus non-MC integrated DNA.

Another aspect of the invention relates to methods for producing and isolating such mini-chromosome-containing plants containing functional, stable, autonomous MCs carrying, for example, MVA pathway, and/or MEP pathway, and/or IFF gene stacks.

Another aspect of the invention relates to methods for using MC-containing plants containing a MC carrying an MVA pathway, and/or MEP pathway, and/or IFF gene stacks for producing chemical and fuel products by appropriate expression of exogenous farnesene metabolic engineering (FME) nucleic acid(s) contained on a MC.

The invention contemplates MCs comprising centromeric nucleotide sequence that when hybridized to 1, 2, 3, 4, 5, 6, 7, 8, 9, 10, 11, 12, 13, 14, 15, 16, 17, 18, 19, 20, 30, 40, 50, 60, 70, 80, 90, 100 or more probes, under hybridization conditions described herein, e.g., low, medium or high stringency, provides relative hybridization scores, as has been previously described, such as in International Patent Application Publication No. WO2011091332.

The MC vector in some embodiments can contain a variety of elements, including: (1) sequences that function as plant centromeres; (2) one or more exogenous nucleic acids; (3) sequences that function as an origin of replication, that can be included in the region that functions as plant centromere, and optional; (4) a bacterial plasmid backbone for propagation of the plasmid in bacteria, though this element may be designed to be removed prior to delivery to a plant cell; (5) sequences that function as plant telomeres (particularly if the MC is linear); (6) optionally, additional “stuffer DNA” sequences that serve to separate the various components on the MC from each other; (7) optionally, “buffer” sequences such as MARs or SARs; (8) optionally, marker sequences of any origin, including but not limited to plant and bacterial origin; (9) optionally, sequences that serve as recombination sites; and (10) optionally, “chromatin packaging sequences” such as cohesion and condensing binding sites.

The centromere in the MC of some embodiments of the present invention can comprise centromere sequences as known in the art, which have the ability to confer to a nucleic acid the ability to segregate to daughter cells during cell division. US Pat. Nos. 6,649,347, 7,119, 250, 7,132,240 describe methods for identifying and isolating centromeres; US Pat. Nos. 7,456,013, 7,235,716, 7,227,057, and 7,226,782 disclose corn, soy, Brassica and tomato centromeres respectively; U.S. Pat. Nos. 7,989,202 and 8,062,885 described crop plant centromere compositions generally; US Patent Application Publication Nos. US20100297769 and US20090222947 also describe corn centromere compositions, international patent application publication nos. WO2011011693, WO2011091332, and WO2011011685 describe sorghum, cotton and sugar cane centromeres, respectively; and international patent application publication no. WO2009134814 describes some algae centromere compositions. Other centromere compositions are known in the art or can be identified using guidance from the aforementioned patents and patent applications. These patent application publications and issued patents are incorporated by reference herein.

For example, for Hevea MC development, Hevea genomic DNA can be isolated from etiolated seedlings. A Bacterial Artificial Chromosome (BAC) library is prepared in a modified pBeIoBAC11 vector. The library is arrayed on nylon filters and hybridized with centromere-specific satellite or centromere-associated retrotransposon sequence probes. To identify probe sequences, Hevea genomic DNA are sequenced. Centromere probes can then be amplified from genomic DNA, cloned and characterized, and FISH analysis, or other appropriate analysis technique used to confirm their centromere localization. For example, about 50 BAC clones obtained from library screening can be characterized at the molecular level and hybridized to Hevea root tip metaphase chromosome spreads. The three BAC clones with highest content of centromere satellite repeats and retrotransposon sequences, and strongest and specific hybridization to centromere regions of metaphase chromosomes can be selected to build mini-chromosomes.

Other expression vectors are well-known to those of skill in the art. In expression vectors, for example, the introduced DNA is operably-linked to elements, such as promoters, that signal to the host cell to transcribe the inserted DNA. Some promoters are exceptionally useful, such as inducible promoters that control gene transcription in response to specific factors. Operably-linking a gene of interest or anti-sense construct to an inducible promoter can control the expression of the gene of interest. Examples of inducible promoters include those that are tissue-specific, which relegate expression to certain cell types, steroid-responsive, or heat-shock reactive. Other desirable inducible promoters include those that are not endogenous to the cells in which the construct is being introduced, but, however, are responsive in those cells when the induction agent is exogenously supplied.

Plant-expressed genes from non-plant sources can be modified to accommodate plant codon usage (such as those sequences presented in Table 7), to insert preferred motifs near the translation initiation ATG codon, to remove sequences recognized in plants as 5′ or 3′ splice sites, or to better reflect plant GC/AT content. Plant genes typically have a GC content of more than 35%, and coding sequences that are rich in A and T nucleotides can be problematic. For example, ATTTA motifs can destabilize mRNA; plant polyadenylation signals such as AATAAA at inappropriate positions within the message can cause premature truncation of transcription; and monocotyledons can recognize AT-rich sequences as splice sites.

Each exogenous nucleic acid or plant-expressed gene can include a promoter, a coding region and a terminator sequence, that can be separated from each other by restriction endonuclease sites or recombination sites or both. Genes can also include introns that can be present in any number and at any position within the transcribed portion of the gene, including the 5′ untranslated sequence, the coding region, and the 3′ untranslated sequence. Introns can be natural plant introns derived from any plant, or artificial introns based on the splice site consensus that has been defined for plant species. Some intron sequences have been shown to enhance expression in plants. Optionally the exogenous nucleic acid can include a plant transcriptional terminator, non-translated leader sequences derived from viruses that enhance expression, a minimal promoter, or a signal sequence controlling the targeting of gene products to plant compartments or organelles.

The coding regions of the exogenous genes can encode any protein, including those polypeptides shown in Tables 1-3 and further described in Tables 4-7, as well as visible marker genes (for example, fluorescent protein genes, other genes conferring a visible phenotype), other screenable or selectable marker genes (for example, conferring resistance to antibiotics, herbicides or other toxic compounds, or encoding a protein that confers a growth advantage to the cell expressing the protein). Multiple genes can be placed on the same vector. The genes can be separated from each other by restriction endonuclease sites, homing endonuclease sites, recombination sites or any combinations thereof. Any number of genes can be present, especially when the vector is a MC. Genes can be in any orientation with respect to one another and with respect to the other elements of the vector (e.g. the centromere in MCs).

Vectors can also contain a bacterial plasmid backbone for propagation of the plasmid in bacteria such as E. coli, A. tumefaciens, or A. rhizogenes. The plasmid backbone can be that of a low-copy vector or mid to high level copy backbone. This backbone can contain the replicon of the F′ plasmid of E. coli. However, other plasmid replicons, such as the bacteriophage P1 replicon, or other low-copy plasmid systems, such as the RK2 replication origin, can also be used. The backbone can include one or several antibiotic-resistance genes conferring resistance to a specific antibiotic to the bacterial cell in that the plasmid is present. The backbone can also be designed so that it can be excised from the vector prior to delivery to a plant cell. The use of flanking restriction enzyme sites or flanking site-specific recombination sites are both useful for constructing a removable backbone.

MC vectors can also contain plant telomeres. An exemplary telomere sequence is tttaggg or its complement. Telomeres stabilize the ends of linear chromosomes and facilitate the complete replication of the extreme termini of the DNA molecule.

Additionally, the vector can contain “stuffer DNA” sequences that serve to separate the various components on the vector. Stuffer DNA can be of any origin, synthetic, prokaryotic or eukaryotic, and from any genome or species, plant, animal, microbe or organelle. Stuffer DNA can range from 10 bp, 20 bp, 30 bp, 40 bp, 50 bp, 60 bp, 70 bp, 80 bp, 90 bp, 100 bp, 150 bp, 200 bp, 300 bp, 400 bp 500 bp, 750 bp, 1000 bp, 2000 bp, 5000 bp, 10 kb, 20 kb, 50 kb, 75 kb, 1 Mb to 10 Mb in length and can be repetitive in sequence, with unit repeats from 10 bp to 1 Mb. Examples of repetitive sequences that can be used as stuffer DNAs include rDNA, satellite repeats, retroelements, transposons, pseudogenes, transcribed genes, microsatellites, tDNA genes, short sequence repeats and combinations thereof. Alternatively, stuffer DNA can consist of unique, non-repetitive DNA of any origin or sequence. The stuffer sequences can also include DNA with the ability to form boundary domains, such as scaffold attachment regions (SARs) or matrix attachment regions (MARs). Stuffer DNA can be entirely synthetic, composed of random sequence, having any base composition, or any A/T or G/C content.

In some embodiments of the invention, the vector is a MC that has a circular structure without telomeres. In other embodiments, the MC has a circular structure with telomeres. In a third embodiment, the MC has a linear structure with telomeres. In other embodiments, the vector is a plasmid. In yet other embodiments, multiple vectors are used, such as multiple plasmids, multiple MCs, or a combination of plasmids and MCs.

Various structural configurations of vector elements are possible. In a MC vector, a centromere can be placed on a MC either between genes or outside a cluster of genes next to a telomere. Stuffer DNAs can be combined with these configurations including stuffer sequences placed inside telomeres, around the centromere between genes or any combination thereof. Thus, a large number of alternative MC and other vector structures are possible, depending on the relative placement of centromere DNA (in the case of MCs), genes, stuffer DNAs, bacterial sequences, telomeres (in the case of MCs), and other sequences. Such variations in architecture are possible both for linear and for circular MCs. Non-MC vectors can also have such architectural variation, but will have absent elements such as functional centromeres and functional telomeres.

### C. Exemplary Plant Promoters, Regulatory Sequences and Targeting Sequences

Constitutive Expression promoters: Exemplary constitutive expression promoters include the ubiquitin promoter, the CaMV 35S promoter (U.S. Pat. Nos. 5,858,742 and 5,322,938); and the actin promoter (e.g., rice, U.S. Pat. No. 5,641,876).

Inducible Expression promoters: Exemplary inducible expression promoters include the chemically regulatable tobacco PR-1 promoter (e.g., tobacco, U.S. Pat. No. 5,614,395; maize, U.S. Pat. No. 6,429,362). Various chemical regulators can be used to induce expression, including the benzothiadiazole, isonicotinic acid, and salicylic acid compounds disclosed in U.S. Pat. Nos. 5,523,311 and 5,614,395. Other promoters inducible by certain alcohols or ketones, such as ethanol, include the alcA gene promoter from Aspergillus nidulan. Glucocorticoid-mediated induction systems can also be used (Aoyama and Chua, 1997). Another class of useful promoters are water-deficit-inducible promoters, e.g., promoters that are derived from the 5′ regulatory region of genes identified as a heat shock protein 17.5 gene (HSP 17.5), an HVA22 gene (HVA22), and a cinnamic acid 4-hydroxylasc gene (CA4H) of Zea mays. Another water-deficit-inducible promoter is derived from the rob-17 promoter. U.S. Pat. No. 6,084,089 discloses cold inducible promoters, U.S. Pat. No. 6,294,714 discloses light inducible promoters, (PEPC is also light inducible, Bansal et al. (1992) Transient expression from cab-m1 and rbcS-m3 promoter sequences is different in mesophyll and bundle sheath cells in maize leaves. PNAS 89 (8) 3654-3658), U.S. Pat. No. 6,140,078 discloses salt inducible promoters, U.S. Pat. No. 6,252,138 discloses pathogen inducible promoters, and U.S. Pat. No. 6,175,060 discloses phosphorus deficiency inducible promoters.

Wound-Inducible Promoters can Also be Used.

Tissue-Specific Promoters: Exemplary promoters that express genes only in certain tissues are useful, such as those disclosed in US Pat. Publication No. 2010-0011460. For example, root-specific expression can be attained using the promoter of the maize metallothionein-like (MTL) gene (U.S. Pat. No. 5,466,785). U.S. Pat. No. 5,837,848 discloses a root-specific promoter. Another exemplary promoter confers pith-preferred expression (maize trpA gene and promoter; WO 93/07278). Leaf-specific expression can be attained, for example, by using the promoter for a maize gene encoding phosphoenol carboxylase. Pollen-specific expression can be conferred by the promoter for the maize calcium-dependent protein kinase (CDPK) gene that is expressed in pollen cells (WO 93/07278). U.S. Pat. Appl. Pub. No. 20040016025 describes tissue-specific promoters. Pollen-specific expression can also be conferred by the tomato LAT52 pollen-specific promoter. U.S. Pat. No. 6,437,217 discloses a root-specific maize RS81 promoter, U.S. Pat. No. 6,426,446 discloses a root specific maize RS324 promoter, U.S. Pat. No. 6,232,526 discloses a constitutive maize A3 promoter, U.S. Pat. No. 6,177,611 that discloses constitutive maize promoters, U.S. Pat. No. 6,433,252 discloses a maize L3 oleosin promoter that are aleurone and seed coat-specific promoters, U.S. Pat. No. 6,429,357 discloses a constitutive rice actin 2 promoter and intron, U.S. patent application Pub. No. 20040216189 discloses an inducible constitutive leaf-specific maize chloroplast aldolase promoter. Other plant tissue specific promoters are disclosed in US Pat. Nos. 7,754,946, 7,323,622, 7,253,276, 7,141,427, 7,816,506, and 7,973,217, and in US Patent Application Publication No. 20100011460. To confer expression to mature stem cells promoters involved in secondary cell wall synthesis (Bell-Lelong et al., 1997; Liang et al., 1989; Maury et al., 1999; Nair et al., 2002) (for example, promoters for sorghum cinnamate 4-hydroxylase, coumarate 3-hydroxylase, and caffeic acid O-methyl transferase).

Optionally a plant transcriptional terminator can be used in place of the plant-expressed gene native transcriptional terminator. Exemplary transcriptional terminators are those that are known to function in plants and include the CaMV 35S terminator, the tml terminator, the nopaline synthase terminator and the pea rbcS E9 terminator. These can be used in both monocotyledons and dicotyledons.

Various intron sequences have been shown to enhance expression. For example, the introns of the maize Adh1 gene can significantly enhance expression, especially intron 1 (Callis et al., 1987). The intron from the maize bronze/gene also enhances expression. Intron sequences have been routinely incorporated into plant transformation vectors, typically within the non-translated leader. U.S. Patent Application Publication 2002/0192813 discloses 5′, 3′ and intron elements useful in the design of effective plant expression vectors.

A number of non-translated leader sequences derived from viruses are also known to enhance expression, and these are particularly effective in dicotyledonous cells (such as. Specifically, leader sequences from Tobacco Mosaic Virus (TMV, the “omega-sequence”), Maize Chlorotic Mottle Virus (MCMV), and Alfalfa Mosaic Virus (AMV) can enhance expression. Other leader sequences known and include: picornavirus leaders, for example, EMCV leader (Encephalomyocarditis 5′ noncoding region); potyvirus leaders, for example, TEV leader (Tobacco Etch Virus); MDMV leader (Maize Dwarf Mosaic Virus); human immunoglobulin heavy-chain binding protein (BiP) leader; untranslated leader from the coat protein mRNA of alfalfa mosaic virus (AMV RNA 4); tobacco mosaic virus leader (TMV); or Maize Chlorotic Mottle Virus leader (MCMV).

A minimal promoter can also be incorporated. Such a promoter has low background activity in plants when there is no transactivator present or when enhancer or response element binding sites are absent. An example is the Bzl minimal promoter, obtained from the bronze/gene of maize. A minimal promoter can also be created by use of a synthetic TATA element. The TATA element allows recognition of the promoter by RNA polymerase factors and confers a basal level of gene expression in the absence of activation.

Sequences controlling the targeting of gene products also can be included. For example, the targeting of gene products to the chloroplast is controlled by a signal sequence found at the amino terminal end of various proteins that is cleaved during chloroplast import to yield the mature protein. These signal sequences can be fused to heterologous gene products to import heterologous products into the chloroplast. DNA encoding for appropriate signal sequences can be isolated from the 5′ end of the cDNAs encoding the RUBISCO protein, the CAB protein, the EPSP synthase enzyme, the GS2 protein or many other proteins that are known to be chloroplast localized. Other gene products are localized to other organelles, such as the mitochondrion and the peroxisome (e.g., (Unger et al., 1989)). Examples of sequences that target to such organelles are the nuclear-encoded ATPases or specific aspartate amino transferase isoforms for mitochondria. Amino terminal and carboxy-terminal sequences are responsible for targeting to the ER, the apoplast, and extracellular secretion from aleurone cells. Amino terminal sequences in conjunction with carboxy terminal sequences can target to the vacuole.

Another element that can be introduced is a matrix attachment region element (MAR), such as the chicken lysozyme A element that can be positioned around an expressible gene of interest to effect an increase in overall expression of the gene and diminish position dependent effects upon incorporation into the plant genome.

Use of Non-Plant Promoter Regions Isolated from Drosophila melanogaster and Saccharomyces cerevisiae to Express Genes in Plants

Promoters can be derived from plant or non-plant species. For example, the nucleotide sequence of the promoter is derived from non-plant species for the expression of genes in plant cells, such as dicotyledon plant cells, such as guayule and Hevea sp.. Non-plant promoters can be constitutive or inducible promoters derived from insects, e.g., Drosophila melanogaster, or from yeast, e.g., Saccharomyces cerevisiae. These non-plant promoters can be operably linked to nucleic acid sequences encoding polypeptides or non-protein-expressing sequences including antisense RNA, miRNA, siRNA, and ribozymes, to form nucleic acid constructs, vectors, and host cells (prokaryotic or eukaryotic), comprising the promoters.

In the methods of the present invention, the promoter can also be a mutant of the promoters having a substitution, deletion, and/or insertion of one or more nucleotides in a native nucleic acid sequence of that element.

The techniques used to isolate or clone a nucleic acid sequence comprising a promoter of interest are known in the art.

Constructing MCs by Site-Specific Recombination

Plant MCs can be constructed using site-specific recombination sequences (for example those recognized by the bacteriophage P1 Cre recombinase, or the bacteriophage lambda integrase, or similar recombination enzymes). A compatible recombination site, or a pair of such sites, is present on both the centromere containing DNA clones and the donor DNA clones. Incubation of the donor clone and the centromere clone in the presence of the recombinase enzyme causes strand exchange to occur between the recombination sites in the two plasmids; the resulting MCs contain centromere sequences as well as MC vector sequences. The DNA molecules formed in such recombination reactions is introduced into E. coli, other bacteria, yeast or plant cells by common methods in the field including, heat shock, chemical transformation, electroporation, particle bombardment, whiskers, or other transformation methods followed by selection for marker genes, including chemical, enzymatic, or color markers present on either parental plasmid, allowing for the selection of transformants harboring MCs.

### F. Transformation of Plant Cells and Plant Regeneration

Various methods can be used to deliver DNA into plant cells. These include biological methods, such as Agrobacterium, E. coli, and viruses; physical methods, such as biolistic particle bombardment, nanocopiea device, the Stein beam gun, silicon carbide whiskers and microinjection; electrical methods, such as electroporation; and chemical methods, such as the use of polyethylene glycol and other compounds that stimulate DNA uptake into cells (Dunwell, 1999) and U.S. Pat. No. 5,464,765.

Agrobacterium-Mediated Delivery

Several Agrobacterium species mediate the transfer of T-DNA that can be genetically engineered to carry a desired piece of DNA into many plant species. Plasmids used for delivery contain the T-DNA flanking the nucleic acid to be inserted into the plant. The major events marking the process of T-DNA mediated pathogenesis are induction of virulence genes, processing and transfer of T-DNA.

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be modified by Agrobacterium and (b) that the modified cells or tissues can be induced to regenerate into whole plants. The third method is transformation of seeds, apices or meristems with Agrobacterium. This method requires exposure of the meristematic cells of these tissues to Agrobacterium and micropropagation of the shoots or plant organs arising from these meristematic cells.

Those of skill in the art are familiar with procedures for growth and suitable culture conditions for Agrobacterium, as well as subsequent inoculation procedures.

Transformation of dicotyledons using Agrobacterium has long been known in the art (e.g., U.S. Pat. No. 8,273,954), and transformation of monocotyledons using Agrobacterium has also been described (WO 94/00977; U.S. Pat. No. 5,591,616; US20040244075).

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis. Exemplary strains include Agrobaclerium tumefaciens strain CSS, a nopaline-type strain that is used to mediate the transfer of DNA into a plant cell, octopine-type strains such as LBA4404 or succinamopine-type strains, e.g., EHA101 or EHA105.

The efficiency of transformation by Agrobacterium can be enhanced by using a number of methods known in the art. For example, the inclusion of a natural wound response molecule such as acetosyringone (AS) to the Agrobaclerium culture can enhance transformation efficiency with Agrobaclerium tumefaciens. Alternatively, transformation efficiency can be enhanced by wounding the target tissue to be modified or transformed. Wounding of plant tissue can be achieved, for example, by punching, maceration, bombardment with microprojectiles, etc.

In addition, transfer of a disarmed Ti plasmid without T-DNA and another vector with T-DNA containing the marker enzyme beta-glucuronidase can be accomplished into three different bacteria other than Agrobacteria which adds to the transformation vector arsenal.

Microprojectile Bombardment Delivery

In this process, the desired nucleic acid is deposited on or in small dense particles, e.g., tungsten, platinum, or gold particles, that are then delivered at a high velocity into the plant tissue or plant cells using a specialized biolistics device, such as are available from Bio-Rad Laboratories (Hercules; CA, USA). The advantage of this method is that no specialized sequences need to be present on the nucleic acid molecule to be delivered into plant cells.

For bombardment, cells in suspension are concentrated on filters or solid culture medium. Alternatively, immature embryos, seedling explants, or any plant tissue or target cells can be arranged on solid culture medium. The cells to be bombarded are positioned at an appropriate distance below the microprojectile stopping plate.

Various biolistics protocols have been described that differ in the type of particle or the manner in that DNA is coated onto the particle. Any technique for coating microprojectiles that allows for delivery of transforming DNA to the target cells can be used. For example, particles can be prepared by functionalizing the surface of a gold oxide particle by providing free amine groups. DNA, having a strong negative charge, binds to the functionalized particles.

Parameters such as the concentration of DNA used to coat microprojectiles can influence the recovery of transformants containing a single copy of the transgene

Other physical and biological parameters can be varied, such as manipulation of the DNA/microprojectile precipitate, factors that affect the flight and velocity of the projectiles, manipulation of the cells before and immediately after bombardment (including osmotic state, tissue hydration and the subculture stage or cell cycle of the recipient cells), the orientation of an immature embryo or other target tissue relative to the particle trajectory, and also the nature of the transforming DNA, such as linearized DNA or intact supercoiled plasmids. Physical parameters such as DNA concentration, gap distance, flight distance, tissue distance, and helium pressure, can be optimized.

The particles delivered via biolistics can be “dry” or “wet.” In the “dry” method, the DNA-coated particles such as gold are applied onto a macrocarrier (such as a metal plate, or a carrier sheet made of a fragile material, such as mylar) and dried. The gas discharge then accelerates the macrocarrier into a stopping screen that halts the macrocarrier but allows the particles to pass through. The particles are accelerated at, and enter, the plant tissue arrayed below on growth media. The media supports plant tissue growth and development and are suitable for plant transformation and regeneration. Those of skill in the art are aware that media and media supplements such as nutrients and growth regulators for use in transformation and regeneration and other culture conditions such as light intensity during incubation, pH, and incubation temperatures can be optimized.

Those of skill in the art can use, devise, and modify selective regimes, media, and growth conditions depending on the plant system and the selective agent. Typical selective agents include antibiotics, such as geneticin (G418), kanamycin, paromomycin; or other chemicals, such as glyphosate or other herbicides.

Vector Transformation with Selectable Marker Gene

Vector-modified cells in bombarded calluses or explants can be isolated using a selectable marker gene. The bombarded tissues are transferred to a medium containing an appropriate selective agent. Tissues are transferred into selection between 0 and about 7 days or more after bombardment. Selection of modified cells can be further monitored by tracking fluorescent marker genes or by the appearance of modified explants (modified cells on explants can be green under light in selection medium, while surrounding non-modified cells are weakly pigmented). In plants that develop through shoot organogenesis (e.g., Brassica, tomato or tobacco), the modified cells can form shoots directly, or alternatively, can be isolated and expanded for regeneration of multiple shoots transgenic for the vector. In plants that develop through embryogenesis (e.g., corn or soybean), additional culturing steps may be necessary to induce the modified cells to form an embryo and to regenerate in the appropriate media.

For selection to be effective, the plant cells or tissue need to be grown on selective medium containing the appropriate concentration of antibiotic or killing agent, and the cells need to be plated at a defined and constant density. The concentration of selective agent and cell density are generally chosen to cause complete growth inhibition of wild type plant tissue that does not express the selectable marker gene; but allowing cells containing the introduced DNA to grow and expand into mini-chromosome-containing clones. This critical concentration of selective agent typically is the lowest concentration at that there is complete growth inhibition of wild type cells, at the cell density used in the experiments. However, in some cases, sub-killing concentrations of the selective agent can be equally or more effective for the isolation of plant cells containing the exogenous DNA, especially in cases where the identification of such cells is assisted by a visible marker gene (e.g., fluorescent protein gene) present on the introduced DNA.

In some species (e.g., tobacco or tomato), a homogenous clone of modified cells can also arise spontaneously when bombarded cells are placed under the appropriate selection. An exemplary selective agent is the neomycin phosphotransferase II (NptII) marker gene that confers resistance to the antibiotics kanamycin, G418 (geneticin) and paramomycin. In other species, or in certain plant tissues or when using particular selectable markers, homogeneous clones may not arise spontaneously under selection; in this case the clusters of modified cells can be manipulated to homogeneity using the visible marker genes present on the vectors as an indication of that cells contain the introduced DNA.

Regeneration of Vector-Containing Plants from Explants to Mature, Rooted Plants

For plants that develop through shoot organogenesis (e.g., sorghum, sugar cane, Brassica, tomato and tobacco), regeneration of a whole plant involves culturing of regenerable explant tissues taken from sterile organogenic callus tissue, seedlings or mature plants on a shoot regeneration medium for shoot organogenesis, and rooting of the regenerated shoots in a rooting medium to obtain intact whole plants with a fully developed root system.

For plant species, such cotton, corn and soybean, regeneration of a whole plant occurs via an embryogenic step that is not necessary for plant species where shoot organogenesis is efficient. In these plants, the explant tissue is cultured on an appropriate media for embryogenesis, and the embryo is cultured until shoots form. The regenerated shoots are cultured in a rooting medium to obtain intact whole plants with a fully developed root system.

Explants are obtained from any tissues of a plant suitable for regeneration. Exemplary tissues include hypocotyls, internodes, roots, cotyledons, petioles, cotyledonary petioles, leaves and peduncles, prepared from sterile seedlings or mature plants.

Explants are wounded (for example with a scalpel or razor blade) and cultured on a shoot regeneration medium (SRM) containing Murashige and Skoog (MS) medium as well as a cytokinin, e.g., 6-benzylaminopurinc (BA), and an auxin, e.g., a-naphthaleneacetic acid (NAA), and an anti-ethylene agent, e.g., silver nitrate (AgNO₃). For example, 2 mg/L of BA, 0.05 mg/L of NAA, and 2 mg/L of AgNO₃ can be added to MS medium for shoot organogenesis. The most efficient shoot regeneration is obtained from longitudinal sections of internode explants.

Shoots regenerated via organogenesis are rooted in a MS medium containing low concentrations of an auxin such as NAA.

To regenerate a whole plant that has been transformed, for example, explants are pre-incubated for 1 to 7 days (or longer) on the shoot regeneration medium prior to bombardment. Following bombardment, explants are incubated on the same shoot regeneration medium for a recovery period up to 7 days (or longer), followed by selection for transformed shoots or clusters on the same medium but with a selective agent appropriate for a particular selectable marker gene.

### G. Analyses of Transformed Plants

MC Autonomy Demonstration by In Situ Hybridization

While not necessary for the embodiments of the invention, it can be desirable to have a delivered MC maintained autonomously in the plant cell. To assess whether the MC is autonomous from the native plant chromosomes or has integrated into the plant genome, in situ hybridizations can be used, such as fluorescent in situ hybridization (FISH). In this assay, mitotic or meiotic tissue, such as root tips or meiocytes from the anther, possibly treated with metaphase arrest agents such as colchicines is obtained, and standard FISH methods are used to label both the centromere and sequences specific to the MC. For example, a Sorghum centromere is labeled using a probe from a sequence that labels all Sorghum centromeres, attached to one fluorescent tag, such as one that emits the red visible spectrum (ALEXA FLUOR® 568, for example (Invitrogen; Carlsbad, Calif.)), and sequences specific to the MC are labeled with another fluorescent tag, such as one emitting in the green visible spectrum (ALEXA FLUOR® 488, for example). All centromere sequences are detected with the first tag; only MCs are detected with both the first and second tag. Chromosomes are stained with a DNA-specific dye including but not limited to DAPI, Hoechst 33258, OliGreen, Giemsa YOYO, and TOTO. An autonomous MC is visualized as a body that shows hybridization signal with both centromere probes and MC specific probes and is separate from the native chromosomes.

Methods of detecting and characterizing MCs and other related techniques, including identifying centromeres for new plants can be found, for example, in U.S. Pat. Nos. 8,062,885 and 8,350,120 and US Patent Application Publication No. 2013007927.

Determination of Gene Expression Levels

The expression level of any gene present on vectors can be determined by several methods, such as for RNA, Northern Blot hybridization, Reverse Transcriptase-PCR, binding levels of a specific RNA-binding protein, in situ hybridization, or dot blot hybridization; or for proteins, Western blot hybridization, Enzyme-Linked Immunosorbant Assay (ELISA), fluorescent quantitation of a fluorescent gene product, enzymatic quantitation of an enzymatic gene product, immunohistochemical quantitation, or spectroscopic quantitation of a gene product that absorbs a specific wavelength of light.

Clonal Propagation of Transgenic Plants

To produce multiple clones of plants from a transgenic plant, any tissue of the plant can be tissue-cultured for shoot organogenesis using regeneration procedures already described. Alternatively, multiple auxiliary buds can be induced from a modified plant by excising the shoot tip, rooting the tip, and subsequently growing the tip into a plant; each auxiliary bud can be rooted and produce a whole plant.

### D. Field Evaluation of Transgenic Plants

Transgenic plant cell lines are regenerated, proliferated (to make genetically-identical replicates of each transgenic line), rooted, acclimated and used in field trials. For seed-bearing plants, seed is collected and segregated.

Descriptor data from typical plants of each transgenic accession plus tissue-cultured and regenerated from wild type and empty vector lines is collected at regular intervals over at least a year or more, depending on the type of plant transformed and is easily determined by one of skill in the art. Descriptors for which data can be collected include:
a. Morphological: flower color and size, seed size and weight, leaf color, leaf size, leaf margin teeth, number of branches from the main stem.
b. Growth: plant height and width, fresh and dry weight.
c. Chemical: farnesene, total resin, and total hydrocarbon content.
d. Phenology: first flower date, 50% bloom date, and seed maturity date (first seed harvest).
e. Seed production: total seed mass and weight
f. Imaging: digital images of entire plants, and of the leaves, flowers and seeds.
   Descriptor data (morphological, chemical, phonological, growth, production, and imaging) are collected, descriptive statistics performed and results analyzed. Seeds from selected transgenic lines that approach or meet the predetermined target are further propagated for large scale field trials. In this experiment, secondary input targets such as water requirements fertilizer requirement, and management practices are typically evaluated.

In the cases of increased terpenoid production, such as farnesene, NIR can be used to follow farnesene accumulation during the growing season. Plants from the field trials can also provide the materials needed for the initial extraction scale-up. Experiments can also be conducted to determine the stability of farnesene post-harvest in whole, chopped and chipped plants, and under a range of storage conditions varying time, temperature and humidity (Coffelt et al., 2009; Cornish et al., 2000a; Cornish et al., 2000b; McMahan et al., 2006).

### E. Processing of Transgenic Plants for Terpenoid Biofuel (Exemplified with Farnasene)

Extraction of Farnesene from Transgenic Feedstock

In previous studies, farnesene has been extracted from plant tissues using solid-phase microextraction (SPME) (Demyttenaere et al., 2004; Zini et al., 2003), subcritical CO₂ extraction (Rout et al., 2008), microwave-assisted solvent extraction (Serrano and Gallego, 2006), and two-stage solvent extraction (Pechous et al., 2005). Ionic liquid methods to extract aromatic and aliphatic hydrocarbons (Arce et al., 2008; Arce et al., 2007) can also be used for farnesene extraction. These techniques are useful on a small scale. While chipped and ground dry plants, sometimes coupled with pellitization, have been effectively extracted using solvents, further disruption or poration of plant cell walls may increase extraction efficiency. The effect of various pretreatment methods can be tested, including mild alkali or acid treatment, ammonia explosion, and steam explosion, on extraction efficiency and product purity. Ultrasound-assisted extraction (Hernanz et al., 2008), liquid-liquid extraction at high pressure, and/or high temperature also may assist in solvent penetration (into the cell wall) and improve farnesene extraction.

Extraction methods can be tested and scaled through three stages: (1) individual plant analyses, (2) 0.5-5 L batch extractions, and (3) pilot scale extraction. Hexane, pentane and chloromethane (Edris et al., 2008; Mookdasanit et al., 2003), have been used as solvents for farnesene extraction, and acetone for resin extraction can also be tested. Alternative solvents, such as ethyl lactate and 2,3 butanediol, which allow large-scale operation at higher temperatures for effective solvent distribution ratio and selectivity. Samples of transgenic plants are dried and ground using lab or hammer mills, depending on the scale required. Following solvent selection, the 0.5-5 L experiments can initially use published biomass to solvent ratios and other parameters (Arce et al., 2007; Lai et al., 2005; Mookdasanit et al., 2003; Pechous et al., 2005; Serrano and Gallego, 2006; Zheng et al., 2004), including those previously described (Ananda and Vadlani, 2010a; Ananda and Vadlani, 2010b), (Oberoi et al., 2010). The best temperature, agitation rate, extraction time, substrate:solvent ratio, moisture content of biomass, and temperature range obtained can be determined by one of skill in the art to develop the design of experiments using response surface methodology (Brijwani et al., 2010). The optimal parameters inform selection of the solvent system (s) in which farnesene exhibits the greatest solubility and the highest partition coefficient. The quality of the extractant can be analyzed with gas chromatography-mass spectrometry (GC-MS), and farnesene content can be quantified using ¹H and ¹³C NMR (Zheng et al., 2004). Pilot studies can provide the relevant data for optimization of β-farnesene extraction in terms of solvent choice, solubility, yield, and solvent recoverability.

Conversion of Farnesene to Farnesane

The β-farnesene-rich material from the extraction process can be hydrogenated via metal catalysis in a high-pressure Parr reactor. Since hydrogenation is an established process for conversion of olefins in chemical industry, various industrial-grade metal catalysts can be used (Gounder and Iglesia, 2011; Knapik et al., 2008; Zhang et al., 2003), such as palladium on carbon, and platinum, copper or nickel supported on alumina (or other acidic support). Catalyst loading (10-90 g/L), farnesene concentration (100-600 g/L), compressed hydrogen flow (40-100 psig), temperature (40-80° C.), and reaction time, can be optimized for efficient farnesane production. Catalytic efficiency can be characterized before and after hydrogenation using Fourier transform infrared spectroscopy (FTIR) and X-ray diffraction, with respect to carbon selectivity, operating parameters (temperature, pressure), reaction time, and final farnesane purity. Reaction completion can be determined using gas chromatography-flame ionization detection (GC-FID). These data inform performance of medium scale (50-1000 L) trials for efficient farnesane production from transgenic plants.

### DEFINITIONS

“Autonomous” means, when referring to MCs, that when delivered to plant cells, at least some MCs are transmitted through mitotic division to daughter cells and are episomal in the daughter plant cells, i.e., are not chromosomally integrated in the daughter plant cells. Daughter plant cells that contain autonomous MCs can be selected for further propagation using, for example, selectable or screenable markers. During the introduction into a cell of a MC, or during subsequent stages of the cell cycle, there may be chromosomal integration of some portion or all of the DNA derived from a MC in some cells. The MC is still characterized as autonomous despite the occurrence of such events if a plant, plant part or plant tissue can be regenerated that contains episomal descendants of the MC distributed throughout its parts, or if gametes or progeny can be derived from the plant that contain episomal descendants of the MC distributed through its parts.

“Centromere” is any DNA sequence that confers an ability to segregate to daughter cells through cell division. This sequence can produce a transmission efficiency to daughter cells ranging from about 1% to about 100%, including to about 5%, 10%, 20%, 30%, 40%, 50%, 60%, 70%, 80%, 90% or about 95% of daughter cells. Variations in transmission efficiency can find important applications within the scope of the invention; for example, MCs carrying centromeres that confer 100% stability could be maintained in all daughter cells without selection, while those that confer 1% stability could be temporarily introduced into a transgenic organism, but later eliminated when desired. In particular embodiments of the invention, the centromere can confer stable transmission to daughter cells of a nucleic acid sequence, including a recombinant construct comprising the centromere, through mitotic or meiotic divisions, including through both mitotic and meiotic divisions. A plant centromere is not necessarily derived from plants, but has the ability to promote DNA transmission to daughter plant cells.

“Circular permutations” refer to variants of a sequence that begin at base n within the sequence, proceed to the end of the sequence, resume with base number one of the sequence, and proceed to base n−1. For this analysis, n can be any number less than or equal to the length of the sequence. For example, circular permutations of the sequence ABCD are: ABCD, BCDA, CDAB, and DABC.

“Control sequences” are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

“Derivatives” are polynucleotide or amino acid sequences formed from native compounds either directly, by modification or partial substitution. “Analogs” are polynucleotide or amino acid sequences that have a structure similar, but not identical to, the native compound but differ from it in respect to certain components or side chains. Analogs may be synthetic or from a different evolutionary origin and may have a similar or opposite metabolic activity compared to wild type. Homologs are polynucleotide sequences or amino acid sequences of a particular gene that are derived from different species.

Derivatives and analogs may be full length or other than full length if the derivative or analog contains a modified polynucleotide or amino acid.

A “homologous polynucleotide sequence” or “homologous amino acid sequence,” or variations thereof, refer to sequences characterized by a homology at the polynucleotide level or amino acid level as discussed above. Homologous polynucleotide sequences encode those sequences coding for isoforms of the polypeptides shown in Tables 1-3 and further described in Tables 4-7. Isoforms can be expressed in different tissues of the same organism as a result of, for example, alternative splicing. Homologous polynucleotide sequences may encode conservative amino acid substitutions, as well as a polypeptide possessing similar biological activity.

“Exogenous” when used in reference to a nucleic acid, for example, refers to any nucleic acid that has been introduced into a recipient cell, regardless of whether the same or similar nucleic acid is already present in such a cell. An “exogenous gene” can be a gene not normally found in the host genome in an identical context, or an extra copy of a host gene. The gene can be isolated from a different species than that of the host genome, or alternatively, isolated from the host genome but operably linked to one or more regulatory regions that differ from those found in the unaltered, native gene. The gene can also be synthesized in vitro.

“Functional” or “activity” when referring to a MC, centromere, nucleic acid, or polypeptide, for example, retains a biological and/or an immunological activity of native or naturally-occurring chromosome, centromere, nucleic acid, or polypeptide, respectively. When used to describe an exogenous nucleic acid carried on a vector, “functional” means that the exogenous nucleic acid can function in a detectable manner when the vector is within a cell, such as a plant cell; exemplary functions of the exogenous nucleic acid include transcription of the exogenous nucleic acid, expression of the exogenous nucleic acid, regulatory control of expression of other exogenous nucleic acids, recognition by a restriction enzyme or other endonuclease, ribozyme or recombinase; providing a substrate for DNA methylation, DNA glycoslation or other DNA chemical modification; binding to proteins such as histones, helix-loop-helix proteins, zinc binding proteins, leucine zipper proteins, MADS box proteins, topoisomerases, helicases, transposases, TATA box binding proteins, viral protein, reverse transcriptases, or cohesins; providing an integration site for homologous recombination; providing an integration site for a transposon, T-DNA or retrovirus; providing a substrate for RNAi synthesis; priming of DNA replication; aptamer binding; or kinetochore binding. If multiple exogenous nucleic acids are present within the vector, the function of one or preferably more of the exogenous nucleic acids can be detected under suitable conditions permitting function. A functional or active polypeptide can be one that retains at least one biological activity, such as an enzymatic activity.

“Isolated,” when referred to a molecule, refers to a molecule that has been identified and separated and/or recovered from a component of its natural environment. Contaminant components of its natural environment are materials that interfere with diagnostic or other use.

A “mini-chromosome” (“MC”) is a recombinant DNA construct including a centromere and capable of transmission to daughter cells. A MC can remain separate from the host genome (as episomes) or can integrate into host chromosomes. The stability of this construct through cell division could range between from about 1% to about 100%, including about 5%, 10%, 20%, 30%, 40%, 50%, 60%, 70%, 80%, 90% and about 95%. The MC construct can be a circular or linear molecule. It can include elements such as one or more telomeres, origin of replication sequences, stuffer sequences, buffer sequences, chromatin packaging sequences, linkers and genes. The number of such sequences included is only limited by the physical size limitations of the construct itself. It can contain DNA derived from a natural centromere, although it can be preferable to limit the amount of DNA to the minimal amount required to obtain a transmission efficiency in the range of 1-100%. The MC can also contain a synthetic centromere composed of tandem arrays of repeats of any sequence, either derived from a natural centromere, or of synthetic DNA. The MC can also contain DNA derived from multiple natural centromeres. The MC can be inherited through mitosis or meiosis, or through both meiosis and mitosis. The term MC specifically encompasses and includes the terms “plant artificial chromosome” or “PLAC,” or engineered chromosomes or micro-chromosomes and all teachings relevant to a PLAC or plant artificial chromosome specifically apply to constructs within the meaning of the term MC.

“Operably linked” is a configuration in that a control sequence, e.g., a promoter sequence, directs transcription or translation of another sequence, for example a coding sequence. For example, a promoter sequence could be appropriately placed at a position relative to a coding sequence such that the control sequence directs the production of a polypeptide encoded by the coding sequence.

“Percent (%) amino acid sequence identity” is defined as the percentage of amino acid residues that are identical with amino acid residues in a sequence, such as those shown in Tables 1-3 and further described in Tables 4-7, in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Publicly available computer software such as BLAST, BLAST2, ALIGN2 or Megalign (DNASTAR) can be used to align polypeptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where

X is the number of amino acid residues scored as identical matches by the sequence alignment program's or algorithm's alignment of A and B

and

Y is the total number of amino acid residues in B.

If the length of amino acid sequence A is not equal to the length of amino acid sequence B, the % amino acid sequence identity of A to B will not equal the % amino acid sequence identity of B to A.

In addition to naturally-occurring allelic variants of the polynucleotides useful in the invention, changes can be introduced into the polynucleotides that incur alterations in the amino acid sequence of the encoded polypeptides but does not alter polypeptide function. For example, amino acid substitutions at “non-essential” amino acid residues can be made. A “non-essential” amino acid residue is a residue that can be altered from the amino acid sequence of the polypeptides shown in Tables 1-3 and further described in Tables 4-7 without altering the polypeptides' biological activity, whereas an “essential” amino acid residue is required for biological activity.

Useful conservative substitutions are shown in Table 8, “Preferred substitutions.” Conservative substitutions whereby an amino acid of one class is replaced with another amino acid of the same type fall within the scope of the subject invention so long as the substitution does not materially alter the biological activity (although in some cases, enhanced biological activity is desirable). If such substitutions result in a change in biological activity, then more substantial changes, indicated in Table 9 as exemplary, are introduced and the products screened for biological activity.

TABLE 8
Preferred substitutions

| | | Preferred |
|---|---|---|
| Original residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Non-conservative substitutions that affect (1) the structure of the polypeptide backbone, such as a β-sheet or α-helical conformation, (2) the charge or (3) hydrophobicity, or (4) the bulk of the side chain of the target site can modify GPCR-like RAIG1 polypeptide function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE 9
Amino acid classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis, cassette mutagenesis, restriction selection mutagenesis or other known techniques can be performed on cloned DNA to produce variants.

“Percent (%) polynucleotide sequence identity” polynucleotide sequences is defined as the percentage of polynucleotides in the sequence of interest that are identical with the polynucleotides in a candidate sequence, after aligning the sequences and introducing gaps, if necessary, to achieve the maximum percent sequence identity. Alignment can be achieved in various ways well-known in the art; for instance, using publicly available software such as BLAST, BLAST-2, ALIGN or Megalign (DNASTAR) software. Those skilled in the art can determine appropriate parameters for measuring alignment, including any necessary algorithms to achieve maximal alignment over the full length of the sequences being compared.

When polynucleotide sequences are aligned, the % polynucleotide sequence identity of a given polynucleotide sequence C to, with, or against a given polynucleotide sequence D (which can alternatively be phrased as a given polynucleotide sequence C that has or comprises a certain % polynucleotide sequence identity to, with, or against a given polynucleotide sequence D) can be calculated as:

% polynucleotide sequence identity=W/Z·100

where

W is the number of polynucleotides scored as identical matches by the sequence alignment program's or algorithm's alignment of C and D

and

Z is the total number of polynucleotides in D.

When the length of polynucleotide sequence C is not equal to the length of polynucleotide sequence D, the % polynucleotide sequence identity of C to D will not equal the % polynucleotide sequence identity of D to C.

“Sorghum” means Sorghum bicolor (primary cultivated species), Sorghum almum, Sorghum am plum, Sorghum angustum, Sorghum rundinaceum, Sorghum brachypodum, Sorghum bulbosum, Sorghum burmahicum, Sorghum controversum, Sorghum drummondii, Sorghum carinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum virgatum, and Sorghum vulgare (including but not limited to the variety Sorghum vulgare var. sudanens also known as sudangrass). Hybrids of these species are also of interest in the present invention as are hybrids with other members of the Family Poaceae.

“Sugar cane” refers to any species or hybrid of the genus Saccharum, including: S. acinaciforme, S. aegyptiacum, S. alopecuroides (Silver Plume Grass), S. alopecuroideum, S. alopecuroidum (Silver Plumegrass), S. alopecurus, S. angustifolium, S. antillarum, S. arenicola, S. argenteum, S. arundinaceum (Hardy Sugar Cane (USA)), S. arundinaceum var. trichophyllum, S. asper, S. asperum, S. atrorubens, S. aureum, S. balansae, S. baldwini, S. baldwinii (Narrow Plumegrass), S. barberi (Cultivated sugar cane), S. barbicostatum, S. beccarii, S. bengalense (Munj Sweetcane), S. benghalense, S. bicorne, S. biflorum, S. boga, S, brachypogon, S. bracteatum, S. brasilianum, S. brevibarbe (Short-Beard Plume Grass), S. brevibarbe var. brevibarbe (Shortbeard Plumegrass), S. brevibarbe var. contortum (Shortbeard Plumegrass), S. brevifolium, S. brunneum, S. caducam, S. canaliculatum, S. capense, S. casi, S. caudatum, S. cayennense, S. cayennense var. gemiimim, S. cayennense var. laxiusculum, S. chinense, S. ciliare, S. coarctatum (Compressed Plumegrass), S. confertum, S. conjugatun, S. contortum, S. contortum var. contortum, S. contractum, S. cotuliferum, S. cylindricum, S. cylindricum var. contractum, S. cylindricum var. longifolium, S. deciduum, S. densum, S. diandrum, S. dissitiflorum, S. distichophyllum, S. dubium, S. ecklonii, S. edule, S. elegans, S. elephantinum, S. erianthoides, S. europaeum, S. exaltatum, S. fasciculatum, S. fastigiatum, S. fatuum, S. filifolium, S. filiforme, S. floridulun, S. formosanum, S. fragile, S. fulvum, S. fuscum, S. giganteum (sugar cane Plume Grass), S. glabrum, S. glaga, S. glaucum, S. glaza, S. grandiflorum, S. griffit ii, S. hildebrandtii, S. hirsutum, S. holcoides, S. holcoides var. warmingianum, S. hookeri, S. hybrid, S. hybridum, S. indum, S. infirmum, S. insulare, S. irritans, S. jaculatorium, S. jamaicense, S. japonicum, S. juncifolium, S. kajkaiense, S. kanashiroi, S. klagha, S. koenigii, S. laguroides, S. longifolium, S. longisetosum, S. longisetosum var. hookeri, S. longisetum, S. lota, S. luzonicum, S. macilentum, S. macrantherum, S. maximum, S. mexicanum, S. modhara, S. monandrum, S. moonja, S. munja, S. munroanum, S. muticum, S. narenga (arenga sugar cane), S. negrosense, S. obscurum, S. occidentale, S. officinale, S. officinalis, S. officinarum (Cultivated sugar cane), S. officinarum ‘Cheribon’, S. officinarum Otaheite’, S. officinarum Tele's Smoke’ (Black Magic Repellent Plant), S. officinarum L. ‘Laukona’, S. officinarum L. ‘Violaceum’, S, officinarum var. brevipedicellatum, S. officinarum var. officinarum, S. officinarum var. violaceum (Burgundy-Leaved sugar cane), S. pallidum, S. paniceum, S. panicosum, S. pappiferum, S. parviflorum, S. pedicellare, S. perrieri, S. polydactylum, S. polystachyon, S. polystachyum, S. porphyrocomum, S. procerum, S. propinquum, S. punctatum, S. rara, S. rarum, S. ravennae (Hardy Pampas Plume Grass), S. repens, S. reptans, S. ridleyi, S. robustum (Wild New Guinean Cane), S. roseum, S. rubicundum, S. rufum, S. sagittatum, S. sanguineum, S. sape, S. sara, S. scindicus, S. semidecumbens, S. sibiricum, S. sikkhnense, S. sinense (Cultivated sugar cane), S. sisca, S. sorghum, S. speciosissimum, S. sphacelatum, S. spicatum, S. spontaneum (Wild Sugar Cane), S. spontaneum var. insulare, S. spontanum, S. stenophyllum, S. stewartii, S. strictum, S. teneriffae, S. ternatum, S. thunbergii, S. tinctorium, S. tridentatum, S. trinii, S. tristachyum, S. velutinum, S. versicolor, S. viguieri, S. villosum, S. violaceum, S. wardii, S. warmingianum, S. williamsii.

“Guayule” means the desert shrub, Parthenium argentatum, native to the southwestern United States and northern Mexico and which produces polymeric isoprene essentially identical to that made by Hevea rubber trees (e.g., Hevea brasiliensis) in Southeast Asia.

“Hevea” means Hevea brasiliensis, the Para rubber tree.

“Hybridizes under low stringency, medium stringency, and high stringency conditions” describes conditions for hybridization and washing. Hybridization is a well-known technique (Ausubel, 1987). Low stringency hybridization conditions means, for example, hybridization in 6× sodium chloride/sodium citrate (SSC) at about 45° C., followed by two washes in 0.5×SSC, 0.1% SDS, at least at 50° C.; medium stringency hybridization conditions means, for example, hybridization in 6×SSC at about 45° C., followed by one or more washes in 0.2×SSC, 0.1%) SDS at 55° C.; and high stringency hybridization conditions means, for example, hybridization in 6×SSC at about 45° C., followed by one or more washes in 0.2×SSC, 0.1% SDS at 65° C. Another non limiting example of stringent hybridization conditions are hybridization in a high salt buffer comprising 6×SSC, 50 mM Tris HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. Another non limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Another non limiting example of low stringency hybridization conditions are hybridization in 35% formamide, 5×SSC, 50 mM Tris HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency that may be used are well known in the art (e.g., as employed for cross species hybridizations).

“Inducible promoter” means a promoter induced by the presence or absence of a biotic or an abiotic factor.

“Plant part” includes pollen, silk, endosperm, ovule, seed, embryo, pods, roots, cuttings, tubers, stems, stalks, fiber (lint), square, boll, fruit, berries, nuts, flowers, leaves, bark, wood, whole plant, plant cell, plant organ, epidermis, vascular tissue, protoplast, cell culture, crown, callus culture, petiole, petal, sepal, stamen, stigma, style, bud, meristem, cambium, cortex, pith, sheath, or any group of plant cells organized into a structural and functional unit. In one preferred embodiment, the exogenous nucleic acid is expressed in a specific location or tissue of a plant, for example, epidermis, vascular tissue, meristem, cambium, cortex, pith, leaf, sheath, flower, root or seed.

“Polypeptide” does not refer to a specific length of the encoded product and, therefore, encompasses peptides, oligopeptides, and proteins. “Exogenous polypeptide” means a polypeptide that is not native to the plant cell, a native polypeptide in that modifications have been made to alter the native sequence, or a native polypeptide whose expression is quantitatively altered as a result of a manipulation of the plant cell by recombinant DNA techniques.

“Promoter” is a DNA sequence that allows the binding of RNA polymerase (including but not limited to RNA polymerase I, RNA polymerase II and RNA polymerase Ill from eukaryotes), and optionally other accessory or regulatory factors, and directs the polymerase to a downstream transcriptional start site of a nucleic acid sequence encoding a polypeptide to initiate transcription. RNA polymerase effectively catalyzes the assembly of messenger RNA complementary to the appropriate DNA strand of the coding region.

A “promoter operably linked to a heterologous gene” is a promoter that is operably linked to a gene or other nucleic acid sequence that is different from the gene to that the promoter is normally operably linked in its native state. Similarly, an “exogenous nucleic acid operably linked to a heterologous regulatory sequence” is a nucleic acid that is operably linked to a regulatory control sequence to that it is not normally linked in its native state.

“Regulatory sequence” refers to any DNA sequence that influences the efficiency of transcription or translation of any gene. The term includes sequences comprising promoters, enhancers and terminators.

“Repeated nucleotide sequence” refers to any nucleic acid sequence of at least 25 bp present in a genome or a recombinant molecule, other than a telomere repeat, that occurs at least two or more times and that are preferably at least 80% identical either in head to tail or head to head orientation either with or without intervening sequence between repeat units.

“Retroelement” or “retrotransposon” refers to a genetic element related to retroviruses that disperse through an RNA stage; the abundant retroelements present in plant genomes contain long terminal repeats (LTR retrotransposons) and encode a polyprotein gene that is processed into several proteins including a reverse transcriptase. Specific retroelements (complete or partial sequences (e.g., “retroelement-like sequence” and “retrotransposon-like sequence”) can be found in and around plant centromeres and can be present as dispersed copies or complex repeat clusters. Individual copies of retroelements can be truncated or contain mutations; intact retrolements are rarely encountered.

“Satellite DNA” refers to short DNA sequences (typically <1000 bp) present in a genome as multiple repeats, mostly arranged in a tandemly repeated fashion, as opposed to a dispersed fashion. Repetitive arrays of specific satellite repeats are abundant in the centromeres of many higher eukaryotic organisms.

“Screenable marker” is a gene whose presence results in an identifiable phenotype. This phenotype can be observed under standard conditions, altered conditions such as elevated temperature, or in the presence of certain chemicals used to detect the phenotype. The use of a screenable marker allows for the use of lower, sub-killing antibiotic concentrations and the use of a visible marker gene to identify clusters of transformed cells, and then manipulation of these cells to homogeneity. Examples of screenable markers include genes that encode fluorescent proteins that are detectable by a visual microscope such as the fluorescent reporter genes DsRed, ZsGreen, ZsYellow, AmCyan, Green Fluorescent Protein (GFP). An additional preferred screenable marker gene is lac.

“Structural gene” is a sequence that codes for a polypeptide or RNA and includes 5′ and 3′ ends. The structural gene can be from the host into which the structural gene is transformed or from another species. A structural gene usually includes one or more regulatory sequences that modulate the expression of the structural gene, such as a promoter, terminator or enhancer. Structural genes often confer some useful phenotype upon an organism comprising the structural gene, for example, herbicide resistance. A structural gene can encode an RNA sequence that is not translated into a protein, for example a tRNA or rRNA gene.

“Synthetic,” when used in the context of a polynucleotide or polypeptide, refers to a molecule that is made using standard synthetic techniques, e.g., using an automated DNA or peptide synthesizer. Synthetic sequence can be a native sequence, or a modified sequence.

“Terpenes” are derived from five-carbon isoprene units, which have the molecular formula C₅H₈. A “sesquiterpene” has 3 isoprene units and has the molecular formula C₁₅H₂₄. “Terpenoids” or “isoprenoids” are terpenes that are biochemically modified, such as by oxidation or rearrangement. A “sesquiterpenoid” has 3 isoprene units, such as sesquiterpene, and is biochemically modified.

“Transformed,” “transgenic,” “modified,” and “recombinant” refer to a host organism such as a plant into which an exogenous or heterologous nucleic acid molecule has been introduced, and includes whole plants, meiocytes, seeds, zygotes, embryos, endosperm, or progeny of such plants that retain the exogenous or heterologous nucleic acid molecule but that have not themselves been subjected to the transformation process.

TABLE OF SELECTED ABBREVIATIONS

| Abbreviation | Definition |
|---|---|
| AACT | Acetoacetyl-CoA thiloase |
| ASE | accelerated solvent extraction |
| β-FS | β-farnesene synthase |
| CCE | carbon capture enhancement |
| CMK | 4-diphosphocytidyl-2-C-methyl-D-erythritol kinase |
| CMS | 2-C-methyl-D-erythritol 4-phosphate cytidylyltransferase |
| DMAPP | dimethylallyl pyrophosphat |
| DXP | deoxyxylulose-5-phosphate |
| DXR | deoxyxylulose-5-phosphate reductoisomerase |
| DXS | 1-deoxy-D-xylulose-5-phosphate synthase |
| FME | farnesene metabolic engineering |
| FPP | farnesyl pyrophosphate |
| FPPS | farnesene diphosphate synthase |
| FDPS | farnesyl diphosphate synthase |
| FTIR | Fourier transform infrared spectroscopy |
| FS | farnesene synthase |
| GC | gas chromatography |
| GC-FID | gas chromatography-flame ionization detection |
| GD, GPP | geranyl diphosphate |
| GPPS | farnesyl diphosphate synthase |
| HDR | hydroxymethylbutenyl diphosphate reductase |
| HDS | 4-hydroxy-3-methylbut-2-en-1-yl diphosphate synthase |
| HMG-CoA | hydroxymethylglutaryl-coenzyme A |
| HMGR | 3-hydroxy-3-methylglutaryl coenzyme A reductase |
| HMGS | 3-hydroxy-3-methylglutaryl coenzyme A synthase |
| HPLC | High-pressure liquid chromatography |
| IPP | isopentenyl pyrophosphate |
| IPPI | isopentenyl-diphosphate delta-isomerase |
| LC/MS | liquid chromatography/mass pectrometry |
| MC, MCs | mini-chromosome(s) |
| MCS | hydroxymethylglutaryl-CoA synthase |
| MEP | methylerthritol phosphate pathway |
| MK | mevalonate kinase |
| MPD | mevalonate phyrophosphate decarboxylase |
| MVA | mevalonic acid pathway |
| NIR | near infrared |
| PMK | phosphomevalonate kinase |
| PMI | phosphomannose isomerase |
| RSM | response surface methodology |
| SPME | solid-phase microextraction |

### EXAMPLES

The following examples are meant to only exemplify the invention, not to limit it in any way. One of skill in the art can envision many variations and methods to practice the invention.

### Example 1

### Identification of Candidate Genes that Encode for MVA and MEP Pathway Enzymes

The various enzymes that are involved in the MVA pathway, the MEP pathway, and FSS pathway can be used to produce farnesene were identified in plants or in microorganisms such as E. coli, fungi, and plants.

The protein sequences of the biochemically characterized genes encoding the MVA or MEP pathway were then used as a query to search publically available protein databases to identify protein homologs. The closest protein sequence with the highest homology to the query sequence from each organism was considered as the putative candidate protein sequence. Tables 1-7 summarize the polypeptides and nucleic acid sequences that were identified and further selected for the embodiments of the invention.

### Example 2

### Quantify Baseline Terpene Profiles in Sorghum Plants to Identify Key Intermediates and Products of Terpene Pathway

Extraction of terpene from plant samples was carried out using Mini-Bead Beater—16 instrument (Biospec Products, Catalog number 607; Bartlesville, Okla., USA). Polypropylene microvial (7 mL, Biospec Products, Catalog number 3205) was used for extraction. Ground leaf/stem/callus (1.5 g), dichloromethane (3.0 mL, Fisher Scientific, catalog number D151SK-4) and 6 chrome-steel beads (3.2 mm diameter, Biospec Products, Catalog number 11079132c) were taken in the microvial and bead beaten for 90 seconds (30 second×3 times). Vials were cooled in ice bath between two consecutive beating cycles. Volume of supernatant collected after extraction was 2 mL. 1 mL of it was transferred to a 2 mL microcentrifuge tube (VWR International, Catalog number 89000-028; Radnor, Pa., USA) and centrifuged for 10 minutes at 4° C. at 10,000 rpm. 500 microL of the centrifuged solution was transferred to GC vial and spiked with 50 microL of 1,2,3-trichlorobenzene (Acros Organics, Catalog number AC13939-2500; Thermo Fisher Scientific, N.J., USA) stock solution in DCM (5 mg/mL).

GC was run in Shimadzu GC 2014 instrument (Shimadzu; Kyoto, Japan) using an Agilent HP-5 column (Agilent Technologies, Inc.; Santa Clara, Calif., USA). The following GC conditions were used for the analysis. 1 microL of samples was injected using a splitless injection mode. Injection port was held at 250° C. and sampling time was 1 minute with Helium as carrier gas. The following flow control mode was used with a Pressure: 103.1 kPa and a total flow of 6.4 mL/minute and a column flow of 1.14 mL/minute. The linear velocity was 29.3 cm/sec with a purge flow of 3.0 mL/minute. The following column temperature gradient was used: 80° C. for 2 minute, increased to 150° C. with a gradient of 3.5° C./minute and held at 150° C. for 15 minute, increased to 250° C. with a gradient of 10° C./minute, held at 250° C. for 2 minute for a total run time of 49 minutes. Flame ionization detector at a temperature of 250° C. was used for detecting compounds that were eluted.

For GC-MS analysis, samples were extracted as for GC analysis except for the following changes. 100 microL of the centrifuged solution was transferred to GC vial, diluted with 100 microL dichloromethane and spiked with 10 microL of 1,2,3-trichlorobenzene (Acros Organics, Catalog number AC13939-2500) stock solution in dichloromethane (5 mg/mL).

GC-MS was run in Agilent 6890N GC with an Agilent 122-5562 DB-5 ms column coupled to an Agilent 5975N quadrupole selective mass detector. The following GC conditions were used for the analysis. 1 microL of samples was injected using a splitless injection mode. Injection port was held at 280° C. and sampling time was 1 minute with Helium carrier gas. The following flow control mode was used with a pressure of 19.02 psi and a total flow of 5.9 mL/minute and a column flow of 1 mL/minute. The linear velocity was maintained at 26 cm/sec with a purge flow of 2.0 mL/minute. The following column temperature gradient was used; 80° C. for 2 minutes then increased to 280° C. with a gradient of 5° C./minute and held at 280° C. for 18 minutes for a total run time of 60 minutes. The following MS conditions were used for data acquisition. Scan acquisition mode with a solvent delay of 9 minutes. Scan parameters we set to detect compounds with low mass of 50 and high mass of 650. The MS quad temperature was maintained at 150° C. and MS source at 230° C.

Metabolites of the MVA pathway were quantified using liquid chromatography triple-quadrupole mass spectrometry (LC-MS/MS). Briefly, flash-frozen plant tissues were triple-ground to a fine powder with liquid nitrogen, extracted overnight in methanol (10 mL/g tissue; aloin [0.2 μg/ml] was added as an internal standard) at room temperature and filtered. Samples were dried and resuspended in methanol, and MVA pathway intermediates were quantified using LC-MS/MS methodologies based on previously published protocols (Nagel et al. [2012] Nonradioactive assay for detecting isoprenyl diphosphate synthase activity in crude plant extracts using liquid chromatography coupled with tandem mass spectrometry. Anal. Biochem. 422: 33-38). The results of LC-MS/MS analyses are summarized in Table 10.

Our data show that, as expected, in both guayule and sorghum MVA pathway intermediates make up only a small fraction of the total fresh weight. Additionally, with the exception of FPP in leaves of the sweet sorghum line Rio (R10), all MVA pathway intermediates are present in guayule (data not shown) at concentrations 3-(e.g. IPP) to 100-(in the case of MVAP in stem tissues) fold more than in sorghum. In most cases, guayule metabolite abundances data correlated with the relative abundance of their cognate transcripts (data not shown).

TABLE 10
LC-MS quantification of MVA pathway intermediates in guayule (AZ101)
and sorghum (R10 and TX430) leaves and stems¹

| Tissue | | MVA | MVAP | MVAPP | IPP | GPP | FPP |
|---|---|---|---|---|---|---|---|
| R10 leaf | % frozen | 1.01E−03 | 0 | 0 | 1.28E−03 | 0 | 5.52E−06 |
| | weight | | | | | | |
| | std. dev. | 2.75E−04 | 0 | 0 | 3.08E−04 | 0 | 7.79E−07 |
| R10 stem | % frozen | 1.00E−05 | 6.40E−07 | 1.61E−05 | 3.77E−04 | 0 | 0 |
| | weight | | | | | | |
| | std. dev. | 8.75E−06 | 1.11E−06 | 8.79E−06 | 5.92E−05 | 0 | 0 |
| TX430 leaf | % frozen | 2.58E−04 | 2.52E−06 | 2.18E−05 | 5.15E−04 | 0 | 0 |
| | weight | | | | | | |
| | std. dev. | 3.87E−05 | 2.21E−06 | 7.82E−06 | 1.16E−04 | 0 | 0 |
| TX430 stem | % frozen | 1.38E−05 | 6.13E−07 | 1.51E−05 | 3.39E−04 | 0 | 0 |
| | weight | | | | | | |
| | std. dev. | 4.20E−06 | 1.06E−06 | 2.11E−06 | 8.35E−05 | 0 | 0 |

¹Metabolite values are presented as % frozen tissue mass, and represent the mean of three biological replicates, with standard deviations. The limits of detection (LOD) in ng loaded onto the column, for each compound were 0.15 for HMG-CoA, MVA, MVAP, MVAPP, and GPP; LOD for IPP and FPP was 0.0075 ng. Zero (0) represents values below LOD. HMG-CoA was below limits of detection in all samples and is therefore not reported.

Elicitors of Sesquiterpene Metabolism in Sorghum

Elicitors such as methyl jasmonate (MeJ), salicylic acid (SA), ethephon and benzothiadiazole (BTH) that are known to induce sesquiterpene metabolism in plants were applied to induce farnesene and other sesquiterpene biosynthesis in sorghum. Rapidly growing young leaves from 40-day old sorghum plants were excised at the base and immediately place in a flask containing 4 mM of SA and 4 mM MeJ. As a control, leaves were treated with water, and each treatment replicated three times. In both experiments, samples collected after induction were immediately frozen in liquid nitrogen and analyzed by GC within 24 hours of collection. Results from GC analysis clearly showed that the sorghum leaf samples were induced by MeJ after 30 hours of induction and multiple compounds with retention time similar to sesquiterpenes were seen in GC chromatogram (FIG. 9). A compound with same retention time as β-farnesene (21.1 min) was produced in samples that were induced by MeJ. The GC-MS analysis confirmed the key sesquiterpenes that are induced in sorghum leaves as farnesene and caryophyllene. We expect transgenic plants over-expressing the key MVA or MEP pathway genes to produce higher levels of farnesene as compared to non-transformed plants when induced.

### Example 3

### Determine the Relative Steady-State Transcript Levels of Endogenous Terpene Pathway Genes in Sorghum Normalized to Respective Housekeeping Genes

Sorghum Microarray Design and Production

Sorghum microarrays were designed (Affymetrix; Santa Clara, Calif., USA). The probes for ˜27,500 genes were designed based on the whole genome sequence of Sorghum bicolor genotype BTx623, available at Phytozome (Paterson A H, et al. (2009). “The Sorghum bicolor genome and the diversification of grasses.” Nature 457, 551-556). The gene sequences were downloaded from the FTP site of Phytozome and parsed into an instruction file format. Overall, we have 150,337 probe selection regions representing the exons and UTRs. Over 1.4 million probes were designed for 27,500 predicted transcripts designed for 150,000 unique exons as well as the microRNA sequences downloaded from noncoding RNA sequence database (Kin T., et al. 2007. fRNAdb: a platform for mining/annotating functional RNA candidates from non-coding RNA sequences. Nucleic Acids Res, 35(Database issue):D145-8).

Selection of Sorghum Tissues for Gene Expression Profiling

Tissues collected from field experiments during 2011 were leveraged for gene expression profiling and discovery of stem-specific promoters. These samples consist of tissues from seedling shoots, seedling roots, shoot meristems, leaves, stems and dissected stem tissues (pith and rind) selected from six diverse genotypes. RNA was isolated from 79 samples and the microarray analysis was conducted by Precision Biomarker Resources, Inc. (Evanston, Ill., USA).

Microarray Data Analysis

Microarray data were analyzed using Partek Genomic Suite 6.6 software (Partek, Inc.; Saint Louis, Mo., USA). The data from CEL files was normalized using the gcRMA algorithm with background adjustments for probe sequence. The log 2 normalized data from exons was used to conduct analysis of variance (ANOVA). The candidate MVA and MEP pathway genes identified from sorghum were analyzed by microarray to determine the relative gene expression levels in various tissues as compared to housekeeping genes actin and ubiquitin. For a given tissue, the gene expression data was normalized as percentage of actin (Sb01g010030) gene expression. The results of the analysis suggest that there was substantial difference in gene expression among the MVA (Table 11) and MEP (Table 12) pathway genes within a tissue and among the tissues. In comparison to HMGR (the known rate-limiting MVA pathway gene in plants), AACT and HMGS genes showed relatively higher expression in various sorghum tissues while the rest of the MVA pathway genes showed similar or lower gene expression. We also observed a similar trend in guayule with higher number of AACT transcripts as compared to HMGR.

TABLE 11
Steady-state transcript levels of sorghum MVA pathway genes
relative to sorghum actin gene transcript¹

| Gene | | | | | | | | |
|---|---|---|---|---|---|---|---|---|
| Name | Gene ID | Root | Shoot | Leaf | Meristem | Internode | Pith | Rind |
| FPPS-1 | Sb03g032280.1 | 6.9 | 38.7 | 205.1 | 23.3 | 19.6 | 23.4 | 19.9 |
| FPPS-2 | Sb09g027190.1 | 21.0 | 10.5 | 8.3 | 15.9 | 17.2 | 28.3 | 17.6 |
| IPPI-1 | Sb02g035700.1 | 8.4 | 10.7 | 30.8 | 5.4 | 4.5 | 8.2 | 5.7 |
| IPPI-2 | Sb09g020370.1 | 3.2 | 7.2 | 23.4 | 6.4 | 9.9 | 14.0 | 10.4 |
| PMK | Sb01g040900.1 | 5.3 | 8.0 | 21.9 | 6.0 | 7.6 | 14.1 | 7.1 |
| MPD | Sb04g035950.1 | 10.4 | 12.3 | 18.7 | 13.4 | 14.5 | 23.9 | 14.1 |
| MK | Sb04g001220.1 | 4.1 | 4.5 | 6.6 | 4.4 | 5.7 | 9.1 | 5.9 |
| HMGR-1 | Sb07g027480.1 | 13.0 | 18.7 | 47.4 | 14.3 | 17.3 | 39.5 | 14.7 |
| HMGR-2 | Sb02g028630.1 | 14.7 | 24.3 | 63.8 | 15.5 | 21.2 | 36.2 | 17.6 |
| HMGS-1 | Sb02g030270.1 | 30.5 | 32.9 | 22.4 | 42.6 | 31.8 | 47.2 | 26.6 |
| HMGS-2 | Sb07g025240.1 | 9.1 | 20.3 | 79.6 | 3.4 | 19.4 | 25.5 | 24.8 |
| HMGS-3 | Sb01g049310.1 | 10.4 | 19.7 | 51.4 | 8.8 | 4.3 | 3.0 | 6.6 |
| AACT-1 | Sb08g023050.1 | 20.5 | 31.6 | 86.0 | 21.1 | 25.6 | 31.0 | 23.3 |
| AACT-2 | Sb01g033360.1 | 12.3 | 12.1 | 19.2 | 9.3 | 17.1 | 14.4 | 10.3 |
| Actin | Sb01g010030 | 100.0 | 100.0 | 100.0 | 100.0 | 100.0 | 100.0 | 100.0 |
| ubiquitin | Sb10g027470 | 62.3 | 97.7 | 233.2 | 50.7 | 100.0 | 264.4 | 163.8 |

¹Data are presented in percentages as compared to actin gene expression

TABLE 12
Steady-state transcript levels of sorghum MEP pathway genes
relative to sorghum actin gene transcript¹

| Gene | | | | | | | | |
|---|---|---|---|---|---|---|---|---|
| Name | Gene ID | Root | Shoot | Leaf | Meristem | Internode | Pith | Rind |
| HDR | Sb01g009140.1 | 3.8 | 18.5 | 112.6 | 4.1 | 9.6 | 19.7 | 13.9 |
| HDS | Sb04g025290.1 | 3.4 | 25.8 | 176.6 | 4.1 | 11.4 | 20.6 | 14.2 |
| MCS | Sb04g031830.1 | 1.2 | 3.8 | 19.8 | 1.3 | 2.1 | 3.5 | 2.4 |
| CMK | Sb03g037310.1 | 2.6 | 14.4 | 87.6 | 4.1 | 6.2 | 8.5 | 6.9 |
| CMS | Sb03g042160.1 | 2.0 | 4.0 | 25.5 | 1.9 | 3.6 | 4.0 | 3.9 |
| DXR | Sb03g008650.1 | 13.5 | 58.9 | 312.5 | 5.1 | 17.1 | 21.8 | 22.6 |
| DXS | Sb09g020140.1 | 3.8 | 30.2 | 152.5 | 6.3 | 15.3 | 17.9 | 17.7 |
| DXS | Sb02g005380.1 | 1.7 | 2.4 | 14.6 | 0.9 | 1.7 | 2.8 | 2.1 |
| DXS | Sb10g002960.1 | 11.0 | 17.9 | 67.5 | 9.8 | 22.6 | 35.2 | 25.2 |
| Actin | Sb01g010030 | 100.0 | 100.0 | 100.0 | 100.0 | 100.0 | 100.0 | 100.0 |
| ubiquitin | Sb10g027470 | 62.3 | 97.7 | 233.2 | 50.7 | 100.0 | 264.4 | 163.8 |

¹Data is presented in percentages as compared to actin gene expression

### Example 4

### Metabolon FME Gene Stack Constructs

We have identified genes necessary to transfer the entire MVA pathway as a putative metabolon (a structural-functional complex formed between sequential enzymes of a metabolic pathway that facilitates substrate channeling from one enzymatic transformation to the next, resulting in high biosynthetic rates) from Saccharomyces cerevisiae and Hevea brasiliensis to improve flux into β-farnesene biosynthesis (See Tables 1-7). Although there is extensive functional characterization of the terpenoid pathway in Hevea, MVA pathway genes (Sando et al (2008) Biosci Biotechnol Biochem 72:2049-60) were selected from this species because of the inherent ability of Hevea to produce substantial amounts of terpenoid compounds. Thus, as a metabolon of physically associated, functionally interacting enzymes, the Hevea MVA pathway represents a significant opportunity to obtain maximal rates of acetyl CoA conversion into terpenoid precursors.

In this approach, seven key enzymes that are essential for the conversion of Acetyl CoA to IPP and DMAPP are over-expressed in addition to FPPS and FS to produce β-farnesene. These include the enzymes acetoacetyl-CoA thiolase (AACT); 3-hydroxy-3-methylglutaryl coenzyme A synthase (HMGS); 3-hydroxy-3-methylglutaryl coenzyme A reductase (HMGR); mevalonate kinase (MK); phosphomevalonate kinase (PMK); mevalonate pyrophosphate decarboxylase (MPD) and isopentenyl-diphosphate delta-isomerase (IPPI), farnesene diphosphate synthase (FPPS) and β-farnesene synthase (β-FS). Because of its ease of transformation, sugar cane was used as a surrogate system to test the MVA pathway metabolon concept to produce β-farnesene. Once the metabolon concept was tested in sugar cane, a limited number of constructs that show promising results were further evaluated in sorghum.

### Example 5

### Design FME Gene Stack Constructs to Test MVA Pathway Metabolon

We engineered the MVA pathway metabolon (nine genes) constructs in sorghum and sugar cane via a combination of gene stacking and co-transformation. To enable rapid gene construction and to accommodate nine genes, we subdivided the genes that encode the MVA pathway into three gene constructs. Construct 1 contained genes that code for the three rate-limiting enzymes (HMGR, FPPS and β-FS) and the selectable marker (NPTII) for selecting transgenic events. Construct 2 contained two genes (AACT and HMGS) that encode enzymes upstream of the key rate-limiting enzyme HMGR. Construct 3 contained four genes (MK, PMK, MPD and IPPI) that encode enzymes downstream of HMGR. A list of constructs designed to engineer the MVA pathway metabolon are shown in Table 13.

TABLE 13
Constructs to express whole MVA pathway

| Construct | | Construct 1 | | Construct 2 | | Construct 3 | |
|---|---|---|---|---|---|---|---|
| Set | Description* | Promoter | Genes | Promoter | Genes | Promoter | Genes |
| So10 | Constitutive expression | Ubiquitin | HMGR | SCBV2 | AACT | PRP3.0 | MK |
| | of complete MVA | | | | | | |
| | pathway from fungi. | | | | | | |
| | | Actin | FPPS | SCBV2 | HMGS | PRP3.0 | PMK |
| | | Ubiquitin | β-FS | | | PRP3.0 | MPD |
| | | YAT | NPTII | | | PRP3.0 | IPPI |
| So4 | Lignifying cell-preferred | OMT1 | HMGR | SCBV2 | AACT | PRP3.0 | MK |
| | expression of complete | | | | | | |
| | MVA pathway from | | | | | | |
| | fungi. | | | | | | |
| | | OMT1 | FPPS | SCBV2 | HMGS | PRP3.0 | PMK |
| | | OMT1 | β-FS | | | PRP3.0 | MPD |
| | | YAT | NPTII | | | PRP3.0 | IPPI |
| So11 | Constitutive expression | Ubiquitin | HMGR | SCBV2 | AACT | PRP3.0 | MK |
| | of complete MVA | | | | | | |
| | pathway from Hevea. | | | | | | |
| | | Actin | FPPS | SCBV2 | HMGS | PRP3.0 | PMK |
| | | Ubiquitin | β-FS | | | PRP3.0 | MPD |
| | | YAT | NPTII | | | PRP3.0 | IPPI |
| So6 | Lignifying cell-preferred | OMT1 | HMGR | SCBV2 | AACT | PRP3.0 | MK |
| | expression of complete | | | | | | |
| | MVA pathway from | | | | | | |
| | Hevea. | | | | | | |
| | | OMT1 | FPPS | SCBV2 | HMGS | PRP3.0 | PMK |
| | | OMT1 | β-FS | | | PRP3.0 | MPD |
| | | YAT | NPTII | | | PRP3.0 | IPPI |
| Control | Vector with selectable | YAT | NPTII | | | | |
| | marker | | | | | | |

*For description of target expressed polypeptides and associated polynucleotides, please see Tables 1-7.

### Example 6

### Introduction of MVA Constructs into Sugar Cane Plant Cells

Sugar cane variety L97-128 was bombarded with the sets of constructs shown in Table 13 using standard protocols (Frame et al., 2000). For bombardment, DNA amount equivalent to 60 billion molecules for each construct was coated on to 1.8 mg of 0.6 μM gold particles and precipitated using 2.5M CaCl₂ and 0.1M spermidine for 2 hrs following standard protocol (Frame et al., 2000). The precipitated DNA-gold particles was dissolved in 36 μl ethanol and delivered into 60 days old sugar cane green or white callus using the Biorad PDS-1000 gene gun (Bio-Rad; Hercules, Calif., USA). Each precipitation was bombarded into 6 plates (10 billion molecules of DNA/shot). The parameters used for bombardment were 7 cm target distance; a vacuum of 27.5 Hg; 1100 psi rupture disc. Next day after bombardment, the calli were transferred on to selection medium (DBC3 medium) containing 20 mg/I geneticin and cultured at 28° C., under light for 2 weeks. Three rounds of selection were followed to obtain the transgenic calli events. The transgenic callus events were regenerated on half MS medium and rooted on half MS medium containing 15 mg/I geneticin. The regenerated transgenic plants were transferred to soil mix in 24 well flat, placed in environmental growth chamber at 28° C. for 5-8 days. The flats were then transferred to green house and placed under a mist bench for one week. The well-grown transgenic plants were finally transplanted into 1.6 gallon pots with soil:peat:perlite (1:1:1) and grown to maturity.

Initial results suggest that ˜90% of the events selected on G418 were positive for the NPTII gene and out of those, ˜25-75% contained all genes of interest depending on the number of genes expected to be present (25% when 9 or more genes are expected to be present in a co-transformation experiments with 3 constructs and 75% or higher when 3 genes are present in a single construct). Selected events were transferred to the greenhouse for plant growth. In total, we generated 339 sugar cane events from 7 experiments with 189 of the events containing all genes of interest. 94 of the events with entire MVA metabolon or with partial set of genes were planted in soil (Table 14).

TABLE 14
Summary of sugar cane transformation experiments

| | | | | # Events |
|---|---|---|---|---|
| | | NPTII PCR+ | All GOI+ | Transferred |
| Construct | Description | Events | Events | to soil |
| So4a | Lignified cell expression, yeast/E. coli MVA + | 36 | 23 | 23 |
| | ScFPPS + Aa FS | | | |
| So4b | Lignified cell expression, yeast MVA metabolon + | 84 | 19 | 19 |
| | ScFPPS + Aa FS | | | |
| So6 | Lignified cell expression, Hevea MVA metabolon + | 32 | 24 | 19 |
| | HbFPPS + Aa FS | | | |
| So10 | Constitutive expression of yeast MVA | 53 | 29 | 14 |
| | metabolon + ScFPPS + Aa FS | | | |
| So11b | Constitutive expression of Hevea MVA | 52 | 29 | 10 |
| | metabolon + HbFPPS + Aa FS) | | | |
| Control | NPTII/GFP | 15 | 15 | 5 |

GOI, genes of interest

### Example 7

### Introduction of MVA Constructs into Sorghum Plant Cells

Grain sorghum inbred line TX430 was transformed by biolistics. Calli were bombarded with 0.6 μm diameter gold particles coated with plasmid DNA (3 μg DNA per shot per construct) at a vacuum of 14 psi inside a PDS-1000/He Biolistic® Particle Delivery System (Bio-Rad). The constructs used and a description of the genes of interest is given in Table 15. To date, we have generated 99 sorghum events from 6 experiments with 32 of the events containing the entire MVA metabolon.

TABLE 15
Summary of sorghum MVA-metabolon experiments

| | | NPTII | | # Events |
|---|---|---|---|---|
| | | PCR+ | All GOI+ | Transferred |
| Construct | Description | Events | Events | to soil |
| Sb4a | Lignified cell expression, yeast/E. coli MVA + | 13 | 4 | 11 |
| | ScFPPS + Aa FS | | | |
| Sb4b | Lignified cell expression, yeast MVA | 21 | 6 | 12 |
| | metabolon + ScFPPS + Aa FS | | | |
| Sb6 | Lignified cell expression, Hevea MVA | 38 | 13 | 31 |
| | metabolon + HbFPPS + Aa FS | | | |
| Sb10 | Constitutive expression of yeast MVA | 9 | 1 | 8 |
| | metabolon + ScFPPS + Aa FS | | | |
| Sb11 | Constitutive expression, Hevea MVA | 10 | 0 | 2 |
| | metabolon + HbFPPS (without Aa FS) | | | |
| Sb11b | Constitutive expression, Hevea MVA | 2 | 1 | 1 |
| | metabolon + HbFPPS + Aa FS | | | |
| Control | NPTII/GFP | 16 | 16 | 4 |

GOI, genes of interest

### Example 8

### Evaluate Sugar Cane Events Containing the MVA Pathway Metabolic Operon for Transgene and Protein Expression, and Sesquiterpene Production

We completed terpene profiling of wild type sugar cane samples by GC and GC-MS analysis. As in the case of sorghum (see Example 2), we induced wild type sugar cane leaves with 4 mM methyl jasmonate for 30 hours to observe any increase in sesquiterpene content. Wild-type sugar cane leaf samples that were induced with MeJ produced higher and measurable levels of farnesene, caryophyllene and other sesquiterpenes as compared to leaves treated with water (FIG. 10). GC-MS analysis confirmed that the compounds that were produced by MeJ induction were caryophyllene and farnesene (data not shown).

### Example 9

### Analysis of Sorghum Transgenic Events by Multi-PLEX PCR Analysis to Determine Presence or Absence of Genes of Interest Comprising the MVA Metabolon Containing the MVA Pathway Metabolic Operon

Multi-PLEX PCR analysis using gene-specific primers was developed to determine the presence or absence of genes for selectable marker NPTII, endogenous gene ADH1 as internal control, genes comprising the entire MVA metabolon (7 genes: AACT, HMGS, HMGR, MK, PMK, MPD and IPPI) and FPPS and FS. The results of the multiplex PCR analysis of events selected for GC analysis from Sb4, Sb6 and Sb10 experiments are shown in Tables 16 to 18. In Sb4b experiment, transgenic events 402, 403, 248 and 251 contained all genes of interest while the event 401 was missing few of the MVA pathway genes and hence do not represent the entire MVA metabolon. In Sb6 experiment, events 233, 244, 406 and 407 contained all genes of interest while some of the other events were missing few of the MVA pathway genes and hence do not represent the entire MVA metabolon. In Sb10 experiment, transgenic event 418 contained all genes of interest while the event 415 was missing few of the MVA pathway genes and hence do not represent the entire MVA metabolon.

TABLE 16
MULTIPLEX PCR result of Sb4 sorghum events selected for GC analysis¹

| Event | | | | | | | | | | | |
|---|---|---|---|---|---|---|---|---|---|---|---|
| ID | adh1 | nptii | sc_aact | sc_hmgs | sc_hmgr | sc_mk | sc_pmk | sc_mpd | sc_ippi | sc_fpps | aa_bfs |
| 402 | 1 | 1 | 1 | 1 | 1 | 1 | 1 | 1 | 1 | 1 | 1 |
| 403 | 1 | 1 | 1 | 1 | 1 | 1 | 1 | 1 | 1 | 1 | 1 |
| 401 | 1 | 1 | 1 | 1 | 1 | 0 | 0 | 0 | 1 | 1 | 1 |
| 248 | 1 | 1 | 1 | 1 | 1 | 1 | 1 | 1 | 1 | 1 | 1 |
| 251 | 1 | 1 | 1 | 1 | 1 | 1 | 1 | 1 | 1 | 1 | 1 |
| Control | | | | | | | | | | | |

¹presence of a gene of interest is denoted by 1 and absence is denoted by 0.

TABLE 17
MULTIPLEX PCR result of Sb6 sorghum events selected for GC analysis¹

| Event | | | | | | | | | | | |
|---|---|---|---|---|---|---|---|---|---|---|---|
| ID | adh1 | nptii | hb_aact | hb_hmgs | hb_hmgr | hb_mk | hb_pmk | hb_mpd | hb_ippi | hb_fpps | aa_bfs |
| 242 | 1 | 1 | 0 | 0 | 1 | 1 | 1 | 1 | 1 | 1 | 1 |
| 236 | 1 | 1 | 0 | 1 | 1 | 0 | 0 | 0 | 0 | 1 | 1 |
| 238 | 1 | 1 | 0 | 0 | 1 | 0 | 0 | 0 | 0 | 1 | 1 |
| 233 | 1 | 1 | 1 | 1 | 1 | 1 | 1 | 1 | 1 | 1 | 1 |
| 232 | 1 | 1 | 0 | 0 | 1 | 1 | 0 | 0 | 0 | 1 | 1 |
| 235 | 1 | 1 | 0 | 0 | 1 | 0 | 0 | 0 | 0 | 1 | 1 |
| 237 | 1 | 1 | 0 | 0 | 1 | 1 | 1 | 1 | 1 | 1 | 1 |
| 407 | 1 | 1 | 1 | 1 | 1 | 1 | 1 | 1 | 1 | 1 | 1 |
| 406 | 1 | 1 | 1 | 1 | 1 | 1 | 1 | 1 | 1 | 1 | 1 |
| 244 | 1 | 1 | 1 | 1 | 1 | 1 | 1 | 1 | 1 | 1 | 1 |
| VC | 1 | 1 | | | | | | | | | |
| WT | | | | | | | | | | | |

¹presence of a gene of interest is denoted by 1 and absence is denoted by 0.

TABLE 18
MULTIPLEX PCR results of Sb10 sorghum events selected for GC analysis¹

| Event ID | adh1 | nptii | sc_aact | sc_hmgs | sc_hmgr | sc_mk | sc_pmk | sc_mpd | sc_ippi | sc_fpps | aa_bfs |
|---|---|---|---|---|---|---|---|---|---|---|---|
| 418 | 1 | 1 | 1 | 1 | 1 | 1 | 1 | 1 | 1 | 1 | 1 |
| 415 | 1 | 1 | 0 | 1 | 1 | 1 | 0 | 0 | 0 | 1 | 1 |
| WT | | | | | | | | | | | |

¹presence of a gene of interest is denoted by 1 and absence is denoted by 0.

### Example 10

### Analysis of Sorghum Transgenic Events for Farnesene and Caryophyllene Production

Terpene profile of transgenic plants containing the entire MVA metabolon and genes necessary for farnesene production (FPPS and FS) were conducted using GC or GC-MS. The key sesquiterpenes farnesene and caryophyllene were quantitated in transgenic events with or without methyl jasmonate induction and compared to controls. The results from various constitutive or tissue preferred promoters are shown in Tables 19-21.

In Sb4b experiment (Table 19), transgenic events 401, 402 and 403 showed 2-3 fold increase in farnesene and caryophyllene content after 4 mM Methyl Jasmonate induction as compared to wild type plants. Increase in farnesene and caryophyllene content (2-4 fold) was also noticed in some transgenic events (402 and 401) without MeJ induction, although at a relatively low level.

In Sb6 experiment (Table 20), transgenic events 242, 236, 238 and 233 showed 2-3 fold increase in farnesene and caryophyllene content after 4 mM Methyl Jasmonate induction as compared to wild type plants. Substantial increase (85 fold) in farnesene content was also noticed in some transgenic events (242 and 236) without MeJ induction, as compared to the control. However, the total fresh weight of farnesene per gm in non-induced tissues is relatively low level as compared to methyl jasmonate induced tissues.

In Sb10 experiment (Table 21), transgenic event 418 that contained all genes of interest showed 4 fold increase in farnesene while there is no major difference in caryophyllene content after 4 mM Methyl Jasmonate induction as compared to wild type plants.

TABLE 19
Farnesene and caryophyllene content in leaves
of Sb4 transgenic sorghum events

| | Methyl Jasmonate induced | | | | Non Induced | | | |
|---|---|---|---|---|---|---|---|---|
| | Caryophyllene | | Farnesene | | Caryophyllene | | Farnesene | |
| | (μg/g | | (μg/g | | (μg/g | | (μg/g | |
| Event ID | leaf) | STDEVP | leaf) | STDEVP | leaf) | STDEVP | leaf) | STDEVP |
| 402 | 15.80 | 3.40 | 10.60 | 0.59 | 4.10 | 1.39 | 0.95 | 0.30 |
| 403 | 16.80 | 6.13 | 10.84 | 1.23 | 2.77 | 1.35 | 0.13 | 0.18 |
| 401 | 9.77 | 3.42 | 7.52 | 0.92 | 4.77 | 1.65 | 0.88 | 0.09 |
| 248 | 5.90 | 2.75 | 0.22 | 0.22 | 3.53 | 3.33 | 1.34 | 0.99 |
| 251 | 3.9 | 0 | 2.9 | 0 | 1.9 | 0.00 | 0.2 | 0.00 |
| Control | 3.40 | 0.79 | 4.10 | 0.78 | 0.73 | 0.54 | 0.37 | 0.33 |

TABLE 20
Farnesene and caryophyllene content in leaves of
Sb6 transgenic sorghum events

| | Methyl Jasmonate (Induced) | | | | Non Induced | | | |
|---|---|---|---|---|---|---|---|---|
| | Caryophyllene | | Farnesene | | Caryophyllene | | Farnesene | |
| | (μg/g | | (μg/g | | (μg/g | | (μg/g | |
| Event ID | leaf) | STDEVP | leaf) | STDEVP | leaf) | STDEVP | leaf) | STDEVP |
| 242 | 11.00 | 1.31 | 10.93 | 4.34 | 0.00 | 0.00 | 1.90 | 1.10 |
| 236 | 6.90 | 1.61 | 10.73 | 3.86 | 0.00 | 0.00 | 1.85 | 0.45 |
| 238 | 11.80 | 4.00 | 9.00 | 3.30 | 0.37 | 0.64 | 0.10 | 0.14 |
| 233 | 4.40 | 1.20 | 8.15 | 3.15 | 0.00 | 0.00 | 0.50 | 0.50 |
| 232 | 6.25 | 1.55 | 6.80 | 1.80 | 0.00 | 0.00 | 0.00 | 0.00 |
| 235 | 4.03 | 1.59 | 5.17 | 0.41 | 0.00 | 0.00 | 0.00 | 0.00 |
| 237 | 2.30 | 0.90 | 4.83 | 2.35 | 0.00 | 0.00 | 0.00 | 0.00 |
| 407 | 8.47 | 2.28 | 3.57 | 0.37 | 3.00 | 0.16 | 0.23 | 0.17 |
| 406 | 6.17 | 1.30 | 3.50 | 0.98 | 2.87 | 0.95 | 0.17 | 0.24 |
| 244 | 8.50 | 2.20 | 1.85 | 0.35 | 0.00 | 0.00 | 0.00 | 0.00 |
| Control | 3.73 | 2.49 | 4.38 | 1.98 | 0.40 | 0.69 | 0.02 | 0.06 |

TABLE 21
Farnesene and caryophyllene content in leaves of
Sb10 transgenic sorghum events

| | Methyl Jasmonate (induced) | | | | Non induced | | | |
|---|---|---|---|---|---|---|---|---|
| | Caryophyllene | | Farnesene | | Caryophyllene | | Farnesene | |
| | (μg/g | | (μg/g | | (μg/g | | (μg/g | |
| Event ID | leaf) | STDEVP | leaf) | STDEVP | leaf) | STDEVP | leaf) | STDEVP |
| 418 | 1.42 | 1.39 | 12.70 | 3.40 | 0.00 | 0.00 | 1.70 | 0.29 |
| 415 | 8.53 | 3.43 | 6.20 | 1.30 | 0.57 | 0.49 | 0.17 | 0.24 |
| WT | 2.35 | 1.32 | 3.55 | 0.28 | 0.55 | 0.62 | 0.08 | 0.12 |

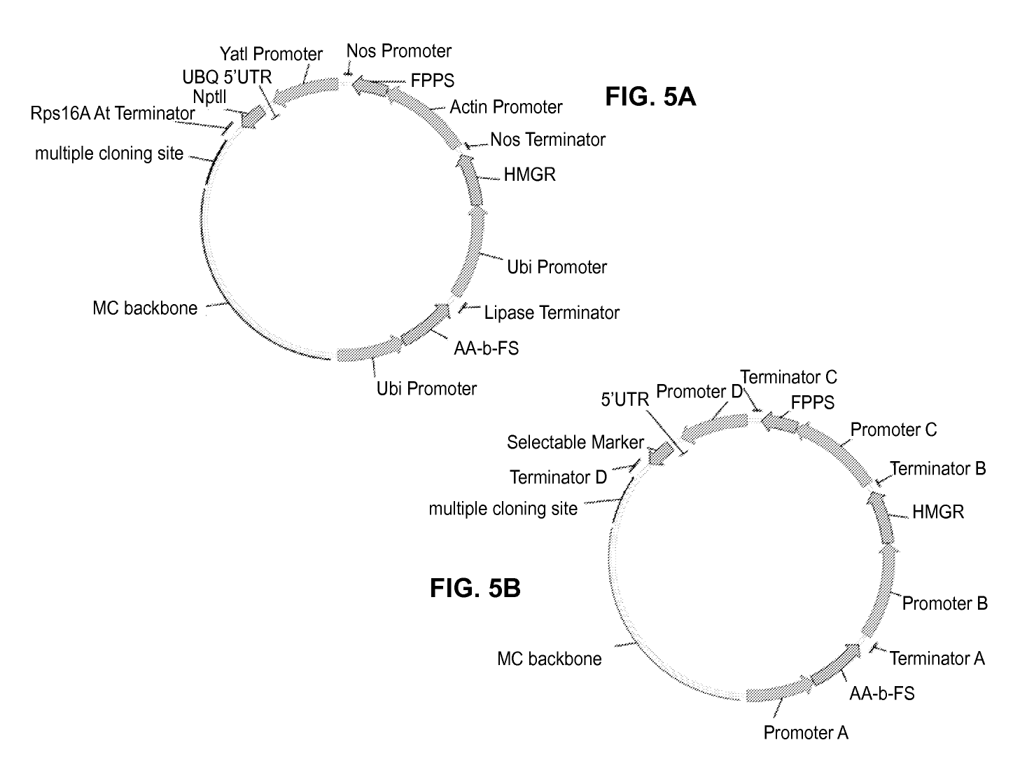

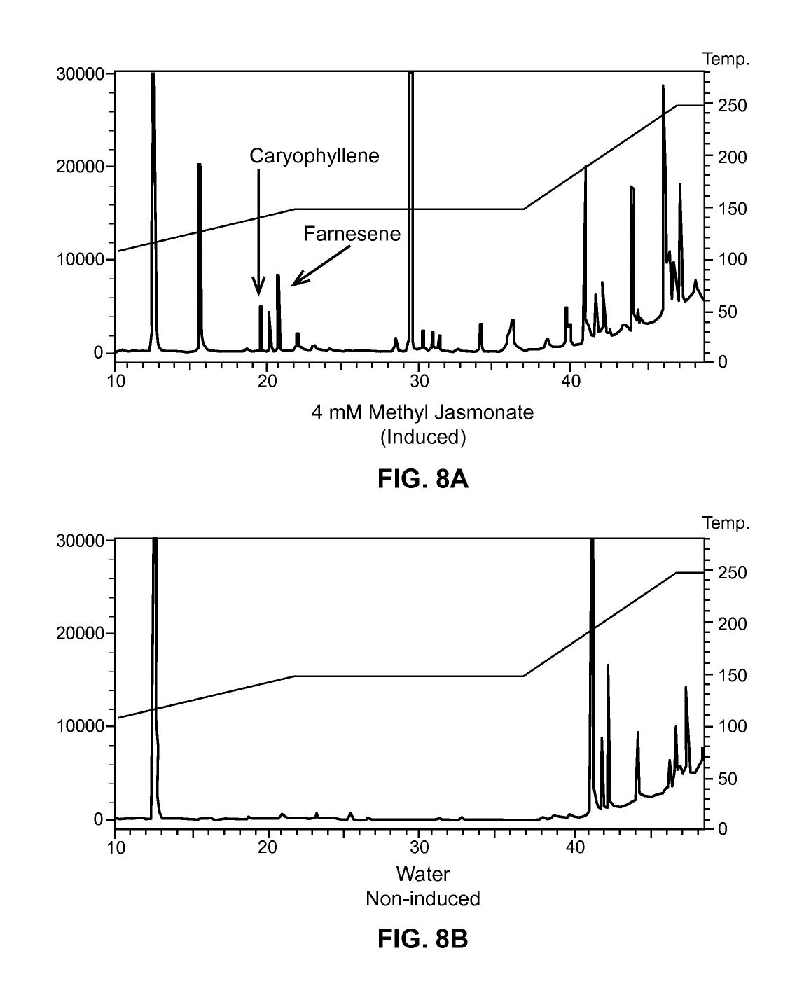

RT-PCR analysis of events that produced higher levels of farnesene showed that the key rate limiting genes FPPS and FS were expressed in some of the events (FIG. 8). In event 233 that contained all genes of the MVA metabolon, except for HMGR the rest of the genes were expressed. However, the higher rate of farnesene content did not correlate to increased transgene expression as in the case of Sb7 (FIG. 5).

### Example 11

### Analysis of Sugarcane Transgenic Events by Multi-PLEX PCR to Determine the Presence or Absence of Genes Comprising the MVA Metabolon

Multi-PLEX PCR analysis using gene specific primers was developed to determine the presence or absence of genes for selectable marker NPTII, endogenous gene ADH1 as internal control, genes comprising the entire MVA metabolon (7 genes; AACT, HMGS, HMGR, MK, PMK, MPD and IPPI) and FPPS and FS. The results of the multiplex PCR analysis of sugarcane events selected for GC analysis from So4b, So6 and So10 experiments are shown in Table 22. In Sb4b experiment, transgenic events 402, 403, 248 and 251 contained all genes of interest while the event 401 was missing few of the MVA pathway genes and hence do not represent the entire MVA metabolon. In Sb6 experiment, events 233, 244, 406 and 407 contained all genes of interest while some of the other events were missing few of the MVA pathway genes and hence do not represent the entire MVA metabolon. In Sb10 experiment, transgenic event 418 contained all genes of interest while the event 415 was missing few of the MVA pathway genes and hence do not represent the entire MVA metabolon.

TABLE 22
MxPCR results of So11b sugarcane events selected for GC analysis¹

| Event | | | | | | | | | | | |
|---|---|---|---|---|---|---|---|---|---|---|---|
| ID | adh1 | nptii | Sc_aact | Sc_hmgs | Sc_hmgr | Sc_mk | Sc_pmk | Sc_mpd | Sc_ippi | Sc_fpps | Aa_bfs |
| 546 | 1 | 1 | 1 | 1 | 1 | 1 | 1 | 0 | 1 | 1 | 1 |
| 548 | 1 | 1 | 1 | 1 | 1 | 1 | 1 | 1 | 1 | 1 | 1 |
| 572 | 1 | 1 | 1 | 1 | 1 | 1 | 1 | 1 | 1 | 1 | 1 |
| VC | 1 | 1 | 0 | 0 | 0 | 0 | 0 | 0 | 0 | 0 | 0 |

¹presence of a gene of interest is denoted by 1 and absence is denoted by 0.

### Example 12

### Analysis of Sugarcane Transgenic Events for Farnesene and Caryophyllene Production

Terpene profile of transgenic plants containing the entire MVA metabolon and genes necessary for farnesene production (FPPS and FS) were conducted using GC or GC-MS. The key sesquiterpenes farnesene and caryophyllene were quantitated in transgenic events with or without methyl jasmonate induction and compared to controls. The results from So11b experiment is shown in Table 23. Transgenic events showed 5-9 fold increase in farnesene and caryophyllene content after 4 mM Methyl Jasmonate induction as compared to control plants. Increase in farnesene and caryophyllene content (2-9 fold) was also noticed in transgenic events (572 and 548) without Methyl Jasmonate induction, although at a relatively low level as compared tissues induced by Methyl Jasmonate.

TABLE 23
Farnesene and caryophyllene content in leaves of So11b transgenic sugarcane events

| | Methyl Jasmonate Induced | | | | Non-Induced | | | |
|---|---|---|---|---|---|---|---|---|
| | | | Farnesene | | Caryophyllene | | Farnesene | |
| Event | Caryophyllene | | (μg/g | | (μg/g | | (μg/g | |
| ID | (μg/g leaf) | STDEVP | leaf) | STDEVP | leaf) | STDEVP | leaf) | STDEVP |
| 546 | 9.70 | 1.00 | 4.95 | 0.05 | 0.57 | 0.49 | 0.17 | 0.24 |
| 548 | 10.05 | 4.95 | 7.05 | 1.45 | 0.00 | 0.00 | 2.80 | 3.28 |
| 572 | 11.67 | 0.91 | 8.57 | 1.53 | 0.00 | 0.00 | 0.70 | 0.29 |
| Control | 1.40 | 1.40 | 0.95 | 0.55 | 1.95 | 0.45 | 0.30 | 0.00 |

### LITERATURE CITATIONS

Ananda, N., and P. V. Vadlani. 2010a. Fiber Reduction and Lipid Enrichment in Carotenoid-Enriched Distillers Dried Grain with Solubles Produced by Secondary Fermentation of Phaffia rhodozyma and Sporobolomyces roseus. Journal of Agricultural and Food Chemistry. 58:12744-12748.
Ananda, N., and P. V. Vadlani. 2010b. Production and optimization of carotenoid-enriched dried distiller's grains with solubles by Phaffia rhodozyma and Sporobolomyces roseus fermentation of whole stillage. Journal of industrial microbiology & biotechnology. 37:1183-1192.
Aoyama, T., and N. H. Chua. 1997. A glucocorticoid-mediated transcriptional induction system in transgenic plants. Plant J. 11:605-612.
Arce, A., M. J. Earle, H. Rodriguez, K. R. Seddon, and A. Soto. 2008. 1-Ethyl-3-methylimidazolium bis{(trifluoromethyl)sulfonyl}amide as solvent for the separation of aromatic and aliphatic hydrocarbons by liquid extraction—extension to C-7- and C-8-fractions. Green Chemistry. 10:1294-1300.
Arce, A., A. Pobudkowska, O. Rodriguez, and A. Soto. 2007. Citrus essential oil terpenless by extraction using 1-ethyl-3-methylimidazolium ethylsulfate ionic liquid: Effect of the temperature. Chemical Engineering Journal. 133:213-218.
Ausubel, F. M. 1987. Current protocols in molecular biology. Greene Publishing Associates;
J. Wiley, order fulfillment, Brooklyn, N. Y.
Media, Pa. 2 v. (loose-leaf) pp.
Bach, T. J., A. Boronat, C. Caelles, A. Ferrer, T. Weber, and A. Wettstein. 1991. Aspects Related to Mevalonate Biosynthesis in Plants. Lipids. 26:637-648.
Bell-Lelong, D. A., J. C. Cusumano, K. Meyer, and C. Chapple. 1997. Cinnamate-4-Hydroxylase Expression in Arabidopsis (Regulation in Response to Development and the Environment). Plant Physiology. 113:729-738.
Board, N. B. 2011. BioDiesel.
Bohlmann, J., and C. I. Keeling. 2008. Terpenoid biomaterials. Plant J. 54:656-669.
Bohlmann, J., Meyer-Gauen, G., Croteau, R. 1998. Plant terpenoid synthases: molecular biology and phylogenetic analysis. Proceedings of the National Academy of Sciences of the United States of America. 95:4126-4133.
Brijwani, K., H. S. Oberoi, and P. V. Vadlani. 2010. Production of a cellulolytic enzyme system in mixed-culture solid-state fermentation of soybean hulls supplemented with wheat bran. Process Biochemistry. 45:120-128.
Callis, J., M. Fromm, and V. Walbot. 1987. Introns increase gene expression in cultured maize cells. Genes Dev. 1:1183-1200.
Cheng, A. X., Y. G. Lou, Y. B. Mao, S. Lu, L. J. Wang, and X. Y. Chen. 2007. Plant terpenoids: Biosynthesis and ecological functions. J Integr Plant Biol. 49:179-186.
Coffelt, T. A., F. S. Nakayama, D. T. Ray, K. Cornish, and C. M. McMahan. 2009. Post-harvest storage effects on guayule latex, rubber, and resin contents and yields. Industrial Crops and Products. 29:326-335.
Cornish, K., M. H. Chapman, J. L. Brichta, and D. J. Scott. 2000a. Effect of postharvest conditions on the yield of hypoallergenic latex from guayule (Parthenium argentatum Gray). Abstr Pap Am Chem S. 219:U191-U191.
Cornish, K., M. H. Chapman, J. L. Brichta, S. H. Vinyard, and F. S. Nakayama. 2000b. Post-harvest stability of latex in different sizes of guayule branches. Industrial Crops and Products. 12:25-32.
Cornish, K., Myers, M. D. and Kelley, S. S.. 2004. Quantification of rubber latex in homogenate and purified samples using near infrared spectroscopy. Industrial Crops and Products 19:283-296.
Crock J, W. M., Croteau R. 1997. Isolation and bacterial expression of a sesquiterpene synthase cDNA clone from peppermint (Mentha×piperita, L.) that produces the aphid alarm pheromone (E)-beta-farnesene. Proc Natl Acad Sci USA. 94:12833-12838.
Cunillera, N., M. Arro, D. Delourme, F. Karst, A. Boronat, and A. Ferrer. 1996. Arabidopsis thaliana contains two differentially expressed farnesyl-diphosphate synthase genes. Journal of Biological Chemistry. 271:7774-7780.
Demyttenaere, J. C. R., R. M. Morina, N. De Kimpe, and P. Sandra. 2004. Use of headspace solid-phase microextraction and headspace sorptive extraction for the detection of the volatile metabolites produced by toxigenic Fusarium species. Journal of Chromatography a. 1027:147-154.
Dunwell, J. M. 1999. Transformation of maize using silicon carbide whiskers. Methods in molecular biology (Clifton, N. J. 111:375-382.
Edris, A. E., R. Chizzola, and C. Franz. 2008. Isolation and characterization of the volatile aroma compounds from the concrete headspace and the absolute of Jasminum sambac (L.) Ait. (Oleaceae) flowers grown in Egypt. European Food Research and Technology. 226:621-626.
Enjuto, M., L. Balcells, N. Campos, C. Caelles, M. Arro, and A. Boronat. 1994. Arabidopsis-Thaliana Contains 2 Differentially Expressed 3-Hydroxy-3-Methylglutaryl-Coa Reductase Genes, Which Encode Microsomal Forms of the Enzyme. Proceedings of the National Academy of Sciences of the United States of America. 91:927-931.
Estevez, J. M., A. Cantero, C. Romero, H. Kawaide, L. F. Jimenez, T. Kuzuyama, H. Seto, Y. Kamiya, and P. Leon. 2000. Analysis of the expression of CLA1, a gene that encodes the 1-deoxyxylulose 5-phosphate synthase of the 2-C-methyl-D-erythritol-4-phosphate pathway in Arabidopsis. Plant Physiology. 124:95-103.
Fischer, C. R., D. Klein-Marcuschamer, and G. Stephanopoulos. 2008. Selection and optimization of microbial hosts for biofuels production. Metabolic Engineering. 10:295-304.
Gounder, R., and E. Iglesia. 2011. Catalytic Alkylation Routes via Carbonium-Ion-Like Transition States on Acidic Zeolites. Chem Cat Chem. 3:1134-1138.
Greenhagen, B. T., P. E. O'Maille, J. P. Noel, and J. Chappell. 2006. Identifying and manipulating structural determinates linking catalytic specificities in terpene synthases. Proceedings of the National Academy of Sciences. 103:9826-9831.
Hernanz, D., V. Gallo, A. F. Recamales, A. J. Melendez-Martinez, and F. J. Heredia. 2008. Comparison of the effectiveness of solid-phase and ultrasound-mediated liquid-liquid extractions to determine the volatile compounds of wine. Talanta. 76:929-935.
Huber D P, P. R., Godard K A, Sturrock R N, Bohlmann J. 2005. Characterization of four terpene synthase cDNAs from methyl jasmonate-induced Douglas-fir, Pseudotsuga menziesii. Phytochemistry. 66:1427-1439.
Knapik, A., A. Drelinkiewicz, A. Waksmundzka-Gora, A. Bukowska, W. Bukowski, and J. Noworol. 2008. Hydrogenation of 2-Butyn-1,4-diol in the Presence of Functional Crosslinked Resin Supported Pd Catalyst. The Role of Polymer Properties in Activity/Selectivity Pattern. Catalysis Letters. 122:155-166.
Kollner, T. G., J. Gershenzon, and J. Degenhardt. 2009. Molecular and biochemical evolution of maize terpene synthase 10, an enzyme of indirect defense. Phytochemistry. 70:1139-1145.
Lai, S. M., I. W. Chen, and M. J. Tsai. 2005. Preparative isolation of terpene trilactones from Ginkgo biloba leaves. Journal of Chromatography a. 1092:125-134.
LEWINSOHN, E., N. DUDAI, Y. TADMOR, I. KATZIR, U. RAVID, E. PUTIEVSKY, and D. M. JOEL. 1998. Histochemical Localization of Citral Accumulation in Lemongrass Leaves (Cymbopogon citratus (DC.) Stapf., Poaceae). Annals of Botany. 81:35-39.
Liang, X. W., M. Dron, C. L. Cramer, R. A. Dixon, and C. J. Lamb. 1989. Differential regulation of phenylalanine ammonia-lyase genes during plant development and by environmental cues. Journal of Biological Chemistry. 264:14486-14492.
Lin, Y., and S. Tanaka. 2006. Ethanol fermentation from biomass resources: current state and prospects. Appl Microbiol Biotechnol. 69:627-642.
Maruyama T, I. M., Honda G. 2001. Molecular cloning, functional expression and characterization of (E)-beta farnesene synthase from Citrus junos. Biol Pharm Bull. 10:1171-1175.
Maury, S., P. Geoffroy, and M. Legrand. 1999. Tobacco O-Methyltransferases Involved in Phenylpropanoid Metabolism. The Different Caffeoyl-Coenzyme A/5-Hydroxyferuloyl-Coenzyme A 3/5-O-Methyltransferase and Caffeic Acid/5-Hydroxyferulic Acid 3/5-O-Methyltransferase Classes Have Distinct Substrate Specificities and Expression Patterns. Plant Physiology. 121:215-224.
McMahan, C. M., K. Cornish, T. A. Coffelt, F. S. Nakayama, R. G. McCoy, J. L. Brichta, and D. T. Ray. 2006. Post-harvest storage effects on guayule latex quality from agronomic trials. Industrial Crops and Products. 24:321-328.
Mookdasanit, J., H. Tamura, T. Yoshizawa, T. Tokunaga, and K. Nakanishi. 2003. Trace volatile components in essential oil of Citrus sudachi by means of modified solvent extraction method. Food Science and Technology Research. 9:54-61.
Nair, R. B., Q. Xia, C. J. Kartha, E. Kurylo, R. N. Hirji, R. Datla, and G. Selvaraj. 2002. Arabidopsis CYP98A3 Mediating Aromatic 3-Hydroxylation. Developmental Regulation of the Gene, and Expression in Yeast. Plant Physiology. 130:210-220.
Newell, R. 2011. Annual Energy Outlook 2011, Reference Case.
Nigam, P. S., and A. Singh. 2011. Production of liquid biofuels from renewable resources. Progress in Energy and Combustion Science. 37:52-68.
Oberoi, H. S., P. V. Vadlani, R. L. Madl, L. Saida, and J. P. Abeykoon. 2010. Ethanol Production from Orange Peels: Two-Stage Hydrolysis and Fermentation Studies Using Optimized Parameters through Experimental Design. Journal of Agricultural and Food Chemistry. 58:3422-3429.
Pechous, S. W., C. B. Watkins, and B. D. Whitaker. 2005. Expression of alpha-farnesene synthase gene AFS1 in relation to levels of alpha-farnesene and conjugated trienols in peel tissue of scald-susceptible ‘Law Rome’ and scald-resistant ‘Idared’ apple fruit. Postharvest Biology and Technology. 35:125-132.
Peralta-Yahya, P., and J. Keasling. 2010. Advanced biofuel production in microbes. Biotechnol J. 5:147-162.
Petrasovits, L. A. P., M. P.; Nielsen, L. K.; Brumbley, S. M. 2007. Production of polyhydroxybutyrate in sugar cane. Plant Biotechnology Journal. 5:162-172.
Picaud S, B. M., Brodelius P E. 2005. Expression, purification and characterization of recombinant (E)-beta-farnesene synthase from Artemisia annua. Phytochemistry. 66:961-967.
Pourbafrani, M., G. Forgacs, I. S. Horvath, C. Niklasson, and M. J. Taherzadeh. 2010. Production of biofuels, limonene and pectin from citrus wastes. Bioresour Technol. 101:4246-4250.
R F A. 2011. Renewable Fuels Association—ethanol facts.
Rout, P. K., S. N. Naika, and Y. R. Rao. 2008. Subcritical CO2 extraction of floral fragrance from Quisqualis indica. Journal of Supercritical Fluids. 45:200-205.
Schnee, C., T. G. Kollner, M. Held, T. C. J. Turlings, J. Gershenzon, and J. Degenhardt. 2006. The products of a single maize sesquiterpene synthase form a volatile defense signal that attracts natural enemies of maize herbivores. Proceedings of the National Academy of Sciences of the United States of America. 103:1129-1134.
Serrano, A., and M. Gallego. 2006. Continuous microwave-assisted extraction coupled on-line with liquid-liquid extraction: Determination of aliphatic hydrocarbons in soil and sediments. Journal of Chromatography a. 1104:323-330.
Tholl, D. 2006. Terpene synthases and the regulation, diversity and biological roles of terpene metabolism. Current Opinion in Plant Biology. 9:1-8.
Unger, E. A., J. M. Hand, A. R. Cashmore, and A. C. Vasconcelos. 1989. Isolation of a cDNA encoding mitochondrial citrate synthase from Arabidopsis thaliana. Plant Mol Biol. 13:411-418.
Van den Broeck, G., Timko, M. P., Kausch, A. P., Cashmore, A. R., Van Montagu, M, Herrera-Estrella, L. 1985. Targeting of a foreign peptide to chloroplasts by fusion to the transit peptide from the small subunit of ribulose 1,5-bisphosphate carboxylase. Nature. 313:358-363.
von Heijne, G., Steppuhn, J., Herrmann, R. G. 1989. Domain structure of mitochondrial and chloroplast targeting peptides. European Journal of Biochemistry. 180:535-545.
Wienk, H. L. J., Wechselberger, R. W., Czisch, M., de Kruijff, B. 2000. Structure, Dynamics, and Insertion of a Chloroplast Targeting Peptide in Mixed Micelles. Biochemistry. 39:8219-8227.
Wu, S., M. Schalk, A. Clark, R. B. Miles, R. Coates, and J. Chappell. 2006. Redirection of cytosolic or plastidic isoprenoid precursors elevates terpene production in plants. Nat Biotechnol. 24:1441-1447.
Yoshikuni, Y., and B.w.t.U.o.C. University of California, San Francisco. 2007. Redesigning enzymes based on the theories of molecular evolution for optimal function in synthetic metabolic pathways. University of California, Berkeley with the University of California, San Francisco.
Zhan, X., D. Wang, M. R. Tuinstra, S. Bean, P. A. Seib, and X. S. Sun. 2003. Ethanol and lactic acid production as affected by sorghum genotype and location. Industrial Crops and Products. 18:245-255.
Zhang, J., X.-Z. Sun, M. Poliakoff, and M. W. George. 2003. Study of the reaction of Rh(acac)(CO)2 with alkenes in polyethylene films under high-pressure hydrogen and the Rh-catalysed hydrogenation of alkenes. Journal of Organometallic Chemistry. 678:128-133.
Zheng, C. H., T. H. Kim, K. H. Kim, Y. H. Leem, and H. J. Lee. 2004. Characterization of potent aroma compounds in Chrysanthemum coronarium L. (Garland) using aroma extract dilution analysis. Flavour and Fragrance Journal. 19:401-405.
Zini, C. A., K. D. Zanin, E. Christensen, E. B. Caramao, and J. Pawliszyn. 2003. Solid-phase microextraction of volatile compounds from the chopped leaves of three species of Eucalyptus. Journal of Agricultural and Food Chemistry. 51:2679-2686.

We claim:

1. A method of increasing production of at least one terpenoid, the method comprising expressing in a plant cell a set of heterologous nucleic acids that encode polypeptides comprising enzymes necessary to carry out the mevalonic acid pathway or the methylerythritol 4-phosphate pathway, wherein production of the at least one terpenoid is increased when compared to a wild-type plant cell not encoding the set of heterologous nucleic acids.

2. The method of claim 1, wherein both the mevalonic acid pathway and the methylerythritol 4-phosphate pathway are expressed from heterologous nucleic acids.

3. The method of claim 1, further comprising expressing at least one heterologous nucleic acid encoding at least one polypeptide selected from the group consisting of isopentenyl-diphosphate delta-isomerase, farnesyl diphosphate synthase, and farnesene synthase.

4. The method of claim 2, further comprising expressing at least one heterologous nucleic acid encoding at least one polypeptide selected from the group consisting of isopentenyl-diphosphate delta-isomerase, farnesyl diphosphate synthase, and farnesene synthase is expressed.

5. The method of claim 1, wherein enzymes from the mevalonic acid pathway, the methylerythritol 4-phosphate, and an isopentenyl-diphosphate delta-isomerase, a farnesyl diphosphate synthase, and a farnesene synthase are expressed.

6. The method of claims 1-5, further comprising exposing the plant cell to an elicitor of sesquiterpene production.

7. The method of claim 6, wherein the elicitor is selected from the group consisting of methyl jasmonate, salicylic acid, ethephon and benzothiadiazole.

8. The method of claim 7, wherein the elicitor is methyl jasmonate.

9. The method of claim 3-5, wherein the isopentenyl-diphosphate delta-isomerase is expressed and is an isopentenyl-diphosphate delta-isomerase I or isopentenyl-diphosphate delta-isomerase II.

10. The method of claim 3-5, wherein the, wherein the farnesene synthase is expressed and is an α-farnesene synthase or a β-farnesene synthase.

11. The method of any of claims 1-5, wherein the at least one terpenoid is a sesquiterpenoid.

12. The method of claim 11, wherein the sesquiterpenoid comprises farnesene.

13. The method of any of claims 1-5, wherein the set of heterologous nucleic acids encoding enzymes of the mevalonic acid pathway comprises nucleic acids encoding a(n):
a. acetyl-CoA acetyltransferase,
b. 3-hydroxy-3-methylglutaryl coenzyme A synthase,
c. 3-hydroxy-3-methylglutaryl-coenzyme A reductase,
d. mevalonate kinase,
e. phosphomevalonate kinase, and
f. mevalonate pyrophosphate decarboxylase;
and wherein the set of heterologous nucleic acids encoding enzymes of the methylerythritol 4-phosphate pathway comprises nucleic acids encoding a(n):
g. 1-deoxy-D-xylulose-5-phosphate synthase,
h. 1-deoxy-D-xylulose 5-phosphate reductoisomerase,
i. 4-diphosphocytidyl-2-C-methyl-D-erythritol synthase,
j. 4-diphosphocytidyl-2-C-methyl-D-erythritol kinase,
k. 2-C-methyl-D-erythritol 2,4-cyclodiphosphate synthase,
l. 4-hydroxy-3-methylbut-2-en-1-yl diphosphate synthase, and
m. 4-hydroxy-3-methyl but-2-enyl diphosphate reductase.

14. The method of claim 13, wherein at least two of the heterologous nucleic acids are introduced into the plant cell on a single recombinant DNA construct.

15. The method of claim 14, wherein the recombinant DNA construct is a mini-chromosome.

16. The method of claim 15, wherein at least the enzymes of the mevalonic acid pathway or the methylerythritol 4-phosphate pathway are comprised on a single mini-chromosome.

17. The method of claim 15 Error! Reference source not found. Error! Reference source not found., wherein the enzymes of the mevalonic acid pathway and the methylerythritol 4-phosphate pathway are comprised on a single mini-chromosome.

18. The method of claim 16 or 17, wherein the mini-chromosome further comprises heterologous nucleic acids encoding polypeptides comprising at least one enzyme selected from the group consisting of isopentenyl-diphosphate delta-isomerase, farnesyl diphosphate synthase and farnesene synthase.

19. The method of claim 13, wherein the set of heterologous nucleic acids encoding enzymes of the mevalonic pathway comprise nucleic acids encoding a(n):
a. acetyl-CoA acetyltransferase having at least 70% sequence identity to at least one amino acid sequence selected from the group consisting of SEQ ID NOs:1-4, 143;
b. 3-hydroxy-3-methylglutaryl coenzyme A synthase having at least 70% sequence identity to at least one amino acid sequence selected from the group consisting of SEQ ID NOs:5-9, 144, 145;
c. 3-hydroxy-3-methylglutaryl-coenzyme A reductase having at least 70% sequence identity to at least one amino acid sequence selected from the group consisting of SEQ ID NOs:10-16, 17-20, 146-150;
d. mevalonate kinase, having at least 70% sequence identity to at least one amino acid sequence selected from the group consisting of SEQ ID NOs:21-26, 151;
e. phosphomevalonate kinase, having at least 70% sequence identity to at least one amino acid sequence selected from the group consisting of SEQ ID NOs:27-33 and
f. mevalonate pyrophosphate decarboxylase having at least 70% sequence identity to at least one amino acid sequence selected from the group consisting of SEQ ID NOs:34-40, 152;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 70% sequence identity to at least one amino acid sequence selected from the group consisting of SEQ ID NOs:41-49, 153, 154, 169, 177-180;
h. 1-deoxy-D-xylulose 5-phosphate reductoisomerase, having at least 70% sequence identity to at least one amino acid sequence selected from the group consisting of SEQ ID NOs:50-58, 155, 156, 170, 181;
i. 4-diphosphocytidyl-2-C-methyl-D-erythritol synthase, having at least 70% sequence identity to at least one amino acid sequence selected from the group consisting of SEQ ID NOs:59-67, 157, 171, 182;
j. 4-diphosphocytidyl-2-C-methyl-D-erythritol kinase, having at least 70% sequence identity to at least one amino acid sequence selected from the group consisting of SEQ ID NOs:68-73, 158, 172, 183;
k. 2-C-methyl-D-erythritol 2,4-cyclodiphosphate synthase, having at least 70% sequence identity to at least one amino acid sequence selected from the group consisting of SEQ ID NOs:74-82, 159, 173, 184;
l. 4-hydroxy-3-methylbut-2-en-1-yl diphosphate synthase, and having at least 70% sequence identity to at least one amino acid sequence selected from the group consisting of SEQ ID NOs:83-89, 160, 174, 185; and
m. 4-hydroxy-3-methylbut-2-enyl diphosphate reductase having at least 70% sequence identity to at least one amino acid sequence selected from the group consisting of SEQ ID NOs:90-97, 161-163, 175, 186.

20. The method of claim 13, wherein the set of heterologous nucleic acids encoding enzymes of the mevalonic acid pathway comprises nucleic acids encoding:
a. acetyl-CoA acetyltransferase having at least 80% sequence identity to at least one amino acid sequence selected from the group consisting of SEQ ID NOs:1-4, 143;
b. 3-hydroxy-3-methylglutaryl coenzyme A synthase having at least 80% sequence identity to at least one amino acid sequence selected from the group consisting of SEQ ID NOs:5-9, 144, 145;
c. 3-hydroxy-3-methylglutaryl-coenzyme A reductase having at least 80% sequence identity to at least one amino acid sequence selected from the group consisting of SEQ ID NOs:10-16, 17-20, 146-150;
d. mevalonate kinase, having at least 80% sequence identity to at least one amino acid sequence selected from the group consisting of SEQ ID NOs:21-26, 151;
e. phosphomevalonate kinase, having at least 80% sequence identity to at least one amino acid sequence selected from the group consisting of SEQ ID NOs:27-33 and
f. mevalonate pyrophosphate decarboxylase having at least 80% sequence identity to at least one amino acid sequence selected from the group consisting of SEQ ID NOs:34-40, 152;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 80% sequence identity to at least one amino acid sequence selected from the group consisting of SEQ ID NOs:41-49, 153, 154, 169, 177-180;
h. 1-deoxy-D-xylulose 5-phosphate reductoisomerase, having at least 80% sequence identity to at least one amino acid sequence selected from the group consisting of SEQ ID NOs:50-58, 155, 156, 170, 181;
i. 4-diphosphocytidyl-2-C-methyl-D-erythritol synthase, having at least 80% sequence identity to at least one amino acid sequence selected from the group consisting of SEQ ID NOs:59-67, 157, 171, 182;
j. 4-diphosphocytidyl-2-C-methyl-D-erythritol kinase, having at least 80% sequence identity to at least one amino acid sequence selected from the group consisting of SEQ ID NOs:68-73, 158, 172, 183;
k. 2-C-methyl-D-erythritol 2,4-cyclodiphosphate synthase, having at least 80% sequence identity to at least one amino acid sequence selected from the group consisting of SEQ ID NOs:74-82, 159, 173, 184;
l. 4-hydroxy-3-methylbut-2-en-1-yl diphosphate synthase, and having at least 80% sequence identity to at least one amino acid sequence selected from the group consisting of SEQ ID NOs:83-89, 160, 174, 185; and
m. 4-hydroxy-3-methylbut-2-enyl diphosphate reductase having at least 80% sequence identity to at least one amino acid sequence selected from the group consisting of SEQ ID NOs:90-97, 161-163, 175, 186.

21. The method of claim 13, wherein the set of heterologous nucleic acids encoding enzymes of the mevalonic acid pathway comprises nucleic acids encoding:
a. acetyl-CoA acetyltransferase having at least 90% sequence identity to at least one amino acid sequence selected from the group consisting of SEQ ID NOs:1-4, 143;
b. 3-hydroxy-3-methylglutaryl coenzyme A synthase having at least 90% sequence identity to at least one amino acid sequence selected from the group consisting of SEQ ID NOs:5-9, 144, 145;
c. 3-hydroxy-3-methylglutaryl-coenzyme A reductase having at least 90% sequence identity to at least one amino acid sequence selected from the group consisting of SEQ ID NOs:10-16, 17-20, 146-150;
d. mevalonate kinase, having at least 90% sequence identity to at least one amino acid sequence selected from the group consisting of SEQ ID NOs:21-26, 151;
e. phosphomevalonate kinase, having at least 90% sequence identity to at least one amino acid sequence selected from the group consisting of SEQ ID NOs:27-33 and
f. mevalonate pyrophosphate decarboxylase having at least 90% sequence identity to at least one amino acid sequence selected from the group consisting of SEQ ID NOs:34-40, 152;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 90% sequence identity to at least one amino acid sequence selected from the group consisting of SEQ ID NOs:41-49, 153, 154, 169, 177-180;
h. 1-deoxy-D-xylulose 5-phosphate reductoisomerase, having at least 90% sequence identity to at least one amino acid sequence selected from the group consisting of SEQ ID NOs:50-58, 155, 156, 170, 181;
i. 4-diphosphocytidyl-2-C-methyl-D-erythritol synthase, having at least 90% sequence identity to at least one amino acid sequence selected from the group consisting of SEQ ID NOs:59-67, 157, 171, 182;
j. 4-diphosphocytidyl-2-C-methyl-D-erythritol kinase, having at least 90% sequence identity to at least one amino acid sequence selected from the group consisting of SEQ ID NOs:68-73, 158, 172, 183;
k. 2-C-methyl-D-erythritol 2,4-cyclodiphosphate synthase, having at least 90% sequence identity to at least one amino acid sequence selected from the group consisting of SEQ ID NOs:74-82, 159, 173, 184;
l. 4-hydroxy-3-methylbut-2-en-1-yl diphosphate synthase, and having at least 90% sequence identity to at least one amino acid sequence selected from the group consisting of SEQ ID NOs:83-89, 160, 174, 185; and
m. 4-hydroxy-3-methylbut-2-enyl diphosphate reductase having at least 90% sequence identity to at least one amino acid sequence selected from the group consisting of SEQ ID NOs:90-97, 161-163, 175, 186.

22. The method of claim 13, wherein the set of heterologous nucleic acids encoding enzymes of the mevalonic acid pathway comprises nucleic acids encoding:
a. acetyl-CoA acetyltransferase having at least 95% sequence identity to at least one amino acid sequence selected from the group consisting of SEQ ID NOs:1-4, 143;
b. 3-hydroxy-3-methylglutaryl coenzyme A synthase having at least 95% sequence identity to at least one amino acid sequence selected from the group consisting of SEQ ID NOs:5-9, 144, 145;
c. 3-hydroxy-3-methylglutaryl-coenzyme A reductase having at least 95% sequence identity to at least one amino acid sequence selected from the group consisting of SEQ ID NOs:10-16, 17-20, 146-150;
d. mevalonate kinase, having at least 95% sequence identity to at least one amino acid sequence selected from the group consisting of SEQ ID NOs:21-26, 151;
e. phosphomevalonate kinase, having at least 95% sequence identity to at least one amino acid sequence selected from the group consisting of SEQ ID NOs:27-33 and
f. mevalonate pyrophosphate decarboxylase having at least 95% sequence identity to at least one amino acid sequence selected from the group consisting of SEQ ID NOs:34-40, 152;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 95% sequence identity to at least one amino acid sequence selected from the group consisting of SEQ ID NOs:41-49, 153, 154, 169, 177-180;
h. 1-deoxy-D-xylulose 5-phosphate reductoisomerase, having at least 95% sequence identity to at least one amino acid sequence selected from the group consisting of SEQ ID NOs:50-58, 155, 156, 170, 181;
i. 4-diphosphocytidyl-2-C-methyl-D-erythritol synthase, having at least 95% sequence identity to at least one amino acid sequence selected from the group consisting of SEQ ID NOs:59-67, 157, 171, 182;
j. 4-diphosphocytidyl-2-C-methyl-D-erythritol kinase, having at least 95% sequence identity to at least one amino acid sequence selected from the group consisting of SEQ ID NOs:68-73, 158, 172, 183;
k. 2-C-methyl-D-erythritol 2,4-cyclodiphosphate synthase, having at least 95% sequence identity to at least one amino acid sequence selected from the group consisting of SEQ ID NOs:74-82, 159, 173, 184;
l. 4-hydroxy-3-methylbut-2-en-1-yl diphosphate synthase, and having at least 95% sequence identity to at least one amino acid sequence selected from the group consisting of SEQ ID NOs:83-89, 160, 174, 185; and
m. 4-hydroxy-3-methylbut-2-enyl diphosphate reductase having at least 95% sequence identity to at least one amino acid sequence selected from the group consisting of SEQ ID NOs:90-97, 161-163, 175, 186.

23. The method of claim 13, wherein the set of heterologous nucleic acids encoding enzymes of the mevalonic acid pathway comprises nucleic acids encoding:
a. acetyl-CoA acetyltransferase having at least 99% sequence identity to at least one amino acid sequence selected from the group consisting of SEQ ID NOs:1-4, 143;
b. 3-hydroxy-3-methylglutaryl coenzyme A synthase having at least 99% sequence identity to at least one amino acid sequence selected from the group consisting of SEQ ID NOs:5-9, 144, 145;
c. 3-hydroxy-3-methylglutaryl-coenzyme A reductase having at least 99% sequence identity to at least one amino acid sequence selected from the group consisting of SEQ ID NOs:10-16, 17-20, 146-150;
d. mevalonate kinase, having at least 99% sequence identity to at least one amino acid sequence selected from the group consisting of SEQ ID NOs:21-26, 151;
e. phosphomevalonate kinase, having at least 99% sequence identity to at least one amino acid sequence selected from the group consisting of SEQ ID NOs:27-33 and
f. mevalonate pyrophosphate decarboxylase having at least 99% sequence identity to at least one amino acid sequence selected from the group consisting of SEQ ID NOs:34-40, 152;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 99% sequence identity to at least one amino acid sequence selected from the group consisting of SEQ ID NOs:41-49, 153, 154, 169, 177-180;
h. 1-deoxy-D-xylulose 5-phosphate reductoisomerase, having at least 99% sequence identity to at least one amino acid sequence selected from the group consisting of SEQ ID NOs:50-58, 155, 156, 170, 181;
i. 4-diphosphocytidyl-2-C-methyl-D-erythritol synthase, having at least 99% sequence identity to at least one amino acid sequence selected from the group consisting of SEQ ID NOs:59-67, 157, 171, 182;
j. 4-diphosphocytidyl-2-C-methyl-D-erythritol kinase, having at least 99% sequence identity to at least one amino acid sequence selected from the group consisting of SEQ ID NOs:68-73, 158, 172, 183;
k. 2-C-methyl-D-erythritol 2,4-cyclodiphosphate synthase, having at least 99% sequence identity to at least one amino acid sequence selected from the group consisting of SEQ ID NOs:74-82, 159, 173, 184;
l. 4-hydroxy-3-methylbut-2-en-1-yl diphosphate synthase, and having at least 99% sequence identity to at least one amino acid sequence selected from the group consisting of SEQ ID NOs:83-89, 160, 174, 185; and
m. 4-hydroxy-3-methylbut-2-enyl diphosphate reductase having at least 99% sequence identity to at least one amino acid sequence selected from the group consisting of SEQ ID NOs:90-97, 161-163, 175, 186.

24. The method of claim 13, wherein the set of heterologous nucleic acids encoding enzymes of the mevalonic acid pathway comprises nucleic acids encoding:
a. acetyl-CoA acetyltransferase is sequence selected from the group consisting of SEQ ID NOs:1-4, 143;
b. 3-hydroxy-3-methylglutaryl coenzyme A synthase is sequence selected from the group consisting of SEQ ID NOs:5-9, 144, 145;
c. 3-hydroxy-3-methylglutaryl-coenzyme A reductase is sequence selected from the group consisting of SEQ ID NOs:10-16, 17-20, 146-150;
d. mevalonate kinase, is sequence selected from the group consisting of SEQ ID NOs:21-26, 151;
e. phosphomevalonate kinase, is sequence selected from the group consisting of SEQ ID NOs:27-33 and
f. mevalonate pyrophosphate decarboxylase is sequence selected from the group consisting of SEQ ID NOs:34-40, 152;
and wherein the set of heterologous nucleic acids encoding enzymes of the methylerythritol 4-phosphate pathway comprise nucleic acid encoding a:
g. 1-deoxy-D-xylulose-5-phosphate synthase, is sequence selected from the group consisting of SEQ ID NOs:41-49, 153, 154, 169, 177-180;
h. 1-deoxy-D-xylulose 5-phosphate reductoisomerase, is sequence selected from the group consisting of SEQ ID NOs:50-58, 155, 156, 170, 181;
i. 4-diphosphocytidyl-2-C-methyl-D-erythritol synthase, is sequence selected from the group consisting of SEQ ID NOs:59-67, 157, 171, 182;
j. 4-diphosphocytidyl-2-C-methyl-D-erythritol kinase, is sequence selected from the group consisting of SEQ ID NOs:68-73, 158, 172, 183;
k. 2-C-methyl-D-erythritol 2,4-cyclodiphosphate synthase, is sequence selected from the group consisting of SEQ ID NOs:74-82, 159, 173, 184;
l. 4-hydroxy-3-methylbut-2-en-1-yl diphosphate synthase, and is sequence selected from the group consisting of SEQ ID NOs:83-89, 160, 174, 185; and
m. 4-hydroxy-3-methylbut-2-enyl diphosphate reductase is sequence selected from the group consisting of SEQ ID NOs:90-97, 161-163, 175, 186.

25. The method of claim 3-5 wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 70% sequence identity to at least one amino acid sequence selected from the group consisting of SEQ ID NOs:98-101, 102-106, 188, 190-192;
b. farnesyl diphosphate synthase, having at least 70% sequence identity to at least one amino acid sequence selected from the group consisting of SEQ ID NOs:107-111, 164, 165, 176, 187, 189; and
c. farnesene synthase, having at least 70% sequence identity to at least one amino acid sequence selected from the group consisting of SEQ ID NOs:112-115, 116-117, 166-168.

26. The method of claim 3-5, wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 80% sequence identity to at least one amino acid sequence selected from the group consisting of SEQ ID NOs:98-101, 102-106, 188, 190-192;
b. farnesyl diphosphate synthase, having at least 80% sequence identity to at least one amino acid sequence selected from the group consisting of SEQ ID NOs:107-111, 164, 165, 176, 187, 189; and
c. farnesene synthase, having at least 80% sequence identity to at least one amino acid sequence selected from the group consisting of SEQ ID NOs:112-115, 116-117, 166-168.

27. The method of claim 3-5, wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 90% sequence identity to at least one amino acid sequence selected from the group consisting of SEQ ID NOs:98-101, 102-106, 188, 190-192;
b. farnesyl diphosphate synthase, having at least 90% sequence identity to at least one amino acid sequence selected from the group consisting of SEQ ID NOs:107-111, 164, 165, 176, 187, 189; and
c. farnesene synthase, having at least 90% sequence identity to at least one amino acid sequence selected from the group consisting of SEQ ID NOs:112-115, 116-117, 166-168.

28. The method of claim 3-5, wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 95% sequence identity to at least one amino acid sequence selected from the group consisting of SEQ ID NOs:98-101, 102-106, 188, 190-192;
b. farnesyl diphosphate synthase, having at least 95% sequence identity to at least one amino acid sequence selected from the group consisting of SEQ ID NOs:107-111, 164, 165, 176, 187, 189; and
c. farnesene synthase, having at least 95% sequence identity to at least one amino acid sequence selected from the group consisting of SEQ ID NOs:112-115, 116-117, 166-168.

29. The method of claim 3-5, wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 99% sequence identity to at least one amino acid sequence selected from the group consisting of SEQ ID NOs:98-101, 102-106, 188, 190-192;
b. farnesyl diphosphate synthase, having at least 99% sequence identity to at least one amino acid sequence selected from the group consisting of SEQ ID NOs:107-111, 164, 165, 176, 187, 189; and
c. farnesene synthase, having at least 99% sequence identity to at least one amino acid sequence selected from the group consisting of SEQ ID NOs:112-115, 116-117, 166-168.

30. The method of claim 3-5, wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, is selected from the group consisting of SEQ ID NOs:98-101, 102-106, 188, 190-192;
b. farnesyl diphosphate synthase, is selected from the group consisting of SEQ ID NOs:107-111, 164, 165, 176, 187, 189; and
c. farnesene synthase, is selected from the group consisting of SEQ ID NOs:112-115, 116-117, 166-168.

31. The method of claim 1, wherein at least one of the heterologous nucleic acids is selected from the group consisting of Archaea, bacteria, fungi, and plantae kingdoms.

32. The method of claim 31, wherein the set of heterologous nucleic acids encode enzymes from the plantae kingdom.

33. The method of claim 32, wherein the set of heterologous nucleic acids encoding enzymes of the mevalonic pathway comprise nucleic acids encoding a(n):
a. acetyl-CoA acetyltransferase having at least 70% sequence identity to SEQ ID NO:4;
b. 3-hydroxy-3-methylglutaryl coenzyme A synthase having at least 70% sequence identity to at least one amino acid sequence selected from the group consisting of SEQ ID NOs:
8-9;
c. 3-hydroxy-3-methylglutaryl-coenzyme A reductase having at least 70% sequence identity to at least one amino acid sequence selected from the group consisting of SEQ ID NOs:15, 16, 20;
d. mevalonate kinase, having at least 70% sequence identity SEQ ID NO:26;
e. phosphomevalonate kinase, having at least 70% sequence identity to at least one amino acid sequence selected from the group consisting of SEQ ID NOs:32-33 and
f. mevalonate pyrophosphate decarboxylase having at least 70% sequence identity to at least one amino acid sequence selected from the group consisting of SEQ ID NOs:39-40;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 70% sequence identity to at least one amino acid sequence selected from the group consisting of SEQ ID NOs:41, 48-49;
h. 1-deoxy-D-xylulose 5-phosphate reductoisomerase, having at least 70% sequence identity to at least one amino acid sequence selected from the group consisting of SEQ ID NOs:50, 56-58;
i. 4-diphosphocytidyl-2-C-methyl-D-erythritol synthase, having at least 70% sequence identity to at least one amino acid sequence selected from the group consisting of SEQ ID NOs:59, 66-67;
j. 4-diphosphocytidyl-2-C-methyl-D-erythritol kinase, having at least 70% sequence identity to at least one amino acid sequence selected from the group consisting of SEQ ID NOs:68, 73;
k. 2-C-methyl-D-erythritol 2,4-cyclodiphosphate synthase, having at least 70% sequence identity to at least one amino acid sequence selected from the group consisting of SEQ ID NOs:74, 80-82;
l. 4-hydroxy-3-methylbut-2-en-1-yl diphosphate synthase, and having at least 70% sequence identity to at least one amino acid sequence selected from the group consisting of SEQ ID NOs:83, 89; and
m. 4-hydroxy-3-methylbut-2-enyl diphosphate reductase having at least 70% sequence identity to at least one amino acid sequence selected from the group consisting of SEQ ID NOs:90, 96-97.

34. The method of claim 32, wherein the set of heterologous nucleic acids encoding enzymes of the mevalonic acid pathway comprises nucleic acids encoding:
a. acetyl-CoA acetyltransferase having at least 80% sequence identity to SEQ ID NO:4;
b. 3-hydroxy-3-methylglutaryl coenzyme A synthase having at least 80% sequence identity to at least one amino acid sequence selected from the group consisting of SEQ ID NOs: 8-9;
c. 3-hydroxy-3-methylglutaryl-coenzyme A reductase having at least 80% sequence identity to at least one amino acid sequence selected from the group consisting of SEQ ID NOs:15, 16, 20;
d. mevalonate kinase, having at least 80% sequence identity SEQ ID NO:26;
e. phosphomevalonate kinase, having at least 80% sequence identity to at least one amino acid sequence selected from the group consisting of SEQ ID NOs:32-33 and
f. mevalonate pyrophosphate decarboxylase having at least 80% sequence identity to at least one amino acid sequence selected from the group consisting of SEQ ID NOs:39-40;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 80% sequence identity to at least one amino acid sequence selected from the group consisting of SEQ ID NOs:41, 48-49;
h. 1-deoxy-D-xylulose 5-phosphate reductoisomerase, having at least 80% sequence identity to at least one amino acid sequence selected from the group consisting of SEQ ID NOs:50, 56-58;
i. 4-diphosphocytidyl-2-C-methyl-D-erythritol synthase, having at least 80% sequence identity to at least one amino acid sequence selected from the group consisting of SEQ ID NOs:59, 66-67;
j. 4-diphosphocytidyl-2-C-methyl-D-erythritol kinase, having at least 80% sequence identity to at least one amino acid sequence selected from the group consisting of SEQ ID NOs:68, 73;
k. 2-C-methyl-D-erythritol 2,4-cyclodiphosphate synthase, having at least 80% sequence identity to at least one amino acid sequence selected from the group consisting of SEQ ID NOs:74, 80-82;
l. 4-hydroxy-3-methylbut-2-en-1-yl diphosphate synthase, and having at least 80% sequence identity to at least one amino acid sequence selected from the group consisting of SEQ ID NOs:83, 89; and
m. 4-hydroxy-3-methylbut-2-enyl diphosphate reductase having at least 80% sequence identity to at least one amino acid sequence selected from the group consisting of SEQ ID NOs:90, 96-97.

35. The method of claim 32, wherein the set of heterologous nucleic acids encoding enzymes of the mevalonic acid pathway comprises nucleic acids encoding:
a. acetyl-CoA acetyltransferase having at least 90% sequence identity to SEQ ID NO:4;
b. 3-hydroxy-3-methylglutaryl coenzyme A synthase having at least 90% sequence identity to at least one amino acid sequence selected from the group consisting of SEQ ID NOs: 8-9;
c. 3-hydroxy-3-methylglutaryl-coenzyme A reductase having at least 90% sequence identity to at least one amino acid sequence selected from the group consisting of SEQ ID NOs:15, 16, 20;
d. mevalonate kinase, having at least 90% sequence identity SEQ ID NO:26;
e. phosphomevalonate kinase, having at least 90% sequence identity to at least one amino acid sequence selected from the group consisting of SEQ ID NOs:32-33 and
f. mevalonate pyrophosphate decarboxylase having at least 90% sequence identity to at least one amino acid sequence selected from the group consisting of SEQ ID NOs:39-40;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 90% sequence identity to at least one amino acid sequence selected from the group consisting of SEQ ID NOs:41, 48-49;
h. 1-deoxy-D-xylulose 5-phosphate reductoisomerase, having at least 90% sequence identity to at least one amino acid sequence selected from the group consisting of SEQ ID NOs:50, 56-58;
i. 4-diphosphocytidyl-2-C-methyl-D-erythritol synthase, having at least 90% sequence identity to at least one amino acid sequence selected from the group consisting of SEQ ID NOs:59, 66-67;
j. 4-diphosphocytidyl-2-C-methyl-D-erythritol kinase, having at least 90% sequence identity to at least one amino acid sequence selected from the group consisting of SEQ ID NOs:68, 73;
k. 2-C-methyl-D-erythritol 2,4-cyclodiphosphate synthase, having at least 90% sequence identity to at least one amino acid sequence selected from the group consisting of SEQ ID NOs:74, 80-82;
l. 4-hydroxy-3-methylbut-2-en-1-yl diphosphate synthase, and having at least 90% sequence identity to at least one amino acid sequence selected from the group consisting of SEQ ID NOs:83, 89; and
m. 4-hydroxy-3-methylbut-2-enyl diphosphate reductase having at least 90% sequence identity to at least one amino acid sequence selected from the group consisting of SEQ ID NOs:90, 96-97.

36. The method of claim 32, wherein the set of heterologous nucleic acids encoding enzymes of the mevalonic acid pathway comprises nucleic acids encoding:
a. acetyl-CoA acetyltransferase having at least 95% sequence identity to SEQ ID NO:4;
b. 3-hydroxy-3-methylglutaryl coenzyme A synthase having at least 95% sequence identity to at least one amino acid sequence selected from the group consisting of SEQ ID NOs: 8-9;
c. 3-hydroxy-3-methylglutaryl-coenzyme A reductase having at least 95% sequence identity to at least one amino acid sequence selected from the group consisting of SEQ ID NOs:15, 16, 20;
d. mevalonate kinase, having at least 95% sequence identity SEQ ID NO:26;
e. phosphomevalonate kinase, having at least 95% sequence identity to at least one amino acid sequence selected from the group consisting of SEQ ID NOs:32-33 and
f. mevalonate pyrophosphate decarboxylase having at least 95% sequence identity to at least one amino acid sequence selected from the group consisting of SEQ ID NOs:39-40;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 95% sequence identity to at least one amino acid sequence selected from the group consisting of SEQ ID NOs:41, 48-49;
h. 1-deoxy-D-xylulose 5-phosphate reductoisomerase, having at least 95% sequence identity to at least one amino acid sequence selected from the group consisting of SEQ ID NOs:50, 56-58;
i. 4-diphosphocytidyl-2-C-methyl-D-erythritol synthase, having at least 95% sequence identity to at least one amino acid sequence selected from the group consisting of SEQ ID NOs:59, 66-67;
j. 4-diphosphocytidyl-2-C-methyl-D-erythritol kinase, having at least 95% sequence identity to at least one amino acid sequence selected from the group consisting of SEQ ID NOs:68, 73;
k. 2-C-methyl-D-erythritol 2,4-cyclodiphosphate synthase, having at least 95% sequence identity to at least one amino acid sequence selected from the group consisting of SEQ ID NOs:74, 80-82;
l. 4-hydroxy-3-methylbut-2-en-1-yl diphosphate synthase, and having at least 95% sequence identity to at least one amino acid sequence selected from the group consisting of SEQ ID NOs:83, 89; and
m. 4-hydroxy-3-methylbut-2-enyl diphosphate reductase having at least 95% sequence identity to at least one amino acid sequence selected from the group consisting of SEQ ID NOs:90, 96-97.

37. The method of claim 32, wherein the set of heterologous nucleic acids encoding enzymes of the mevalonic acid pathway comprises nucleic acids encoding:
a. acetyl-CoA acetyltransferase having at least 99% sequence identity to SEQ ID NO:4;
b. 3-hydroxy-3-methylglutaryl coenzyme A synthase having at least 99% sequence identity to at least one amino acid sequence selected from the group consisting of SEQ ID NOs: 8-9;
c. 3-hydroxy-3-methylglutaryl-coenzyme A reductase having at least 99% sequence identity to at least one amino acid sequence selected from the group consisting of SEQ ID NOs:15, 16, 20;
d. mevalonate kinase, having at least 99% sequence identity SEQ ID NO:26;
e. phosphomevalonate kinase, having at least 99% sequence identity to at least one amino acid sequence selected from the group consisting of SEQ ID NOs:32-33 and
f. mevalonate pyrophosphate decarboxylase having at least 99% sequence identity to at least one amino acid sequence selected from the group consisting of SEQ ID NOs:39-40;
and wherein the set of heterologous nucleic acids encoding enzymes of the methylerythritol 4-phosphate pathway comprise nucleic acid encoding a:
g. 1-deoxy-D-xylulose-5-phosphate synthase, having at least 99% sequence identity to at least one amino acid sequence selected from the group consisting of SEQ ID NOs:41, 48-49;
h. 1-deoxy-D-xylulose 5-phosphate reductoisomerase, having at least 99% sequence identity to at least one amino acid sequence selected from the group consisting of SEQ ID NOs:50, 56-58;
i. 4-diphosphocytidyl-2-C-methyl-D-erythritol synthase, having at least 99% sequence identity to at least one amino acid sequence selected from the group consisting of SEQ ID NOs:59, 66-67;
j. 4-diphosphocytidyl-2-C-methyl-D-erythritol kinase, having at least 99% sequence identity to at least one amino acid sequence selected from the group consisting of SEQ ID NOs:68, 73;
k. 2-C-methyl-D-erythritol 2,4-cyclodiphosphate synthase, having at least 99% sequence identity to at least one amino acid sequence selected from the group consisting of SEQ ID NOs:74, 80-82;
l. 4-hydroxy-3-methylbut-2-en-1-yl diphosphate synthase, and having at least 99% sequence identity to at least one amino acid sequence selected from the group consisting of SEQ ID NOs:83, 89; and
m. 4-hydroxy-3-methylbut-2-enyl diphosphate reductase having at least 99% sequence identity to at least one amino acid sequence selected from the group consisting of SEQ ID NOs:90, 96-97.

38. The method of claim 32, wherein the set of heterologous nucleic acids encoding enzymes of the mevalonic acid pathway comprises nucleic acids encoding:
a. acetyl-CoA acetyltransferase is sequence selected from the group consisting of SEQ ID NO:4;
b. 3-hydroxy-3-methylglutaryl coenzyme A synthase is sequence selected from the group consisting of SEQ ID NOs: 8-9;
c. 3-hydroxy-3-methylglutaryl-coenzyme A reductase is sequence selected from the group consisting of SEQ ID NOs:15, 16, 20;
d. mevalonate kinase, is sequence selected from the group consisting of SEQ ID NOs:26;
e. phosphomevalonate kinase, is sequence selected from the group consisting of SEQ ID NOs:32-33 and
f. mevalonate pyrophosphate decarboxylase is sequence selected from the group consisting of SEQ ID NOs:39-40;
and wherein the set of heterologous nucleic acids encoding enzymes of the methylerythritol 4-phosphate pathway comprise nucleic acid encoding a:
g. 1-deoxy-D-xylulose-5-phosphate synthase, is sequence selected from the group consisting of SEQ ID NOs:41, 48-49;
h. 1-deoxy-D-xylulose 5-phosphate reductoisomerase, is sequence selected from the group consisting of SEQ ID NOs:50, 56-58;
i. 4-diphosphocytidyl-2-C-methyl-D-erythritol synthase, is sequence selected from the group consisting of SEQ ID NOs:59, 66-67;
j. 4-diphosphocytidyl-2-C-methyl-D-erythritol kinase, is sequence selected from the group consisting of SEQ ID NOs:68, 73;
k. 2-C-methyl-D-erythritol 2,4-cyclodiphosphate synthase, is sequence selected from the group consisting of SEQ ID NOs:74, 80-82;
l. 4-hydroxy-3-methylbut-2-en-1-yl diphosphate synthase, and is sequence selected from the group consisting of SEQ ID NOs:83, 89; and
m. 4-hydroxy-3-methylbut-2-enyl diphosphate reductase is sequence selected from the group consisting of SEQ ID NOs:90, 96-97.

39. The method of claim 3-5, wherein the heterologous nucleic acids encoding the isopentenyl-diphosphate delta-isomerase, the farnesyl diphosphate synthase; and the farnesene synthase are enzymes selected from the group consisting of Archaea, bacteria, fungi, and plantae kingdoms.

40. The method of claim 39, wherein the enzymes are from the plantae kingdom.

41. The method of claim 40 wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 70% sequence identity to at least one amino acid sequence selected from the group consisting of SEQ ID NOs:98-101, 102-106, 188, 190-192;
b. farnesyl diphosphate synthase, having at least 70% sequence identity to at least one amino acid sequence selected from the group consisting of SEQ ID NOs:107-111, 164, 165, 176, 187, 189; and
c. farnesene synthase, having at least 70% sequence identity to at least one amino acid sequence selected from the group consisting of SEQ ID NOs:112-115, 116-117, 166-168.

42. The method of claim 40 wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 80% sequence identity to at least one amino acid sequence selected from the group consisting of SEQ ID NOs:98-101, 102-106, 188, 190-192;
b. farnesyl diphosphate synthase, having at least 80% sequence identity to at least one amino acid sequence selected from the group consisting of SEQ ID NOs:107-111, 164, 165, 176, 187, 189; and
c. farnesene synthase, having at least 80% sequence identity to at least one amino acid sequence selected from the group consisting of SEQ ID NOs:112-115, 116-117, 166-168.

43. The method of claim 40 wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 90% sequence identity to at least one amino acid sequence selected from the group consisting of SEQ ID NOs:98-101, 102-106, 188, 190-192;
b. farnesyl diphosphate synthase, having at least 90% sequence identity to at least one amino acid sequence selected from the group consisting of SEQ ID NOs:107-111, 164, 165, 176, 187, 189; and
c. farnesene synthase, having at least 90% sequence identity to at least one amino acid sequence selected from the group consisting of SEQ ID NOs:112-115, 116-117, 166-168.

44. The method of claim 40 wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 95% sequence identity to at least one amino acid sequence selected from the group consisting of SEQ ID NOs:98-101, 102-106, 188, 190-192;
b. farnesyl diphosphate synthase, having at least 95% sequence identity to at least one amino acid sequence selected from the group consisting of SEQ ID NOs:107-111, 164, 165, 176, 187, 189; and
c. farnesene synthase, having at least 95% sequence identity to at least one amino acid sequence selected from the group consisting of SEQ ID NOs:112-115, 116-117, 166-168.

45. The method of claim 40 wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, having at least 99% sequence identity to at least one amino acid sequence selected from the group consisting of SEQ ID NOs:98-101, 102-106, 188, 190-192;
b. farnesyl diphosphate synthase, having at least 99% sequence identity to at least one amino acid sequence selected from the group consisting of SEQ ID NOs:107-111, 164, 165, 176, 187, 189; and
c. farnesene synthase, having at least 99% sequence identity to at least one amino acid sequence selected from the group consisting of SEQ ID NOs:112-115, 116-117, 166-168.

46. The method of claim 40 wherein the heterologous nucleic acids encoding the isopentenyl-diphosphate delta-isomerase, the farnesyl diphosphate synthase; and the farnesene synthase are encoded by a nucleic acid encoding a(n):
a. isopentenyl-diphosphate delta-isomerase, is selected from the group consisting of SEQ ID NOs:98-101, 102-106, 188, 190-192;
b. farnesyl diphosphate synthase, is selected from the group consisting of SEQ ID NOs:107-111, 164, 165, 176, 187, 189; and
c. farnesene synthase, is selected from the group consisting of SEQ ID NOs:112-115, 116-117, 166-168.

47. The method of claim 1, wherein the plant cell is a cell from a plant selected from the group consisting of a green algae, a vegetable crop plant, a fruit crop plant, a vine crop plant, a field crop plant, a biomass plant, a bedding plant, and a tree.

48. The method of claim 47, wherein the plant is selected from the group consisting of corn, soybean, Brassica, tomato, sorghum, sugar cane, Hevea, miscanthus, guayle, switchgrass, wheat, barley, oat, rye, wheat, rice, beet, green algae and cotton.

49. The method of claim 48, wherein the plant is sorghum, sugar cane, Hevea, or guayle.

50. The method of claim 1, further comprising isolating the farnesene.

51. The method of claim 50, wherein the isolated farnesene is further processed into farnesane.

52. A plant cell made by any of the methods of claims 1-2.

53. A method of increasing production of at least one terpenoid in a plant, the method comprising of making a plant that comprises at least one plant cell made by claim 52, wherein at least one terpenoid is increased when compared to a plant not comprising at least one plant cell made by claim 52.

54. A plant comprising a plant cell of claim 52.

55. A fuel comprising a terpenoid made according to any of claims 1-2, 53, or made by a plant cell of claim 52 or by a plant of claim 54.

56. The fuel of claim 55, wherein the terpenoid is a sesquiterpenoid.

57. The fuel of claim 56, wherein the sesquiterpenoid is farnesene.
